(19) United States
(12) Patent Application Publication
(10) Pub. No.: US 2026/0235953 A1
(43) Pub. Date: Aug. 13, 2026

(54) ONIUM SALT MONOMER, POLYMER, CHEMICALLY AMPLIFIED RESIST COMPOSITION, AND PATTERNING METHOD

(71) Applicant: SHIN-ETSU CHEMICAL CO., LTD., Tokyo (JP)

(72) Inventors: Daisuke KORI, Joetsu-shi (JP); Takahiro Suzuki, Joetsu-shi (JP)

(73) Assignee: SHIN-ETSU CHEMICAL CO., LTD., Tokyo (JP)

(21) Appl. No.: 19/529,941

(22) Filed: Feb. 4, 2026

(30) Foreign Application Priority Data
Feb. 12, 2025 (JP) 2025-021119

(51) Int. Cl. G03F 7/039; G03F 7/004; G03F 7/20

(52) U.S. Cl. CPC G03F 7/0392; G03F 7/0046; G03F 7/0397; G03F 7/2041

(57) ABSTRACT

An onium salt monomer to be used for a chemically amplified resist composition that exhibits superior solubility in organic solvents and has superior EL, LWR, CDU and the like with high sensitivity and contrast, and has superior etching resistance with durability against pattern collapse even in fine patterning. An onium salt monomer represented by formula (a):

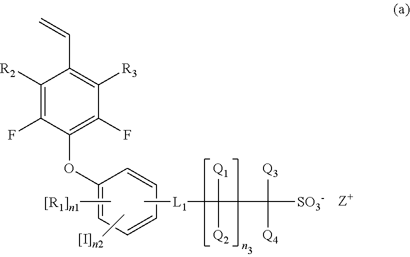

wherein R₁ is a halogen atom, or hydrocarbyl group; R₂ and R₃ are each hydrogen or fluorine atoms; n1 is 0 to 3, n2 is 1 to 4, and n3 is 0 to 4; L₁ is a single bond, an ether bond, an ester bond, a sulfonic ester bond, a sulfonamide bond, a carbonate bond, or carbamate bond; Q₁ and Q₂ are each a hydrogen atom, a fluorine atom, or fluorinated saturated hydrocarbyl group; Q₃ and Q₄ are each a fluorine atom or a fluorinated saturated hydrocarbyl group; and Z⁺ is an onium cation.

### TECHNICAL FIELD

The present invention relates to an onium salt monomer, a polymer, a chemically amplified resist composition, and a patterning method.

### BACKGROUND ART

Achievement of higher integration levels and higher speed in LSIs has been accelerating miniaturization of pattern rules. In particular, the expansion of the flash memory market and the increase in memory capacity are driving such miniaturization. Mass production of 65-nm node devices has been carried out by ArF lithography as a state-of-the-art miniaturization technique, and preparations for mass production of 45-nm node devices by next-generation Arf immersion lithography are currently under way. Candidates for producing next-generation 32-nm node devices include immersion lithography with use of liquid having a higher refractive index than water and an ultra-high NA lens including a high-refractive-index lens and a high-refractive-index resist film in combination, extreme ultraviolet (EUV) lithography with a wavelength of 13.5 nm, and double exposure with ArF lithography (double-patterning lithography), and examination therefor is ongoing.

As miniaturization proceeds and the diffraction limit of light is approached, the contrast of light decreases. The reduced contrast of light causes positive-type resist films to undergo reduction in the resolution of hole patterns and trench patterns and in focus margins.

In association with the miniaturization of patterns, the line width roughness (LWR) of line patterns and the critical dimension uniformity (CDU) of hole patterns have become matters of concern. Influences of the uneven distribution or aggregation of a base polymer or an acid generator and those of acid diffusion have been pointed out. Furthermore, thinner resist films tend to have higher LWR, and the degradation of LWR due to thinning associated with the progression of miniaturization has become a serious problem.

Resist compositions for EUV lithography need to achieve high sensitivity, high resolution, and low LWR at the same time. Decreasing the acid diffusion length results in lower LWR, but in lower sensitivity. For example, lowering the post-exposure bake (PEB) temperature results in lower LWR, but in lower sensitivity. Likewise, increasing the amount of a quencher to be added results in lower LWR, but in lower sensitivity. It is needed to break down the trade-off relationship between sensitivity and LWR.

To inhibit acid diffusion, a resist compound containing a repeating unit derived from an onium salt of a sulfonic acid having a polymerizable unsaturated bond has been proposed (Patent Document 1). Such a compound, what is called a polymer-bound acid generator, is characterized by its very short acid diffusion length due to the generation of polymeric sulfonic acid through exposure. Alternatively, enhanced sensitivity is achieved by using an acid generator at a higher proportion. Also for an acid generator of addition type, increasing the amount thereof to be added results in higher sensitivity, but in this case also in an increased acid diffusion length. Because acids unevenly diffuse, increased acid diffusion leads to degradation in LWR and CDU. It can be said that polymeric acid generators have high performance in terms of the balance of sensitivity, LWR, and CDU.

Iodine atoms have been found to have an effect of generating secondary electrons therefrom during exposure because iodine atoms exhibit very large absorption of EUV at a wavelength of 13.5 nm, gathering attention in the field of EUV lithography. Patent Document 2 discloses a photo-acid generator obtained by introducing an iodine atom into an anion, and Patent Document 3 discloses a polymerizable group-containing photo-acid generator obtained by introducing an iodine atom into an anion. These have been found to contribute to improvement in lithographic performance to some degree, whereas introduction of iodine atoms causes reduction in solubility in organic solvents, and problems including precipitation in solvents are concerned; hence, improvement in workability is demanded.

### CITATION LIST

### Patent Literature

Patent Document 1: JP 4425776 B
Patent Document 2: JP 6720926 B
Patent Document 3: JP 6973274 B

### SUMMARY OF INVENTION

### Technical Problem

For chemically amplified resist compositions that are catalyzed by acids, development of a resist composition is demanded that has much higher sensitivity, enables improvement in the LWR of lines and the CDU of holes, and is superior even in etching resistance after patterning. Furthermore, development of a monomer or polymer superior in solubility in solvents is demanded for broader process margins in manufacture.

The present invention has been made in view of the aforementioned circumstances, and an object of the present invention is to provide: an onium salt monomer to be used for a chemically amplified resist composition that exhibits superior solubility in organic solvents and is superior in lithographic performance including exposure latitude (EL), LWR, and CDU with high sensitivity in photolithography with use of a high-energy ray, and also has superior etching resistance with durability against pattern collapse even in fine patterning; a polymer containing a repeating unit derived from the onium salt monomer; a chemically amplified resist composition containing the polymer; and a patterning method with the chemically amplified resist composition.

### Solution to Problem

To solve the above problems, the present invention provides an onium salt monomer represented by the following formula (a):

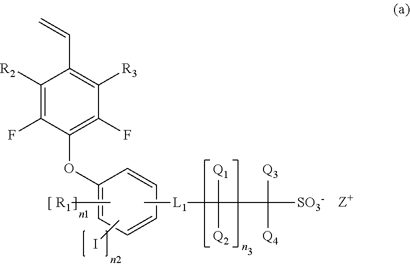

wherein R₁ is a halogen atom being not iodine, a cyano group, a nitro group, or a hydrocarbyl group having 1 to 20 carbon atoms and optionally containing a heteroatom; R₂ and R₃ are each a hydrogen atom or a fluorine atom; n1 is an integer of 0 to 3, n2 is an integer of 1 to 4, and n3 is an integer of 0 to 4; L₁ is a single bond, an ether bond, an ester bond, a sulfonic ester bond, a sulfonamide bond, a carbonate bond, or a carbamate bond; Q₁ and Q₂ are each independently a hydrogen atom, a saturated hydrocarbyl group having 1 to 6 carbon atoms, a fluorine atom, or a fluorinated saturated hydrocarbyl group having 1 to 6 carbon atoms; Q₃ and Q₄ are each independently a hydrogen atom, a saturated hydrocarbyl group having 1 to 6 carbon atoms, a fluorine atom, or a fluorinated saturated hydrocarbyl group having 1 to 6 carbon atoms; and Z⁺ is an onium cation.

Such an onium salt monomer serves as an onium salt monomer to be used for a chemically amplified resist composition that exhibits superior solubility in organic solvents and is superior in lithographic performance including exposure latitude (EL), LWR, and CDU with high sensitivity and high contrast particularly in photolithography with use of a high-energy ray such as KrF excimer laser light, ArF excimer laser light, an electron beam (EB), and EUV, and also has superior etching resistance with durability against pattern collapse even in fine patterning.

Moreover, the onium salt monomer of the present invention is preferably represented by the following formula (a1):

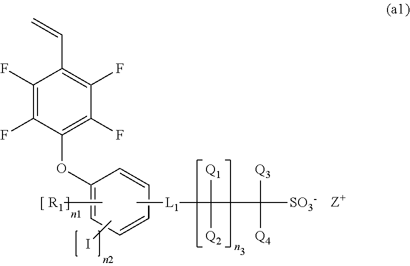

wherein n1 to n3, R₁, L₁, Q₁ to Q₄, and Z⁺ are as specified above.

In this case, the onium salt monomer of the present invention is more preferably represented by the following formula (a2):

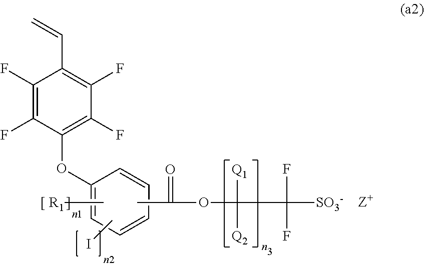

wherein n1 to n3, R₁, Q₁, Q₂, and Z⁺ are as specified above.

Such a configuration allows the effects of the present invention to be more sufficiently exerted.

In addition, Z⁺ is preferably a sulfonium cation represented by the following formula (cation-1) or an iodonium cation represented by the following formula (cation-2):

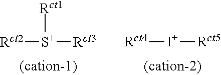

wherein R^{ct1} to R^{ct5} are each independently a halogen atom or a hydrocarbyl group having 1 to 30 carbon atoms and optionally containing a heteroatom; and R^{ct1} and R^{ct2} are optionally bound together to form a ring together with the sulfur atom to which the two groups are bound.

Such a cation moiety allows the effects of the present invention to be more sufficiently exerted.

The present invention also provides a polymer containing a repeating unit derived from the onium salt monomer.

The polymer of the present invention serves as a polymer-bound photo-acid generator that functions as a photo-acid generator and also as a base polymer in a chemically amplified resist composition, and gives a chemically amplified resist composition that exhibits superior solubility in organic solvents and is superior in lithographic performance including exposure latitude (EL), LWR, and CDU with high sensitivity and high contrast particularly in photolithography with use of a high-energy ray such as KrF excimer laser light, ArF excimer laser light, an electron beam (EB), and EUV, and also has superior etching resistance with durability against pattern collapse even in fine patterning.

The polymer of the present invention may further contain a repeating unit represented by the following formula (b1) or (b2):

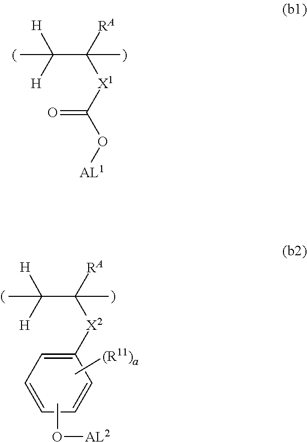

wherein R^{A} groups are each independently a hydrogen atom, a fluorine atom, a methyl group, or a trifluoromethyl group;
X¹ is a single bond, a phenylene group, a naphthylene group, *—C(═O)—O—X¹¹—, or *—C(═O)—NH—X¹¹—, wherein the phenylene group or naphthylene group is optionally substituted with an alkoxy group having 1 to 10 carbon atoms and optionally containing a fluorine atom or with a halogen atom, X¹¹ is a saturated hydrocarbylene group having 1 to 10 carbon atoms, a phenylene group, or a naphthylene group, and the saturated hydrocarbylene group optionally contains a hydroxy group, an ether bond, an ester bond, or a lactone ring;
X² is a single bond, *—C(═O)—O—, or *—C(═O)—NH—;
* indicates bonding to a carbon atom of a main chain;
AL¹ and AL² are each independently an acid-unstable group;
R¹¹ is a halogen atom, a cyano group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; and
a is an integer of 0 to 4.

The polymer of the present invention may further contain a repeating unit represented by the following formula (c1):

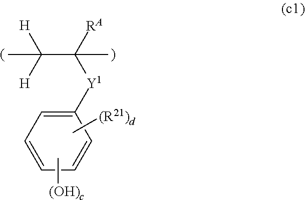

wherein R^{A} is a hydrogen atom, a fluorine atom, a methyl group, or a trifluoromethyl group;
Y¹ is a single bond, *—C(═O)—O—, or *—C(═O)—NH—, wherein * indicates bonding to a carbon atom of a main chain;
R²¹ is a halogen atom, a nitro group, a cyano group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; and
c is an integer of 1 to 4, and d is an integer of 0 to 3, provided that 1≤c+d≤5 is satisfied.

The polymer of the present invention may further contain a repeating unit represented by the following formula (d1):

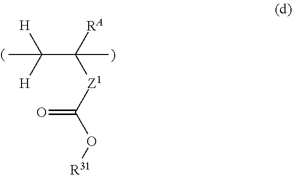

wherein R^{A} is a hydrogen atom, a fluorine atom, a methyl group, or a trifluoromethyl group;
Z¹ is a single bond, a phenylene group, a naphthylene group, *—C(═O)—O—Z¹¹—, or *—C(═O)—NH—Z¹¹—, wherein the phenylene group or naphthylene group is optionally substituted with an alkoxy group having 1 to 10 carbon atoms and optionally containing a fluorine atom or with a halogen atom; * indicates bonding to a carbon atom of a main chain; Z ¹¹ is a saturated hydrocarbylene group having 1 to 10 carbon atoms, a phenylene group, or a naphthylene group, and the saturated hydrocarbylene group optionally contains a hydroxy group, an ether bond, an ester bond, or a lactone ring; and
R³¹ is a hydrogen atom or a group having 1 to 20 carbon atoms and containing at least one or more structures selected from a hydroxy group being not a phenolic hydroxy group, a cyano group, a carbonyl group, a carboxy group, an ether bond, an ester bond, a sulfonic ester bond, a carbonate bond, a lactone ring, a sultone ring, and a carboxylic anhydride (—C(═O)—O—C(═O)—).

The polymer of the present invention is allowed to have required performance to meet the purpose imparted through copolymerization of various repeating units.

The present invention also provides a chemically amplified resist composition containing (A) a base polymer containing the polymer.

The chemically amplified resist composition of the present invention exhibits superior solubility in organic solvents and is superior in lithographic performance including exposure latitude (EL), LWR, and CDU with high sensitivity and high contrast particularly in photolithography with use of a high-energy ray such as KrF excimer laser light, ArF excimer laser light, an electron beam (EB), and EUV, and also has superior etching resistance with durability against pattern collapse even in fine patterning.

The chemically amplified resist composition of the present invention can further contain any one or more of (B) an organic solvent, (C) a quencher, (D) an acid generator, (E) a surfactant, and (F) a dissolution inhibitor.

The chemically amplified resist composition of the present invention is allowed to have required performance to meet the purpose imparted through inclusion of various components.

The present invention also provides a patterning method including:
a step of forming a resist film on a substrate by using the chemically amplified resist composition; a step of exposing the resist film to a high-energy ray; and a step of developing the exposed resist film by using a developer.

The patterning method of the present invention enables satisfactory formation of fine patterns with use of the chemically amplified resist composition of the present invention.

Moreover, ArF excimer laser light having a wavelength of 193 nm, KrF excimer laser light having a wavelength of 248 nm, an electron beam, or an extreme ultraviolet ray having a wavelength of 3 to 15 nm is preferably used as the high-energy ray.

Such a high-energy ray can be particularly suitably used in the patterning method of the present invention.

### Advantageous Effects of Invention

A resist film containing a polymer containing a repeating unit derived from the onium salt monomer represented by the formula (a) has good solubility in organic solvents imparted by the action of fluorine substituents in the styrene structure, and is characterized by small acid diffusion because of the large atomic weight of iodine atoms. This can successfully prevent reduction in resolution due to blur caused by acid diffusion, giving improved LWR and CDU. Because of the very large absorption of EUV by iodine atoms at a wavelength of 13.5 nm, secondary electrons are generated from iodine atoms during exposure, resulting in high sensitivity. This enables construction of a chemically amplified resist composition with high sensitivity and improved LWR and CDU. Moreover, the aromatic ring acts as a good etching-resistant group, thus being suitable for fine patterning.

### DESCRIPTION OF EMBODIMENTS

As described above, development of an onium salt monomer to be used for a chemically amplified resist composition that exhibits superior solubility in organic solvents and is superior in lithographic performance including exposure latitude (EL), LWR, and CDU with high sensitivity and high contrast particularly in photolithography with use of a high-energy ray such as KrF excimer laser light, ArF excimer laser light, an electron beam (EB), and EUV, and also has superior etching resistance with durability against pattern collapse even in fine patterning, a polymer containing a repeating unit derived from the onium salt monomer, a chemically amplified resist composition containing the polymer, and a patterning method with the chemically amplified resist composition has been demanded.

The present inventors have diligently examined to achieve the object, and found that a chemically amplified resist composition that exhibits superior solubility in organic solvents, has high sensitivity, is improved in LWR and CDU, has high contrast and thus high resolution, and is superior also in etching resistance is obtained by using a polymer as a polymer-bound acid generator, the polymer containing a repeating unit derived from an onium salt containing a fluorosulfonate anion having a styrene structure, as a polymerizable group, with fluorine substituents and having an aromatic ring structure substituted with an iodine atom, completing the present invention.

Specifically, the present invention relates to an onium salt monomer represented by the following formula (a):

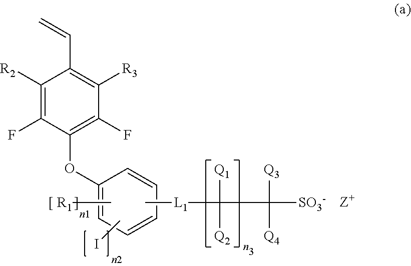

wherein R₁ is a halogen atom being not iodine, a cyano group, a nitro group, or a hydrocarbyl group having 1 to 20 carbon atoms and optionally containing a heteroatom; R₂ and R₃ are each a hydrogen atom or a fluorine atom; n1 is an integer of 0 to 3, n2 is an integer of 1 to 4, and n3 is an integer of 0 to 4; L₁ is a single bond, an ether bond, an ester bond, a sulfonic ester bond, a sulfonamide bond, a carbonate bond, or a carbamate bond; Q₁ and Q₂ are each independently a hydrogen atom, a saturated hydrocarbyl group having 1 to 6 carbon atoms, a fluorine atom, or a fluorinated saturated hydrocarbyl group having 1 to 6 carbon atoms; Q₃ and Q₄ are each independently a hydrogen atom, a saturated hydrocarbyl group having 1 to 6 carbon atoms, a fluorine atom, or a fluorinated saturated hydrocarbyl group having 1 to 6 carbon atoms; and Z⁺ is an onium cation.

The following describes the present invention in detail, but the present invention is not limited by the description.

### [Onium Salt Monomer]

The onium salt monomer of the present invention is represented by the following formula (a):

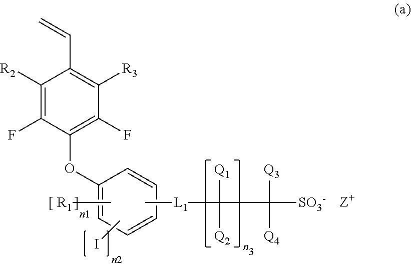

wherein R₁ is a halogen atom being not iodine, a cyano group, a nitro group, or a hydrocarbyl group having 1 to 20 carbon atoms and optionally containing a heteroatom; R₂ and R₃ are each a hydrogen atom or a fluorine atom; n1 is an integer of 0 to 3, n2 is an integer of 1 to 4, and n3 is an integer of 0 to 4; L₁ is a single bond, an ether bond, an ester bond, a sulfonic ester bond, a sulfonamide bond, a carbonate bond, or a carbamate bond; Q₁ and Q₂ are each independently a hydrogen atom, a saturated hydrocarbyl group having 1 to 6 carbon atoms, a fluorine atom, or a fluorinated saturated hydrocarbyl group having 1 to 6 carbon atoms; Q₃ and Q₄ are each independently a hydrogen atom, a saturated hydrocarbyl group having 1 to 6 carbon atoms, a fluorine atom, or a fluorinated saturated hydrocarbyl group having 1 to 6 carbon atoms; and Z⁺ is an onium cation.

In the formula (a1), n1 is an integer of 0 to 3, and n1 is preferably 0 or 1, and further preferably 0 for raw material procurement. n2 is an integer of 1 to 4, preferably 1 or 2, and further preferably 1 for raw material procurement. More iodine atoms in the anion structure result in enhanced absorption particularly to EUV, but in poor solubility in solvents, which may disadvantageously cause precipitation in the resist composition, and for this reason n2 is preferably controlled within a proper range. n3 is an integer of 0 to 4, preferably 0, 1, 2, or 3, and more preferably 1.

In the formula (a), the styrene structure having fluorine substituents and bound via an ether bond in the aromatic ring having an iodine atom as a substituent in the anion is preferably bound to the ortho position of the carbon atom to which L₁ is bound. Such positional relationship results in loosened aggregation structure of molecules through steric hindrance, and the trade-off of deterioration of solubility in solvents that is caused by introduction of an iodine atom can be overcome. In addition, introduction of a styrene structure via an ether bond is expected to give enhanced thermal resistance. Moreover, the fluorine substituents impart solubility in solvents by their action, and the inclusion of a plurality of fluorine atoms is expected to give an effect of enhancing sensitivity to EUV.

In the formula (a), R₁ is a halogen atom being not iodine, a cyano group, a nitro group, or a hydrocarbyl group having 1 to 20 carbon atoms and optionally containing a heteroatom. The halogen atom is a fluorine atom, a chlorine atom, a bromine atom. The hydrocarbyl group may be each saturated or unsaturated, and may be each linear, branched, or cyclic. Specific examples thereof include an alkyl group having 1 to 20 carbon atoms such as a methyl group, an ethyl group, a n-propyl group, an isopropyl group, a n-butyl group, an isobutyl group, a sec-butyl group, a tert-butyl group, a n-pentyl group, a n-hexyl group, a n-octyl group, a n-nonyl group, a n-decyl group, an undecyl group, a dodecyl group, a tridecyl group, a tetradecyl group, a pentadecyl group, a heptadecyl group, an octadecyl group, a nonadecyl group, and an icosyl group; a cyclic saturated hydrocarbyl group having 3 to 20 carbon atoms such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; an alkenyl group having 2 to 20 carbon atoms such as a vinyl group, an allyl group, a propenyl group, a butenyl group, and a hexenyl group; a cyclic unsaturated hydrocarbyl group having 3 to 20 carbon atoms such as a cyclohexenyl group; an aryl group having 6 to 20 carbon atoms such as a phenyl group and a naphthyl group; an aralkyl group having 7 to 20 carbon atoms such as a benzyl group, a 1-phenylethyl group, and a 2-phenylethyl group; and a group given by combining any of these. Some or all of the hydrogen atoms of the hydrocarbyl group may be each replaced with a group containing a heteroatom such as an oxygen atom, a sulfur atom, a nitrogen atom, and a halogen atom, some of the —CH₂— moieties of the hydrocarbyl group may be each replaced with a group containing a heteroatom such as an oxygen atom, a sulfur atom, and a nitrogen atom, and as a result, for example, a hydroxy group, a cyano group, a fluorine atom, a chlorine atom, a bromine atom, an iodine atom, a carbonyl group, an ether bond, an ester bond, a sulfonic ester bond, a carbonate bond, a lactone ring, a sultone ring, a carboxylic anhydride (—C(═O)—O—C(═O)—), or a haloalkyl group may be contained. If n1 is 2 or 3, R₁ groups are identical to or different from each other.

If n1 is 2 or 3, a plurality of R₁ groups are optionally bound together to form a ring together with the carbon atoms of the aromatic ring or aromatic rings to which the two groups are bound. Specific examples of the ring formed in this case include a cyclopropane ring, a cyclobutane ring, a cyclopentane ring, a cyclohexane ring, a norbornane ring, and an adamantane ring. Some or all of the hydrogen atoms in the ring may be each replaced with a group containing a heteroatom such as an oxygen atom, a sulfur atom, a nitrogen atom, and a halogen atom, some of the —CH₂— moieties in the ring may be each replaced with a group containing a heteroatom such as an oxygen atom, a sulfur atom, and a nitrogen atom, and as a result, for example, a hydroxy group, a fluorine atom, a chlorine atom, a bromine atom, an iodine atom, a cyano group, a carbonyl group, an ether bond, an ester bond, a sulfonic ester bond, a carbonate bond, a lactone ring, a sultone ring, a carboxylic anhydride (—C(═O)—O—C(═O)—), or a haloalkyl group may be contained.

In the formula (a), L₁ is a single bond, an ether bond, an ester bond, a sulfonic ester bond, a sulfonamide bond, a carbonate bond, or a carbamate bond, preferably a single bond, an ether bond, an ester bond, or a sulfonic ester bond, and further preferably an ester bond.

In the formula (a), Q¹ and Q² are each independently a hydrogen atom, a saturated hydrocarbyl group having 1 to 6 carbon atoms, a fluorine atom, or a fluorinated saturated hydrocarbyl group having 1 to 6 carbon atoms. The fluorinated saturated hydrocarbyl group having 1 to 6 carbon atoms is preferably a trifluoromethyl group.

In the formula (a), Q³ and Q⁴ are each independently a hydrogen atom, a saturated hydrocarbyl group having 1 to 6 carbon atoms, a fluorine atom, or a fluorinated saturated hydrocarbyl group having 1 to 6 carbon atoms. The fluorinated saturated hydrocarbyl group having 1 to 6 carbon atoms is preferably a trifluoromethyl group. Q³ and Q⁴ are further preferably each a fluorine atom.

Preferred specific examples of the partial structure represented by —[C(Q₁)(Q₂)]ₙ₃—C(Q₃)(Q₄)—SO₃⁻ in the formula (a) are those shown below, but are not limited thereto. In the following formulae, * indicates bonding to L₁.

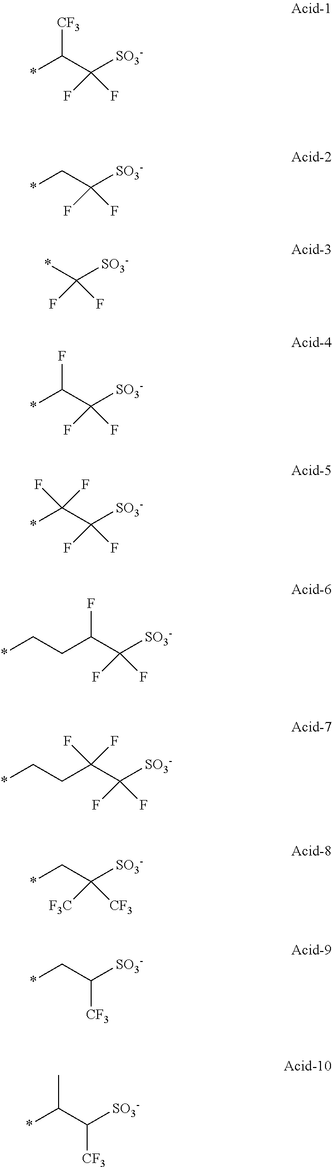

Among these, Acid-1 to Acid-7 are preferred, and Acid-1 to Acid-3, Acid-6, and Acid-7 are more preferred.

The onium salt monomer represented by the formula (a) is preferably represented by the following formula (a1):

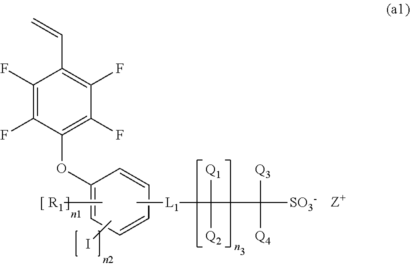

wherein n1 to n3, R₁, L₁, Q₁ to Q₄, and Z⁺ are as specified above.

As described above, not only the configuration in which every substituent in the aromatic ring of the styrene moiety is a fluorine substituent is effective for raw material procurement and imparting solubility in solvents, but also the introduction of many fluorine atoms is expected to result in improvement in performance such as enhanced sensitivity in EUV exposure and an effect of preventing attachment of undesired matters in developing. Moreover, the improved solubility of the monomer in solvents is expected to lead to reduction of the monomer remaining in polymer synthesis and contribute to the solubility of the polymer itself in solvents, and also highly expected to give an effect of preventing defects such as improvement in terms of scum, which causes problems in lithography.

The onium salt monomer represented by the formula (a) is more preferably represented by the following formula (a2):

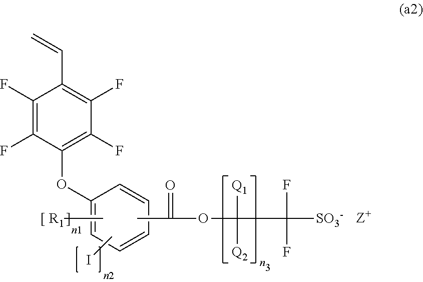

wherein n1 to n3, R₁, Q₁, Q₂, and Z⁺ are as specified above.

As described above, the configuration in which the bond structure between the anion moiety and the aromatic ring moiety having iodine as a substituent is an ester bond is preferred for raw material procurement. As illustrated in the general formula shown below, it is further preferable that the ester bond between the aromatic ring moiety having iodine as a substituent and the anion moiety and the ether bond between that aromatic ring moiety and the fluorostyrene moiety having fluorine substituents be adjacent to each other in the aromatic ring, in other words, the ether bond be present at the ortho position of the carbon atom to which the ester is bound. As described above, further improved solubility is successfully achieved by using the steric hindrance between the aromatic rings.

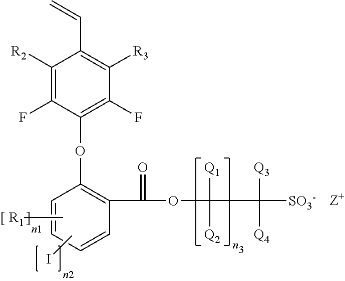

Specific examples of the anion of the onium salt monomer represented by the formula (a) are those shown below, but are not limited thereto. In the following formulae, R₂ and R₃ are each a hydrogen atom or a fluorine atom, Q₁ is as specified above, and the bonding positions of substituents in each aromatic ring may be interchanged therein.

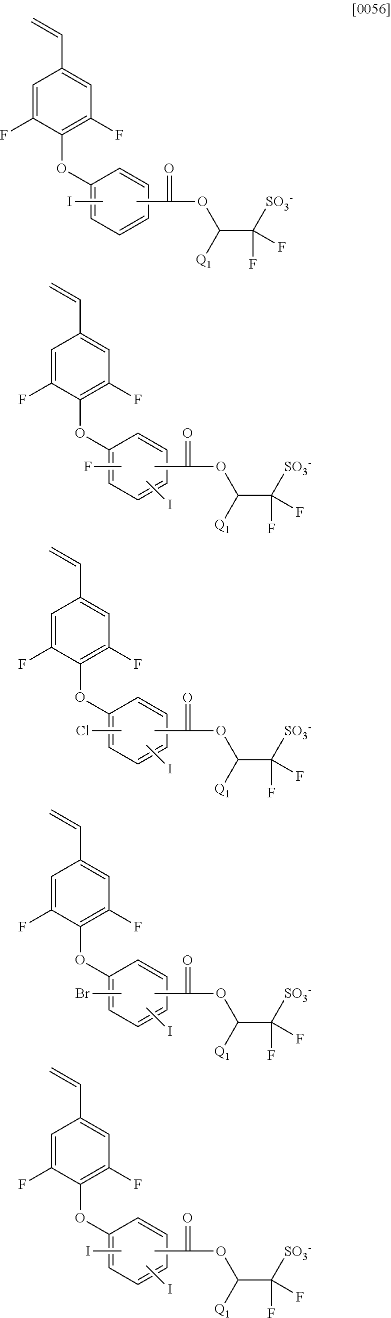

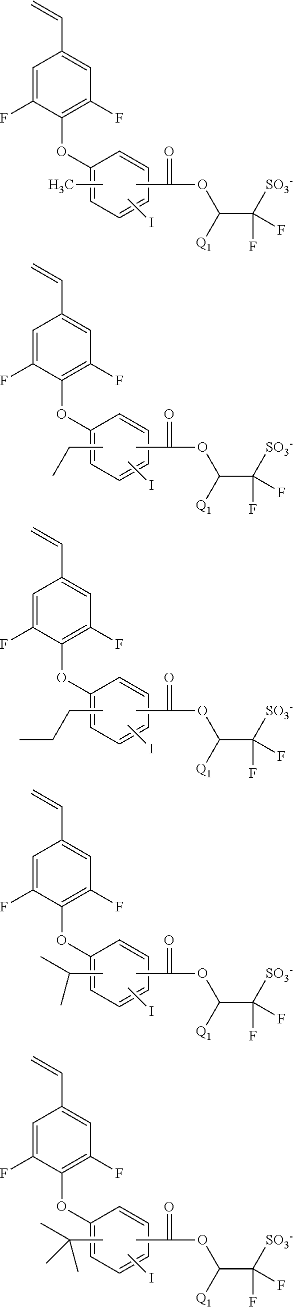

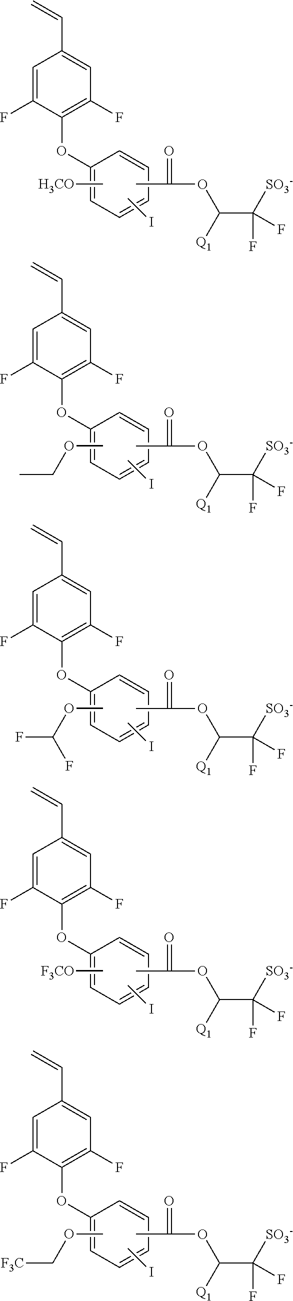

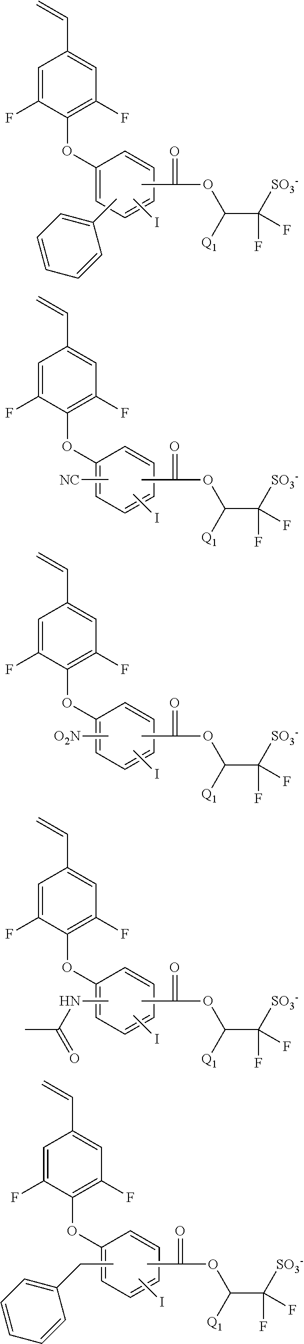

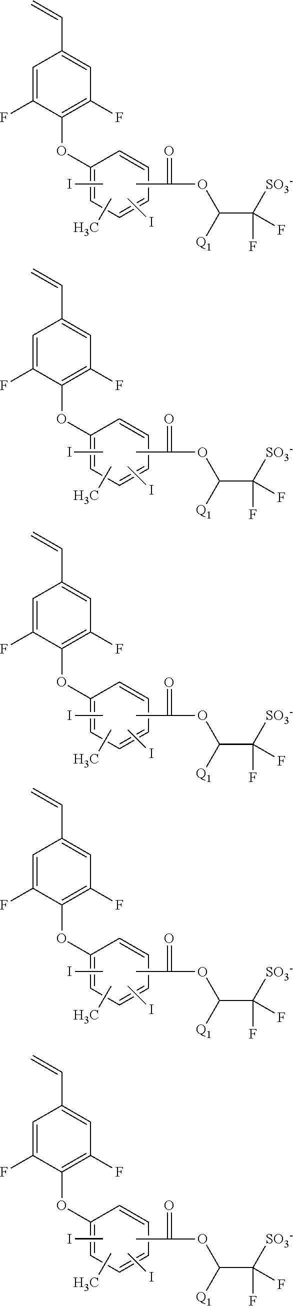

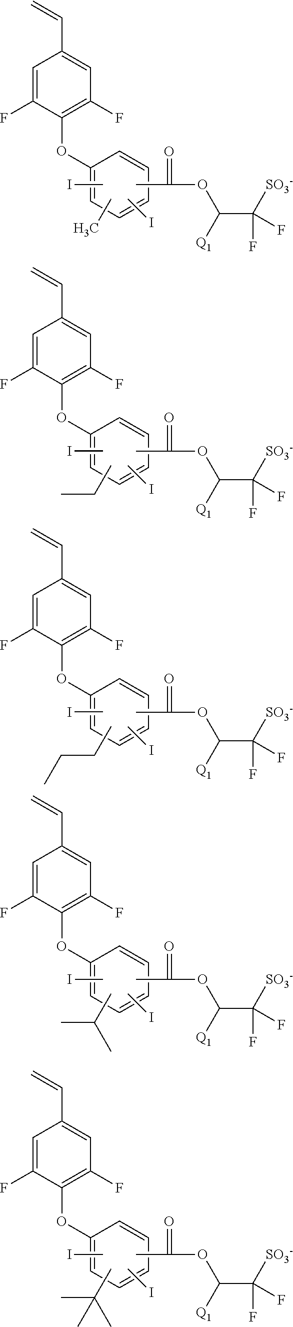

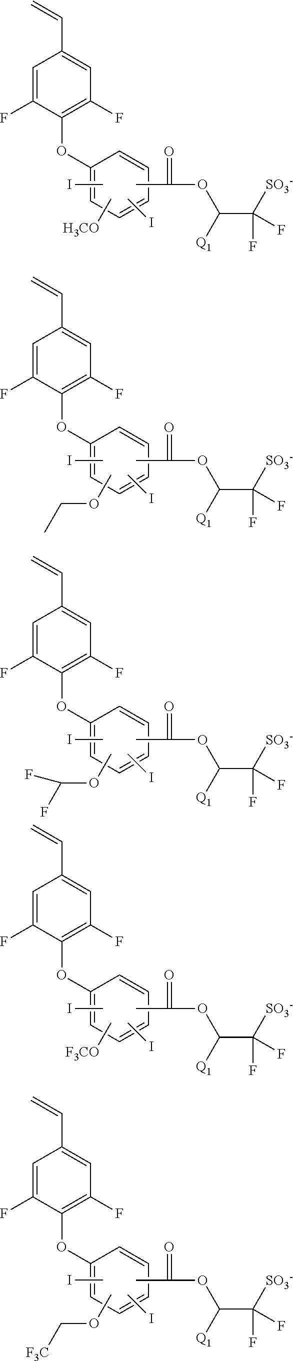

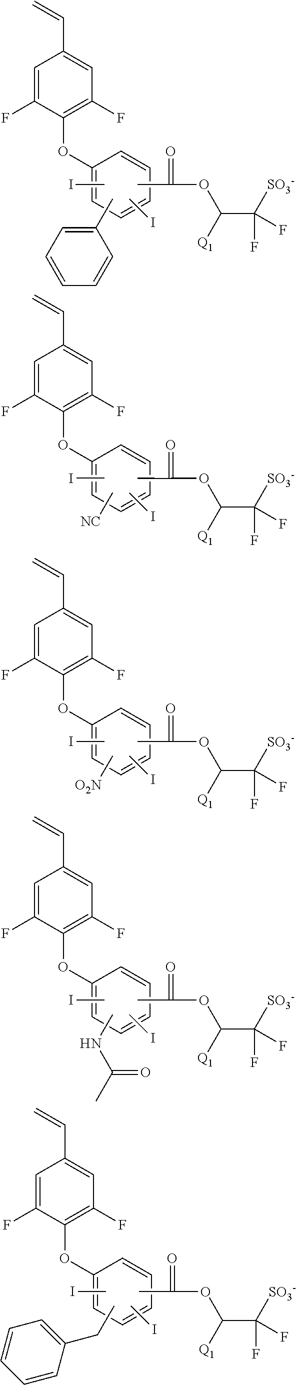

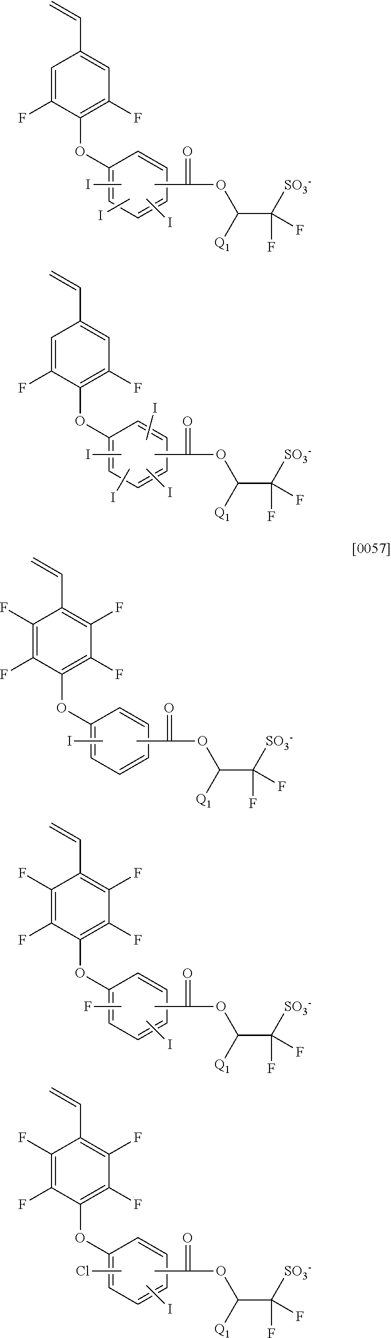

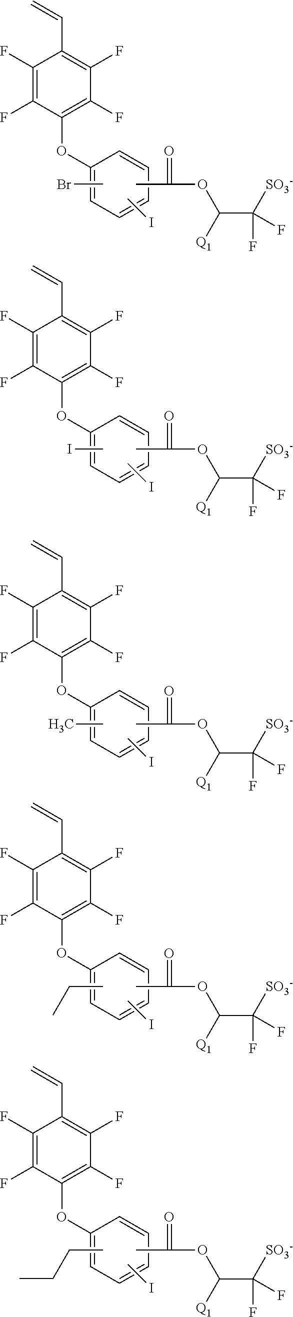

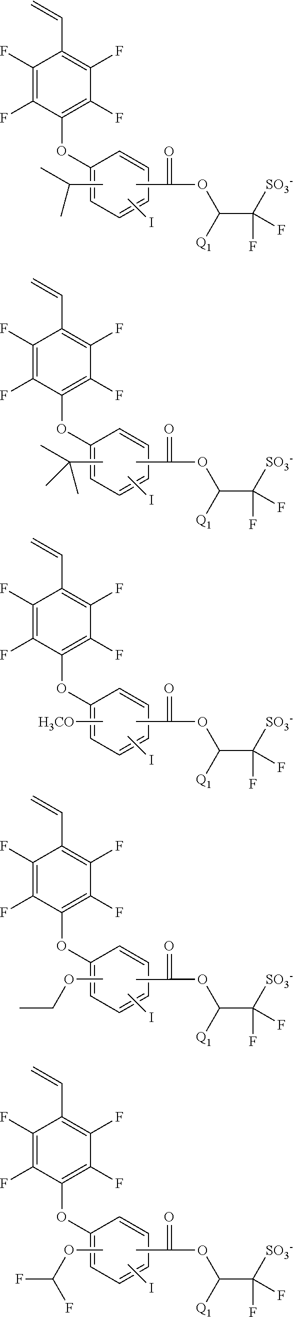

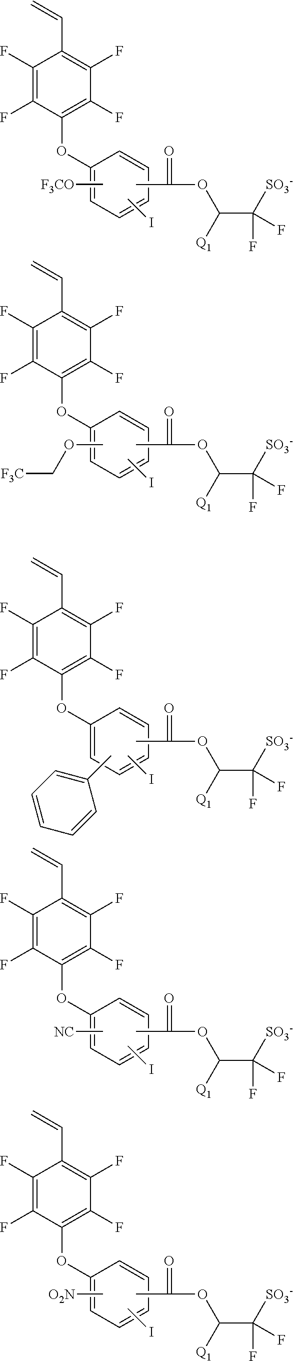

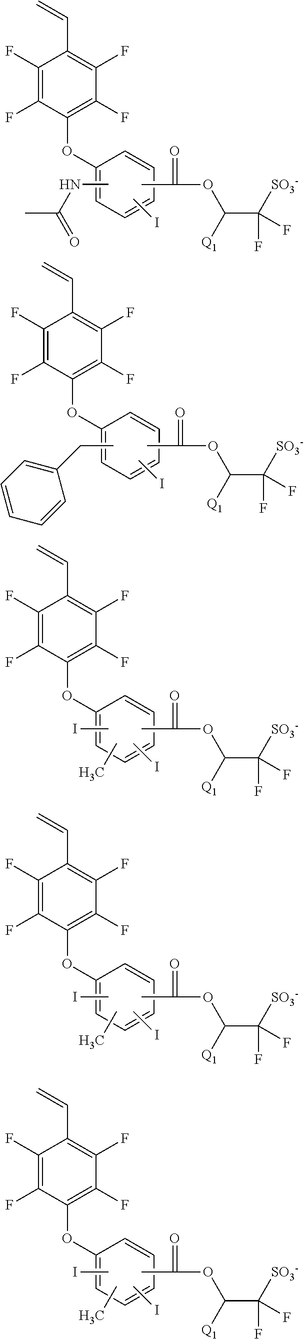

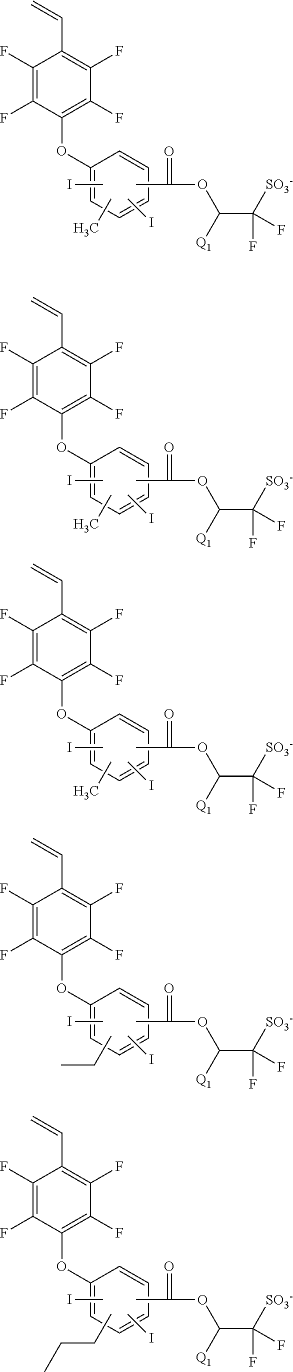

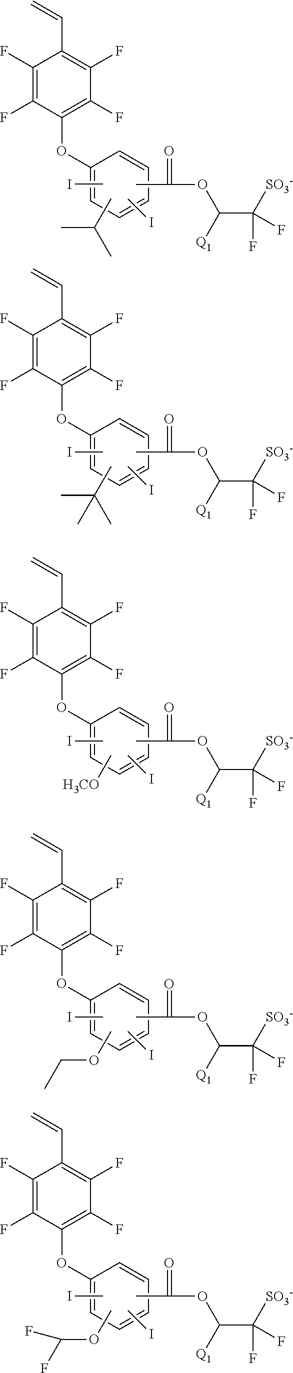

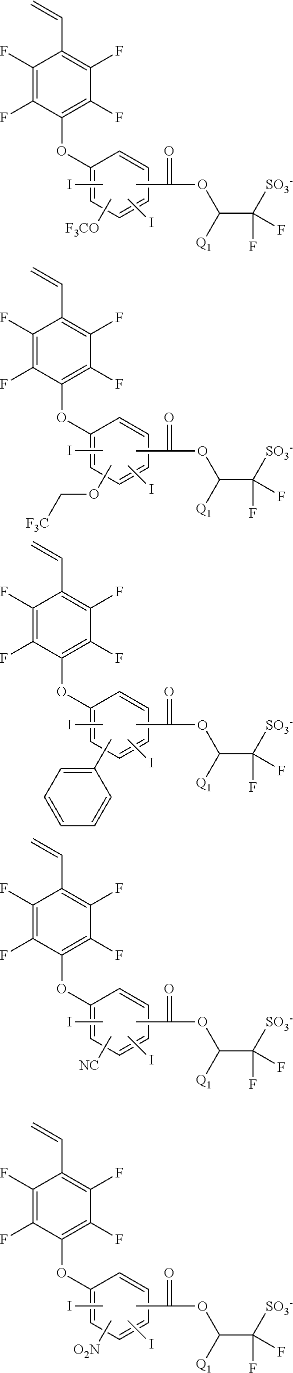

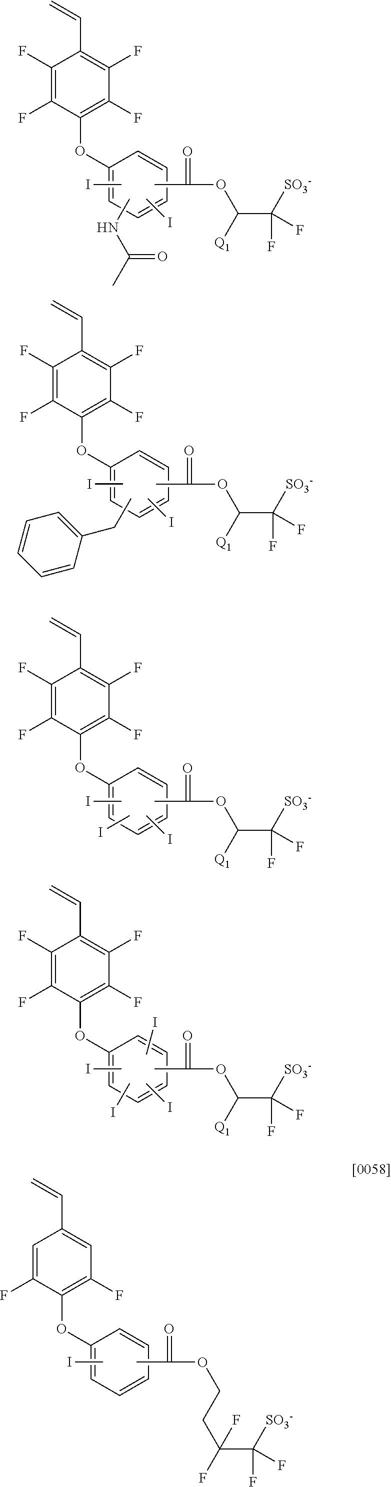

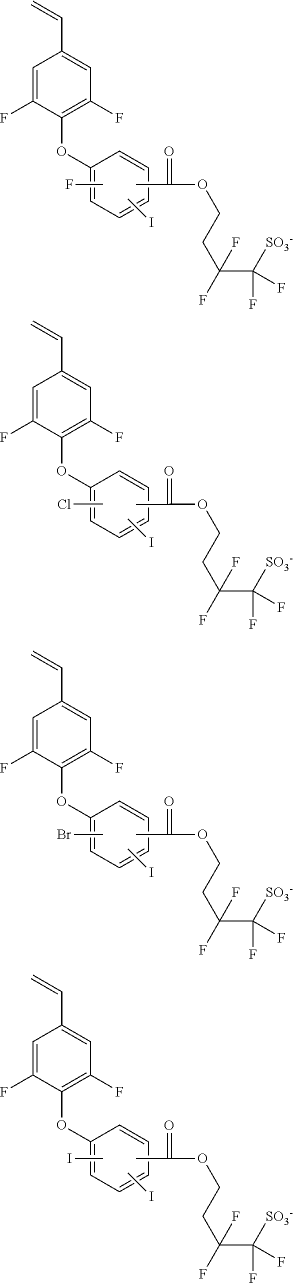

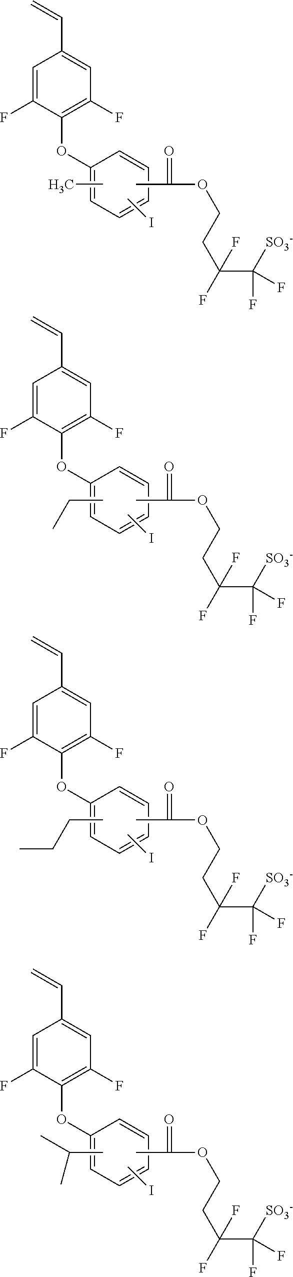

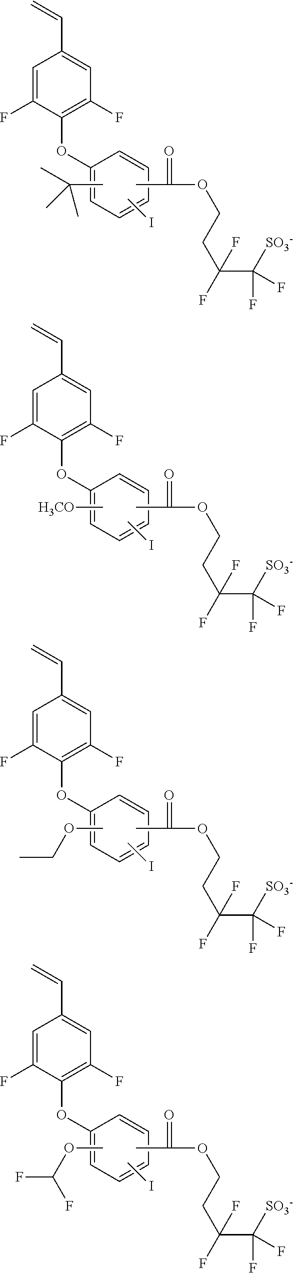

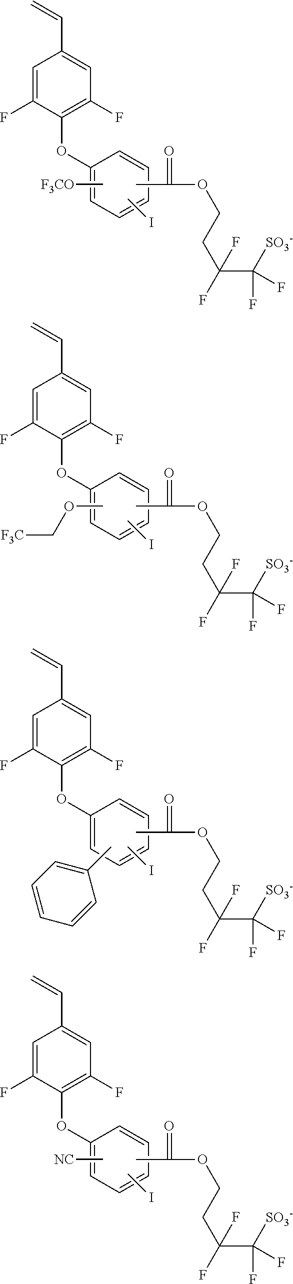

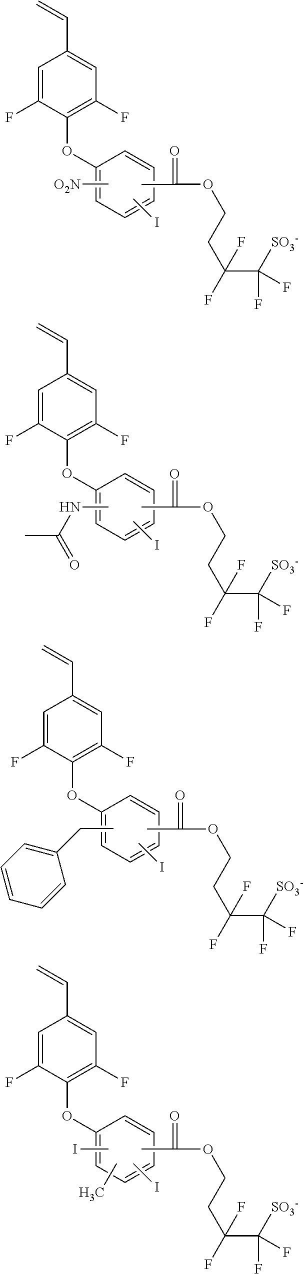

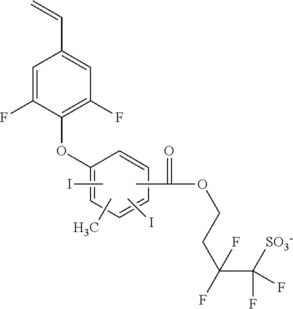

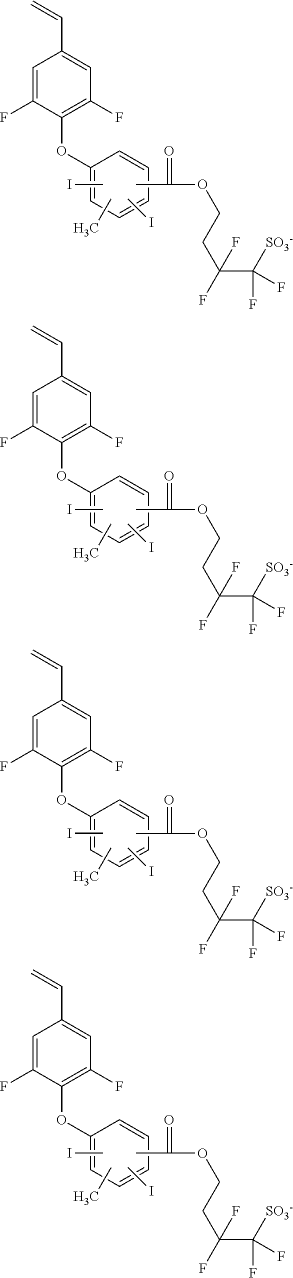

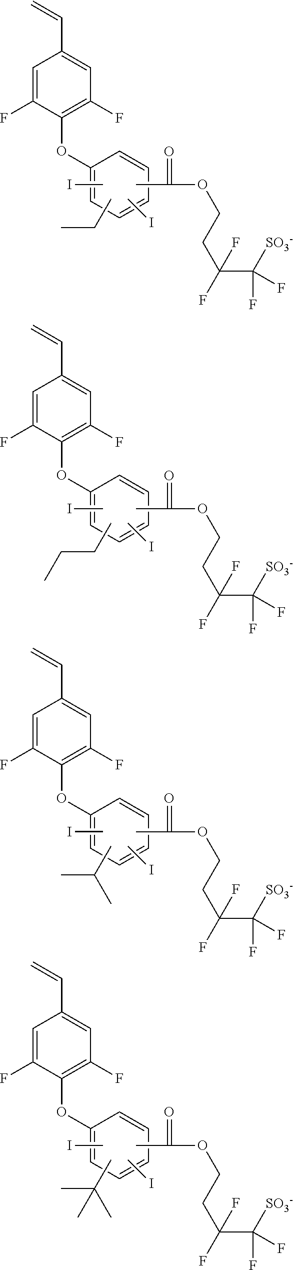

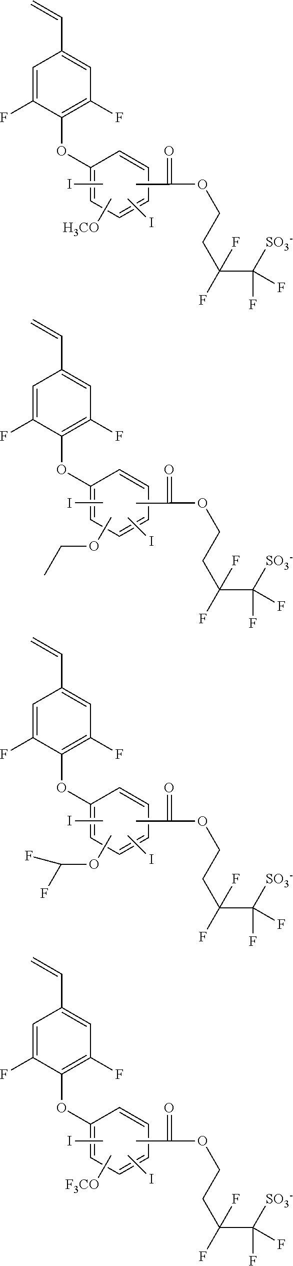

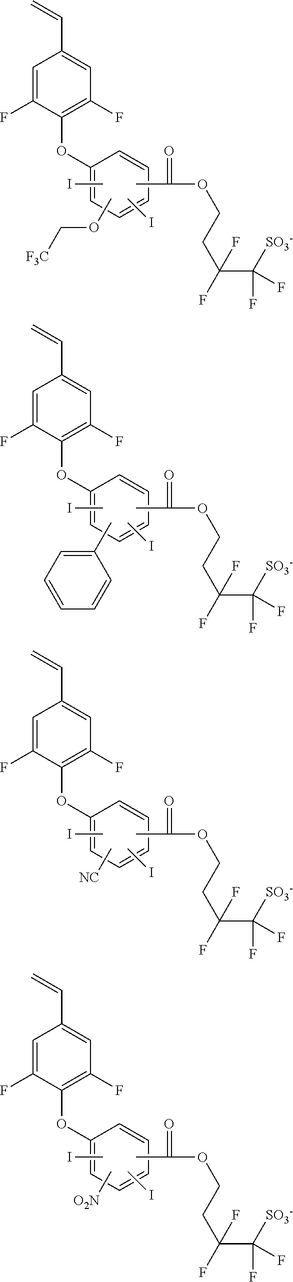

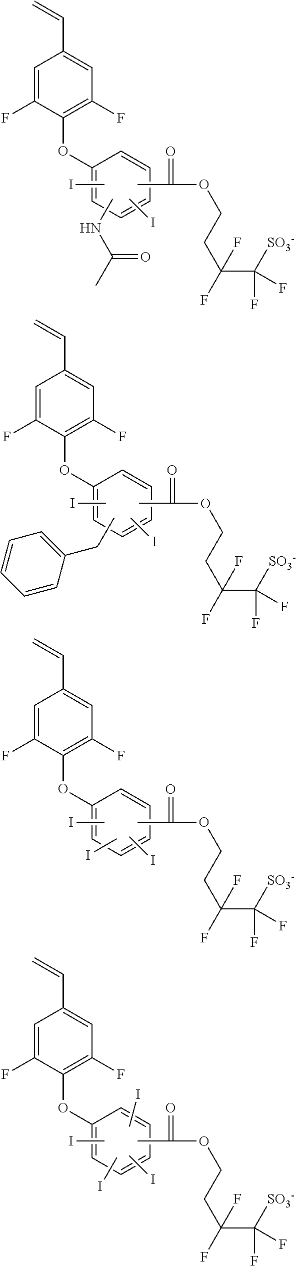

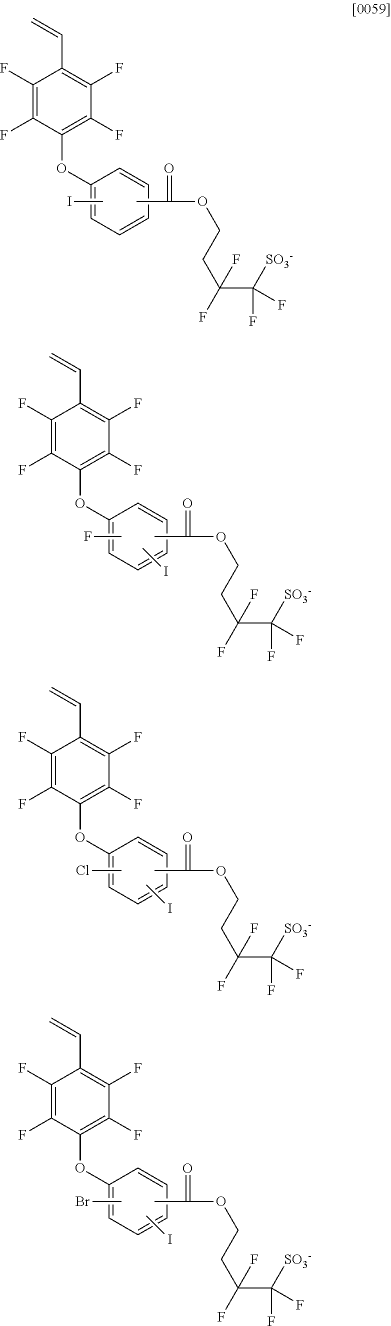

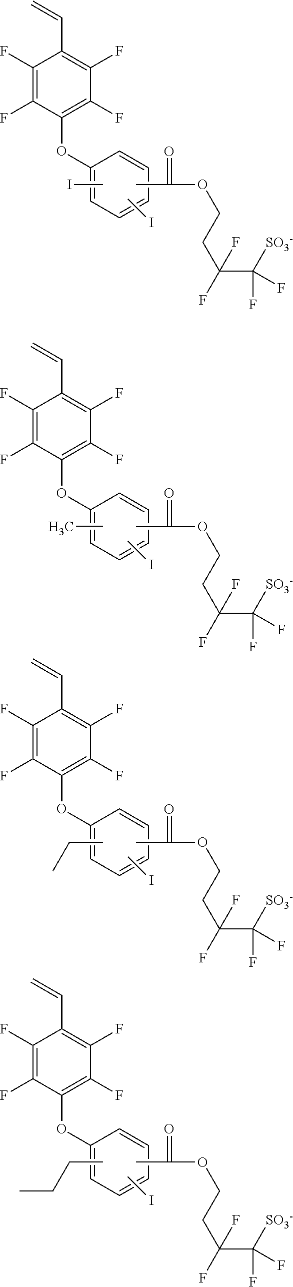

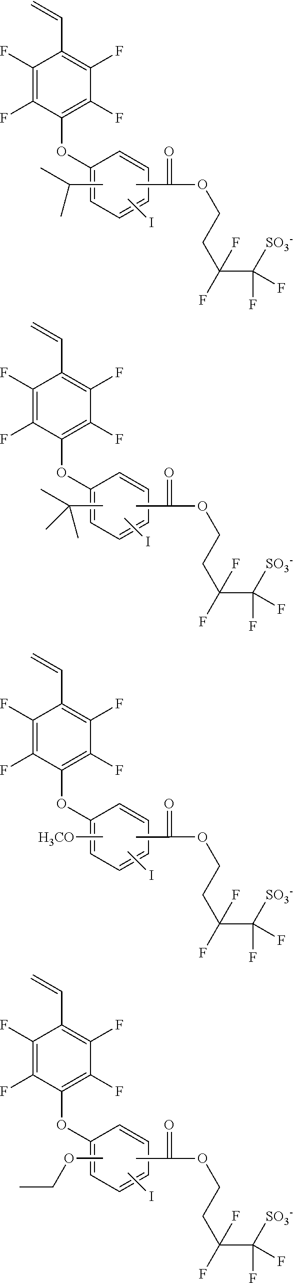

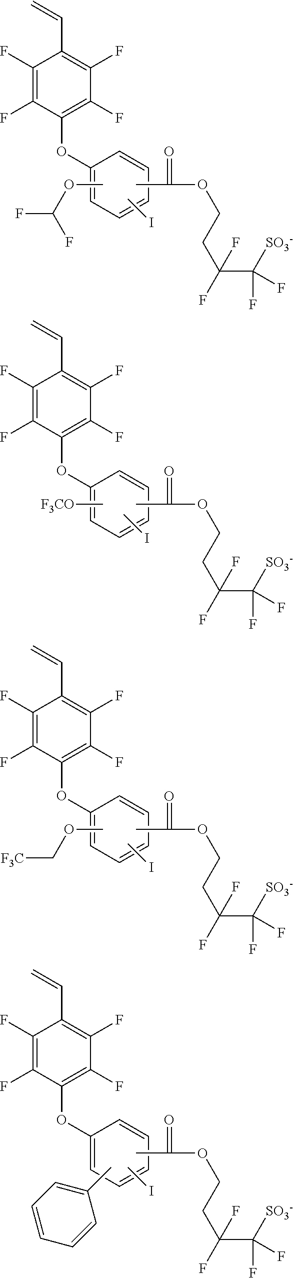

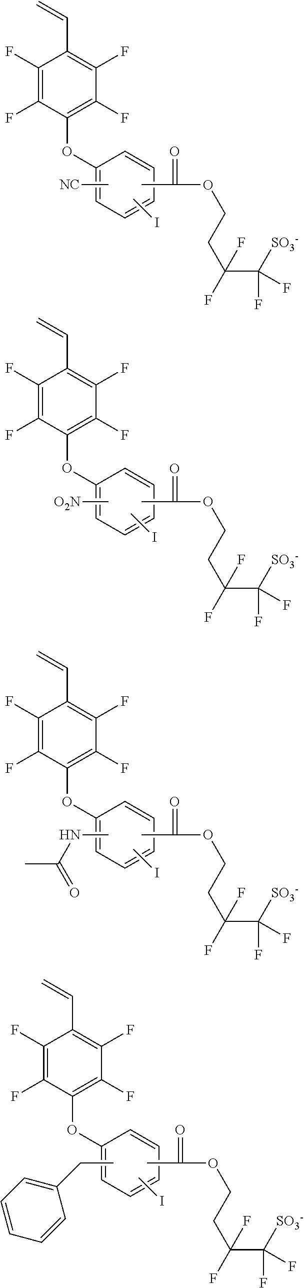

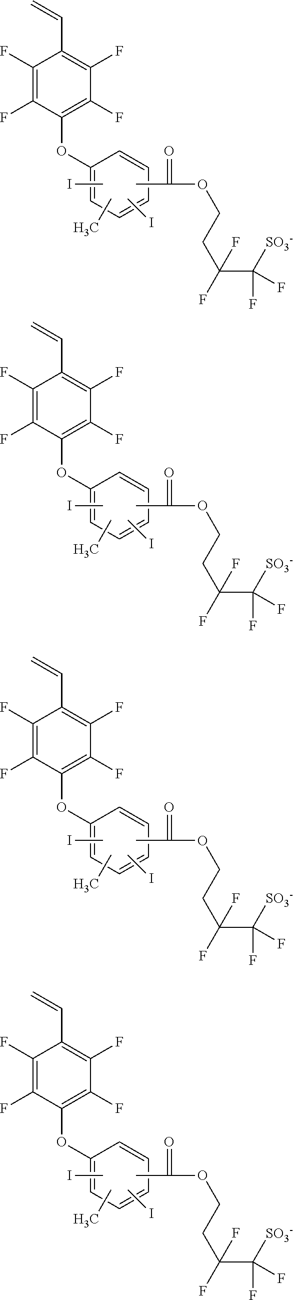

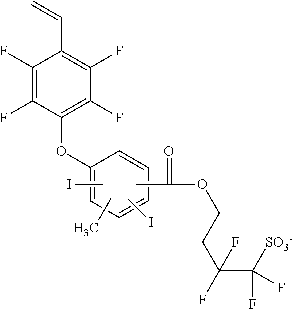

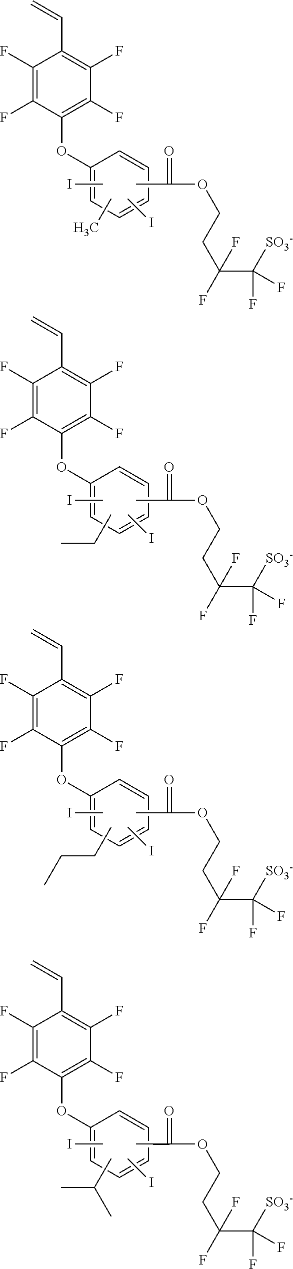

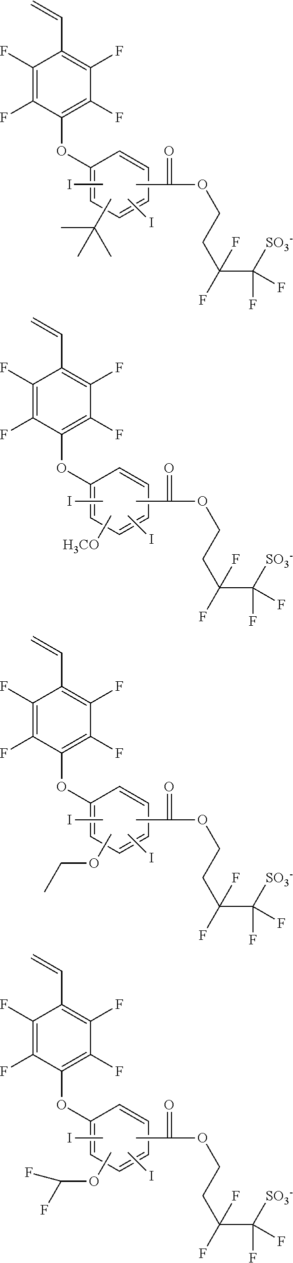

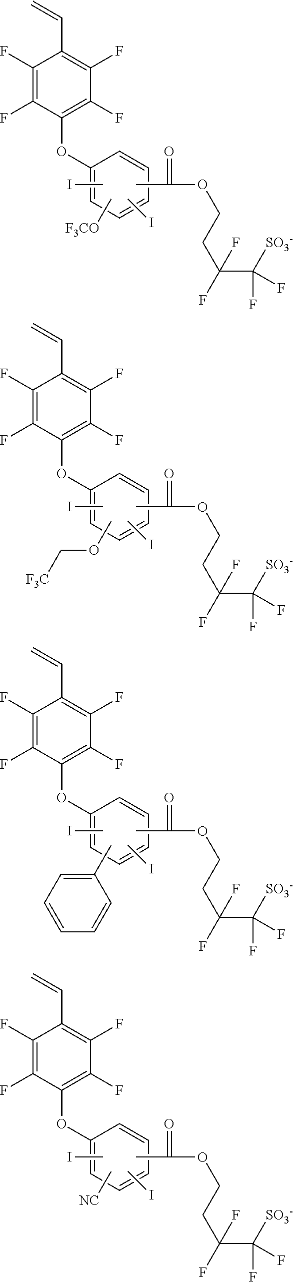

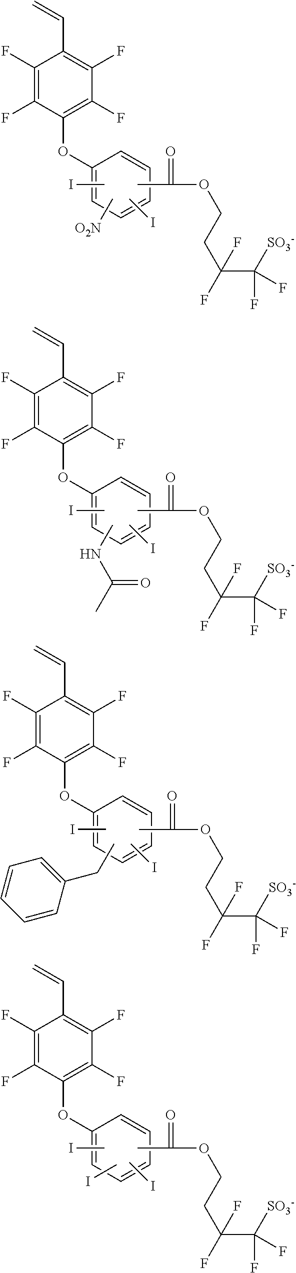

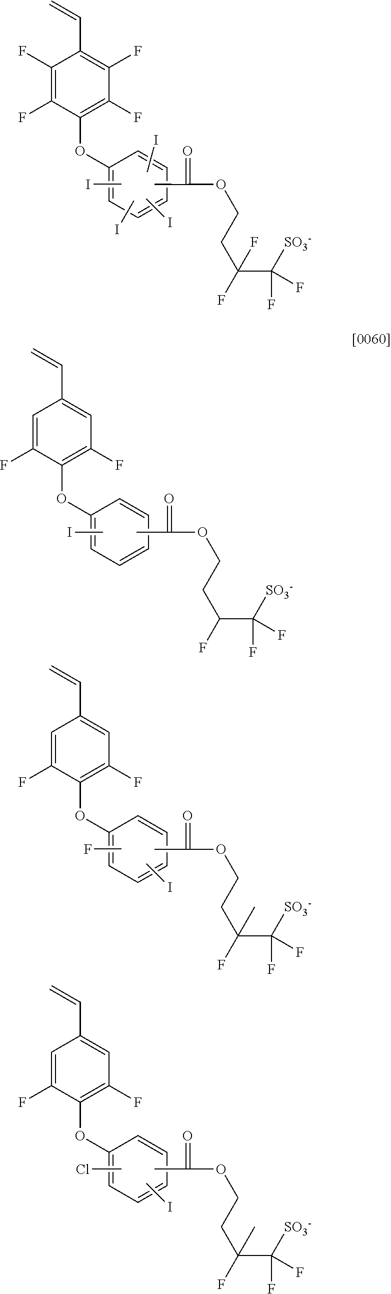

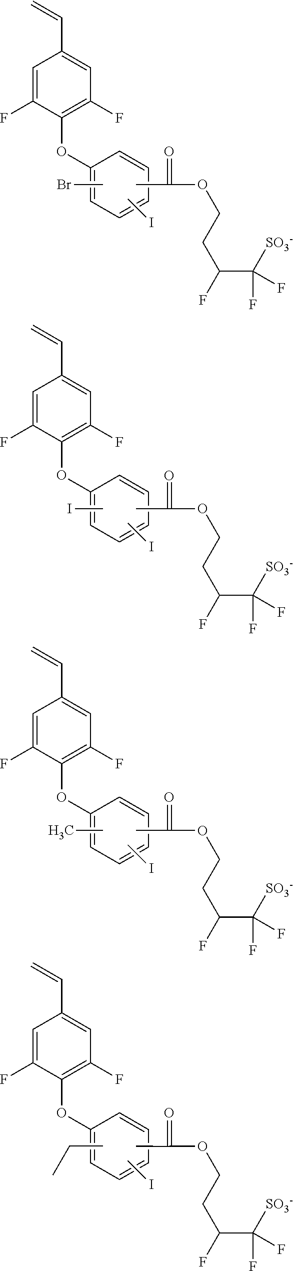

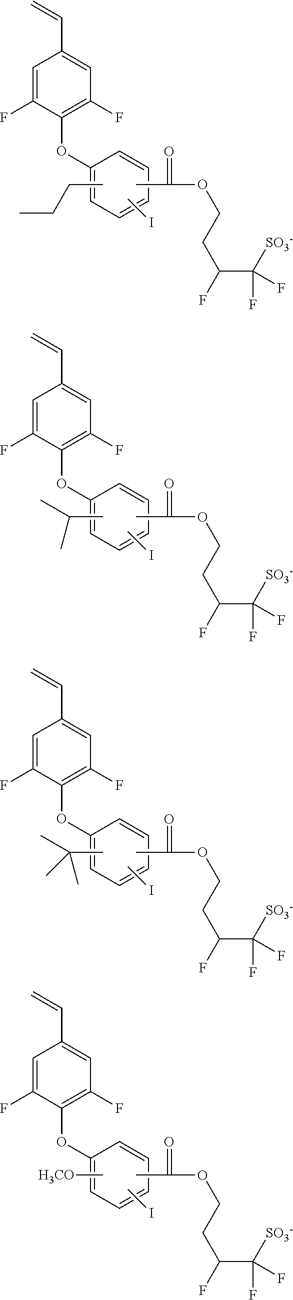

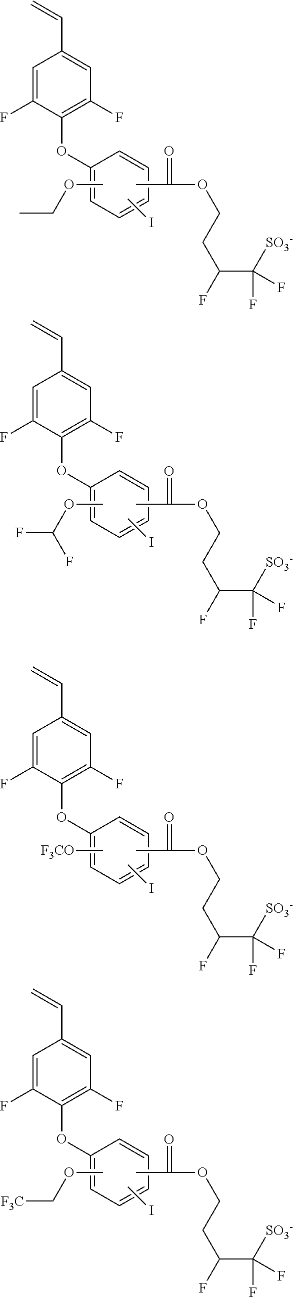

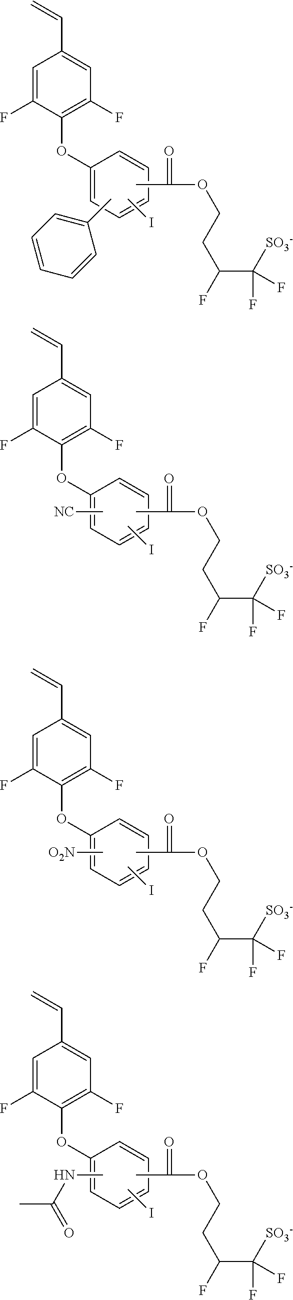

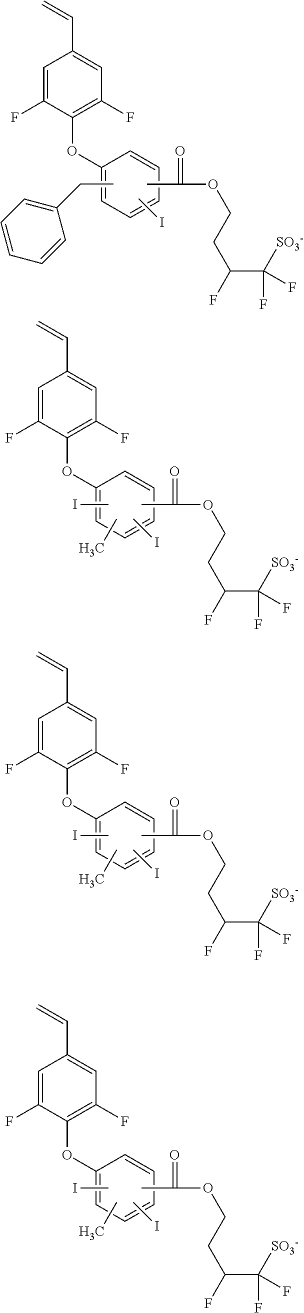

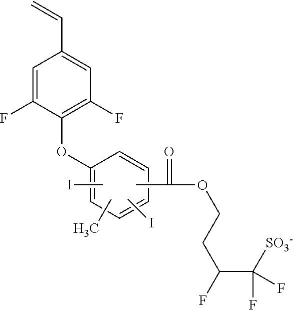

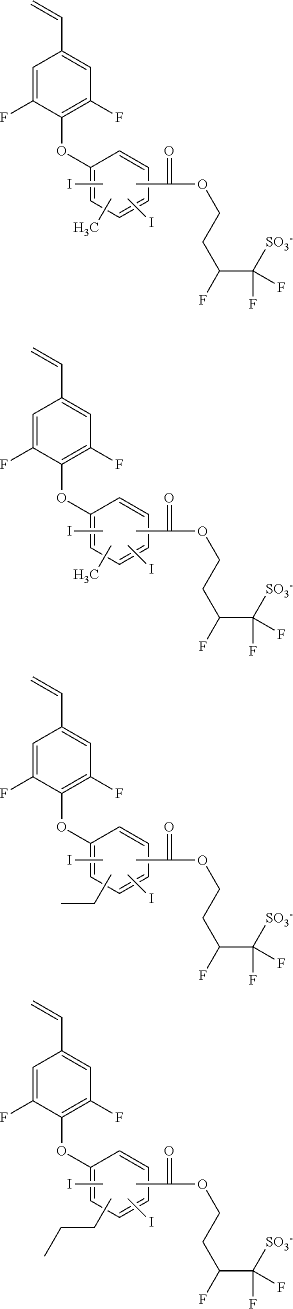

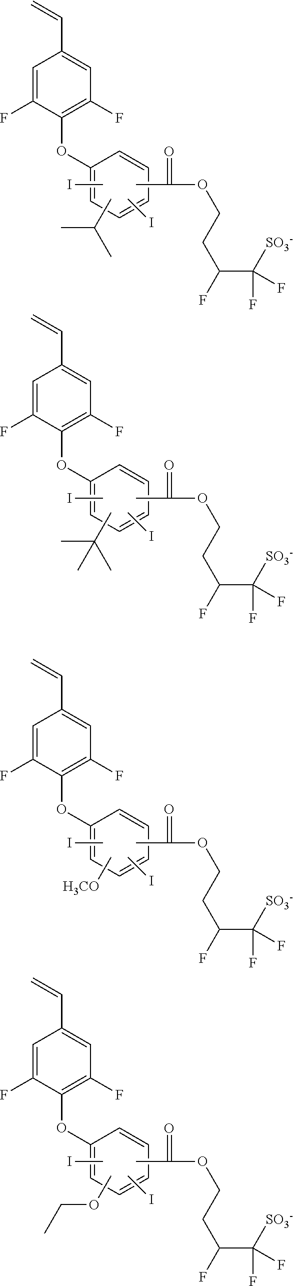

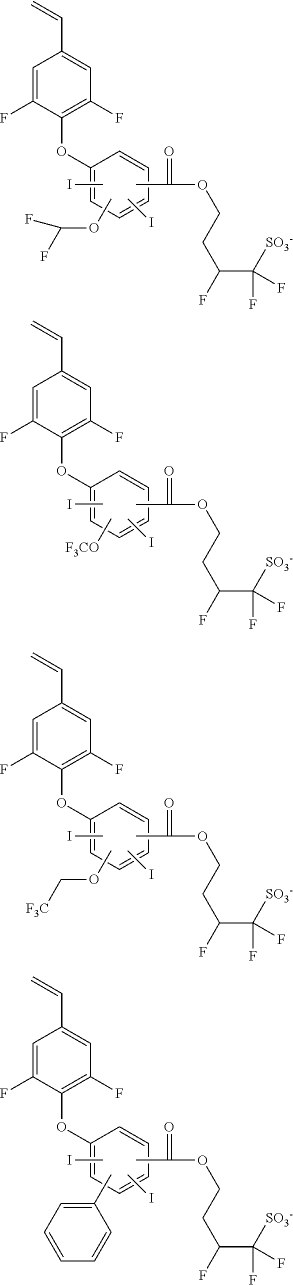

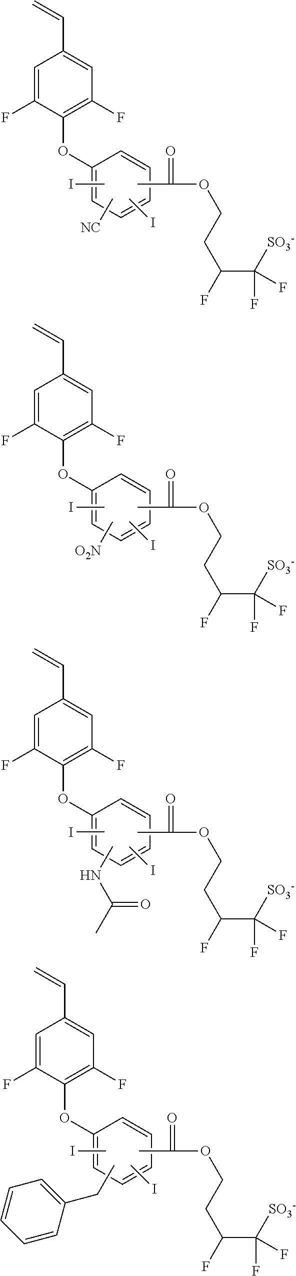

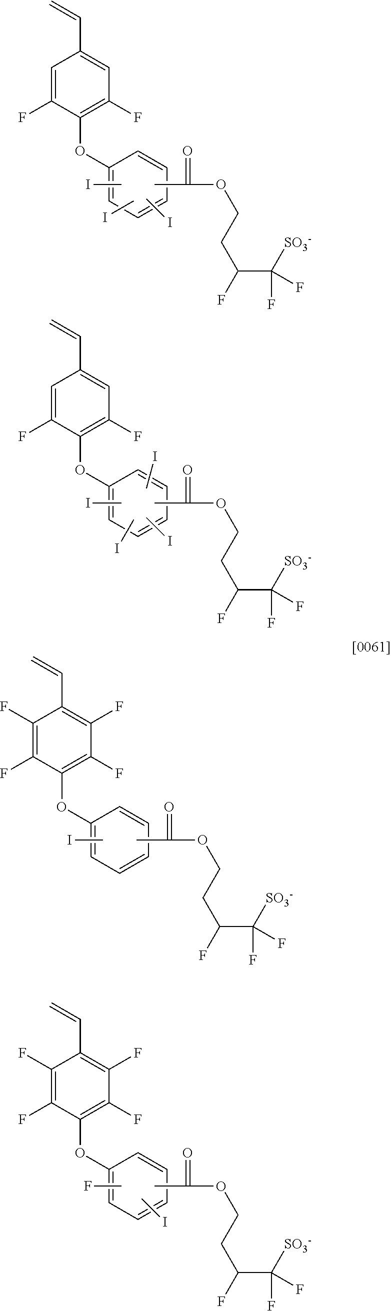

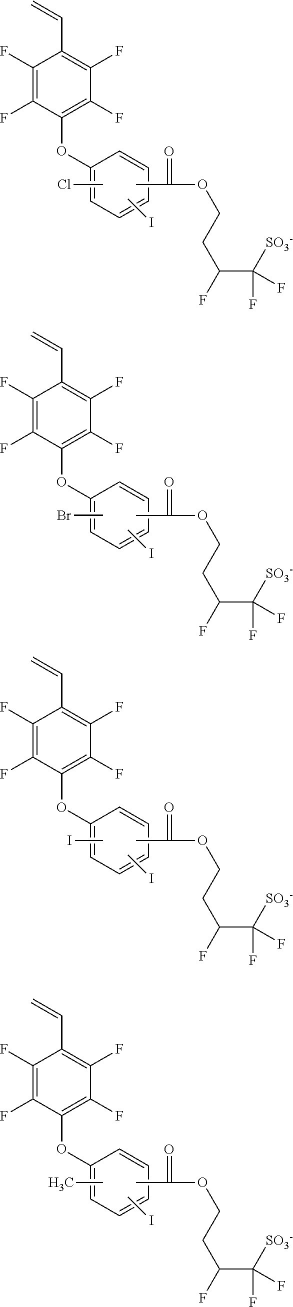

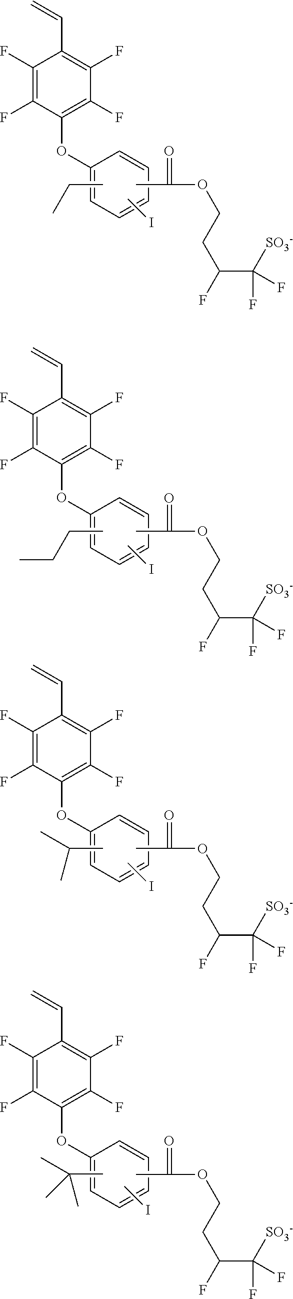

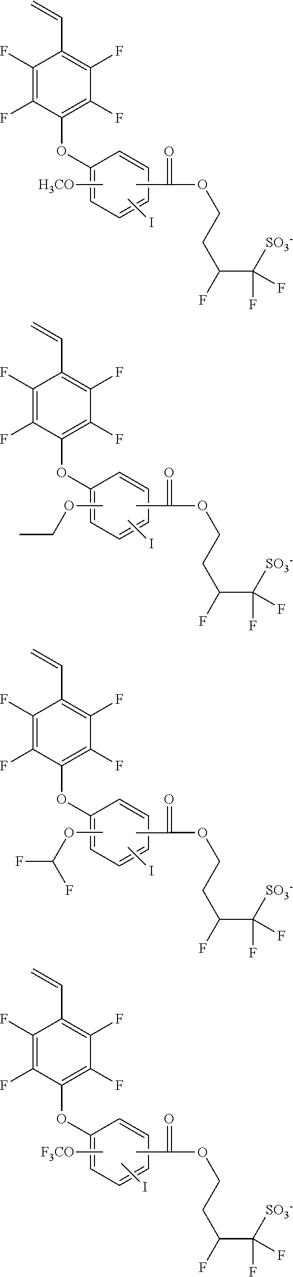

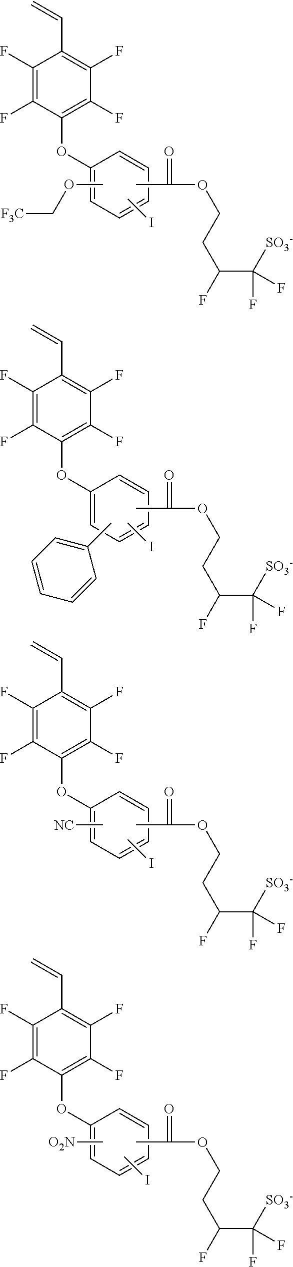

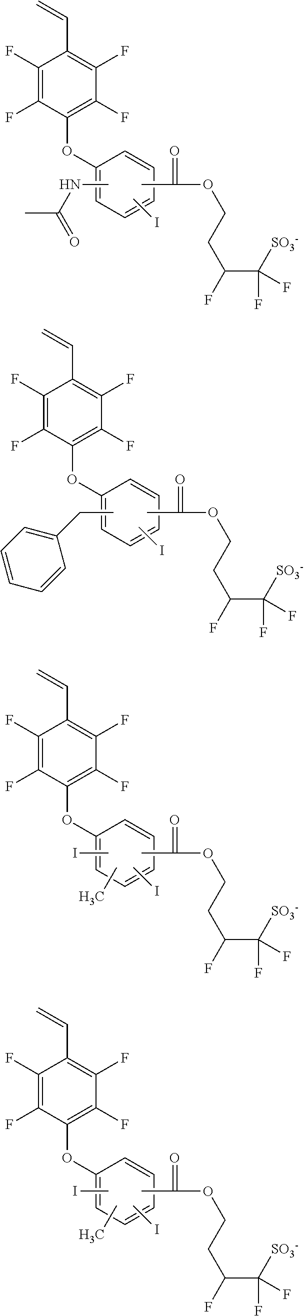

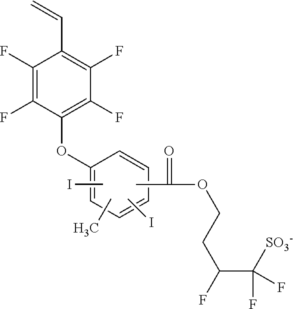

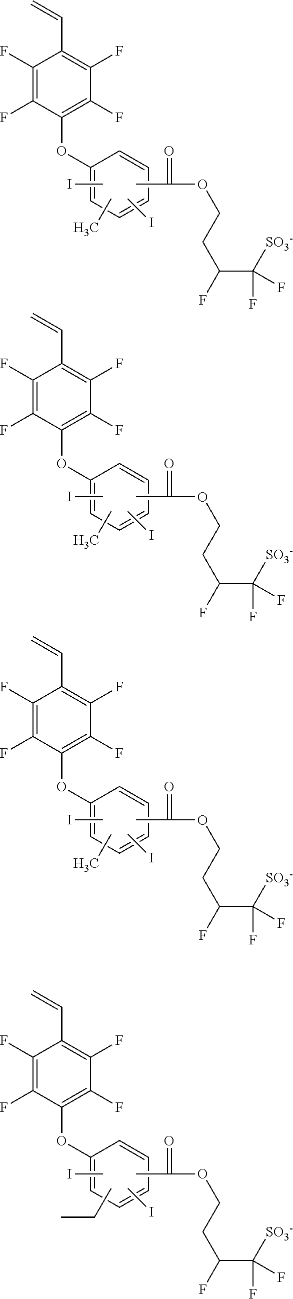

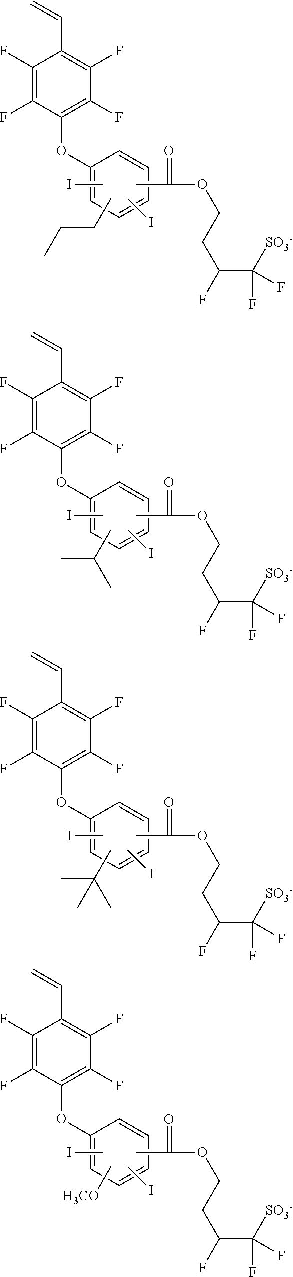

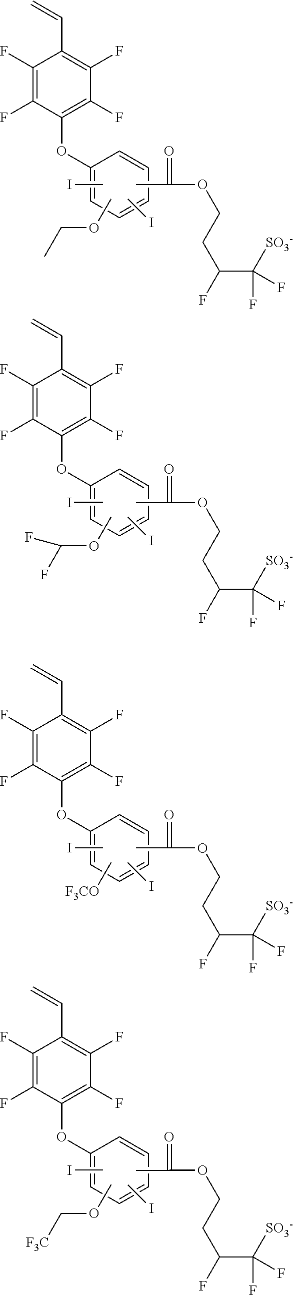

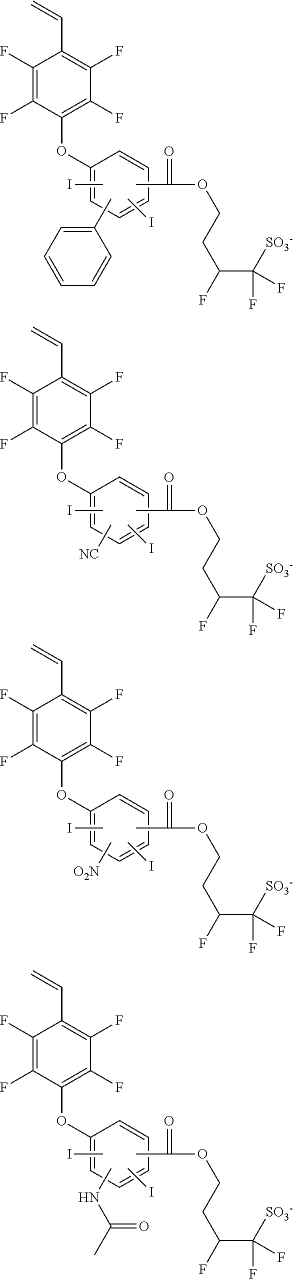

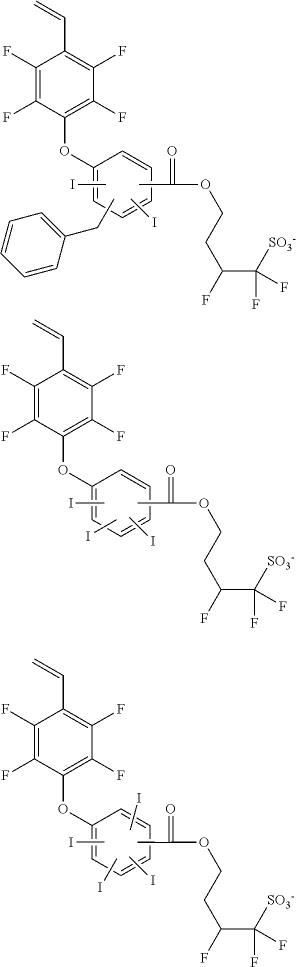

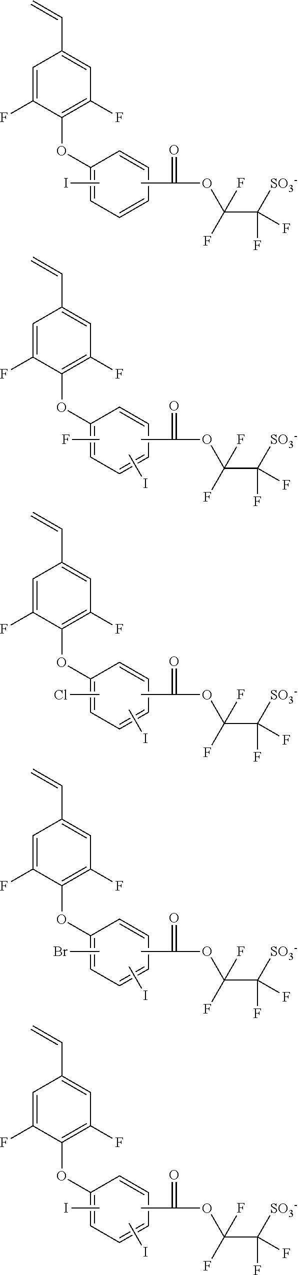

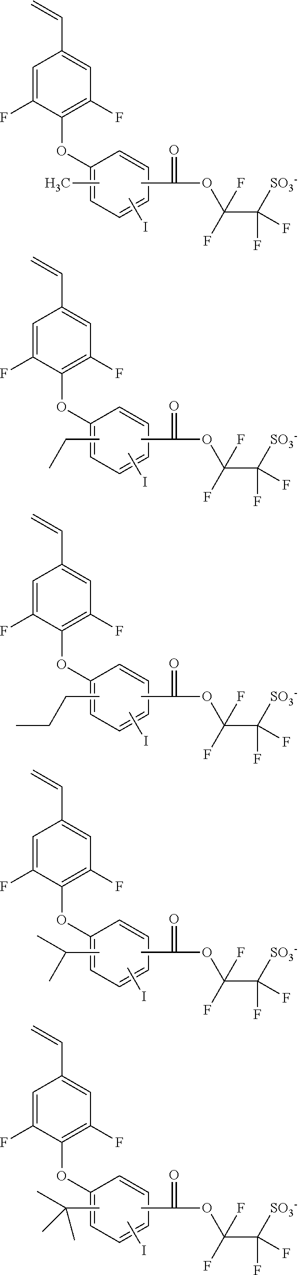

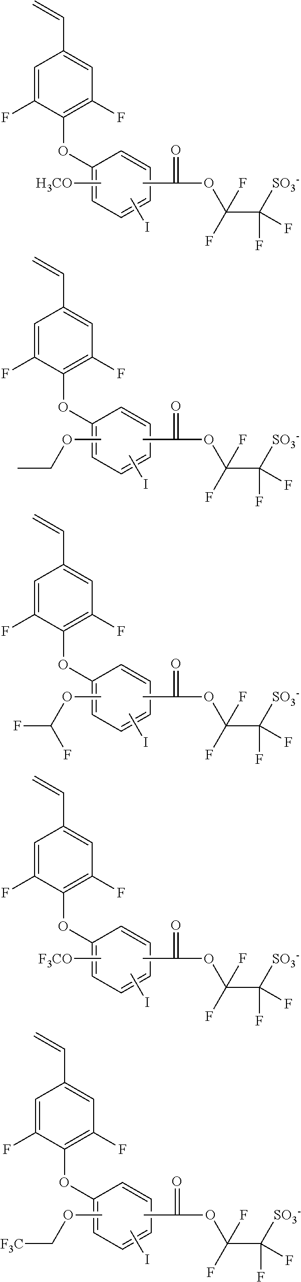

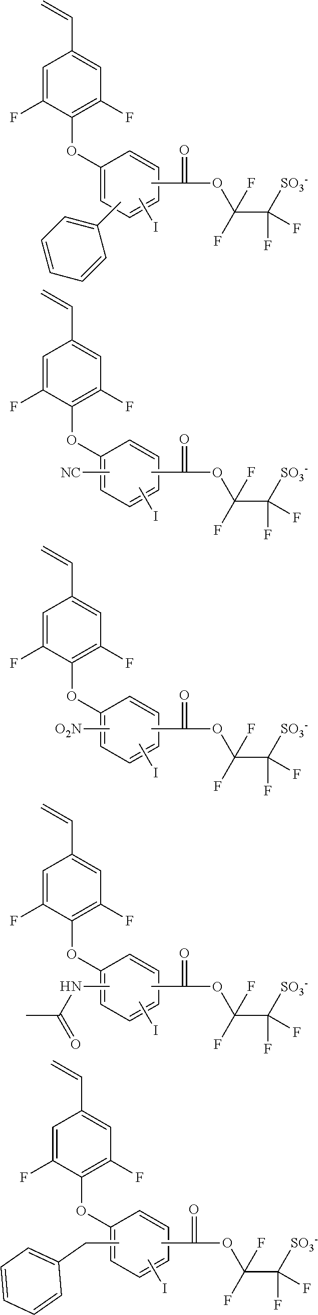

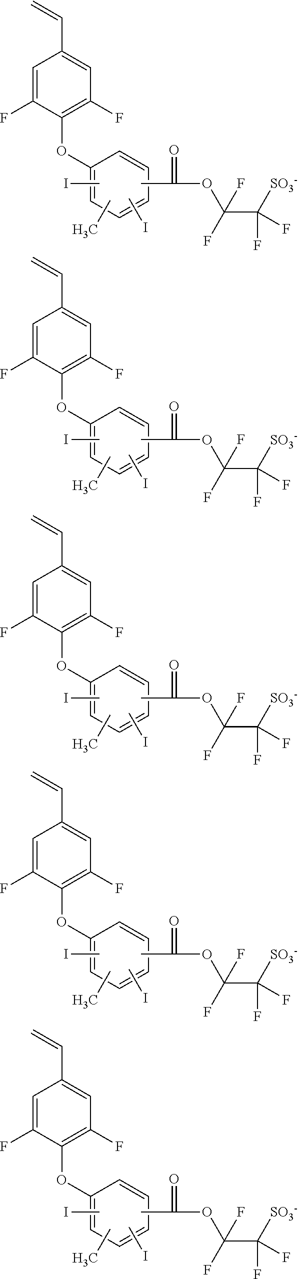

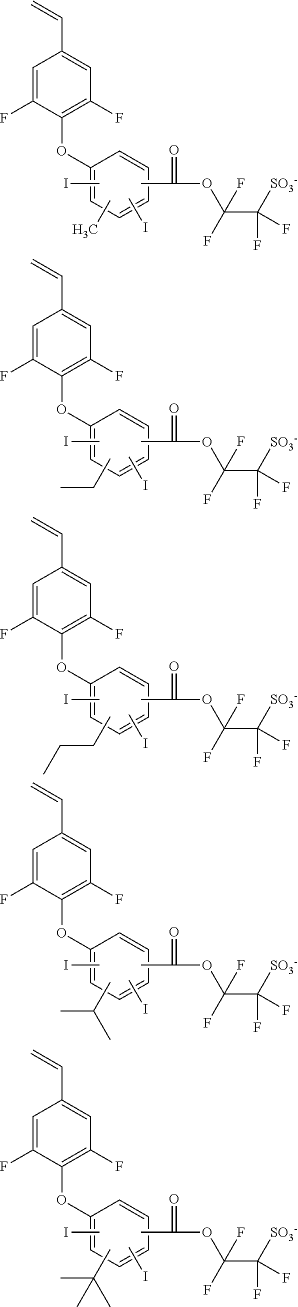

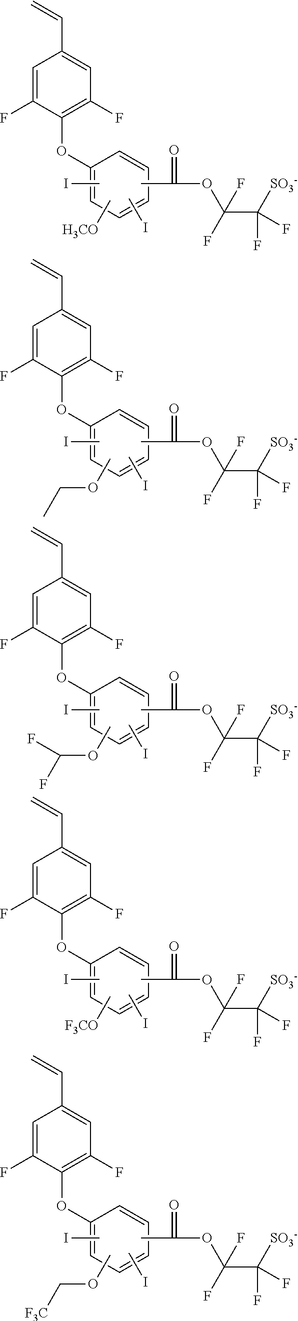

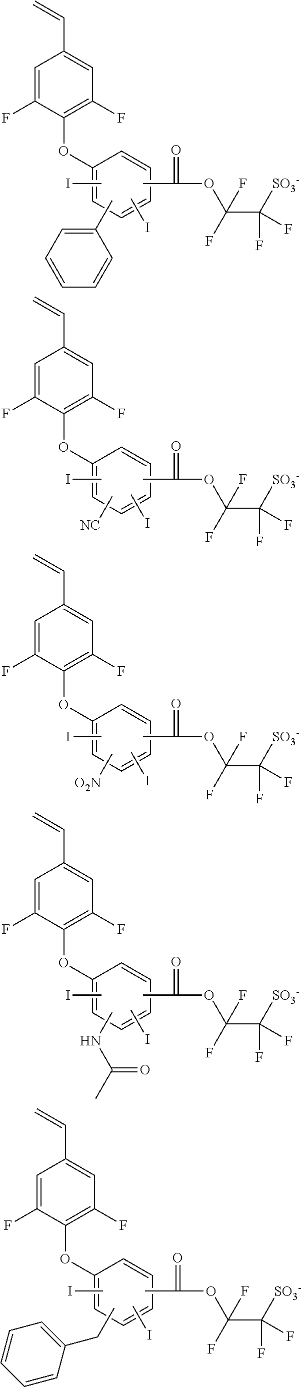

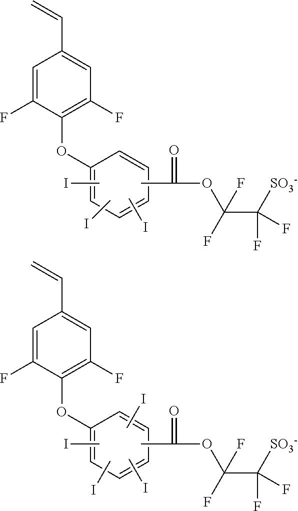

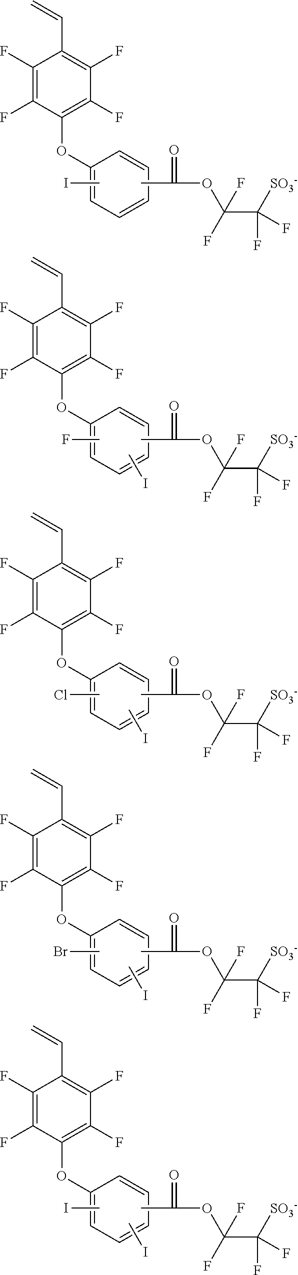

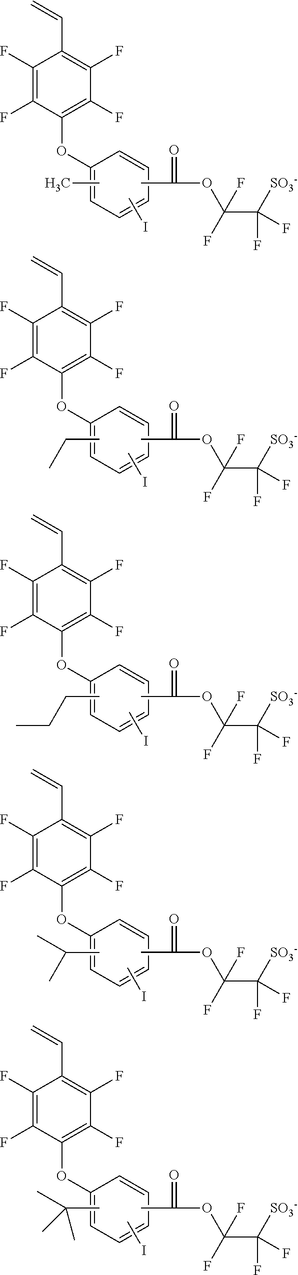

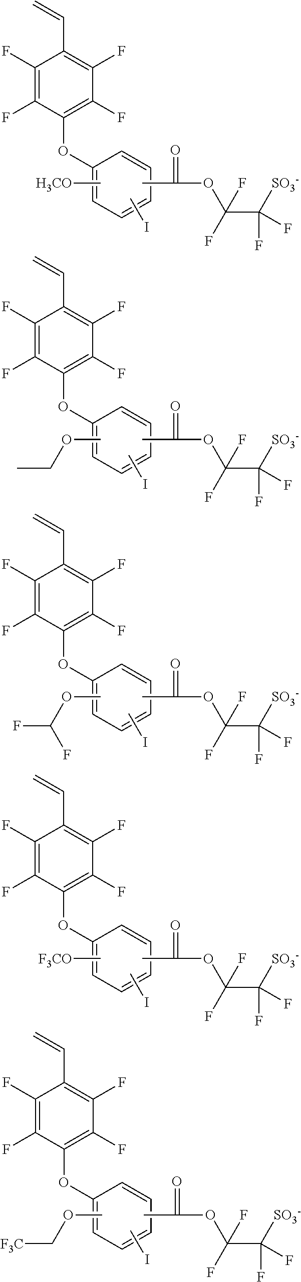

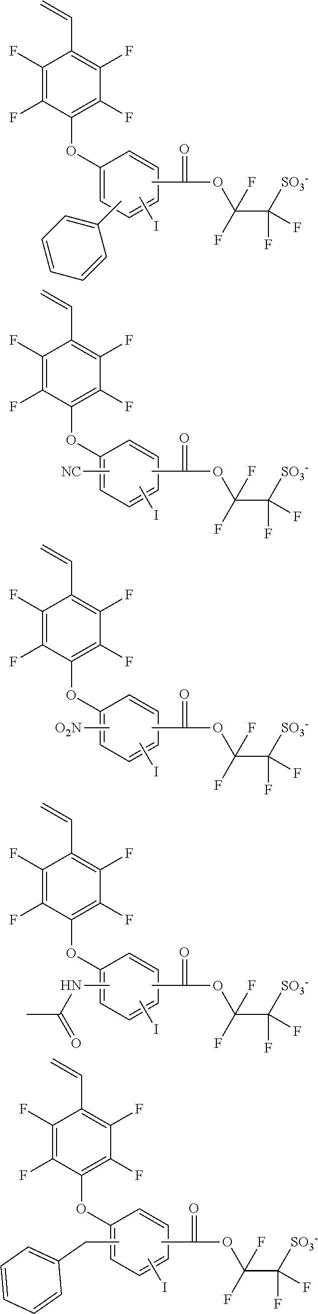

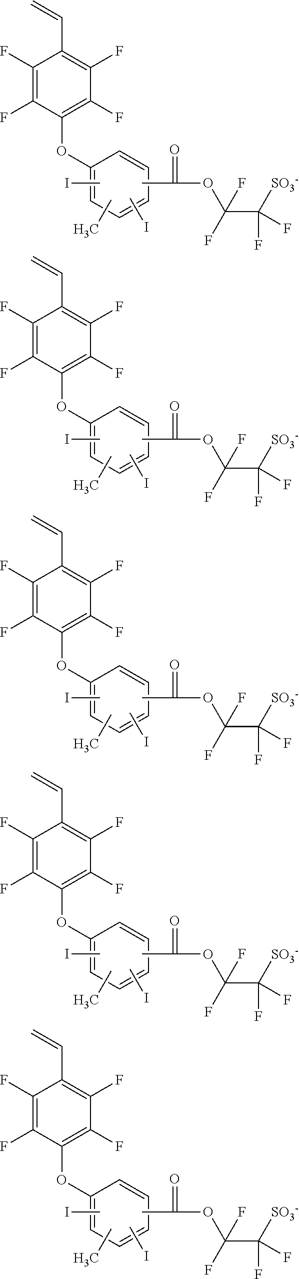

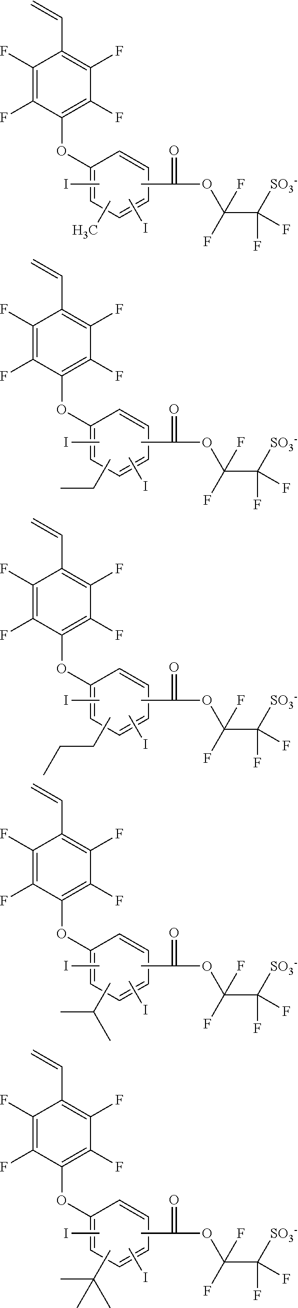

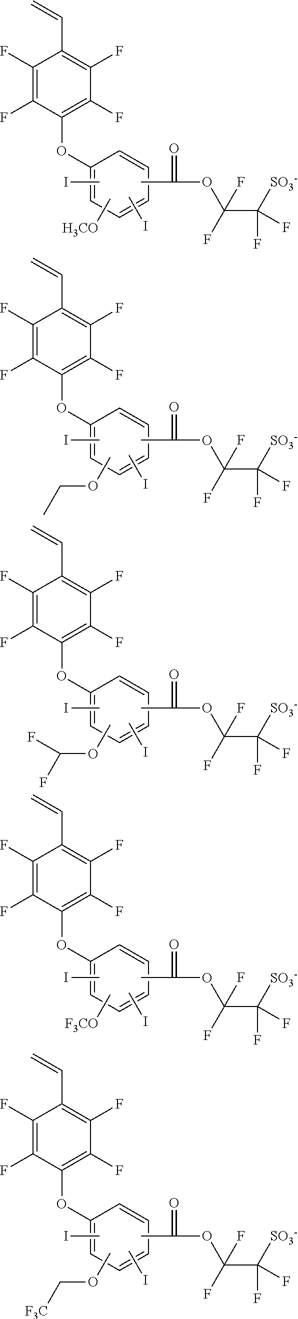

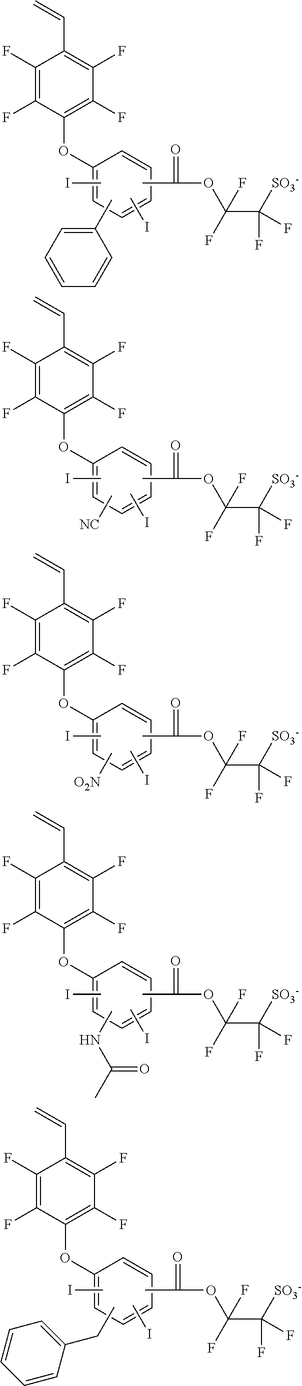

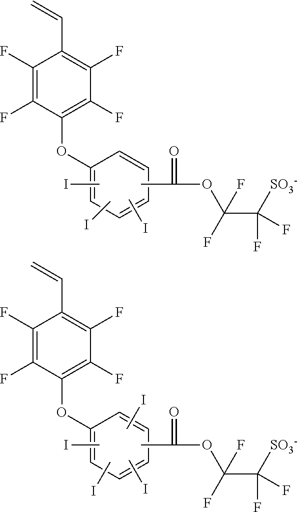

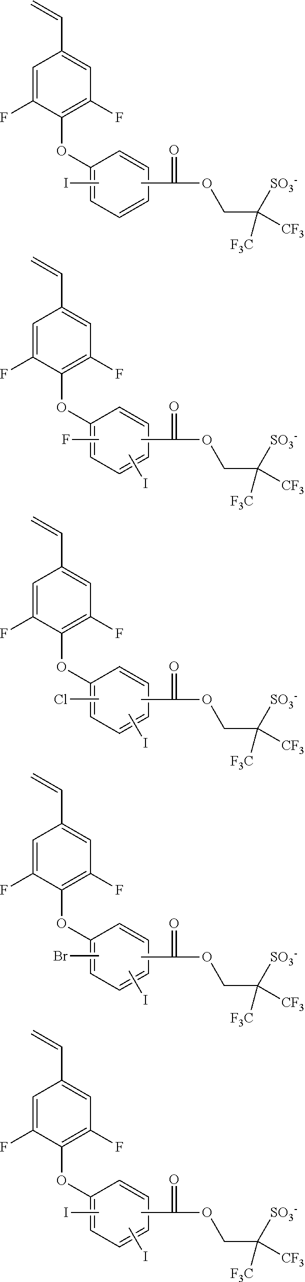

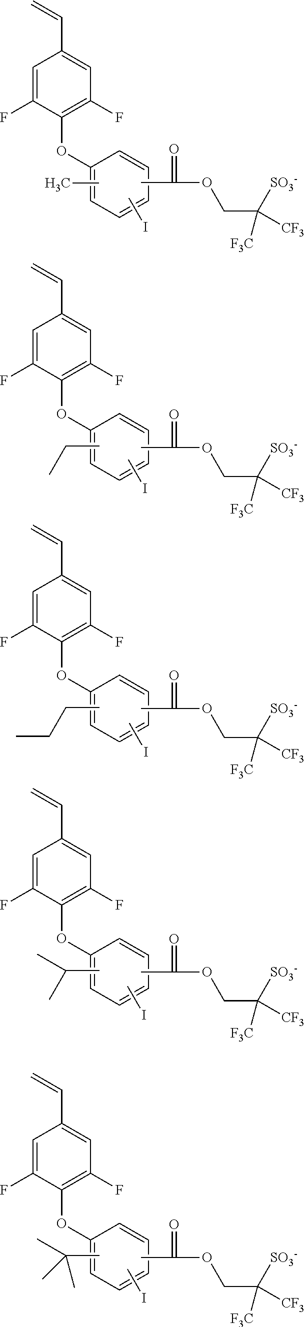

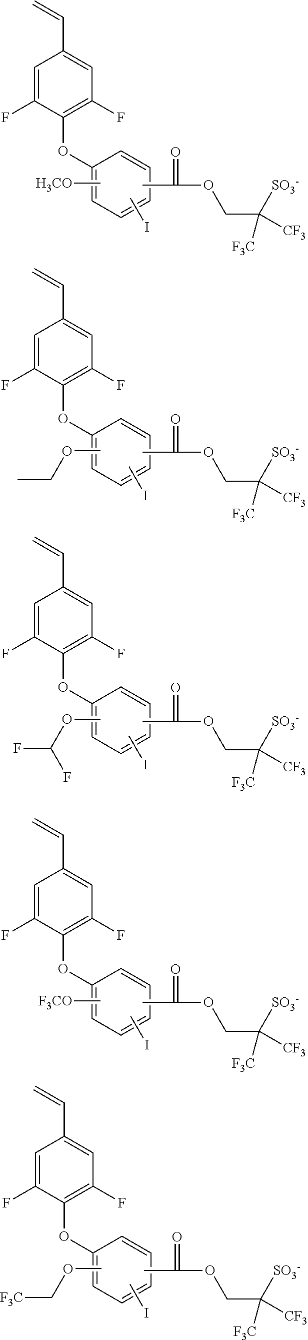

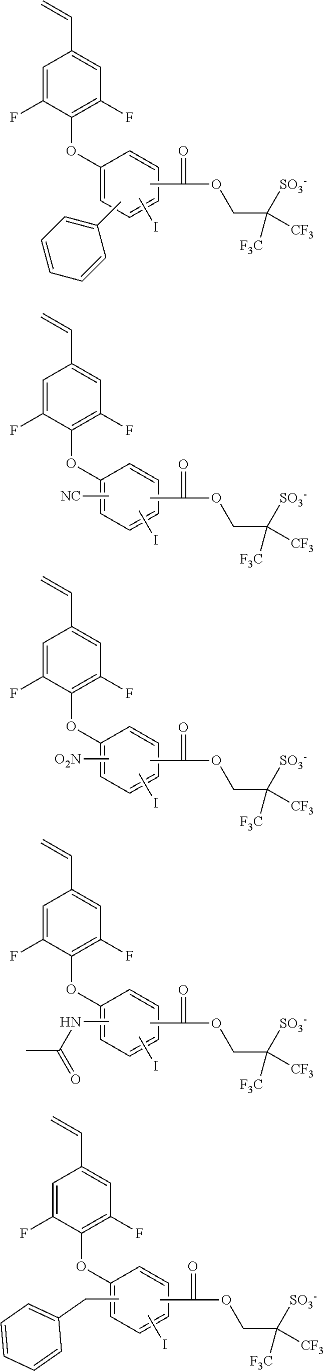

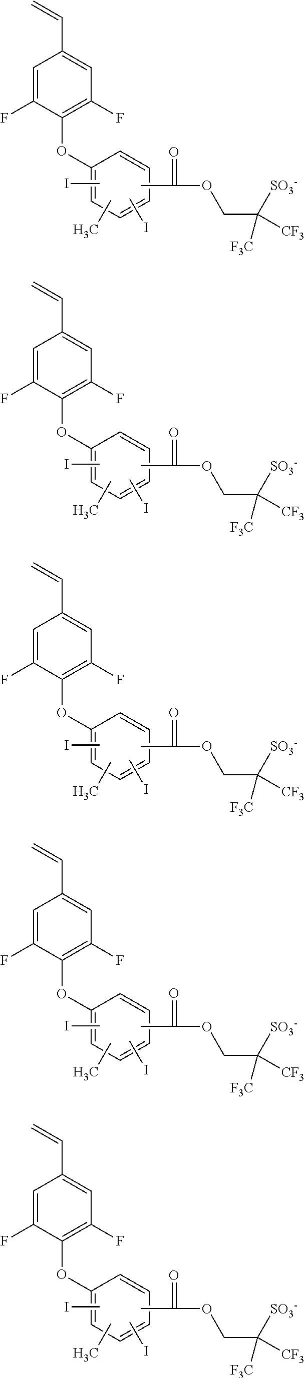

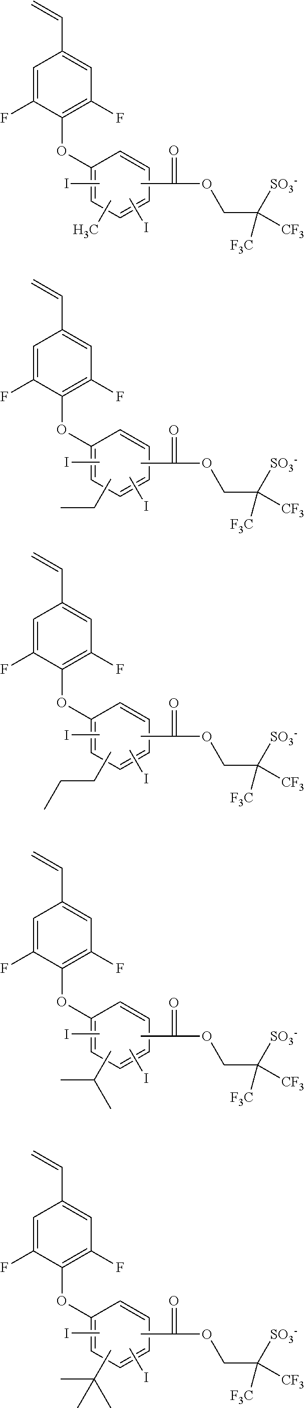

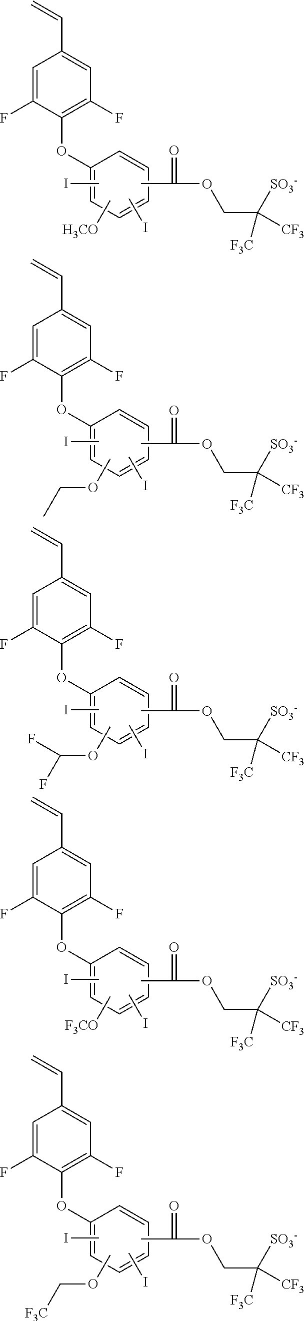

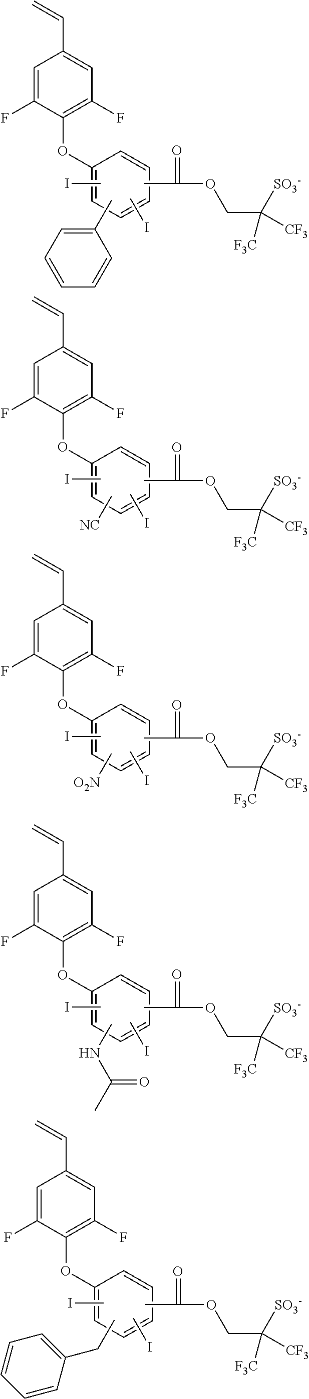

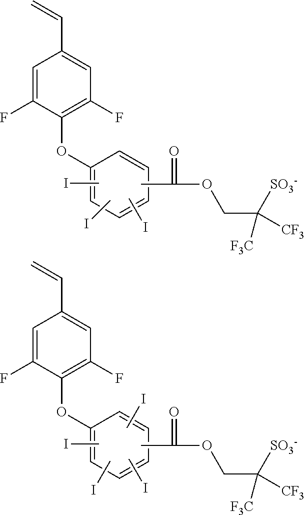

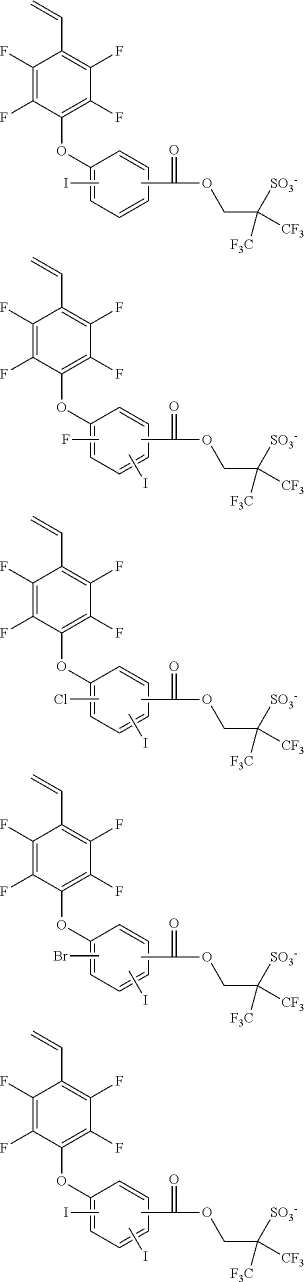

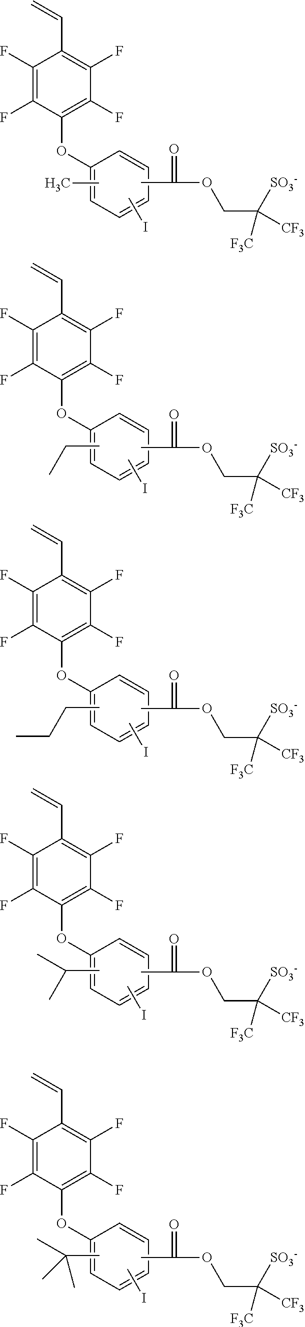

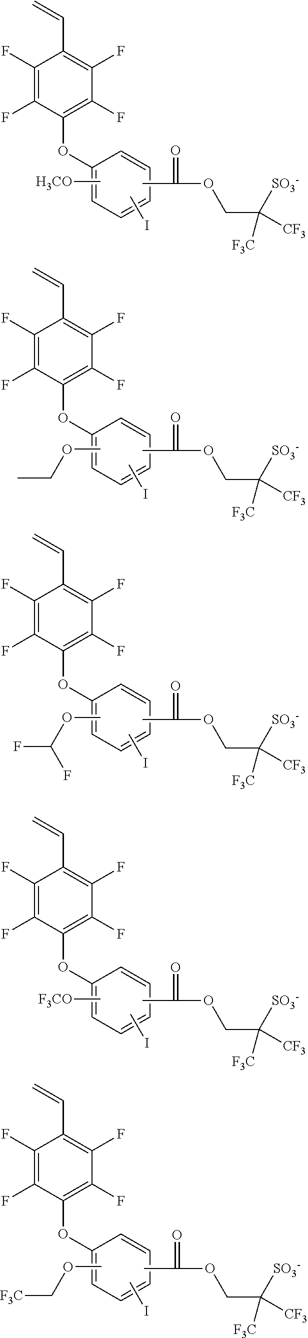

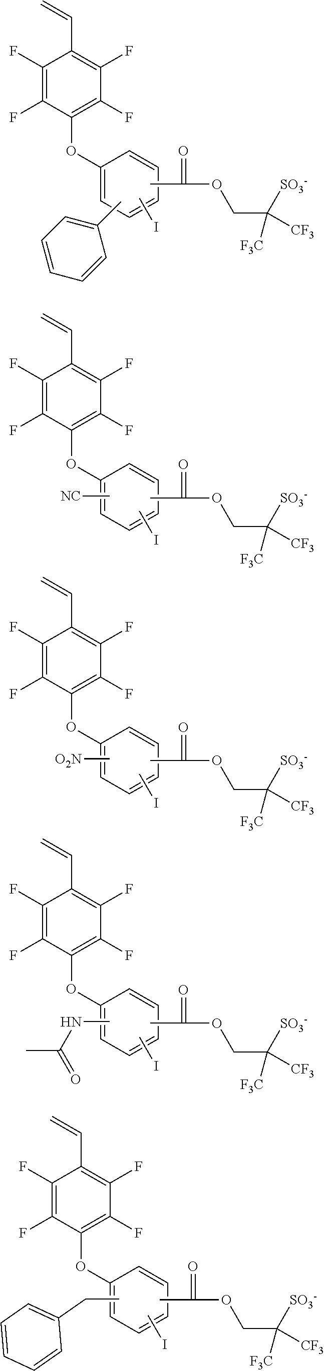

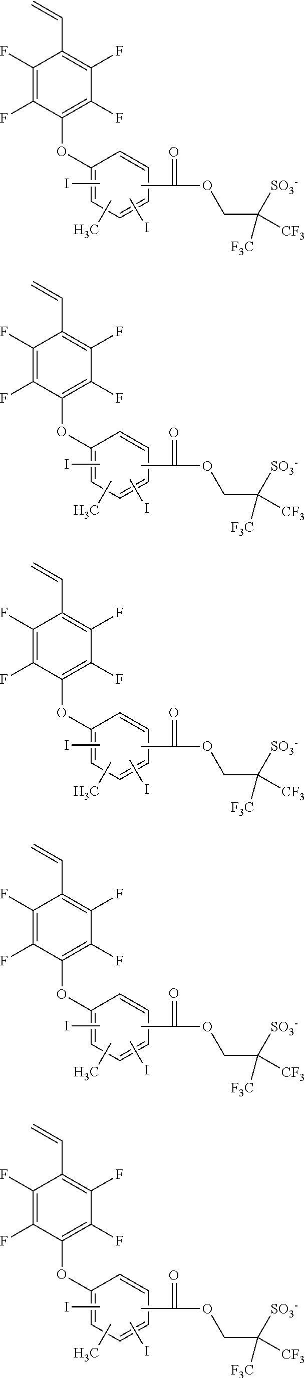

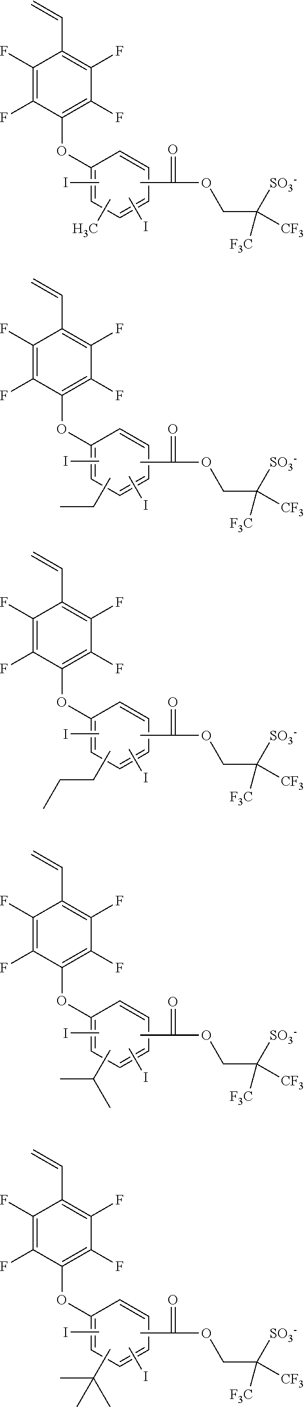

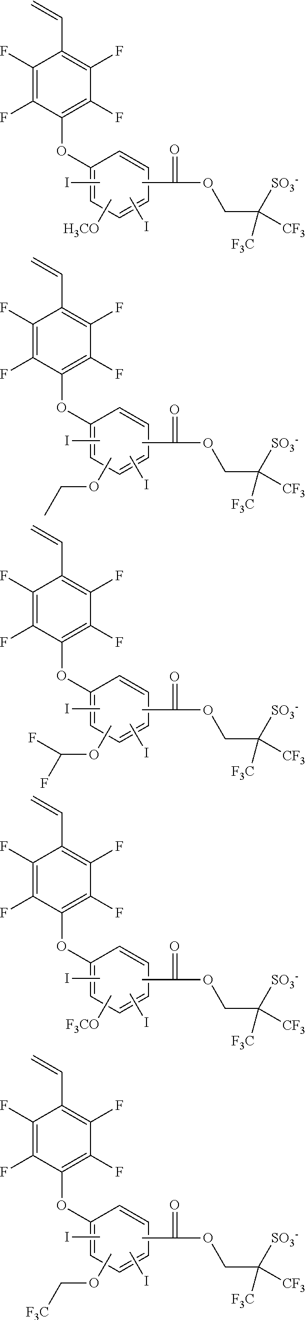

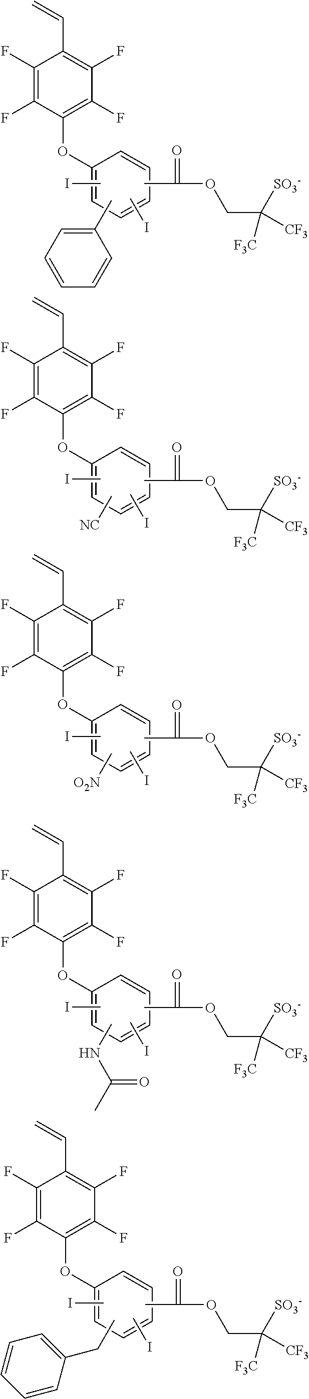

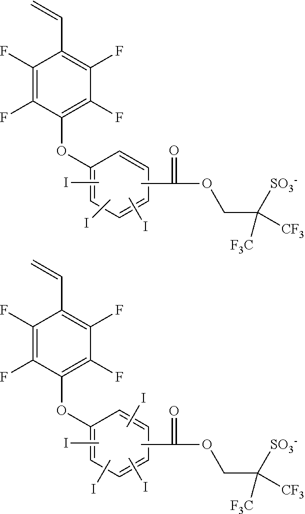

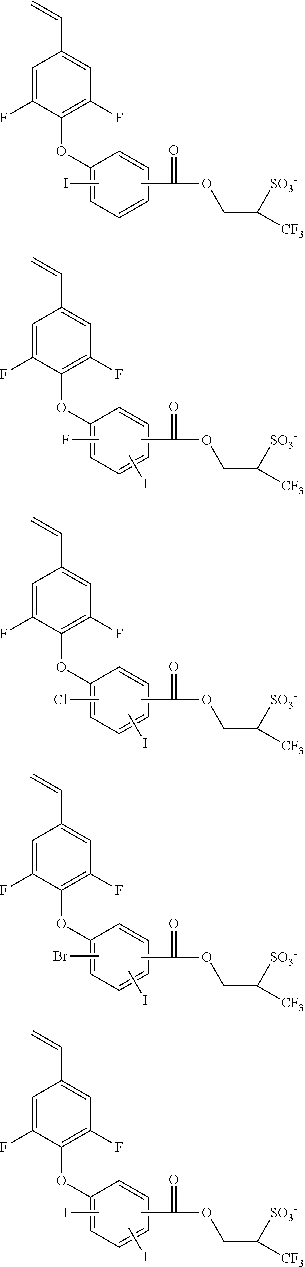

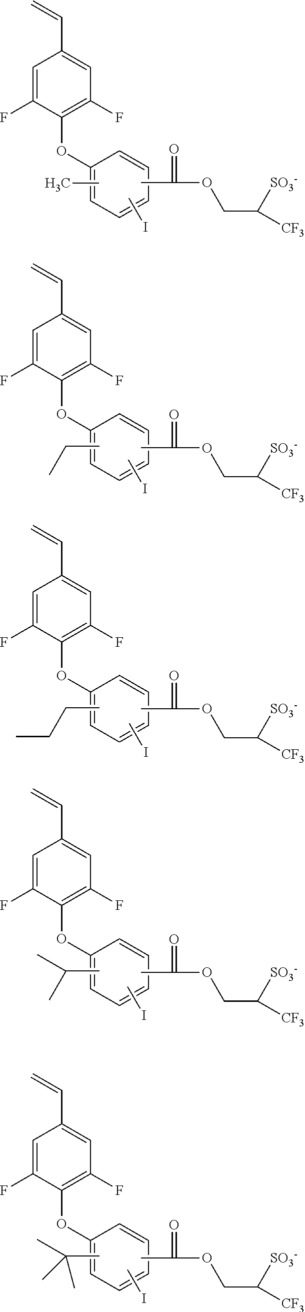

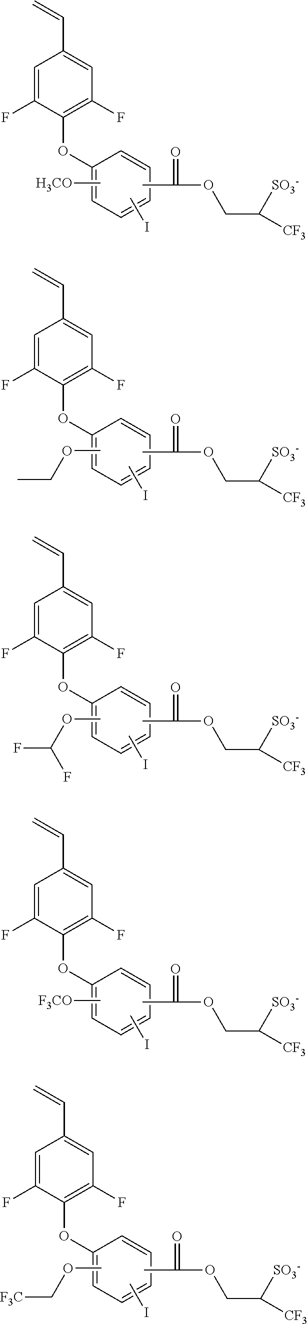

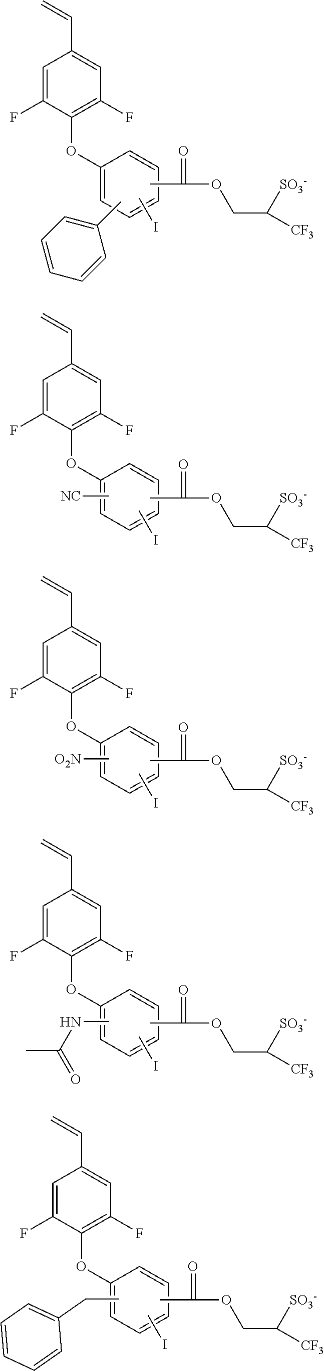

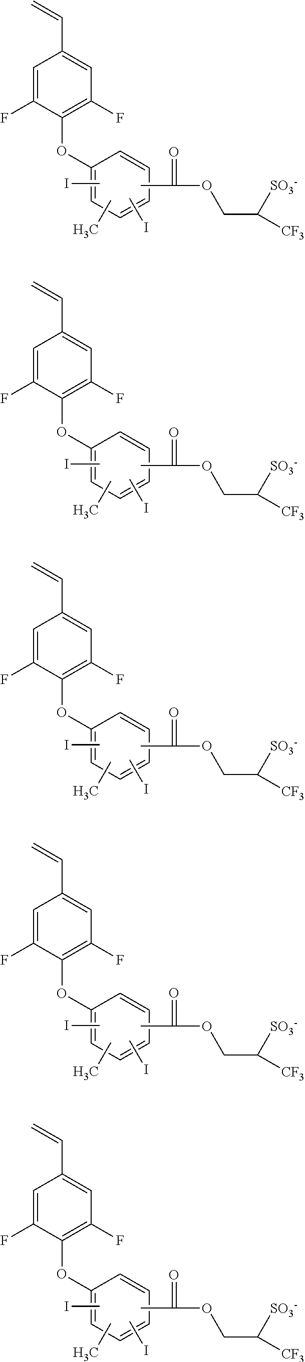

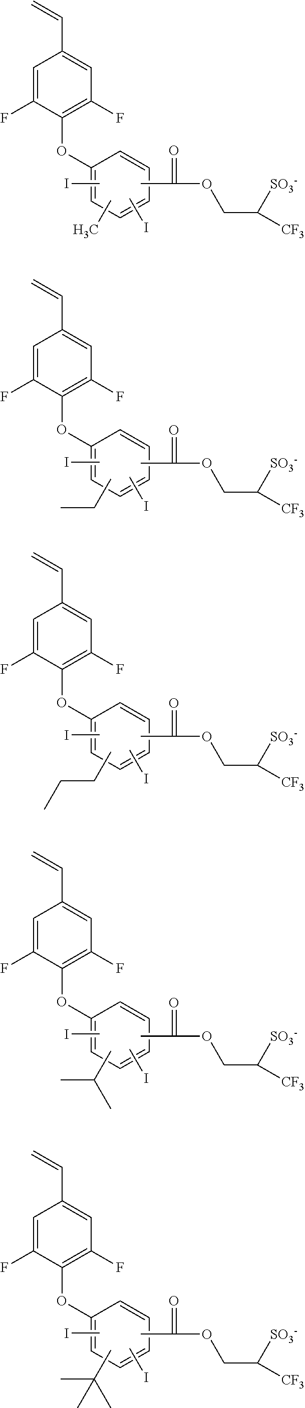

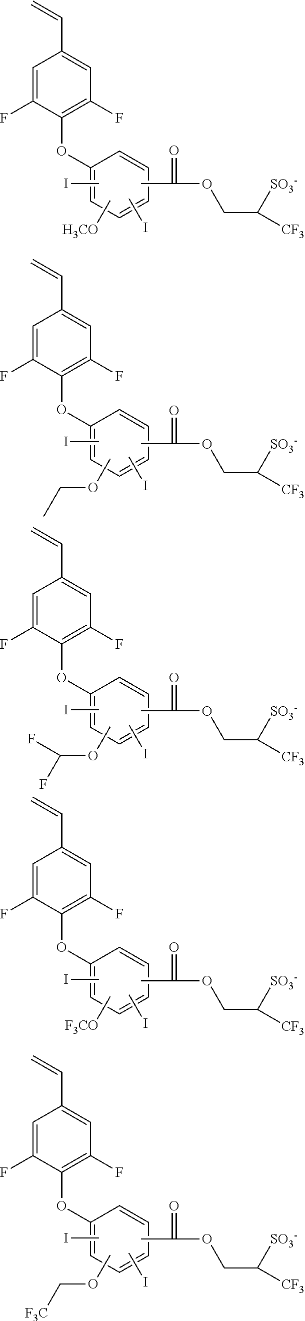

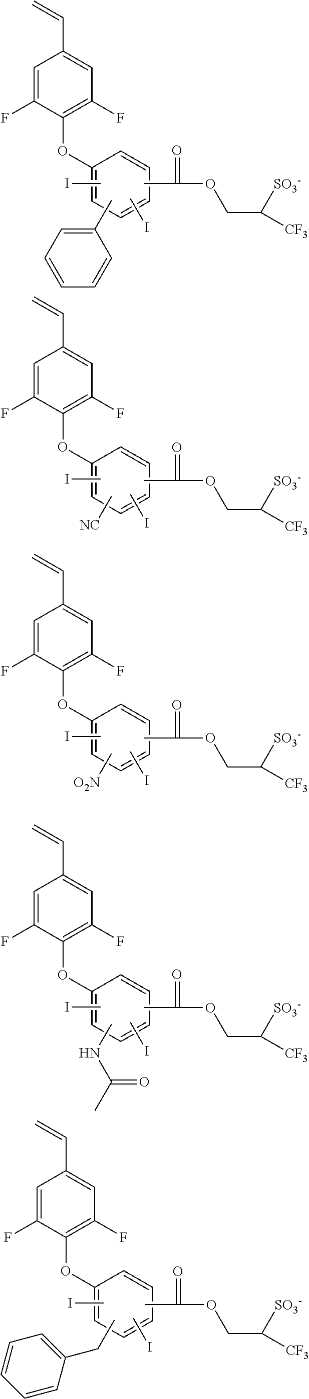

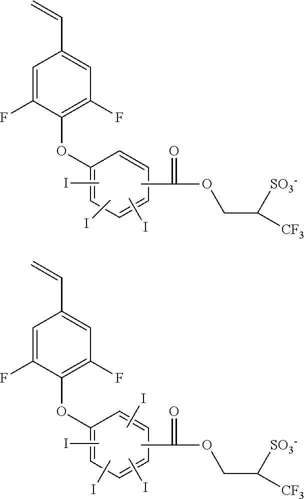

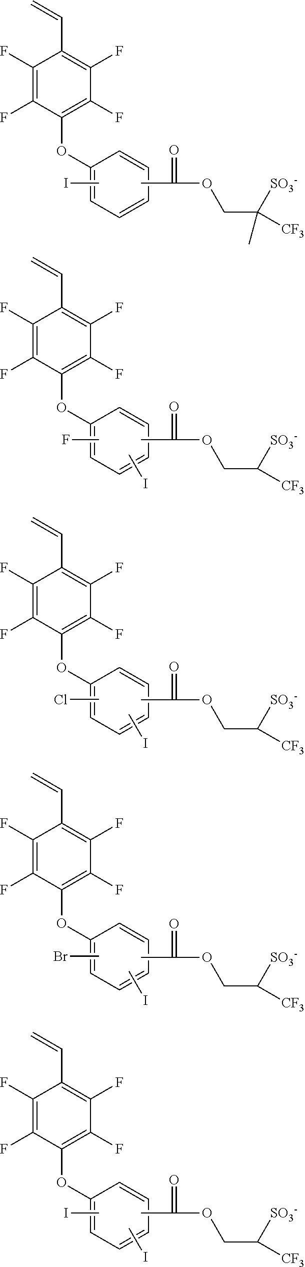

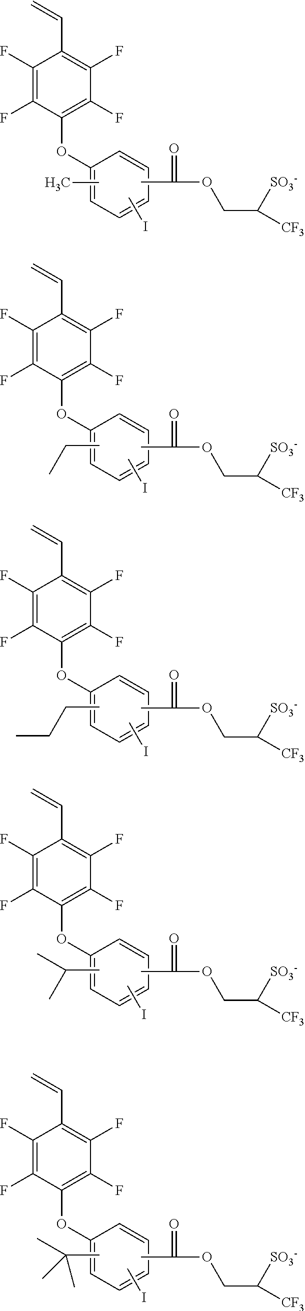

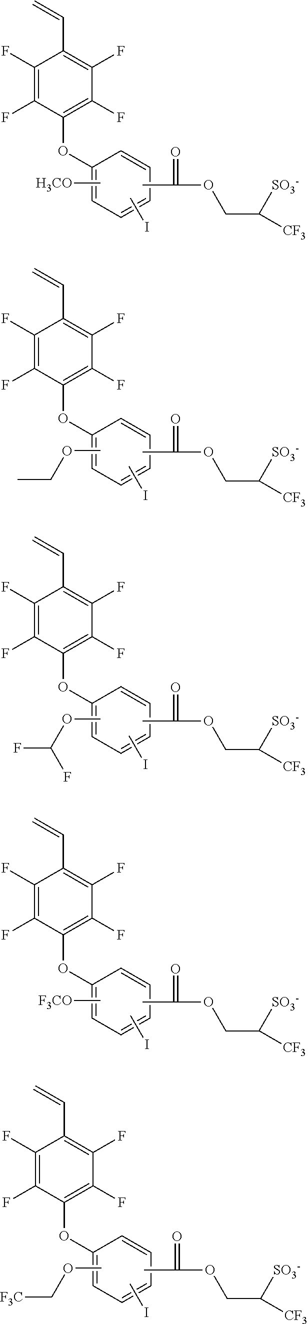

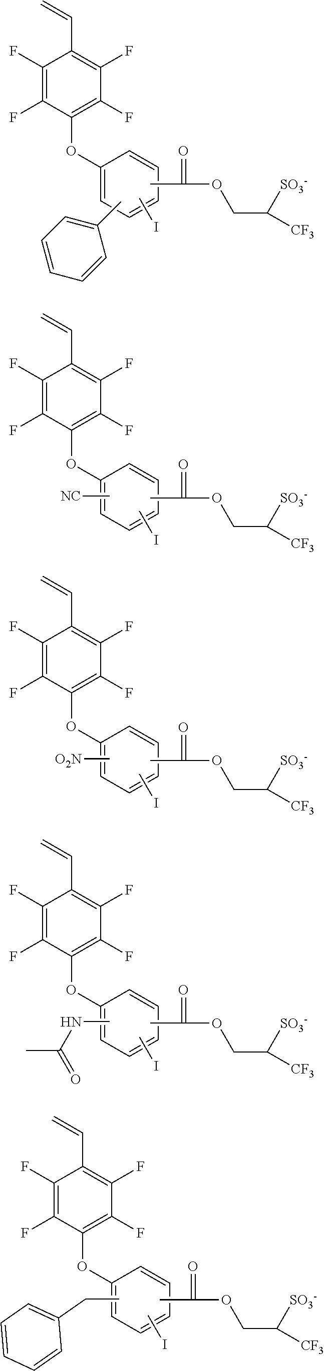

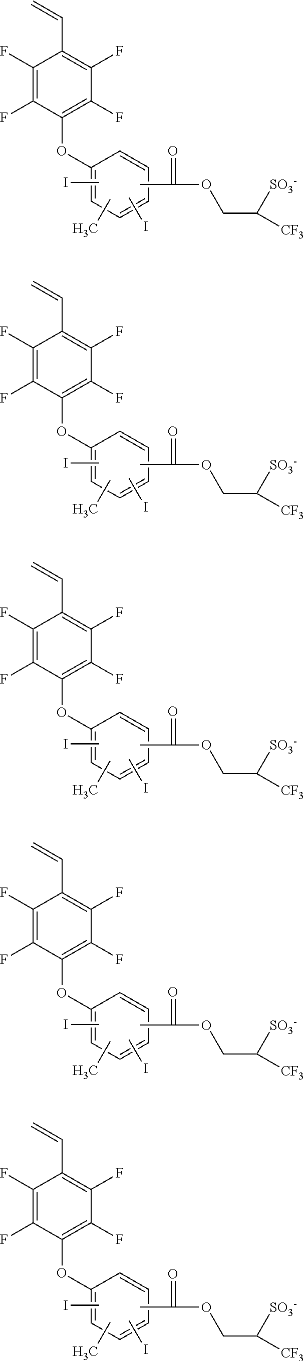

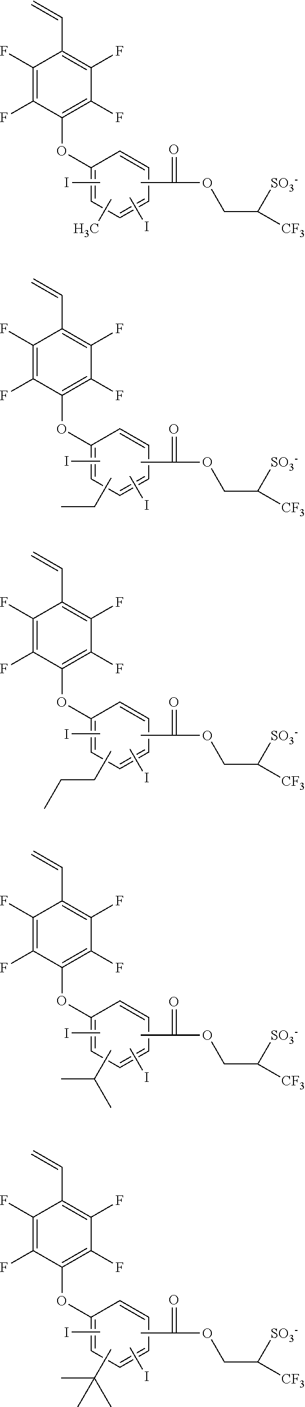

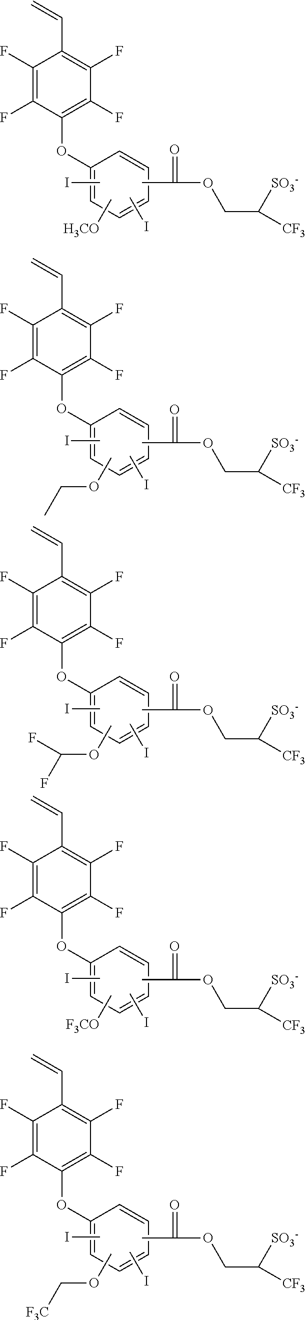

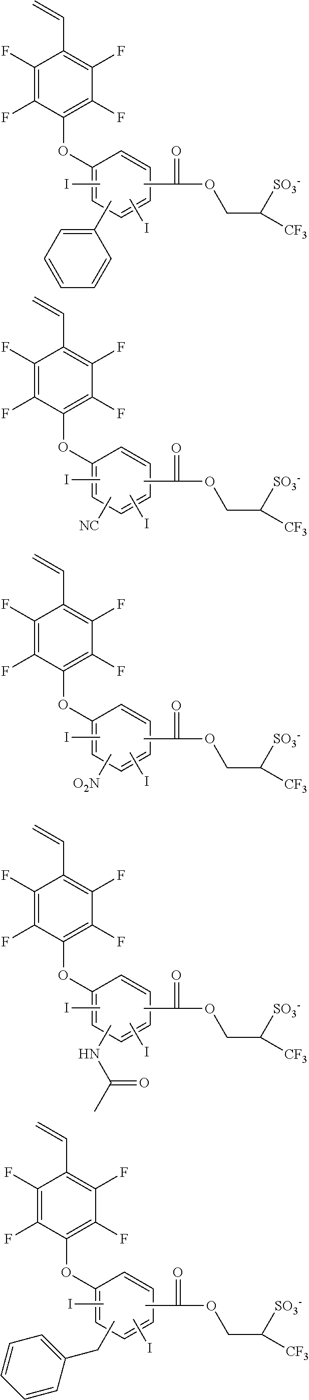

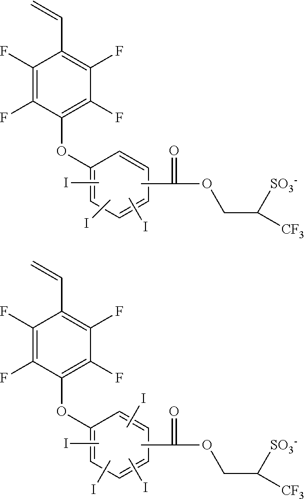

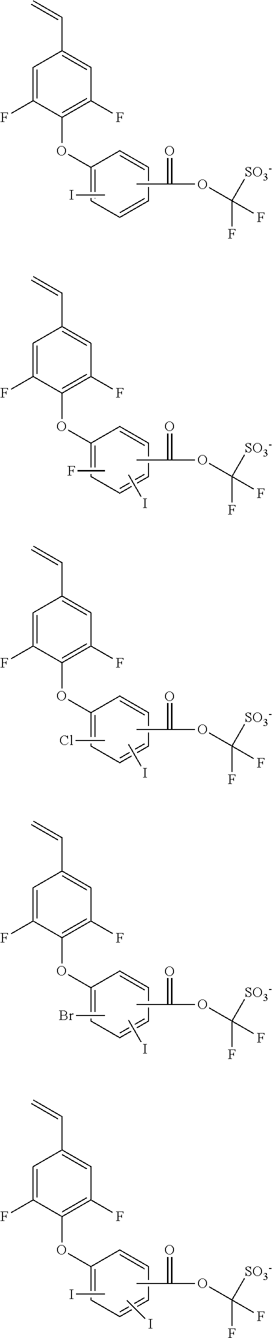

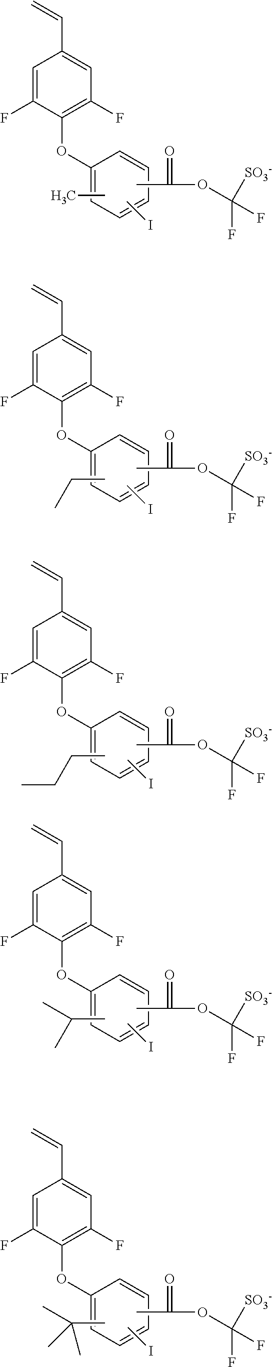

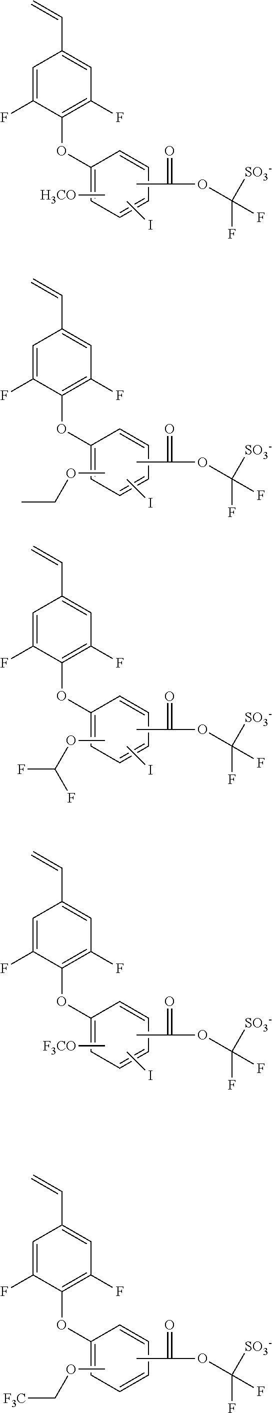

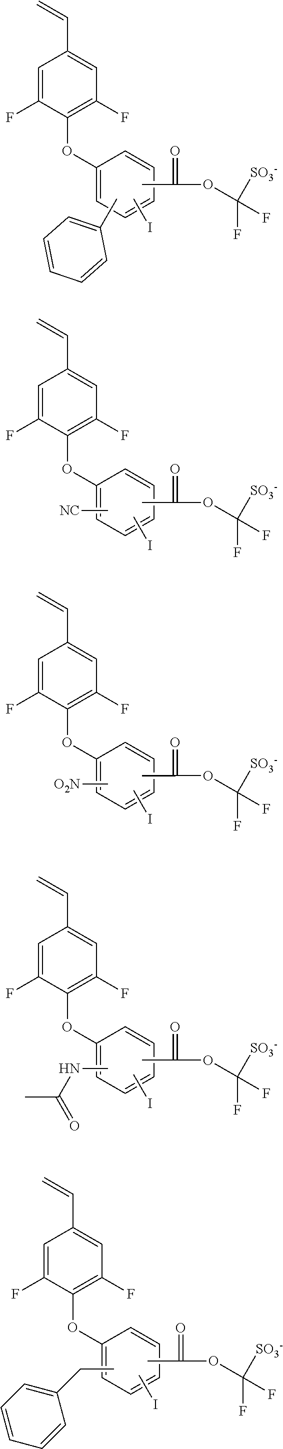

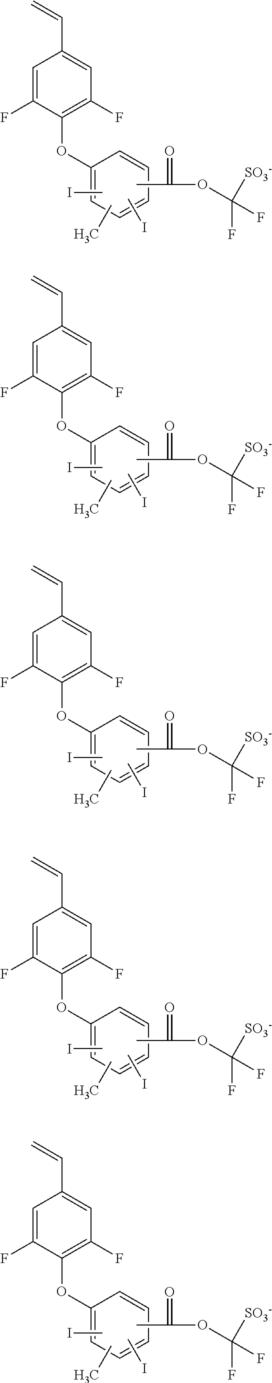

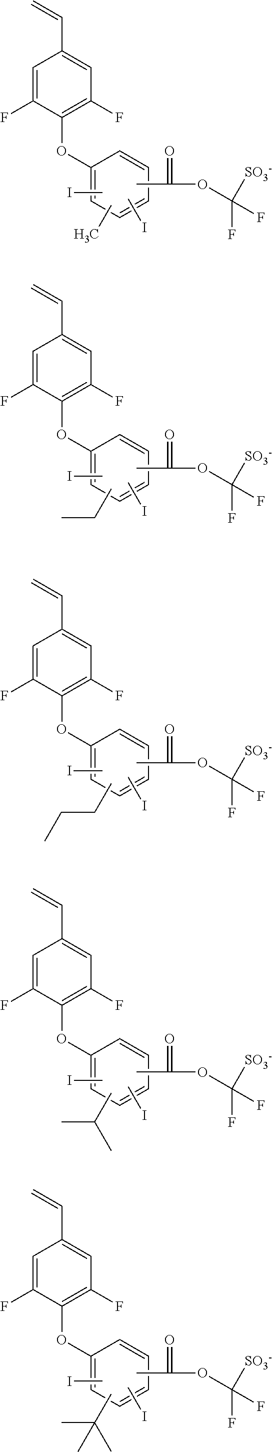

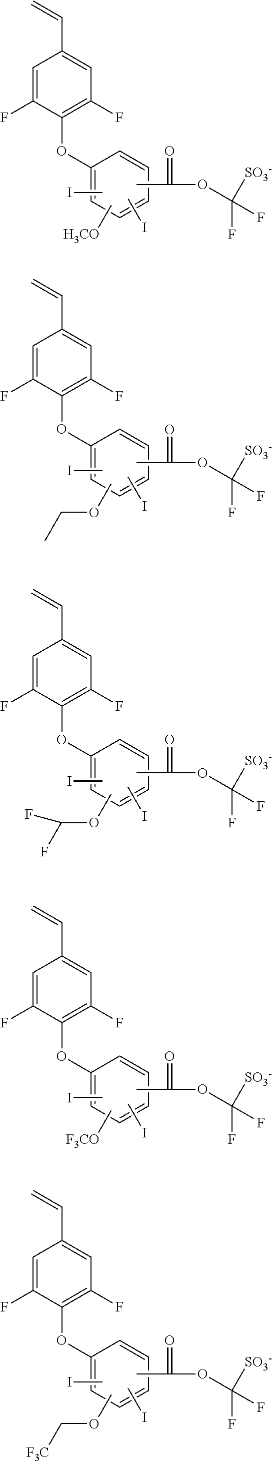

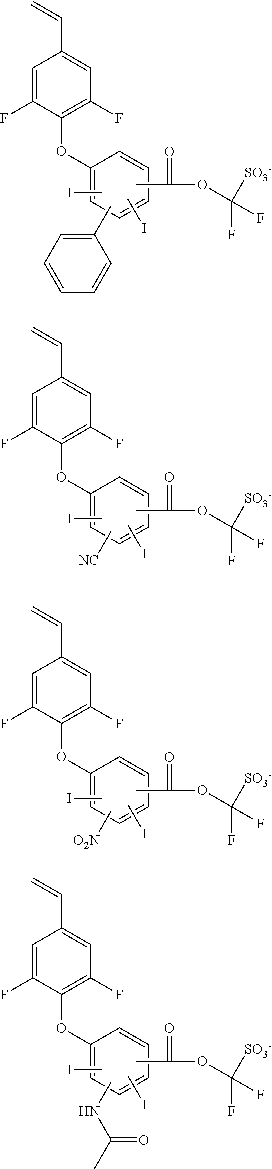

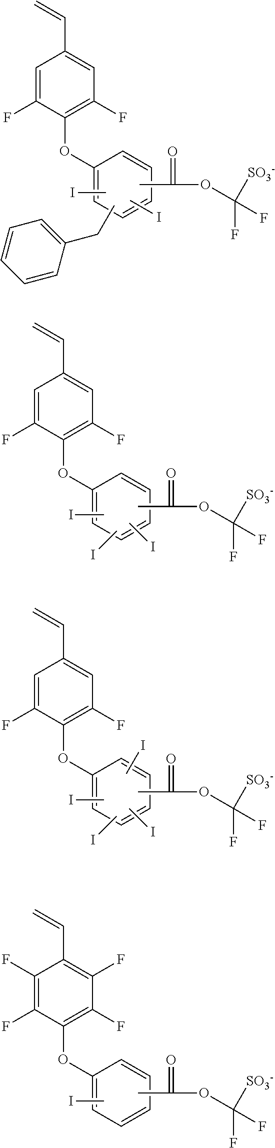

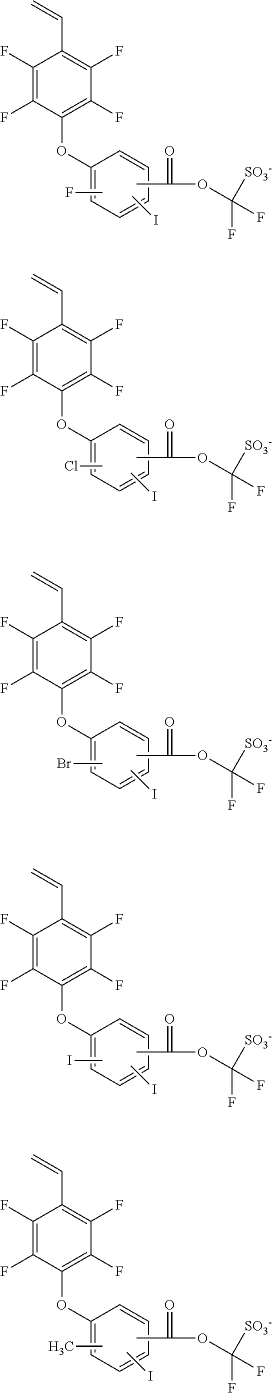

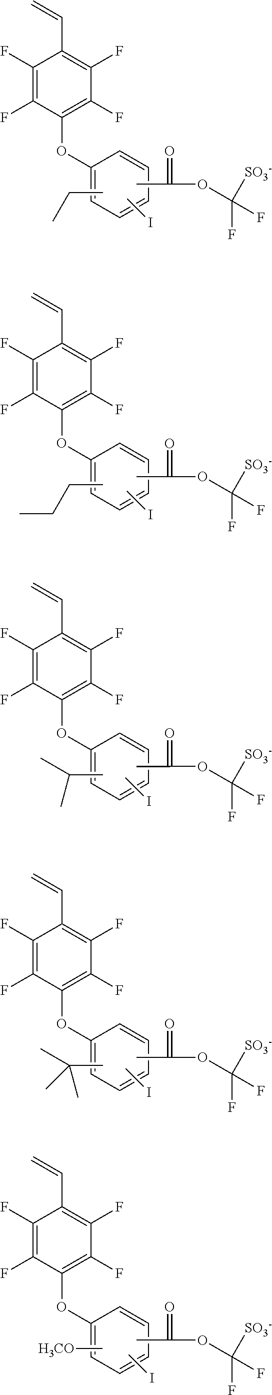

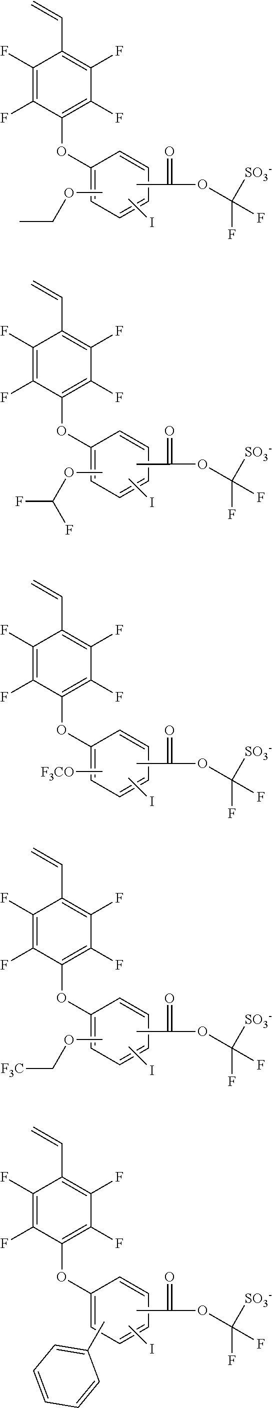

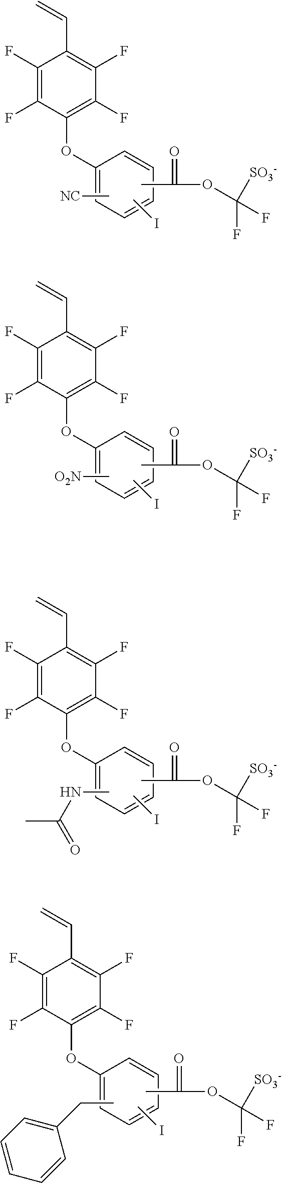

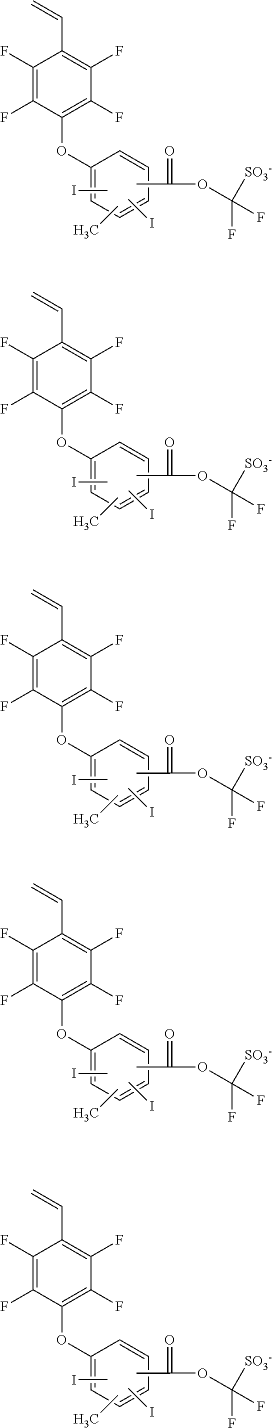

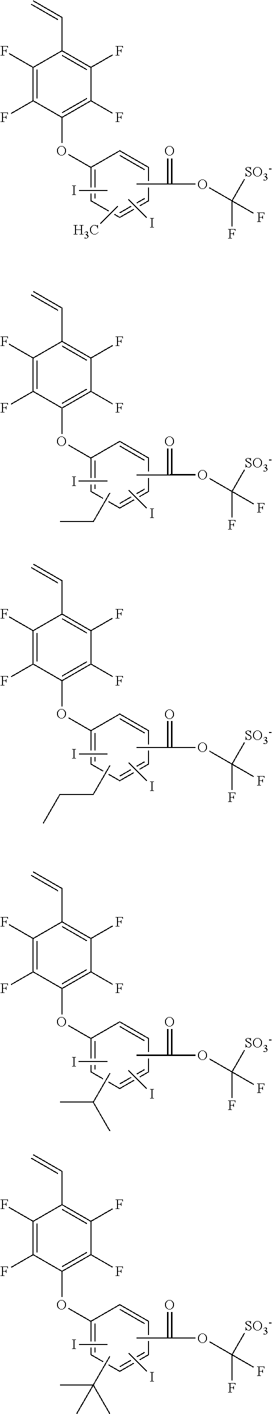

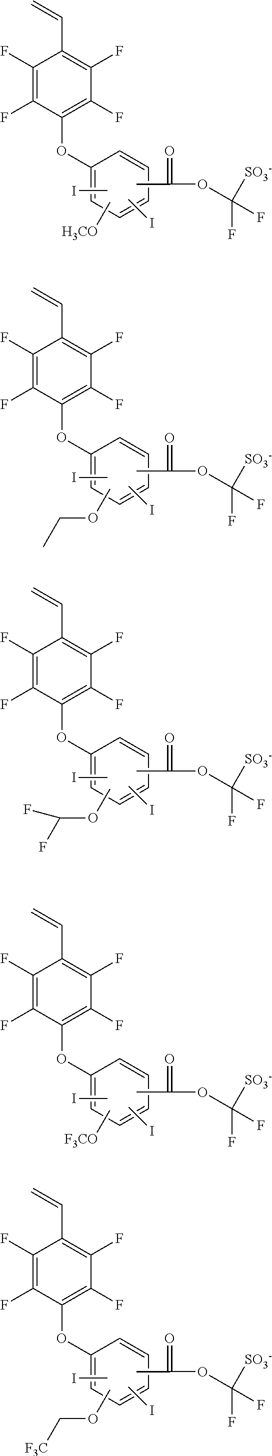

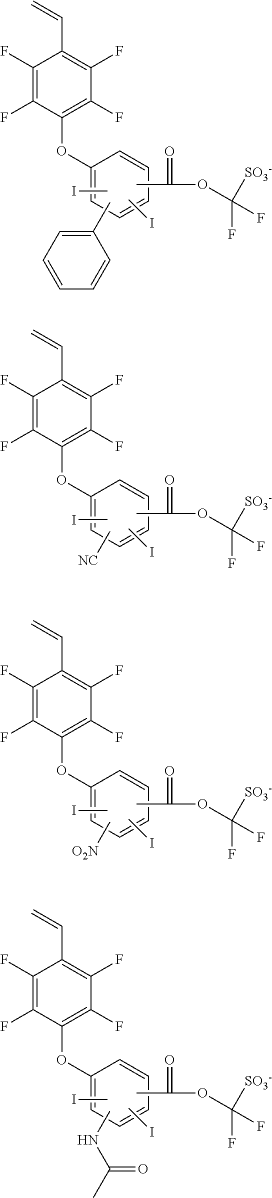

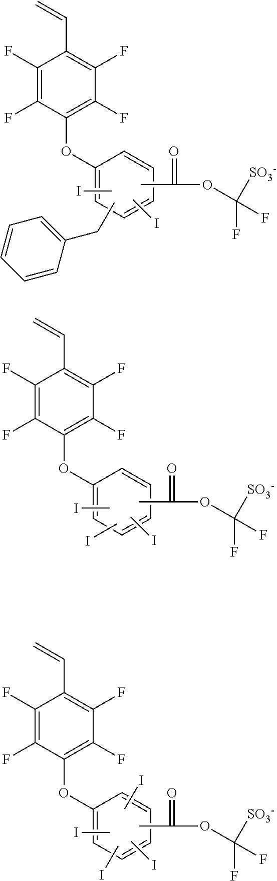

In the formula (a), Z⁺ is an onium cation. The onium cation is preferably a sulfonium cation represented by the following formula (cation-1) or an iodonium cation represented by the following formula (cation-2):

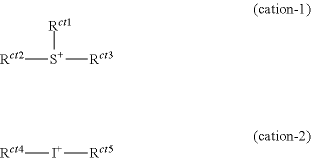

In the formulae (cation-1) and (cation-2), R^{ct1} to R^{ct5} are each independently a halogen atom or a hydrocarbyl group having 1 to 30 carbon atoms and optionally containing a heteroatom.

Specific examples of the halogen atom represented by any of R^{ct1} to R^{ct5} include a fluorine atom, a chlorine atom, a bromine atom, and an iodine atom.

The hydrocarbyl group represented by any of R^{ct1} to R^{ct5} may be saturated or unsaturated, and may be linear, branched, or cyclic. Specific examples thereof include an alkyl group having 1 to 30 carbon atoms such as a methyl group, an ethyl group, a n-propyl group, an isopropyl group, a n-butyl group, an isobutyl group, a sec-butyl group, and a tert-butyl group; a cyclic saturated hydrocarbyl group having 3 to 30 carbon atoms such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; an alkenyl group having 2 to 30 carbon atoms such as a vinyl group, an allyl group, a propenyl group, a butenyl group, and a hexenyl group; a cyclic unsaturated hydrocarbyl group having 3 to 30 carbon atoms such as a cyclohexenyl group; an aryl group having 6 to 30 carbon atoms such as a phenyl group, a naphthyl group, and a thienyl group; an aralkyl group having 7 to 30 carbon atoms such as a benzyl group, a 1-phenylethyl group, and a 2-phenylethyl group; and a group given by combining any of these, and an aryl group is preferred. Some or all of the hydrogen atoms of the hydrocarbyl group may be each replaced with a group containing a heteroatom such as an oxygen atom, a sulfur atom, a nitrogen atom, and a halogen atom, some of the —CH₂— moieties of the hydrocarbyl group may be each replaced with a group containing a heteroatom such as an oxygen atom, a sulfur atom, and a nitrogen atom, and as a result, for example, a hydroxy group, a fluorine atom, a chlorine atom, a bromine atom, an iodine atom, a cyano group, a nitro group, a carbonyl group, an ether bond, an ester bond, a sulfonic ester bond, a carbonate bond, a lactone ring, a sultone ring, a carboxylic anhydride (—C(═O)—O—C(═O)—), or a haloalkyl group may be contained.

Further, R^{ct1} and R^{ct2} are optionally bound together to form a ring together with the sulfur atom to which the two groups are bound. Specific examples of the structure of the ring in this case include those represented by the following formulae:

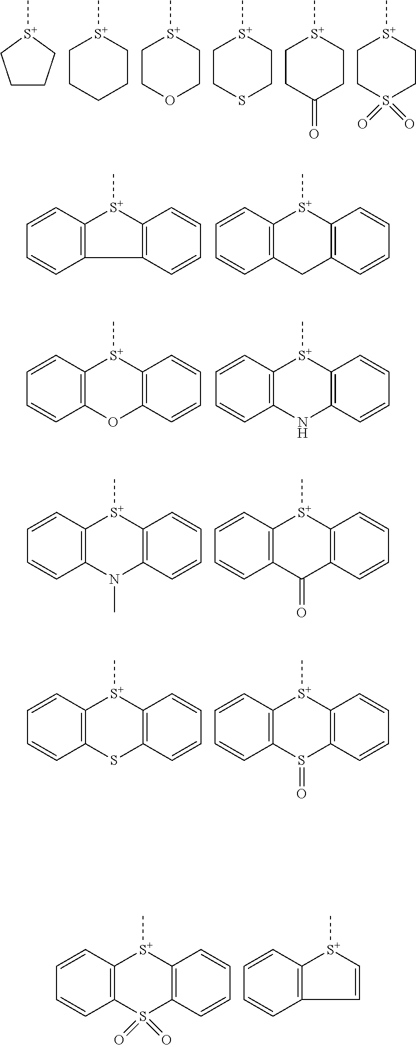

wherein each dashed line indicates bonding to R^{ct3}.

Specific examples of the sulfonium cation represented by the formula (cation-1) include, but are not limited to, those shown in the following:

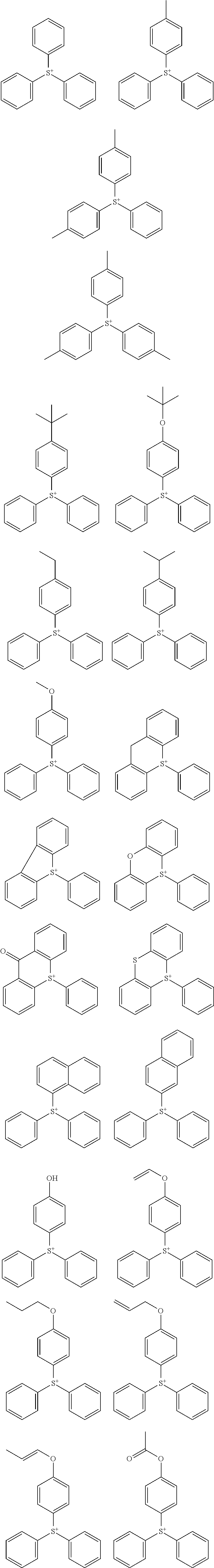

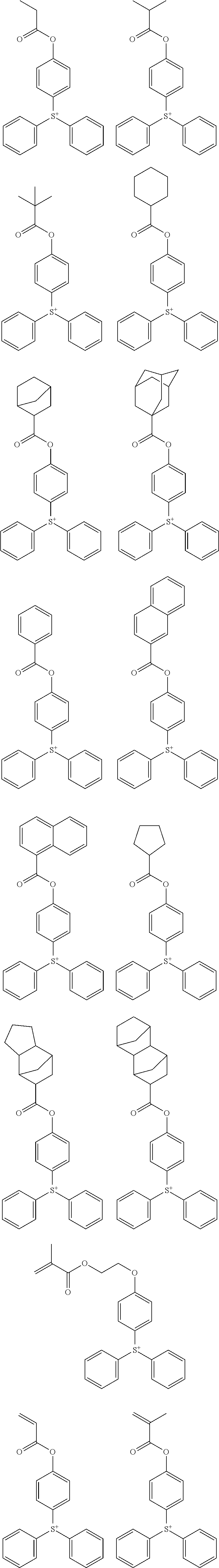

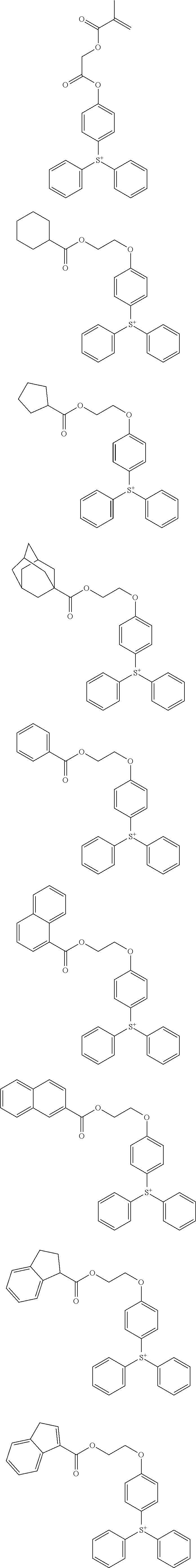

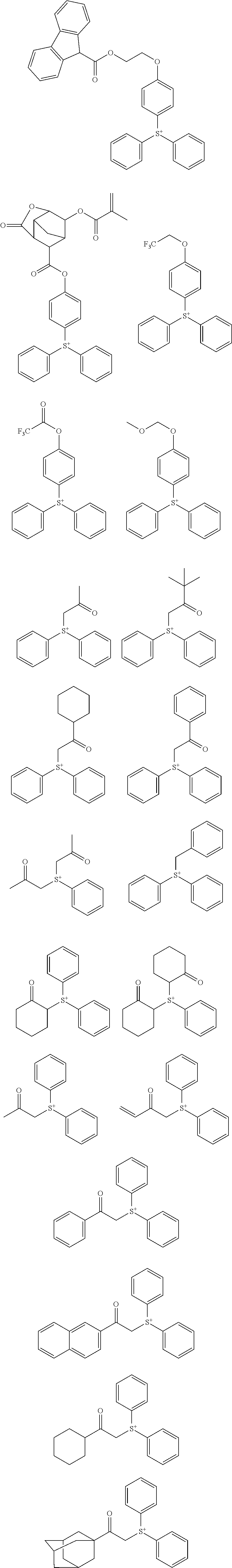

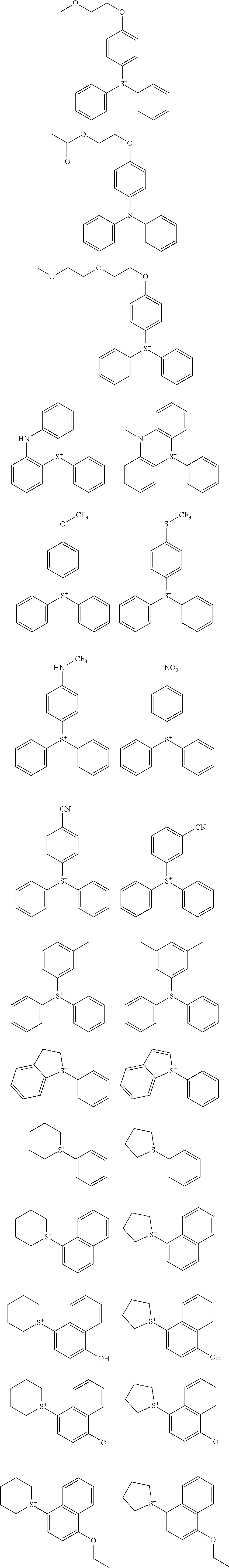

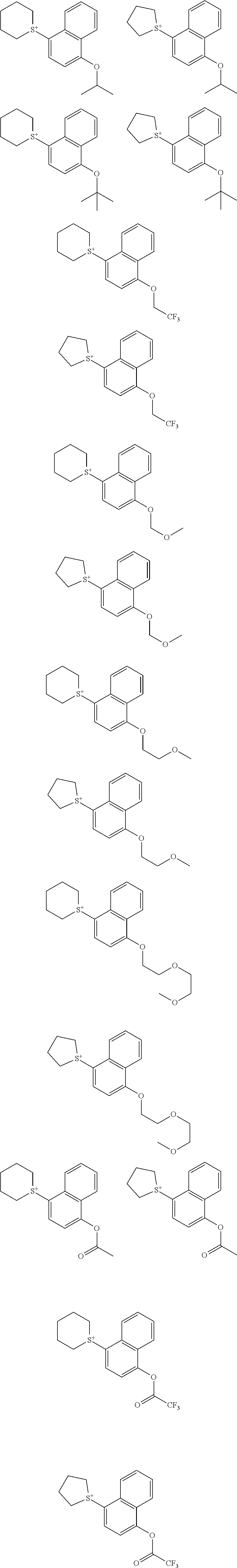

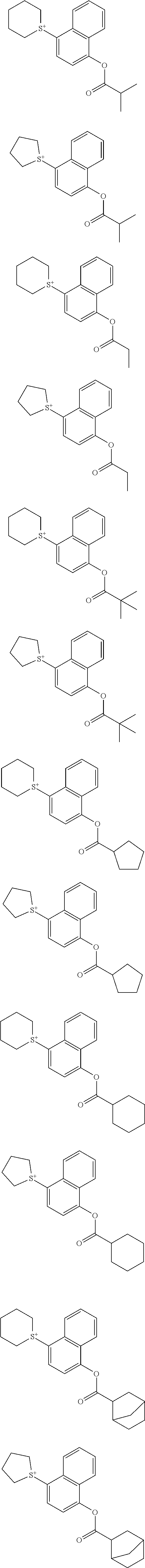

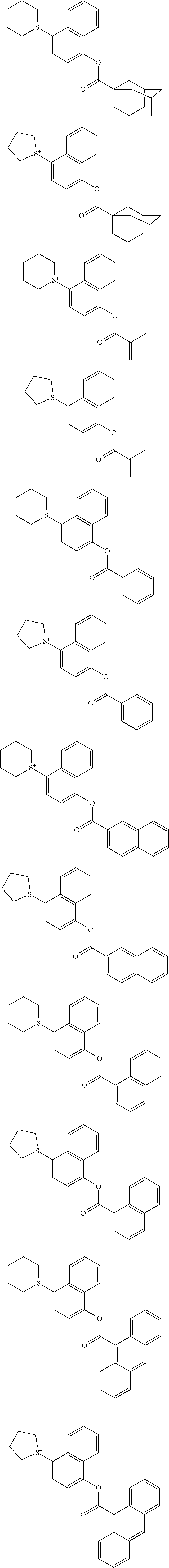

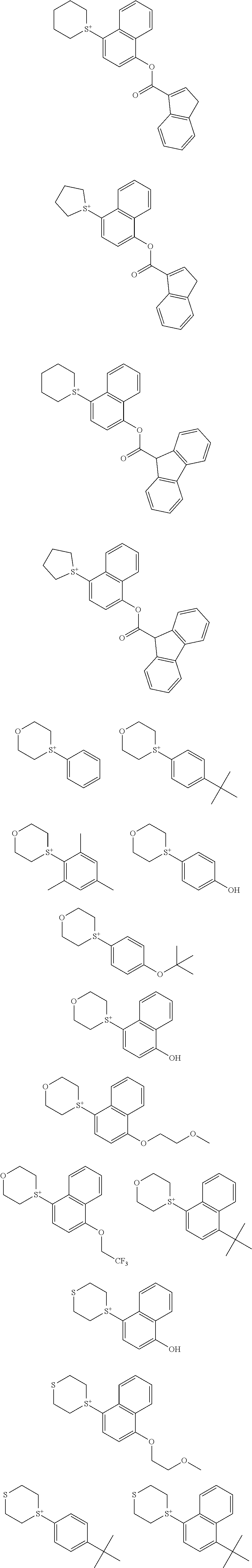

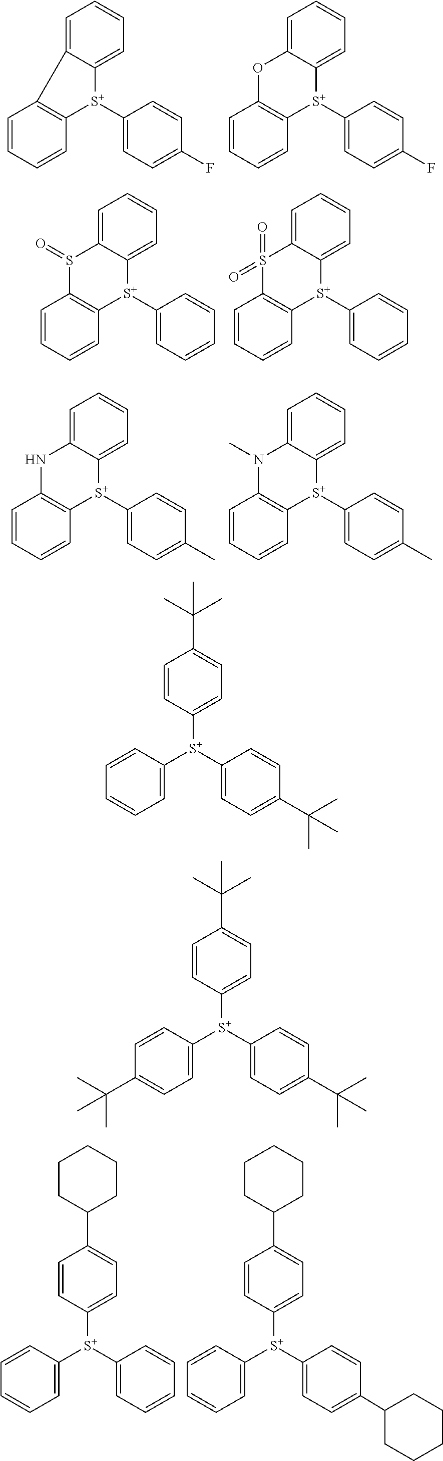

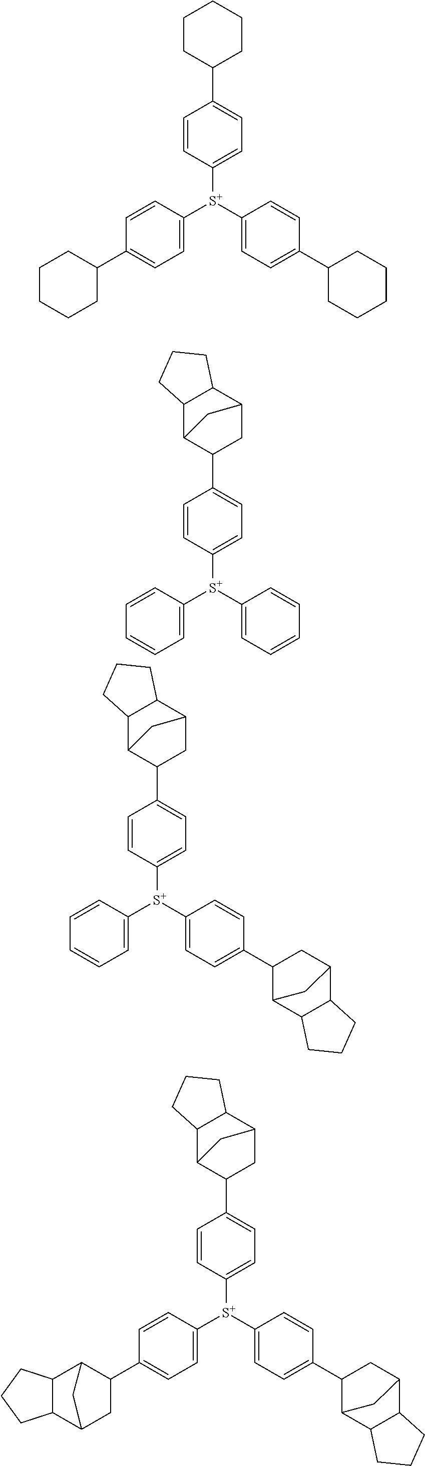 [SMILES: c1cc([S+](c2ccc(C3CCCCC3)cc2)c2ccc(C3CCCCC3)cc2)ccc1C1CCCCC1 ; c1ccc([S+](c2ccccc2)c2ccc(C3CC4CC3C3CCCC43)cc2)cc1 ; c1ccc([S+](c2ccc(C3CC4CC3C3CCCC43)cc2)c2ccc(C3CC4CC3C3CCCC43)cc2)cc1 ; c1cc([S+](c2ccc(C3CC4CC3C3CCCC43)cc2)c2ccc(C3CC4CC3C3CCCC43)cc2)ccc1C1CC2CC1C1CCCC21]

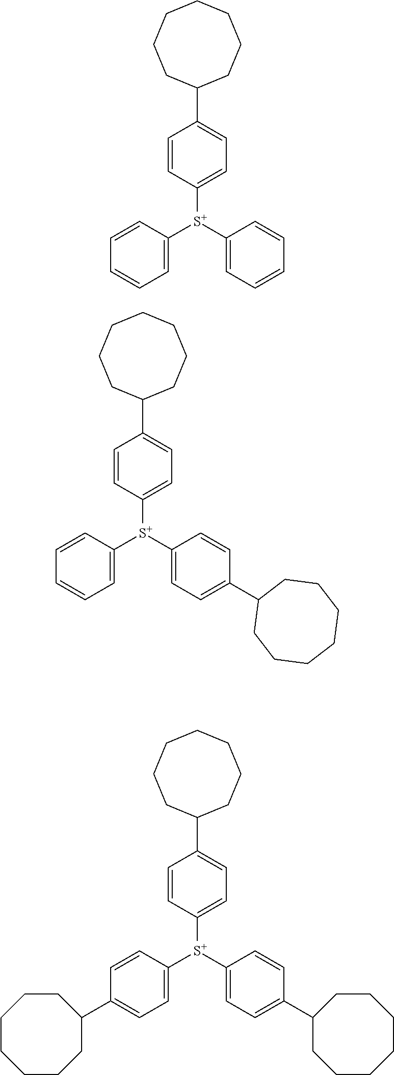 [SMILES: c1ccc([S+](c2ccccc2)c2ccc(C3CCCCCCC3)cc2)cc1 ; c1ccc([S+](c2ccc(C3CCCCCCC3)cc2)c2ccc(C3CCCCCCC3)cc2)cc1 ; c1cc([S+](c2ccc(C3CCCCCCC3)cc2)c2ccc(C3CCCCCCC3)cc2)ccc1C1CCCCCCC1]

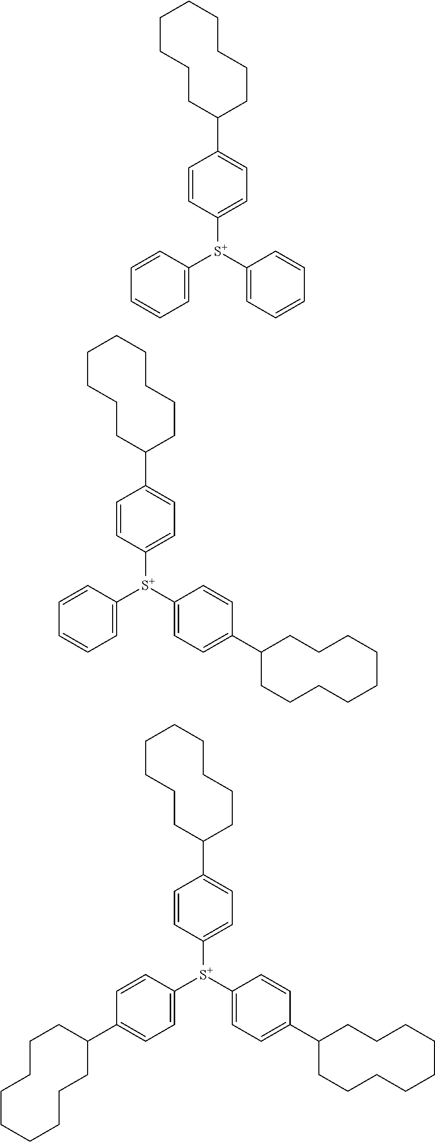 [SMILES: c1ccc([S+](c2ccccc2)c2ccc(C3CCCCCCCCC3)cc2)cc1 ; c1ccc([S+](c2ccc(C3CCCCCCCCC3)cc2)c2ccc(C3CCCCCCCCC3)cc2)cc1 ; c1cc([S+](c2ccc(C3CCCCCCCCC3)cc2)c2ccc(C3CCCCCCCCC3)cc2)ccc1C1CCCCCCCCC1]

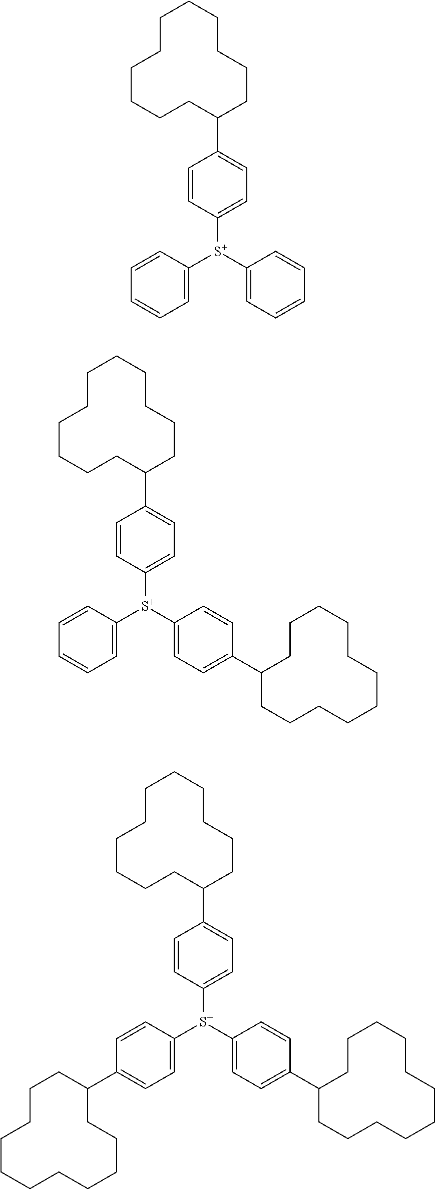 [SMILES: c1ccc([S+](c2ccccc2)c2ccc(C3CCCCCCCCCCC3)cc2)cc1 ; c1ccc([S+](c2ccc(C3CCCCCCCCCCC3)cc2)c2ccc(C3CCCCCCCCCCC3)cc2)cc1 ; c1cc([S+](c2ccc(C3CCCCCCCCCCC3)cc2)c2ccc(C3CCCCCCCCCCC3)cc2)ccc1C1CCCCCCCCCCC1]

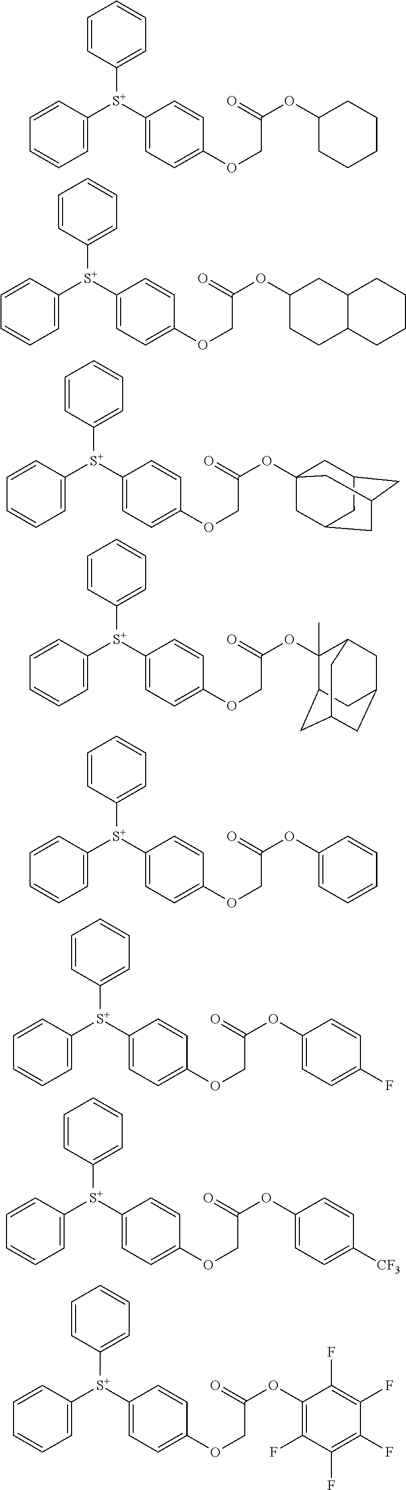

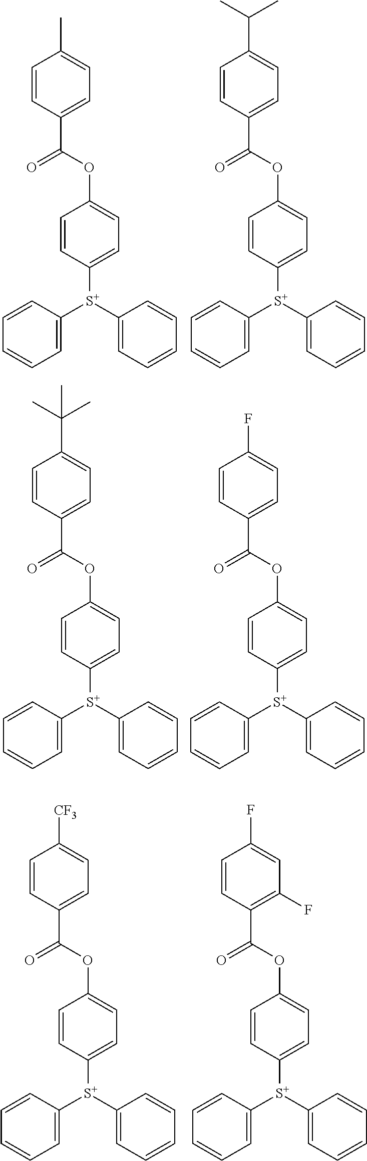 [SMILES: Cc1ccc(C(=O)Oc2ccc([S+](c3ccccc3)c3ccccc3)cc2)cc1 ; CC(C)c1ccc(C(=O)Oc2ccc([S+](c3ccccc3)c3ccccc3)cc2)cc1 ; CC(C)(C)c1ccc(C(=O)Oc2ccc([S+](c3ccccc3)c3ccccc3)cc2)cc1 ; O=C(Oc1ccc([S+](c2ccccc2)c2ccccc2)cc1)c1ccc(F)cc1 ; O=C(Oc1ccc([S+](c2ccccc2)c2ccccc2)cc1)c1ccc(C(F)(F)F)cc1 ; O=C(Oc1ccc([S+](c2ccccc2)c2ccccc2)cc1)c1ccc(F)cc1F]

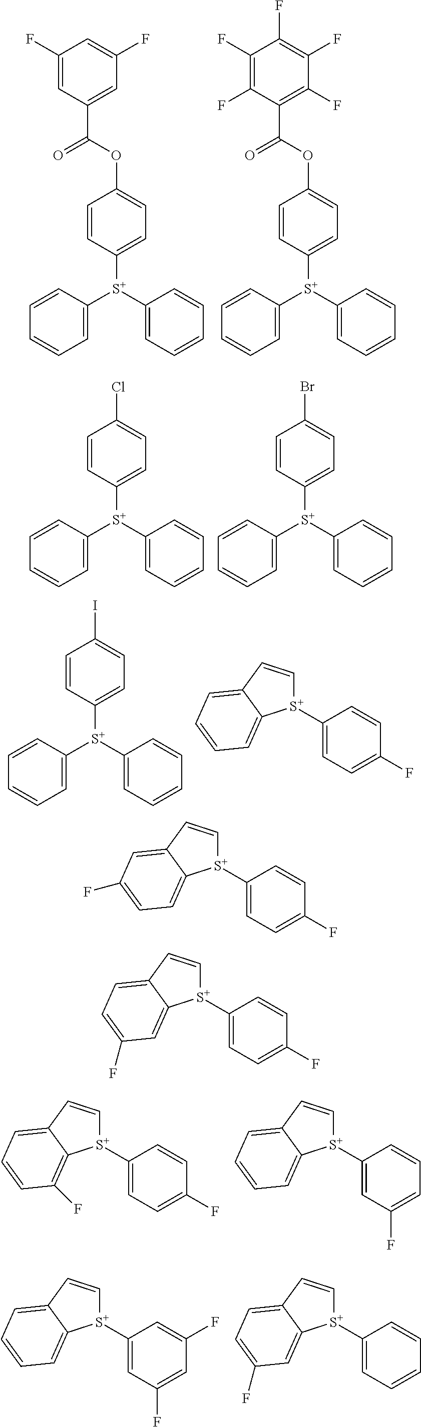

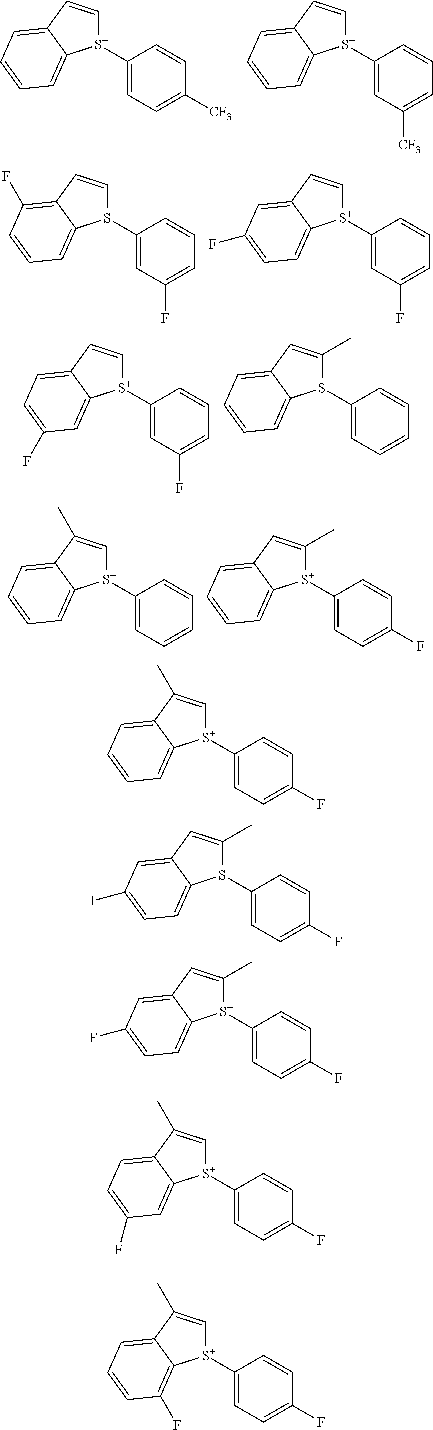

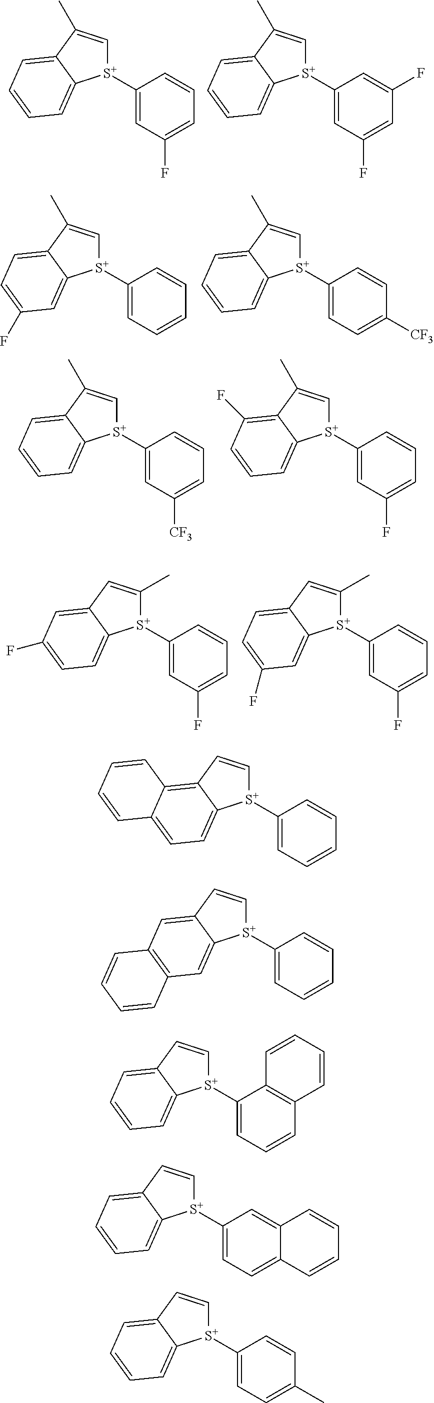

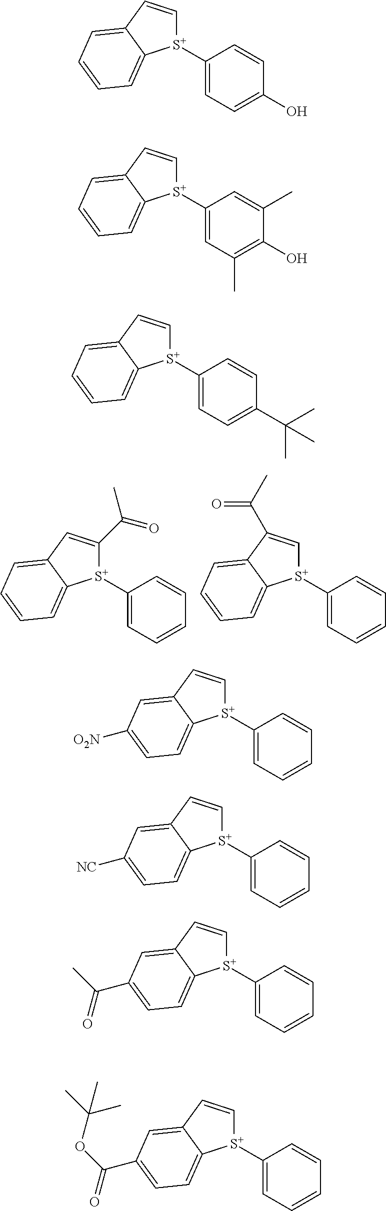

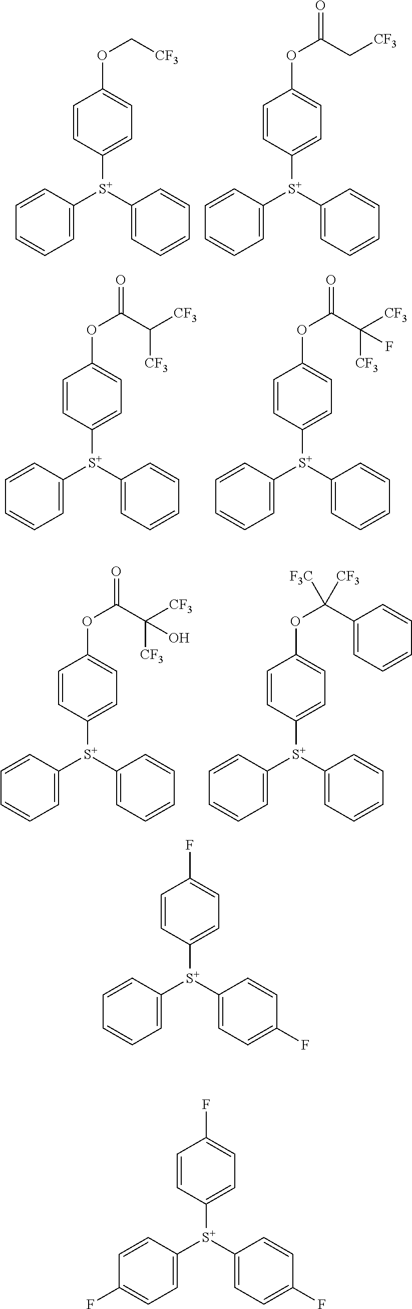

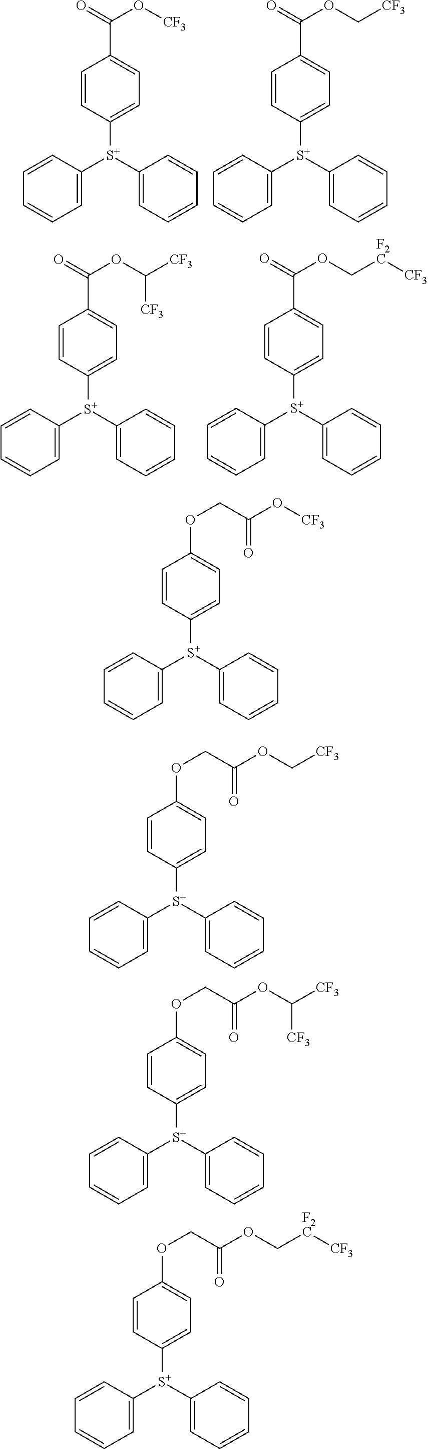

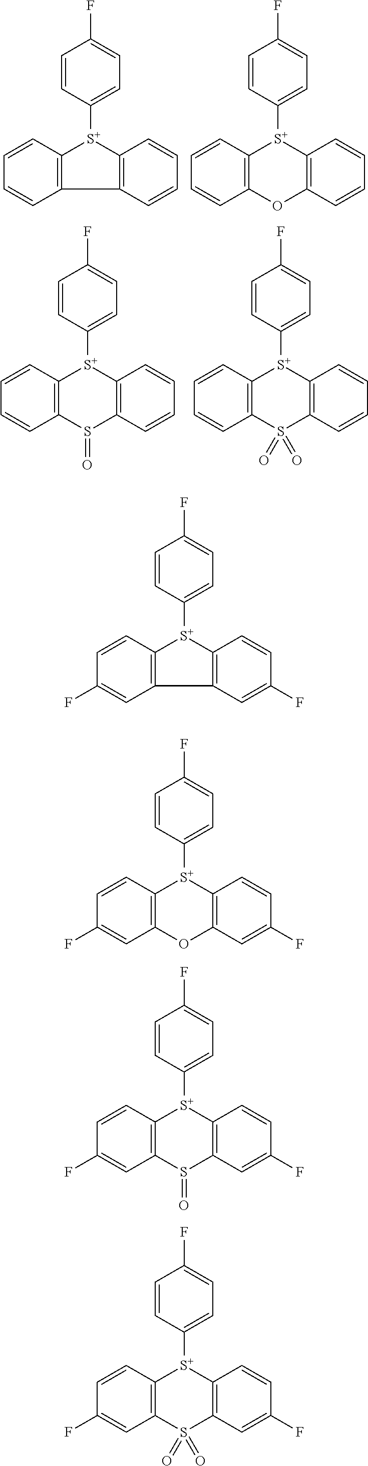

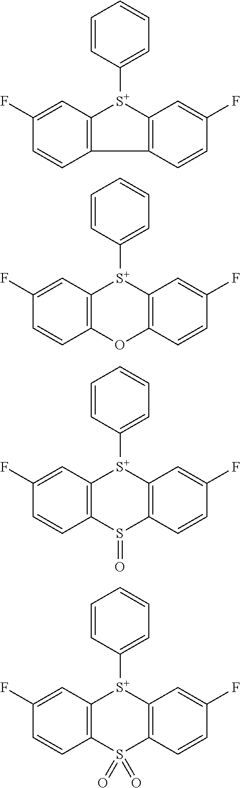 [SMILES: Fc1ccc2c3ccc(F)cc3[s+](-c3ccccc3)c2c1 ; Fc1ccc2c(c1)[S+](c1ccccc1)c1cc(F)ccc1O2 ; O=S1c2ccc(F)cc2[S+](c2ccccc2)c2cc(F)ccc21 ; O=S1(=O)c2ccc(F)cc2[S+](c2ccccc2)c2cc(F)ccc21]

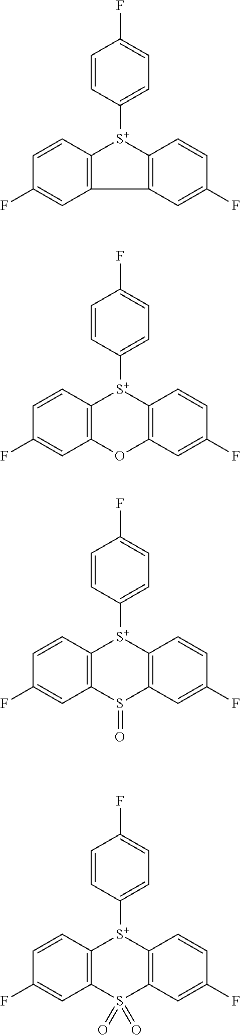 [SMILES: Fc1ccc(-[s+]2c3ccc(F)cc3c3cc(F)ccc32)cc1 ; Fc1ccc([S+]2c3ccc(F)cc3Oc3cc(F)ccc32)cc1 ; O=S1c2cc(F)ccc2[S+](c2ccc(F)cc2)c2ccc(F)cc21 ; O=S1(=O)c2cc(F)ccc2[S+](c2ccc(F)cc2)c2ccc(F)cc21]

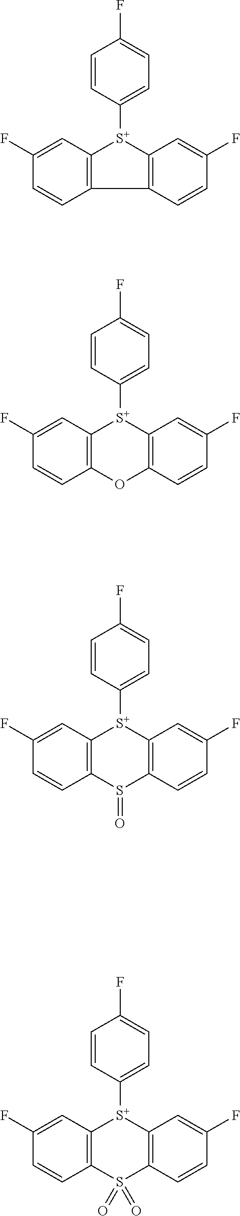 [SMILES: Fc1ccc(-[s+]2c3cc(F)ccc3c3ccc(F)cc32)cc1 ; Fc1ccc([S+]2c3cc(F)ccc3Oc3ccc(F)cc32)cc1 ; O=S1c2ccc(F)cc2[S+](c2ccc(F)cc2)c2cc(F)ccc21 ; O=S1(=O)c2ccc(F)cc2[S+](c2ccc(F)cc2)c2cc(F)ccc21]

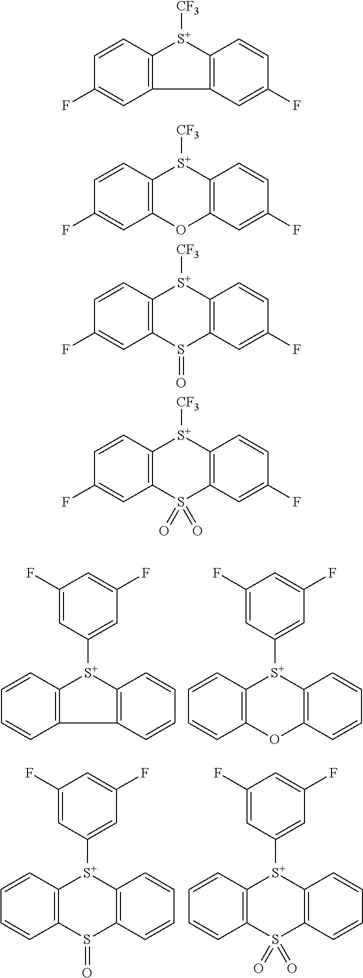

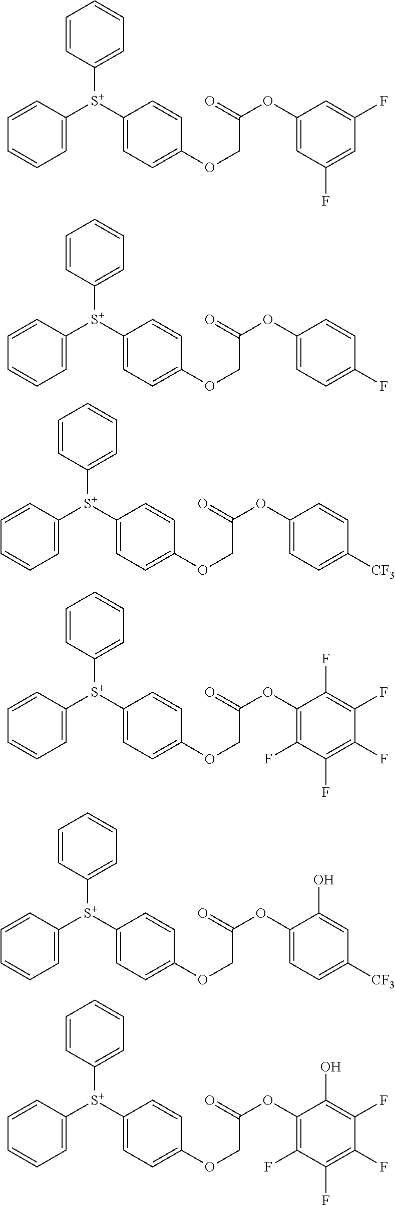 [SMILES: O=C(COc1ccc([S+](c2ccccc2)c2ccccc2)cc1)Oc1cc(F)cc(F)c1 ; O=C(COc1ccc([S+](c2ccccc2)c2ccccc2)cc1)Oc1ccc(F)cc1 ; O=C(COc1ccc([S+](c2ccccc2)c2ccccc2)cc1)Oc1ccc(C(F)(F)F)cc1 ; O=C(COc1ccc([S+](c2ccccc2)c2ccccc2)cc1)Oc1c(F)c(F)c(F)c(F)c1F ; O=C(COc1ccc([S+](c2ccccc2)c2ccccc2)cc1)Oc1ccc(C(F)(F)F)cc1O ; O=C(COc1ccc([S+](c2ccccc2)c2ccccc2)cc1)Oc1c(O)c(F)c(F)c(F)c1F]

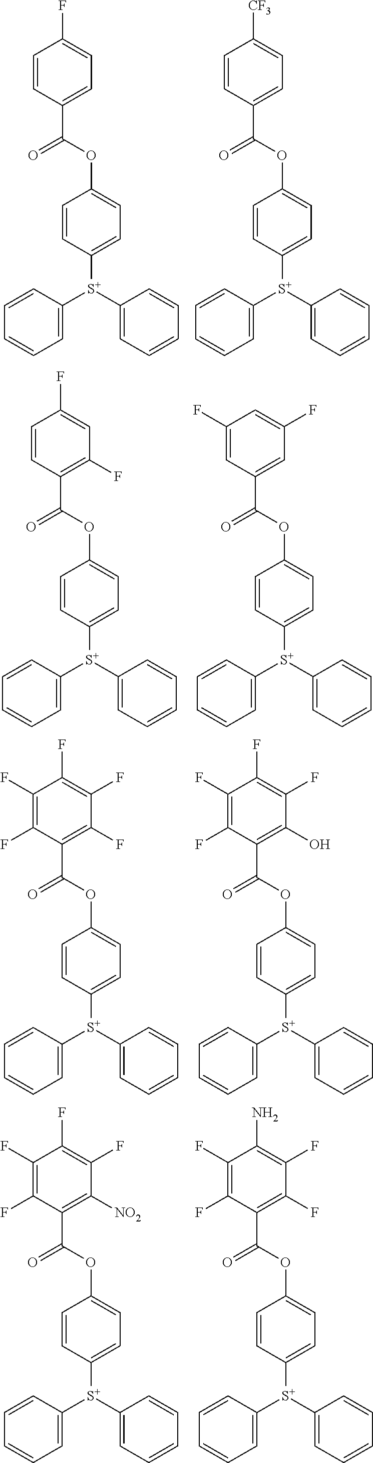

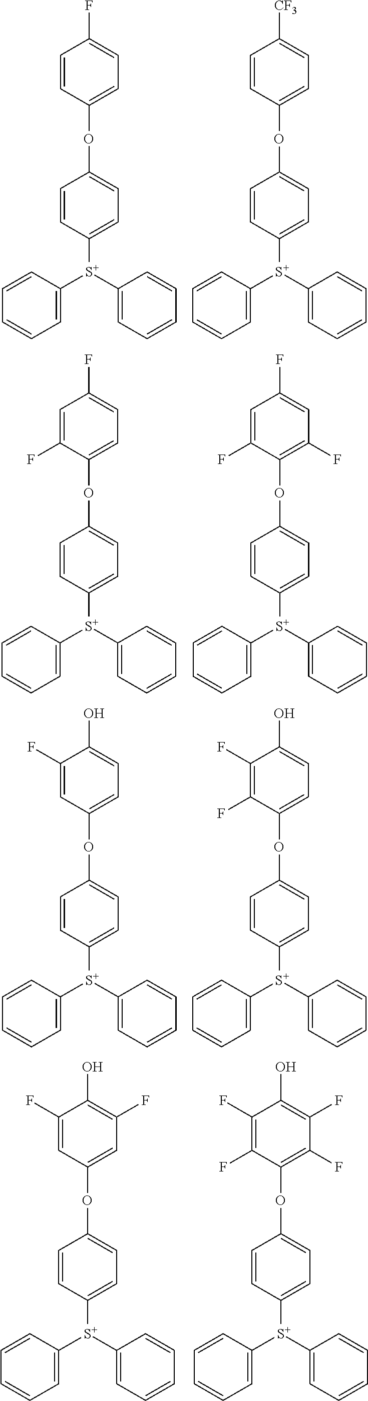

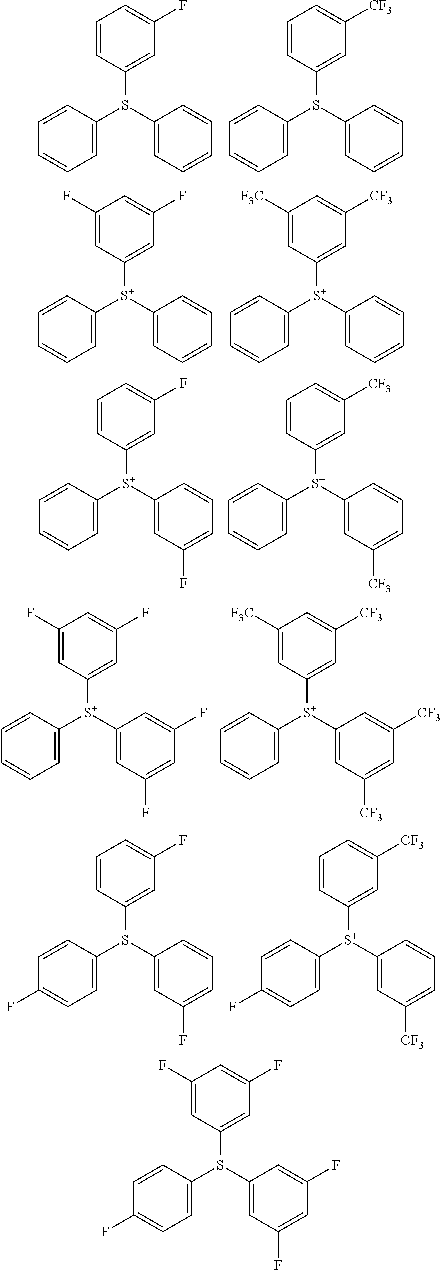

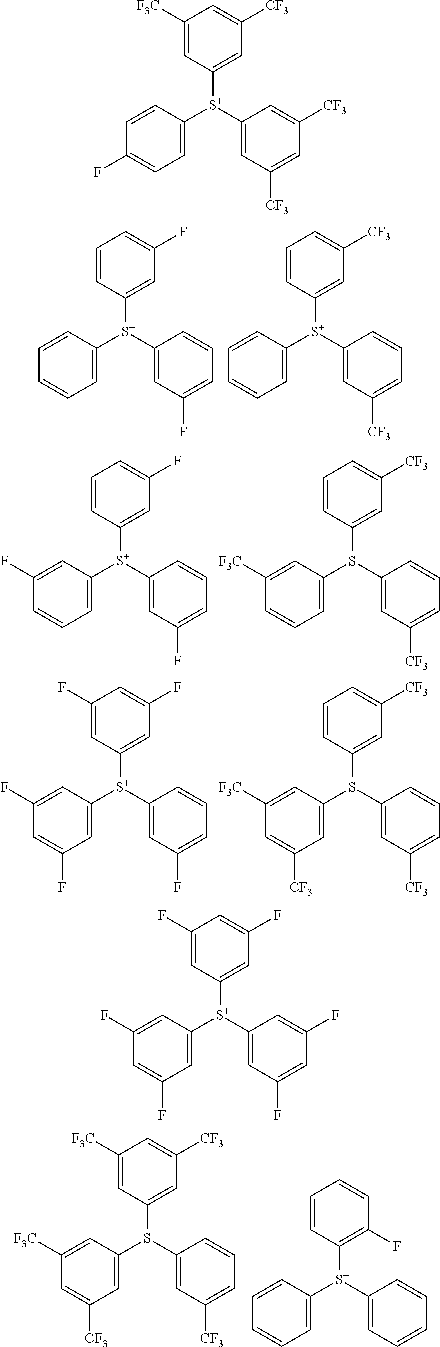

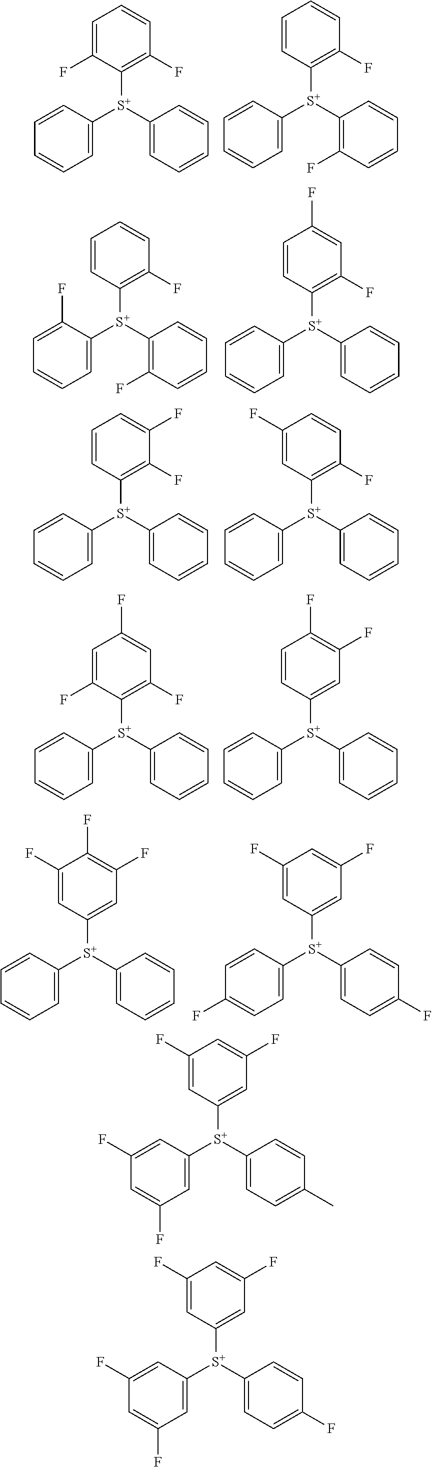

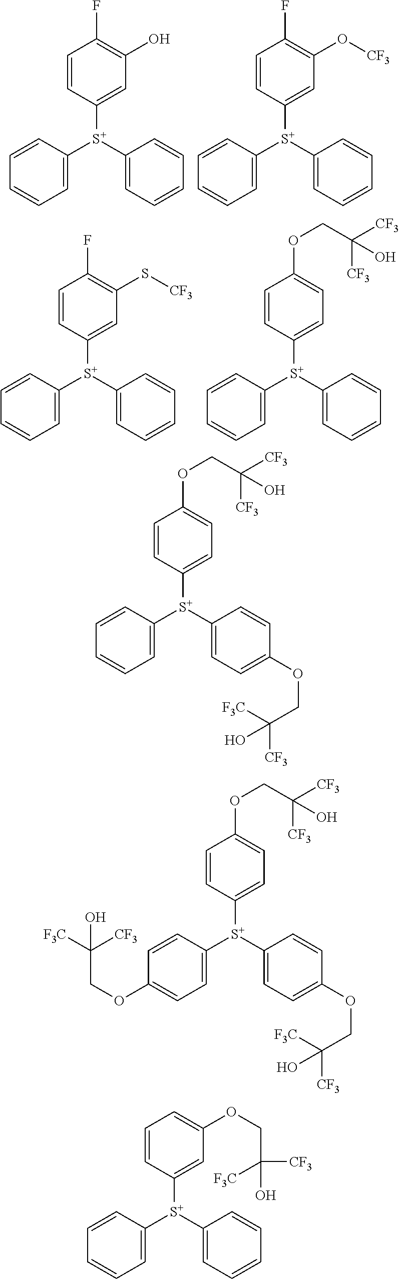

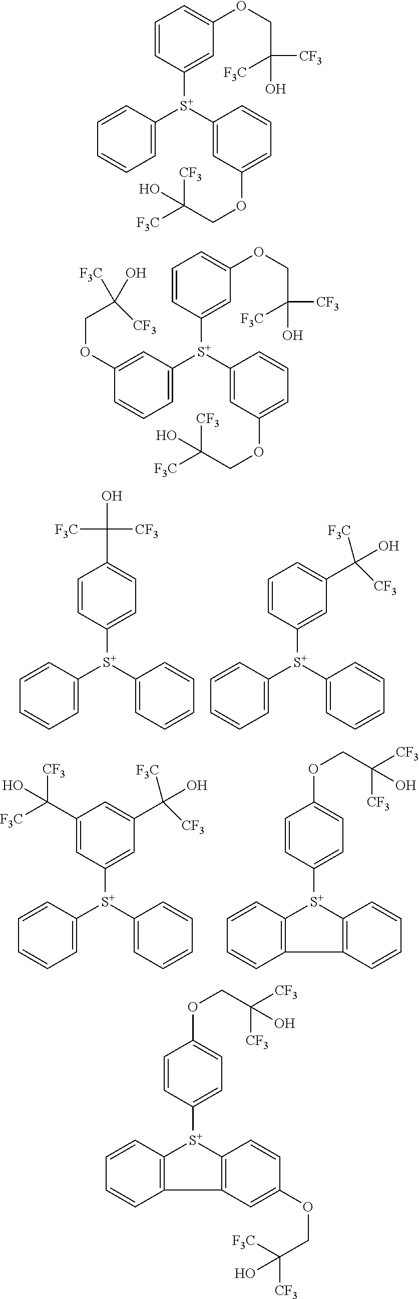

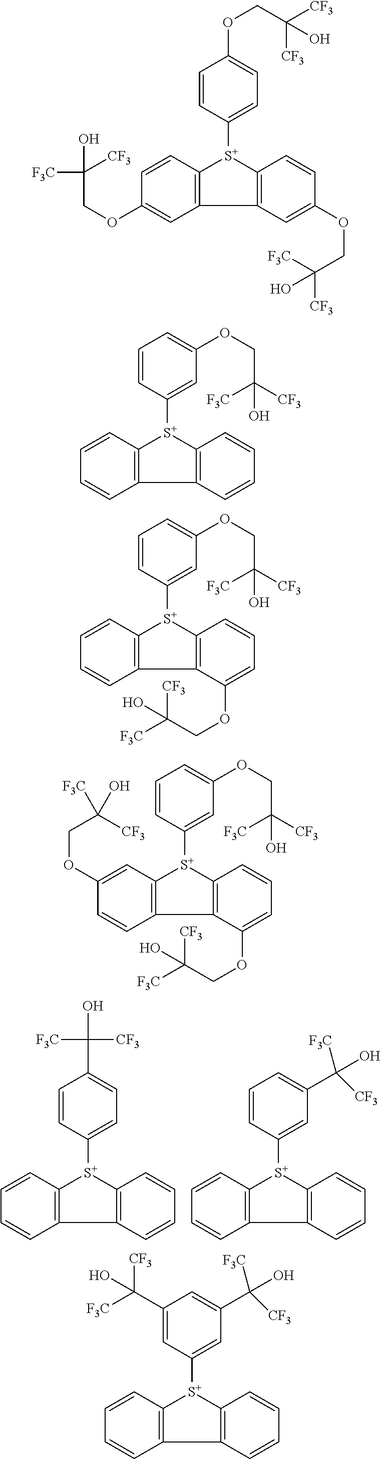

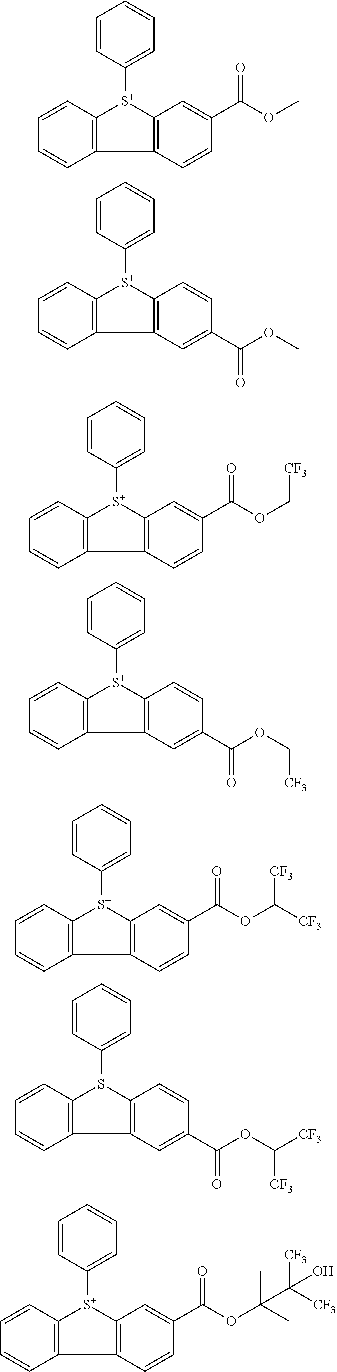

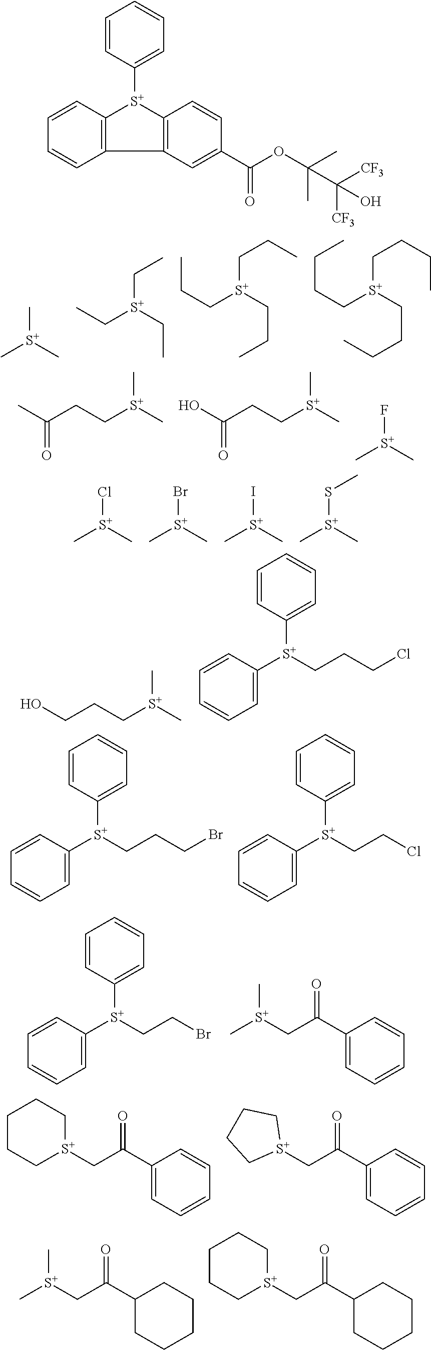

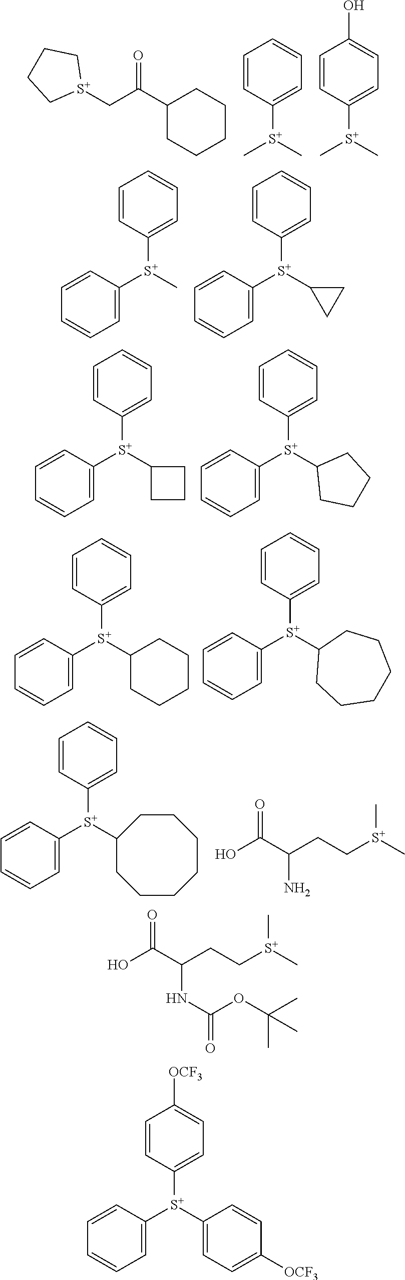

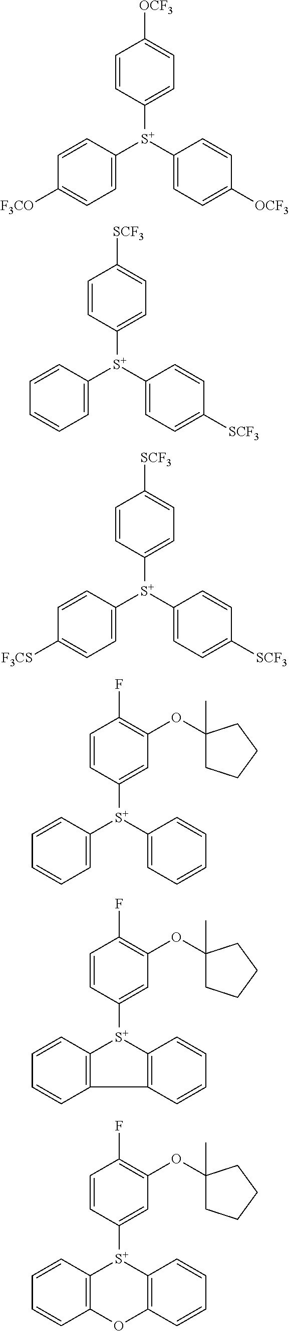

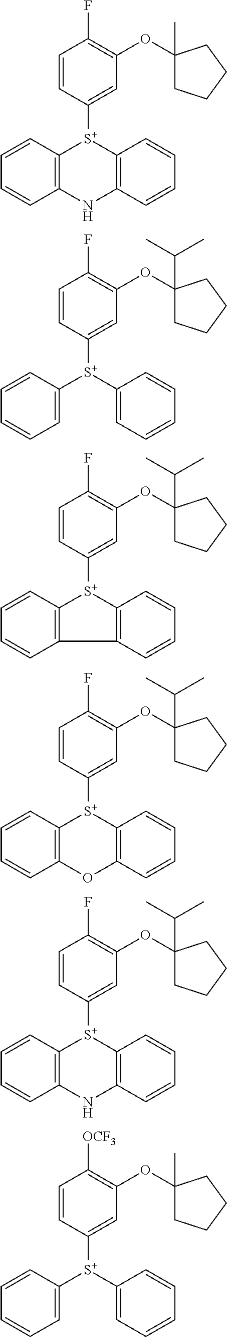 [SMILES: CC1(Oc2cc([S+]3c4ccccc4Nc4ccccc43)ccc2F)CCCC1 ; CC(C)C1(Oc2cc([S+](c3ccccc3)c3ccccc3)ccc2F)CCCC1 ; CC(C)C1(Oc2cc(-[s+]3c4ccccc4c4ccccc43)ccc2F)CCCC1 ; CC(C)C1(Oc2cc([S+]3c4ccccc4Oc4ccccc43)ccc2F)CCCC1 ; CC(C)C1(Oc2cc([S+]3c4ccccc4Nc4ccccc43)ccc2F)CCCC1 ; CC1(Oc2cc([S+](c3ccccc3)c3ccccc3)ccc2OC(F)(F)F)CCCC1]

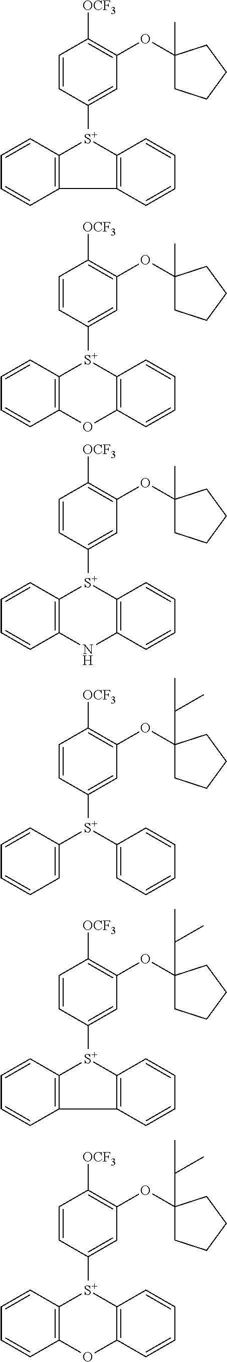 [SMILES: CC1(Oc2cc(-[s+]3c4ccccc4c4ccccc43)ccc2OC(F)(F)F)CCCC1 ; CC1(Oc2cc([S+]3c4ccccc4Oc4ccccc43)ccc2OC(F)(F)F)CCCC1 ; CC1(Oc2cc([S+]3c4ccccc4Nc4ccccc43)ccc2OC(F)(F)F)CCCC1 ; CC(C)C1(Oc2cc([S+](c3ccccc3)c3ccccc3)ccc2OC(F)(F)F)CCCC1 ; CC(C)C1(Oc2cc(-[s+]3c4ccccc4c4ccccc43)ccc2OC(F)(F)F)CCCC1 ; CC(C)C1(Oc2cc([S+]3c4ccccc4Oc4ccccc43)ccc2OC(F)(F)F)CCCC1]

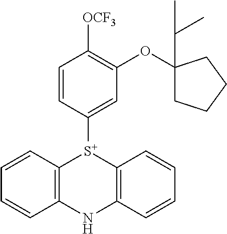 [SMILES: CC(C)C1(Oc2cc([S+]3c4ccccc4Nc4ccccc43)ccc2OC(F)(F)F)CCCC1]

Specific examples of the iodonium cation represented by the formula (cation-2) include, but are not limited to, those shown below.

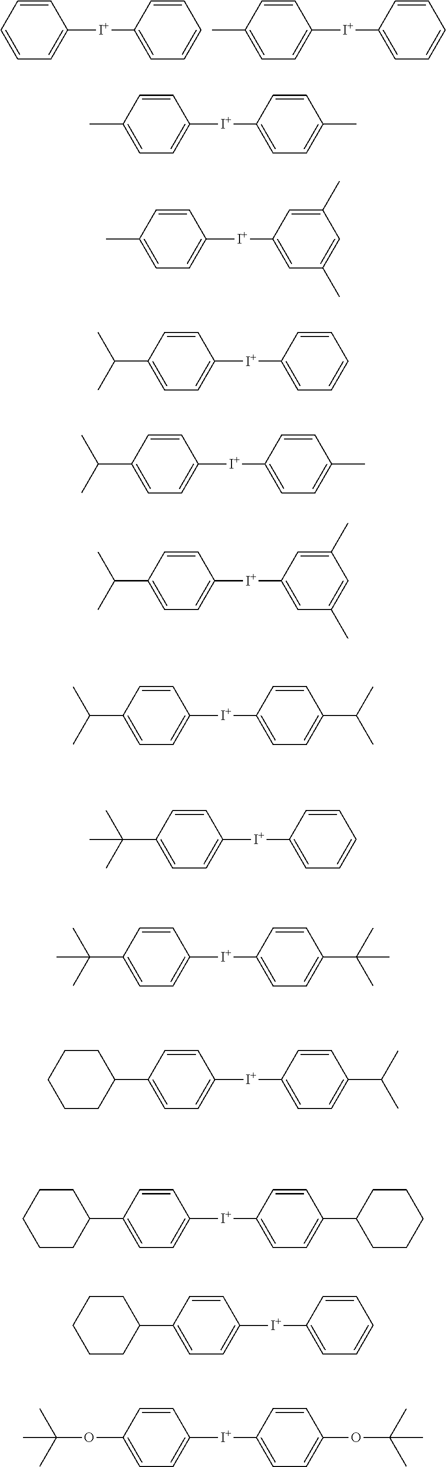

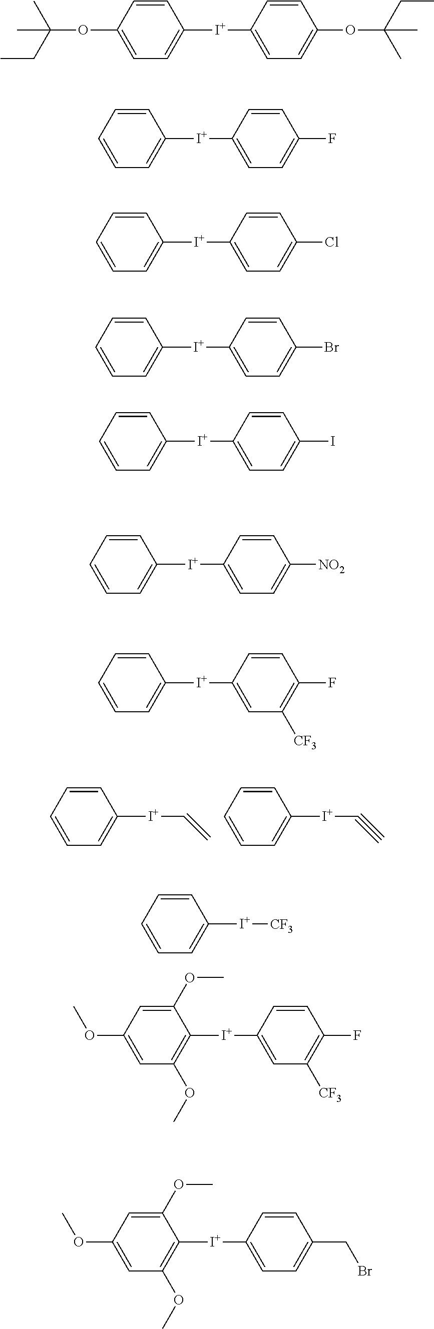

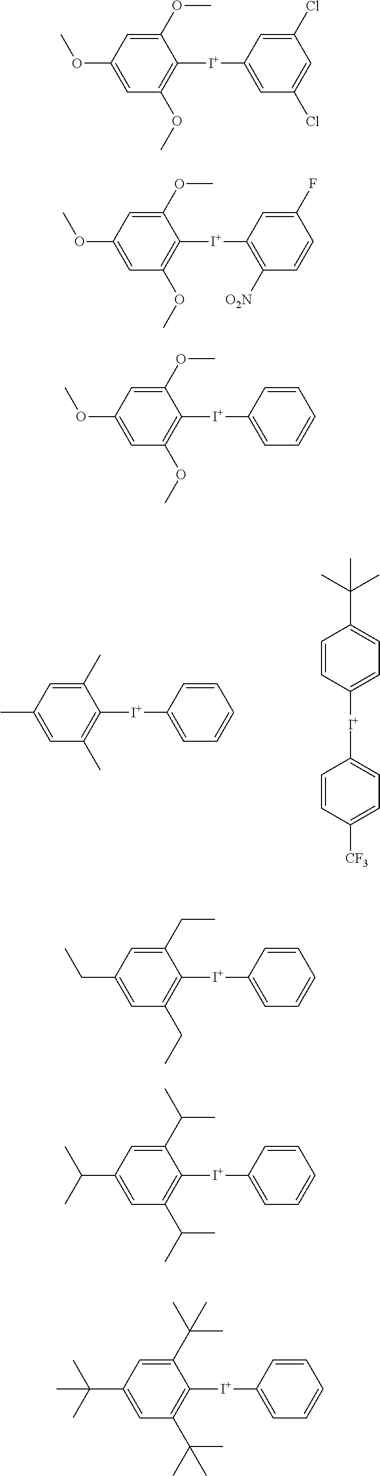

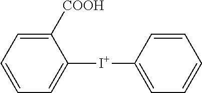 [SMILES: O=C(O)c1ccccc1[I+]c1ccccc1]

Specific examples of the onium salt monomer represented by the formula (a) include any combinations of the anions and cations shown above.

The onium salt monomer represented by the formula (a) can be synthesized, for example, by the method as described in JP 5201363 B for a sulfonium salt having a polymerizable anion, but the synthesis is not limited thereto.

### [Polymer]

The polymer of the present invention contains a repeating unit derived from the onium salt monomer represented by the formula (a) (hereinafter, also referred to as repeating unit a).

The polymer of the present invention is a polymer-bound photo-acid generator that functions as a photo-acid generator and at the same time as a base polymer in chemically amplified resist compositions. The polymer of the present invention is structurally characterized by, for example, containing a repeating unit having an onium salt structure derived from the onium salt monomer of the present invention, wherein the onium salt structure contains a fluorosulfonate anion having a benzene or naphthalene structure directly bound to the main chain and having an aromatic ring structure substituted with an iodine atom. Iodine atoms generate secondary electrons during exposure because iodine atoms exhibit very large absorption of EUV at a wavelength of 13.5 nm, and the energy of secondary electrons transfers to an acid generator to promote the decomposition, resulting in high sensitivity. The polymerizable group consisting of styrene structure has higher rigidity than polymerizable groups including methacrylate, and the resulting polymer has enhanced glass transition temperature (Tg). The interaction of aromatic rings (n-n stacking effect) in each base polymer molecule or between base polymer molecules is expected to cause base polymer molecules to be regularly arranged, exhibiting resistance to pattern collapse due to developer even in fine patterning. Also in the etching process after fine patterning, superior etching resistance is exhibited by virtue of the inclusion of aromatic rings directly bound to the main chain. In the aromatic ring substituted with an iodine atom, the substituting iodine atom is preferably bound to the ortho position of the carbon atom to which the polymerizable group is bound, and this is expected to inhibit the rotation of the bond axis between the aromatic ring of the polymerizable group and the aromatic ring substituted with an iodine atom. As a result, the excessive acid diffusion of a generated acid is inhibited to enable patterning resistant to pattern collapse with superiority in the LWR of line patterns and in the CDU of hole patterns, and this is particularly suitable for a material of chemically amplified positive-type resist compositions. Furthermore, the fluorine substituents introduced into the aromatic ring of the styrene moiety are not only expected to exert an effect of improving the solubility in solvents, but also expected to give enhanced sensitivity because of the absorption of EUV by fluorine itself. When being polymerized, the onium salt monomer of the present invention is expected to allow the polymer itself to have enhanced solubility in solvents, thus being expected to exert an effect of preventing not only defects due to solubility in solvents, but also the attachment or the like of patterns after resist development, wherein the attachment or the like is prevented with a more proper water-repellent effect by the action of the fluorine substituents.

The polymer may further contain a repeating unit represented by formula (b1) shown below (hereinafter, also referred to as repeating unit b1) or a repeating unit represented by formula (b2) shown below (hereinafter, also referred to as repeating unit b2).

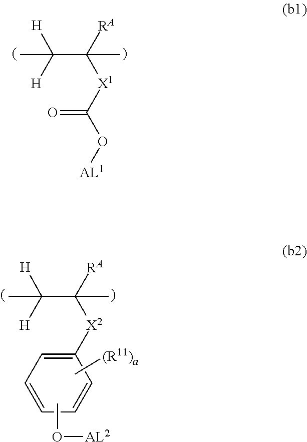

wherein R^{A} groups are each independently a hydrogen atom, a fluorine atom, a methyl group, or a trifluoromethyl group;
X¹ is a single bond, a phenylene group, a naphthylene group, *—C(═O)—O—X¹¹—, or *—C(═O)—NH—X¹¹—, wherein the phenylene group or naphthylene group is optionally substituted with an alkoxy group having 1 to 10 carbon atoms and optionally containing a fluorine atom, or with a halogen atom, X¹¹ is a saturated hydrocarbylene group having 1 to 10 carbon atoms, a phenylene group, or a naphthylene group, and the saturated hydrocarbylene group optionally contains a hydroxy group, an ether bond, an ester bond, or a lactone ring;
X² is a single bond, *—C(═O)—O—, or *—C(═O)—NH—;
* indicates bonding to a carbon atom of a main chain;
AL¹ and AL² are each independently an acid-unstable group.
R¹¹ is a halogen atom, a cyano group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; and
a is an integer of 0 to 4.

In the formulae (b1) and (b2), R^{A} groups are each independently a hydrogen atom, a fluorine atom, a methyl group, or a trifluoromethyl group.

In the formula (b1), X¹ is a single bond, a phenylene group, a naphthylene group, *—C(═O)—O—X¹¹—, or *—C(═O)—NH—X¹¹—, wherein the phenylene group or naphthylene group is optionally substituted with an alkoxy group having 1 to 10 carbon atoms and optionally containing a fluorine atom or with a halogen atom. X¹¹ is a saturated hydrocarbylene group having 1 to 10 carbon atoms, a phenylene group, or a naphthylene group, and the saturated hydrocarbylene group optionally contains a hydroxy group, an ether bond, an ester bond, or a lactone ring. * indicates bonding to a carbon atom of a main chain.

In the formula (b2), X² is a single bond, *—C(═O)—O—, or *—C(═O)—NH—. * indicates bonding to a carbon atom of a main chain. R^{1l} is a halogen atom, a cyano group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom. a is an integer of 0 to 4, and preferably 0 or 1.

In the formulae (b1) and (b2), AL¹ and AL² are each independently an acid-unstable group. Specific examples of the acid-unstable group include, but are not limited to, those described in JP 2013-80033 A and JP 2013-83821 A.

Specific typical examples of the acid-unstable group are those represented by the following formulae (AL-1) to (AL-3)

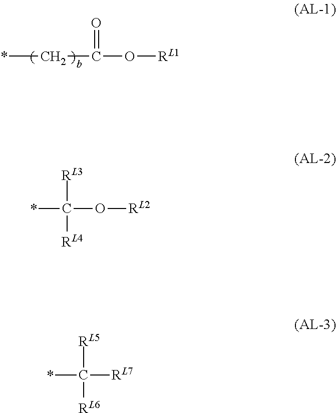

wherein * indicates bonding.

In the formulae (AL-1) and (AL-2), R^{L1} and R^{L2} are each independently a hydrocarbyl group having 1 to 40 carbon atoms, and may contain a heteroatom such as an oxygen atom, a sulfur atom, a nitrogen atom, a fluorine atom, and an iodine atom. The hydrocarbyl group may be linear, branched, or cyclic. The hydrocarbyl group is preferably one having 1 to 20 carbon atoms.

In the formula (AL-1), b is an integer of 0 to 10, and is preferably an integer of 1 to 5.

In the formula (AL-2), R^{L3} and R^{L4} are each independently a hydrogen atom or a hydrocarbyl group having 1 to 20 carbon atoms, and may contain a heteroatom such as an oxygen atom, a sulfur atom, a nitrogen atom, a fluorine atom, and an iodine atom. The hydrocarbyl group may be linear, branched, or cyclic. Any two of R^{L2}, R^{L3}, and R^{L4} are optionally bound together to form a ring having 3 to 20 carbon atoms together with the carbon atom to which the two are bound or together with the carbon atom and oxygen atom to which the two are bound. The ring is preferably a ring having 4 to 16 carbon atoms, and particularly preferably an aliphatic ring.

In the formula (AL-3), R^{L5}, R^{L6}, and R^{L7} are each independently a hydrocarbyl group having 1 to 20 carbon atoms, and may contain a heteroatom such as an oxygen atom, a sulfur atom, a nitrogen atom, a fluorine atom, and an iodine atom. The hydrocarbyl group may be linear, branched, or cyclic. Any two of R^{L5}, R^{L6}, and R^{L7} are optionally bound together to form a ring having 3 to 20 carbon atoms together with the carbon atom to which the two are bound. The ring is preferably a ring having 4 to 16 carbon atoms, and particularly preferably an aliphatic ring.

Specific examples of repeating unit b1 include, but are not limited to, those shown below. In the following formulae, R^{A} and AL¹ are as specified above:

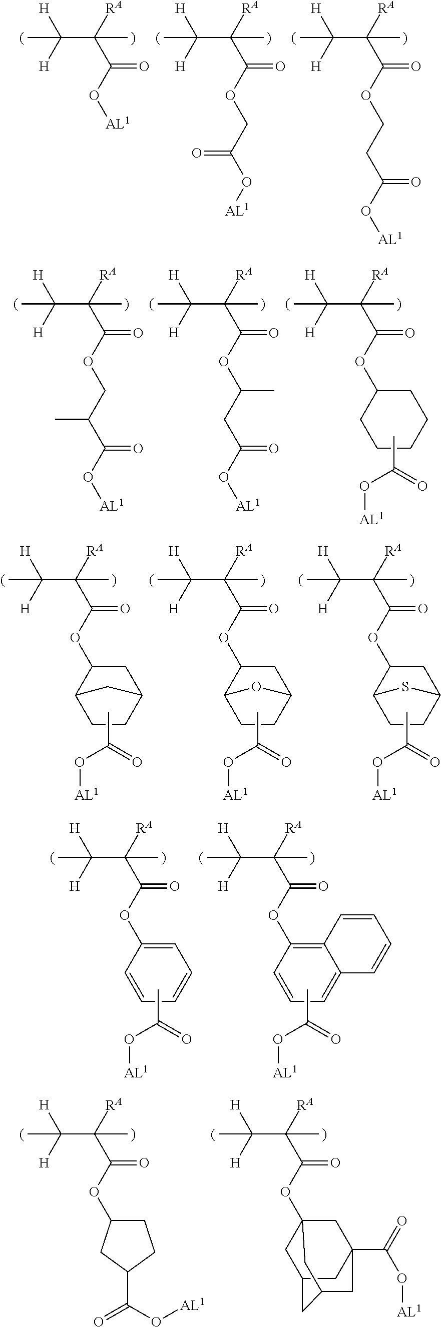

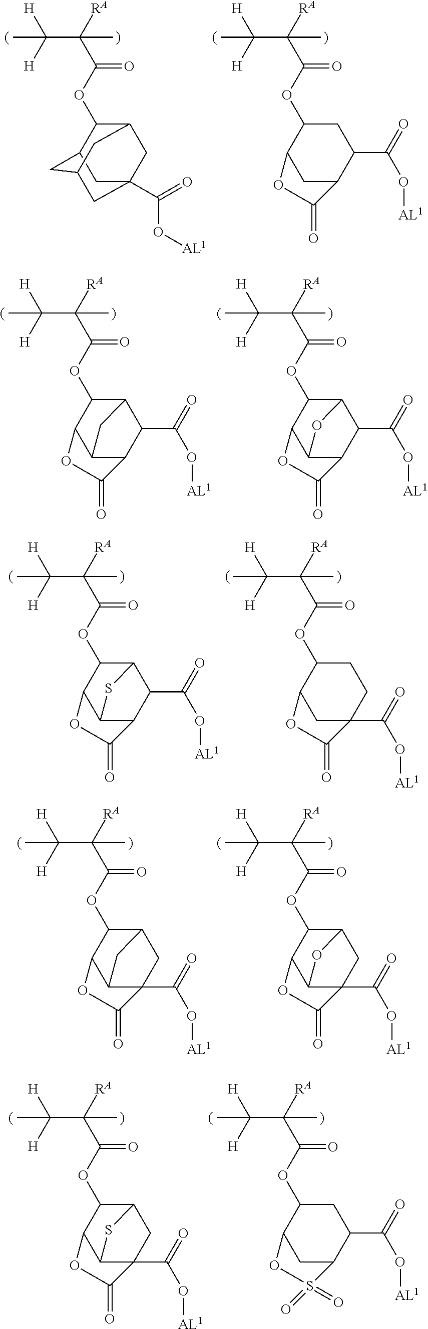

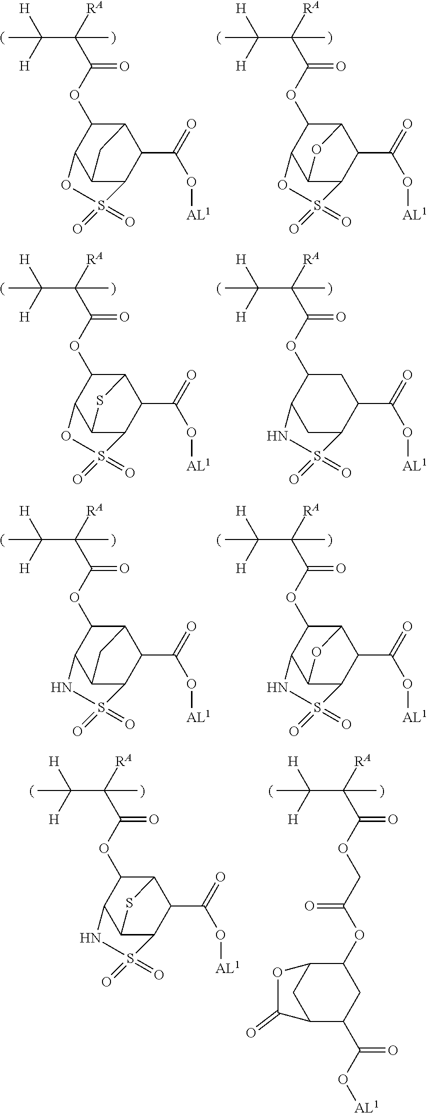

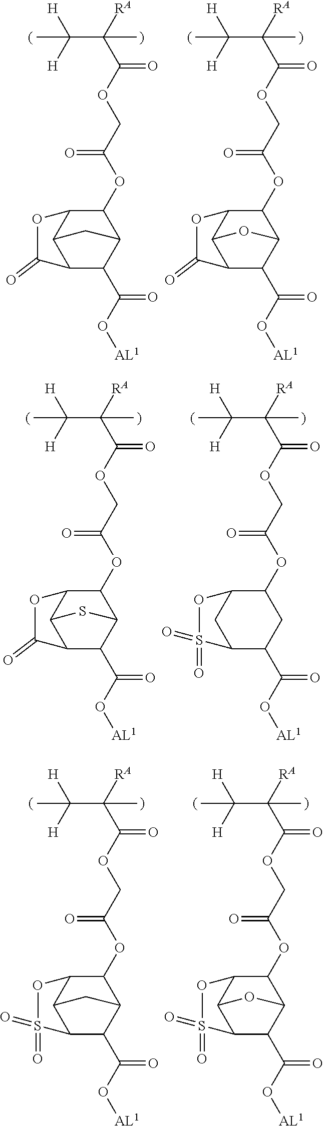

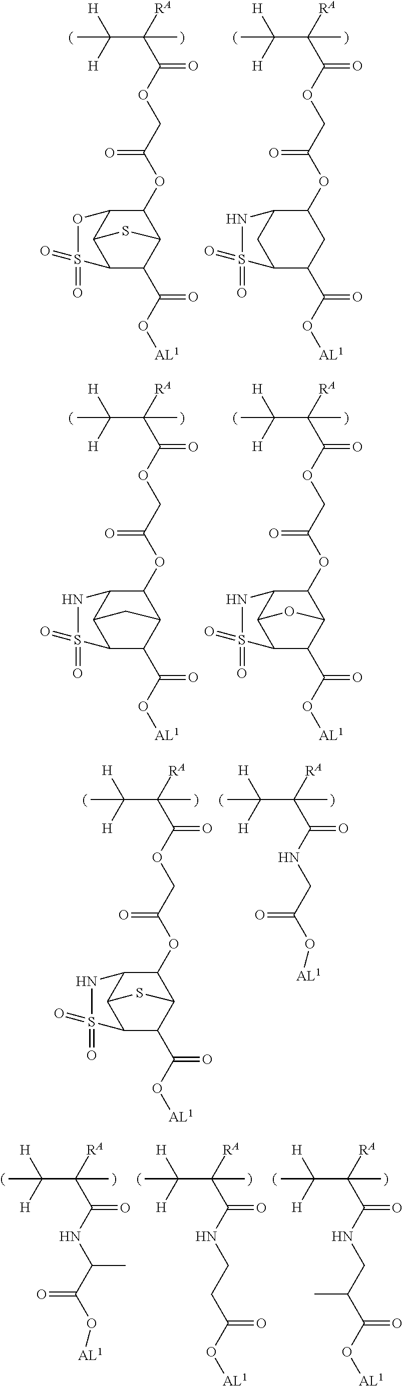

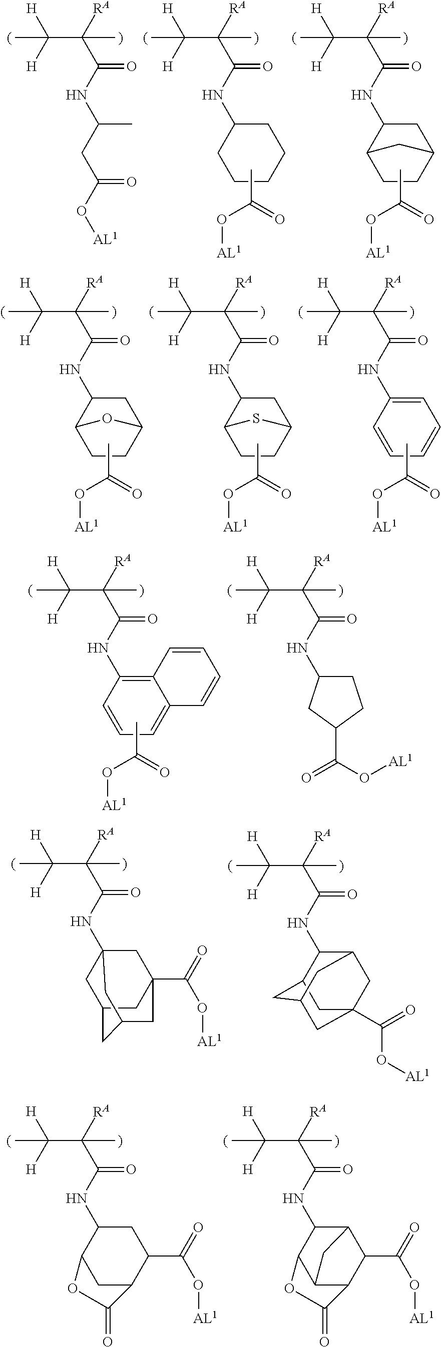

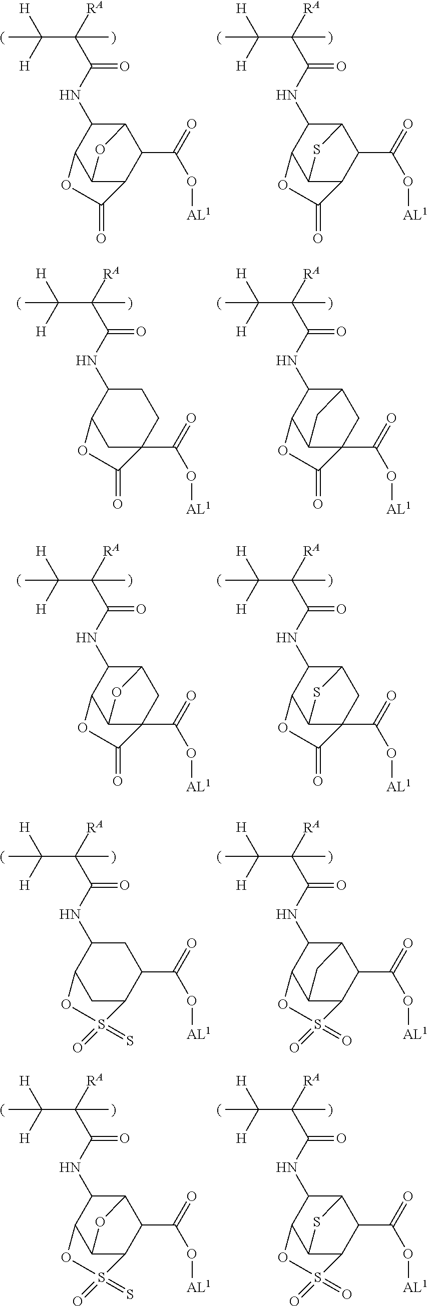

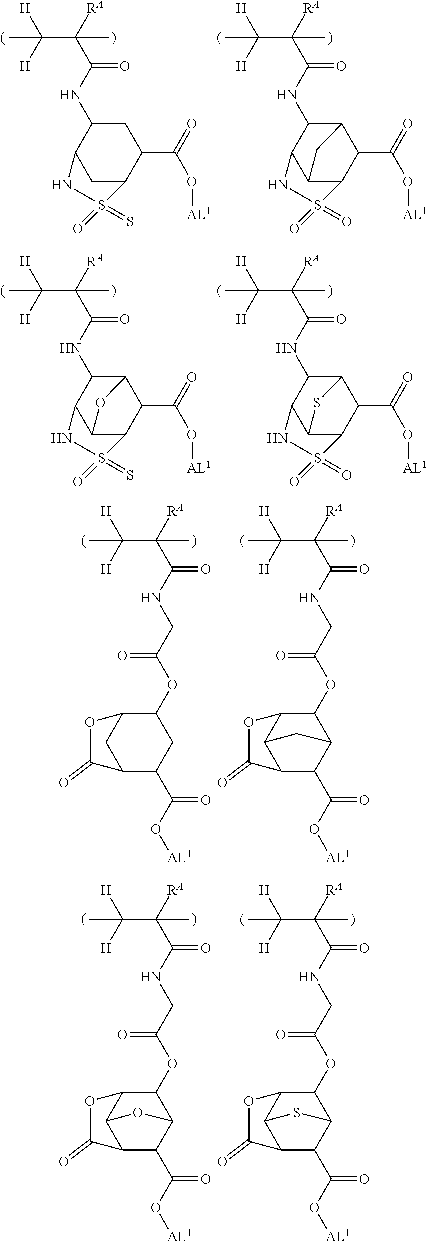

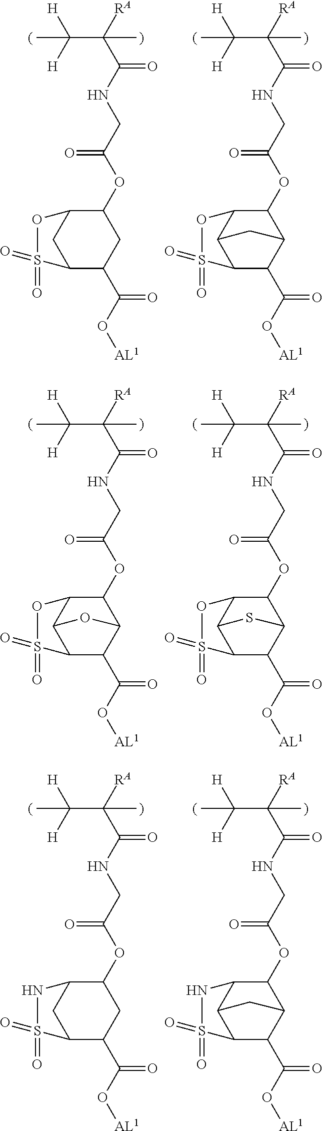

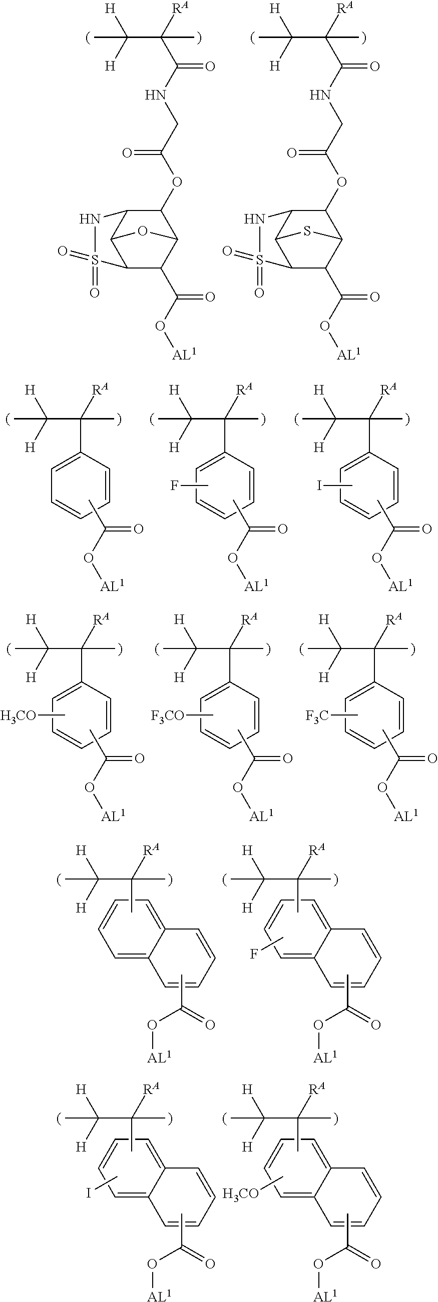

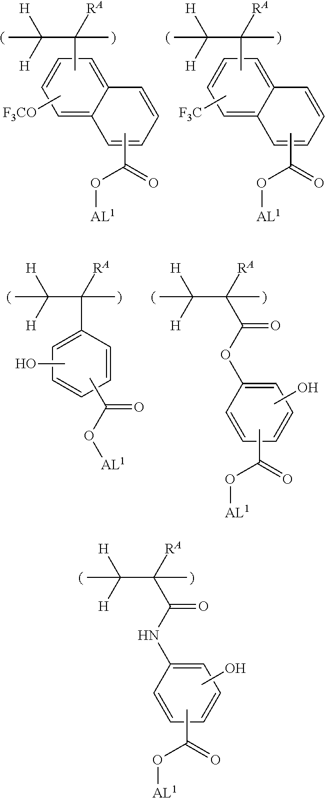

Specific examples of repeating unit b2 include, but are not limited to, those shown below. In the following formulae, R^{A} and AL² are as specified above.

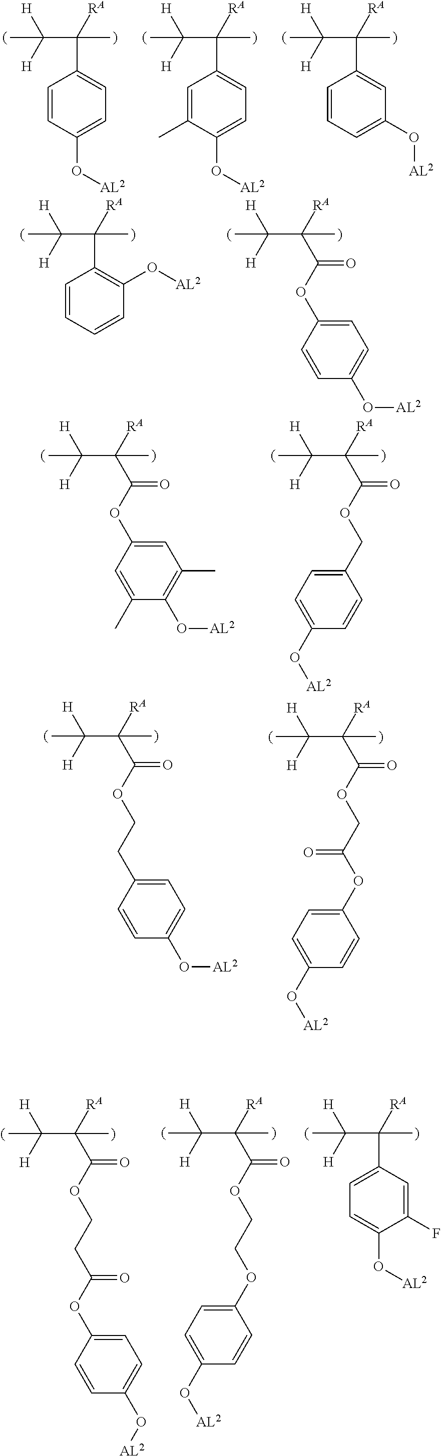

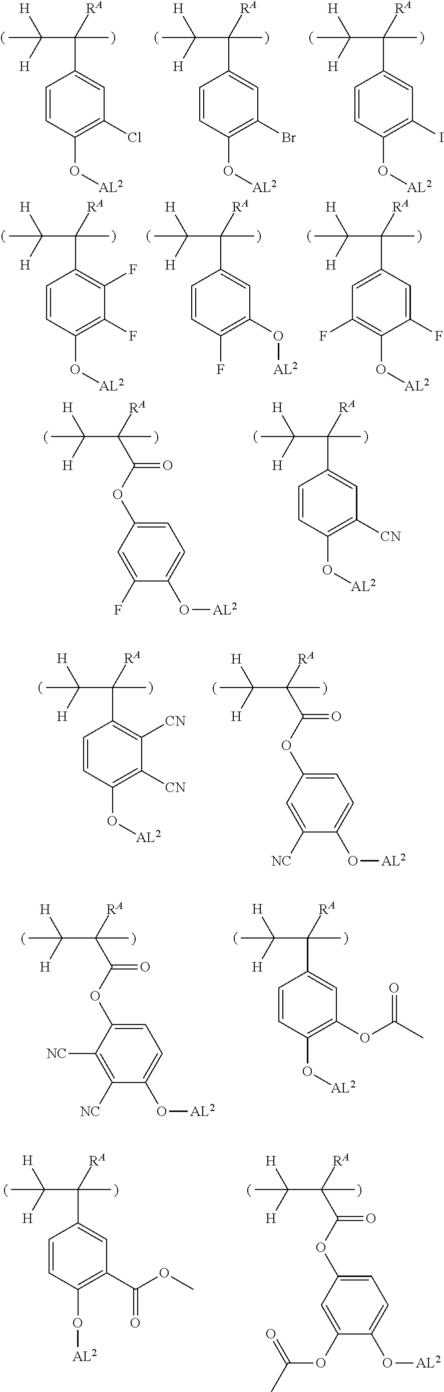

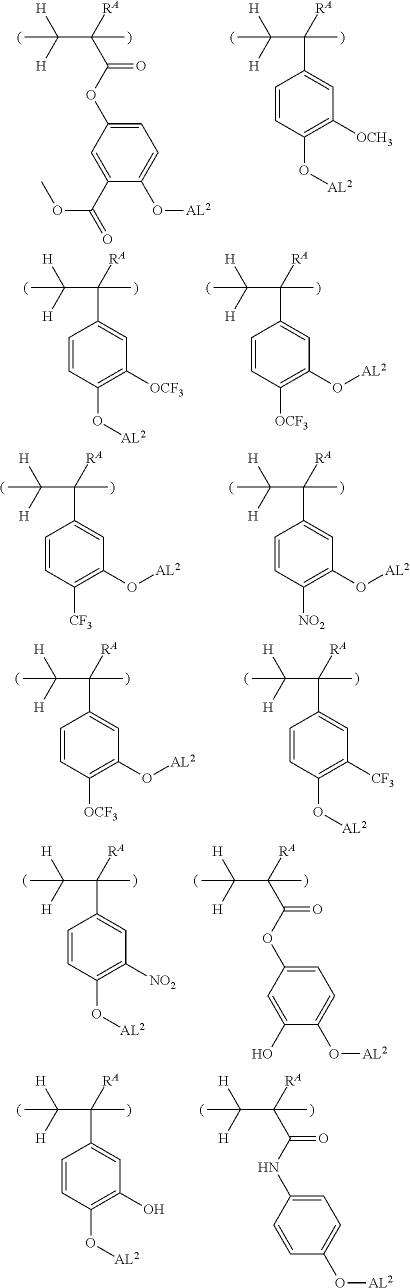

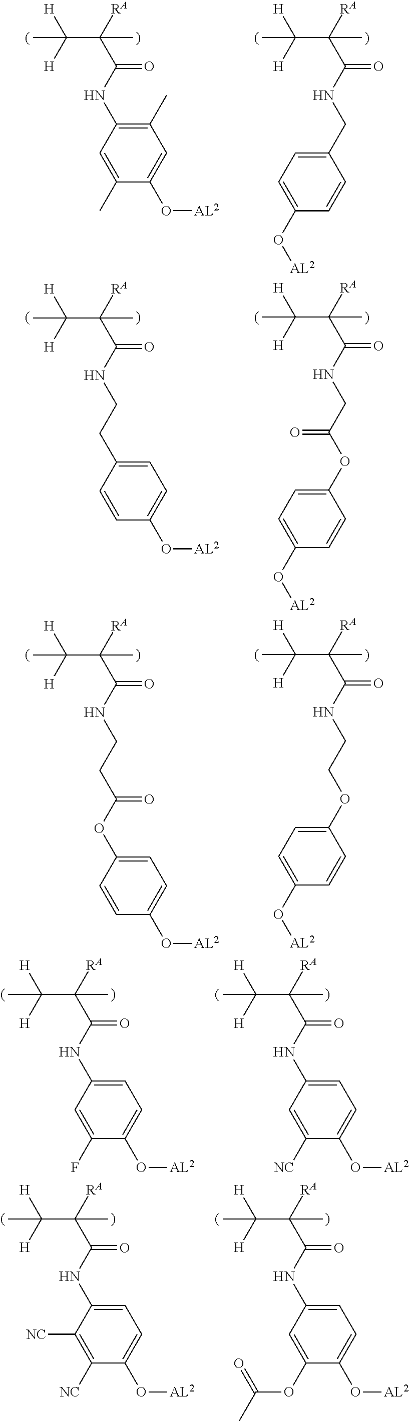

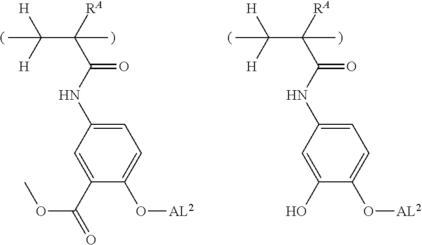

The base polymer preferably further contains a repeating unit represented by the following formula (c1) (hereinafter, also referred to as repeating unit c1):

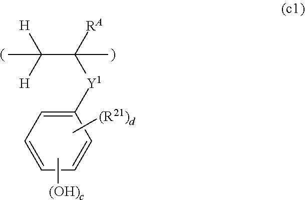

wherein R^{A} is a hydrogen atom, a fluorine atom, a methyl group, or a trifluoromethyl group;
Y¹ is a single bond, *—C(═O)—O—, or *—C(═O)—NH—, wherein * indicates bonding to a carbon atom of a main chain;
R²¹ is a halogen atom, a nitro group, a cyano group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; and
c is an integer of 1 to 4, and d is an integer of 0 to 3, provided that 1≤c+d≤5 is satisfied.

In the formula (c1), R^{A} is a hydrogen atom, a fluorine atom, a methyl group, or a trifluoromethyl group. Y¹ is a single bond, *—C(═O)—O—, or *—C(═O)—NH—. * indicates bonding to a carbon atom of a main chain. R²¹ is a halogen atom, a nitro group, a cyano group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom. c is an integer of 1 to 4. d is an integer of 0 to 3. It should be noted that 1≤c+d≤5 is satisfied.

Specific examples of repeating unit c1 include, but are not limited to, those shown below. In the following formulae, R^{A} is as specified above.

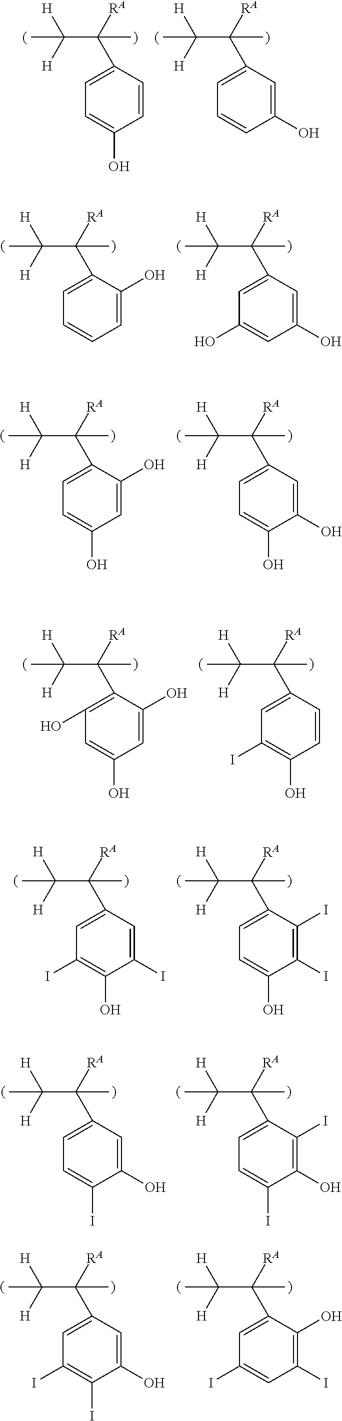

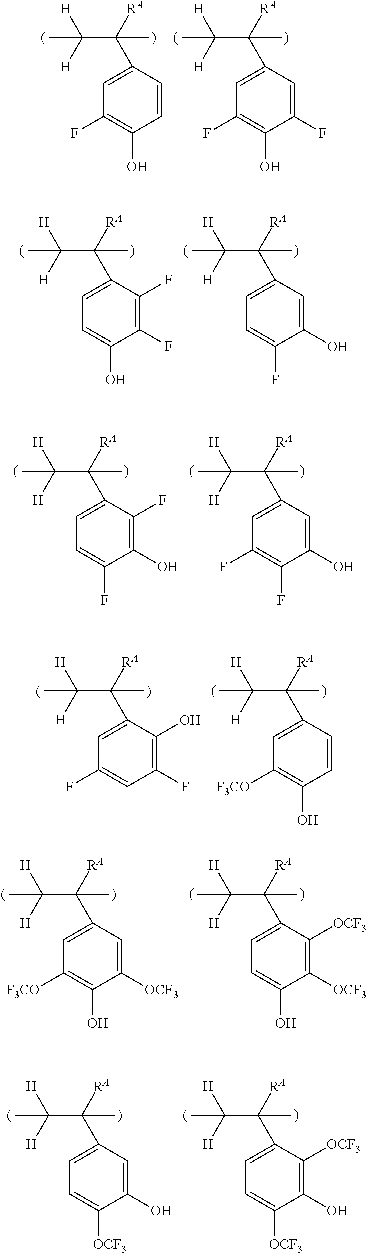

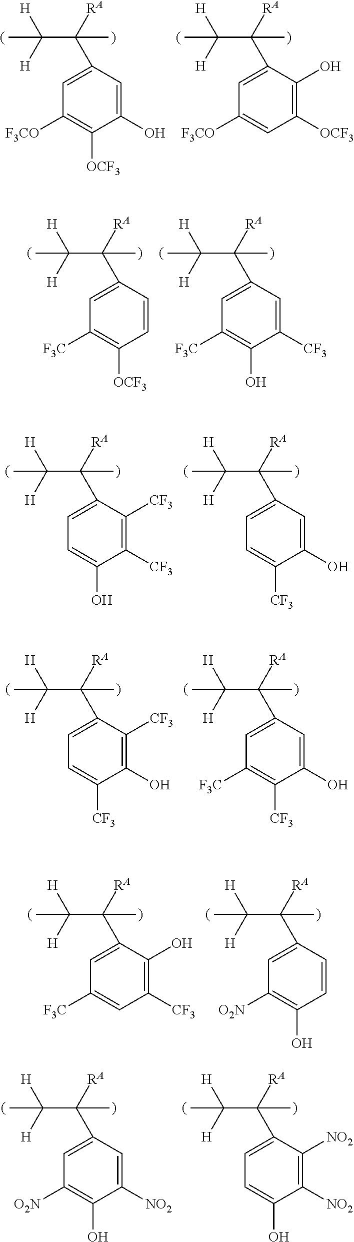

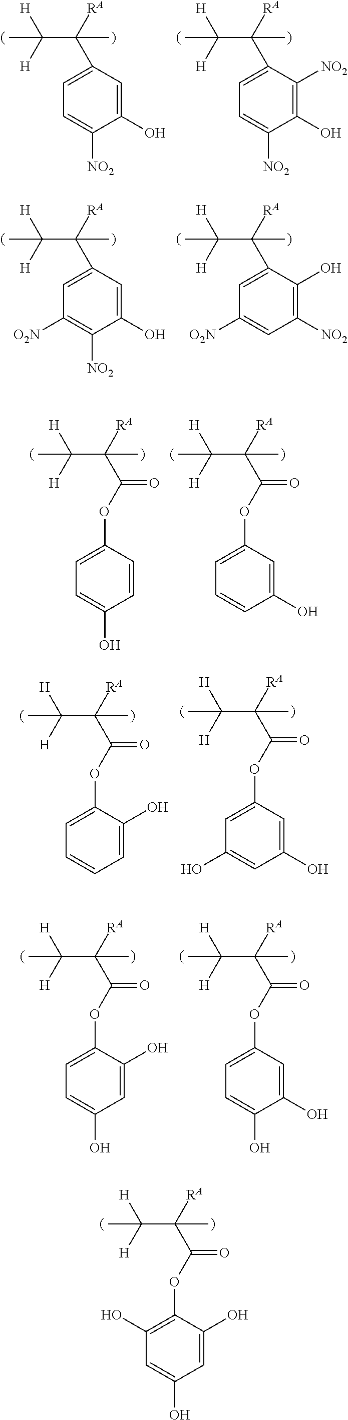

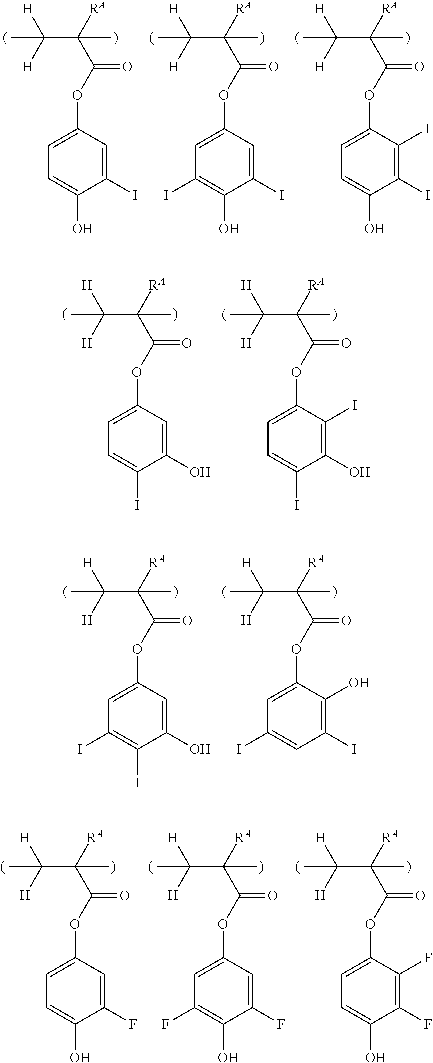

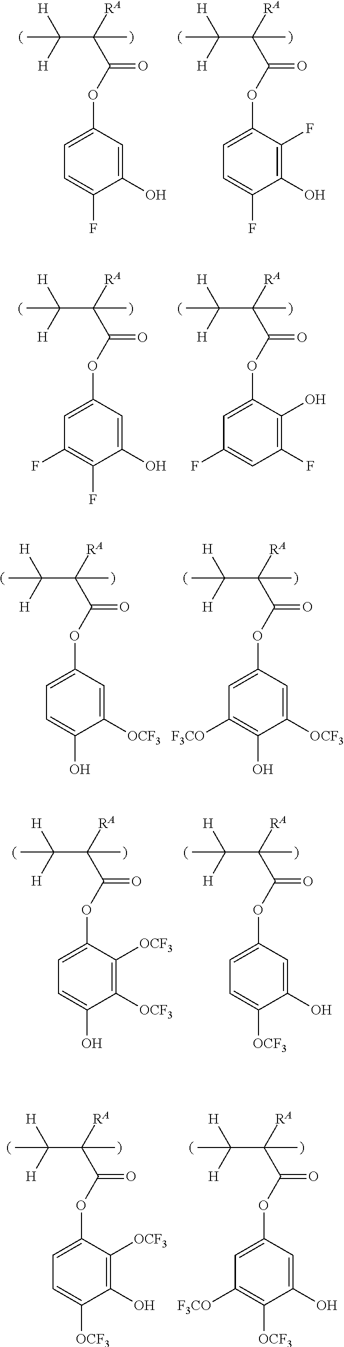

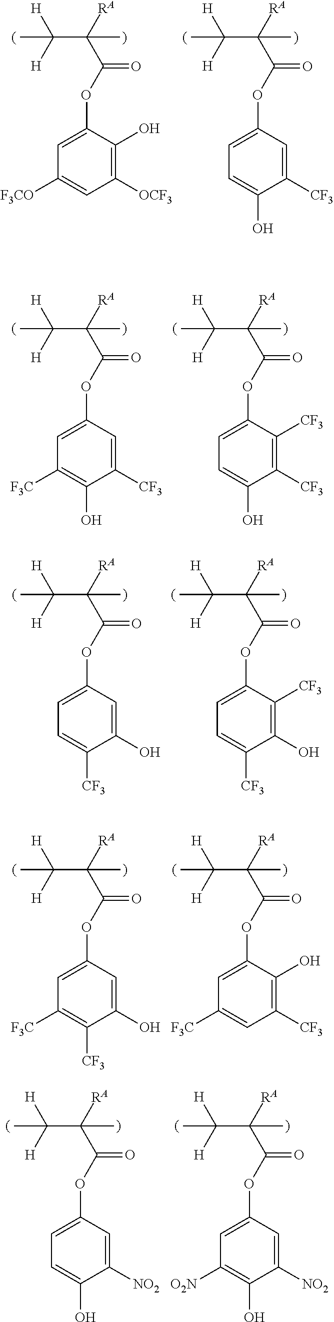

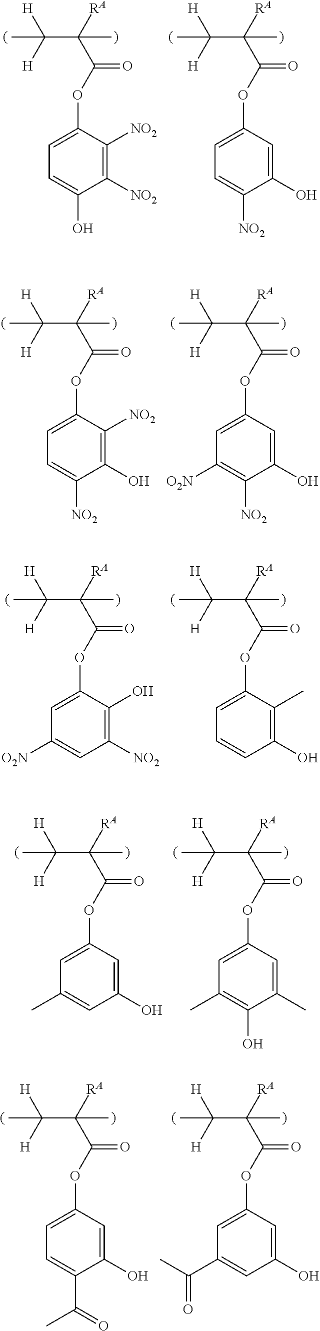

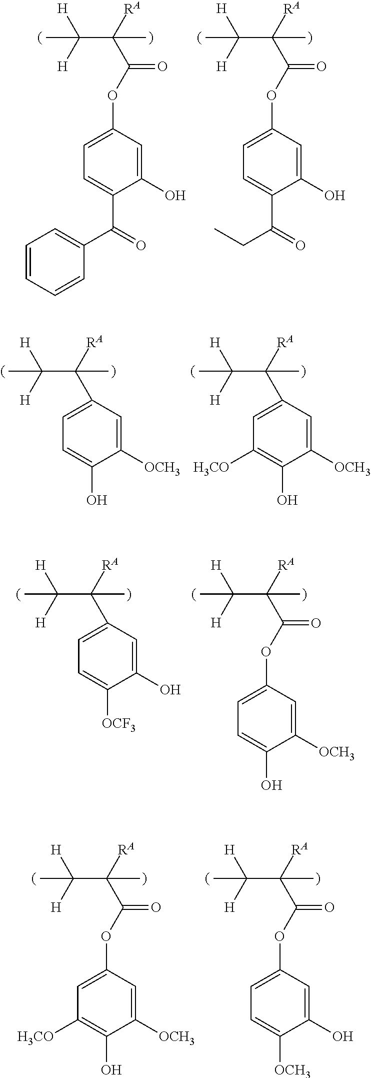

The base polymer preferably further contains a repeating unit represented by the following formula (d1) (hereinafter, also referred to as repeating unit d1):

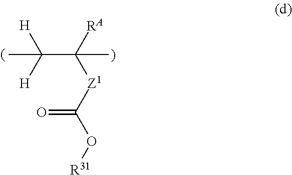

wherein R^{A} is a hydrogen atom, a fluorine atom, a methyl group, or a trifluoromethyl group;
Z¹ is a single bond, a phenylene group, a naphthylene group, *—C(═O)—O—Z¹¹—, or *—C(═O)—NH—Z¹¹—, or wherein the phenylene group or naphthylene group is optionally substituted with an alkoxy group having 1 to 10 carbon atoms and optionally containing a fluorine atom, or with a halogen atom, * indicates bonding to a carbon atom of a main chain, Z ¹¹ is a saturated hydrocarbylene group having 1 to 10 carbon atoms, a phenylene group, or a naphthylene group, and the saturated hydrocarbylene group optionally contains a hydroxy group, an ether bond, an ester bond, or a lactone ring; and
R³¹ is a hydrogen atom or a group having 1 to 20 carbon atoms and containing at least one or more structure selected from a hydroxy group being not a phenolic hydroxy group, a cyano group, a carbonyl group, a carboxy group, an ether bond, an ester bond, a sulfonic ester bond, a carbonate bond, a lactone ring, a sultone ring, and a carboxylic anhydride (—C(═O)—O—C(═O)—).

In the formula (d1), R^{A} is a hydrogen atom, a fluorine atom, a methyl group, or a trifluoromethyl group. Z¹ is a single bond, a phenylene group, a naphthylene group, *—C(═O)—O—Z¹¹—, or *—C(═O)—NH—Z¹¹—, wherein the phenylene group or naphthylene group is optionally substituted with an alkoxy group having 1 to 10 carbon atoms and optionally containing a fluorine atom or with a halogen atom. * indicates bonding to a carbon atom of a main chain. Z ¹¹ is a saturated hydrocarbylene group having 1 to 10 carbon atoms, a phenylene group, or a naphthylene group, and the saturated hydrocarbylene group optionally contains a hydroxy group, an ether bond, an ester bond, or a lactone ring. R³¹ is a hydrogen atom or a group having 1 to 20 carbon atoms and containing at least one or more structure selected from a hydroxy group being not a phenolic hydroxy group, a cyano group, a carbonyl group, a carboxy group, an ether bond, an ester bond, a sulfonic ester bond, a carbonate bond, a lactone ring, a sultone ring, and a carboxylic anhydride (—C(═O)—O—C(═O)—).

Specific examples of repeating unit d1 include, but are not limited to, those shown below. In the following formulae, R^{A} is as specified above:

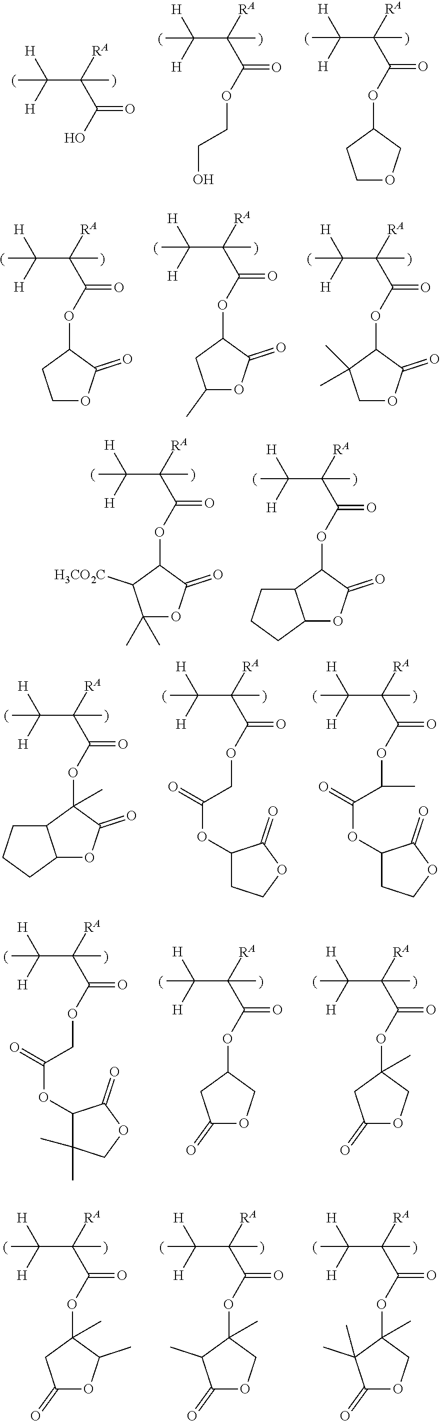

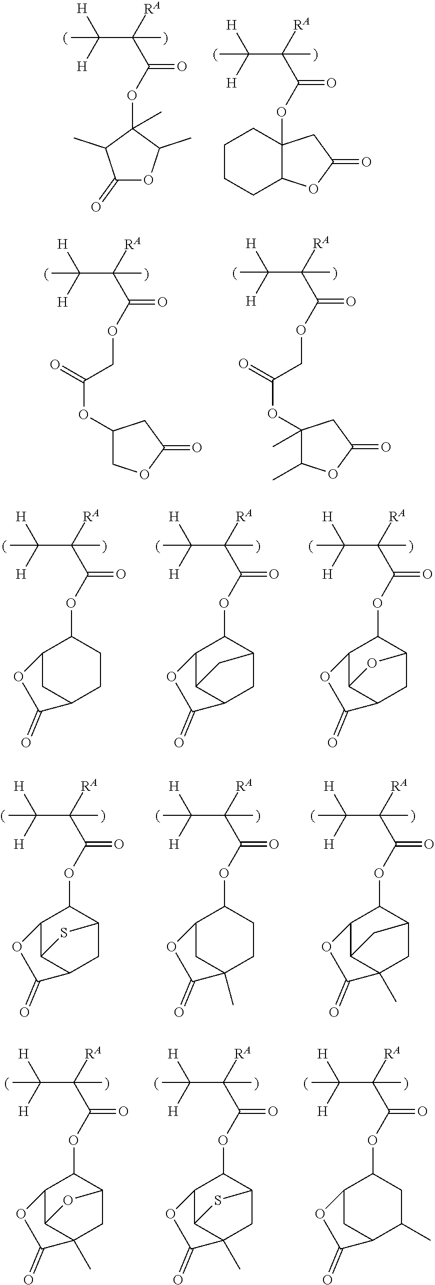

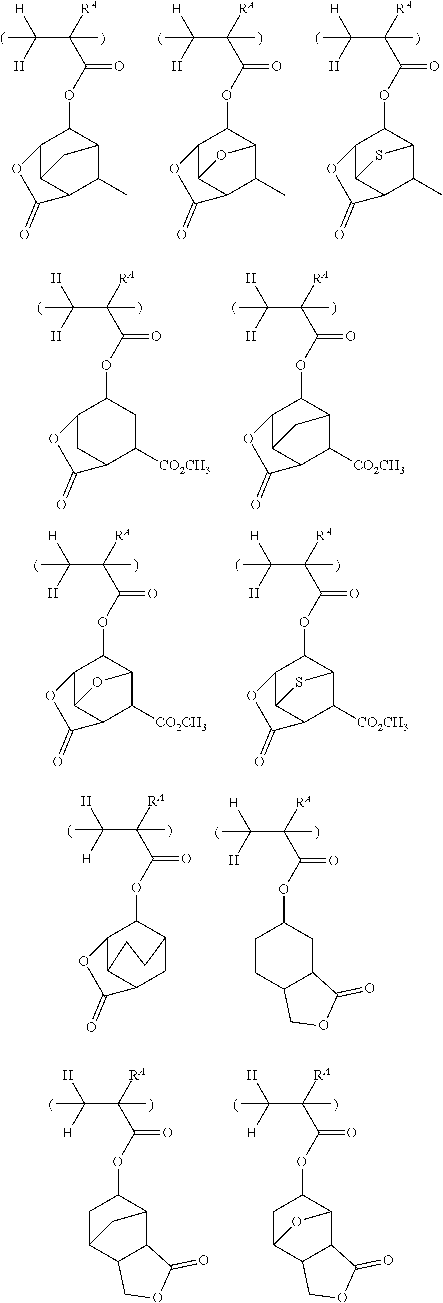

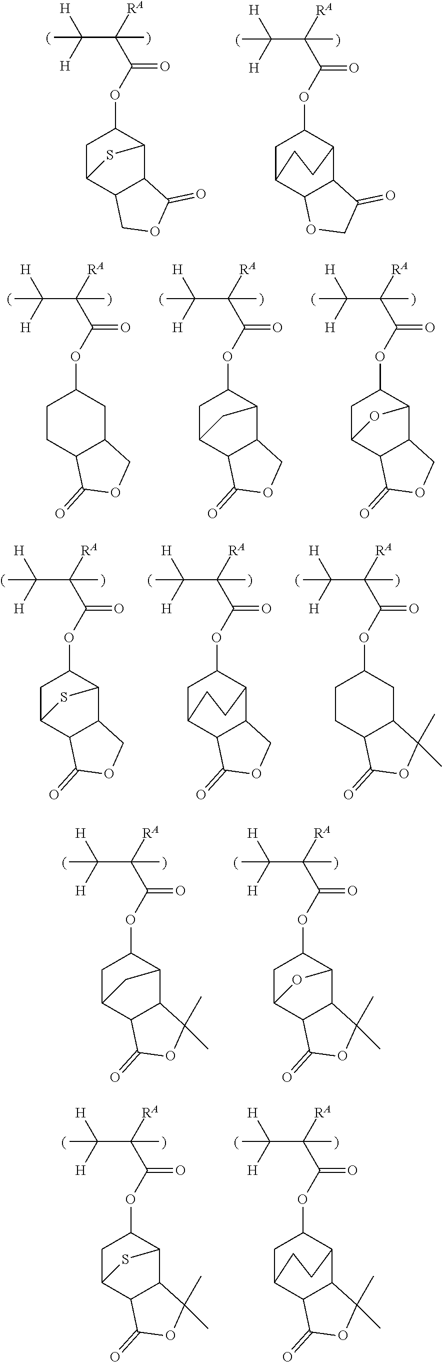

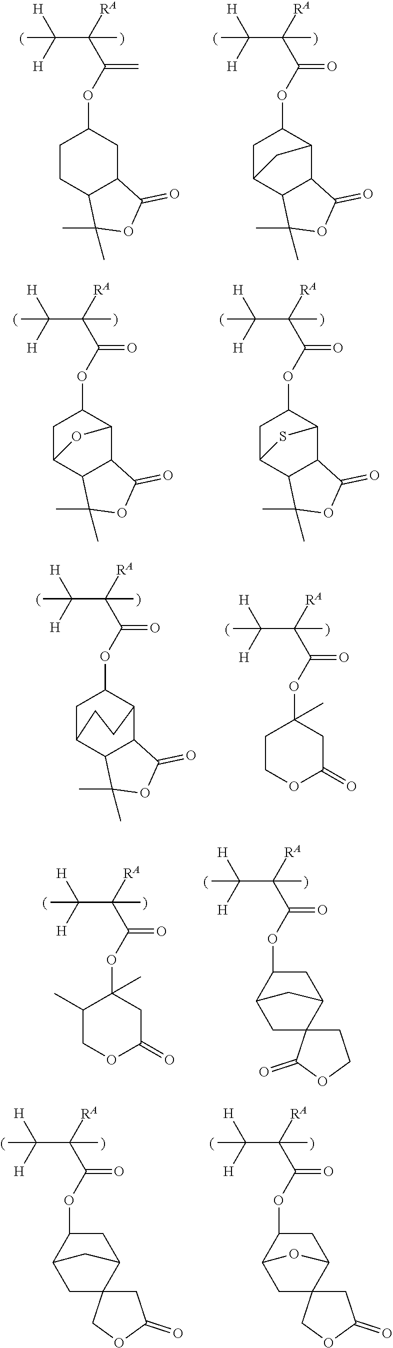

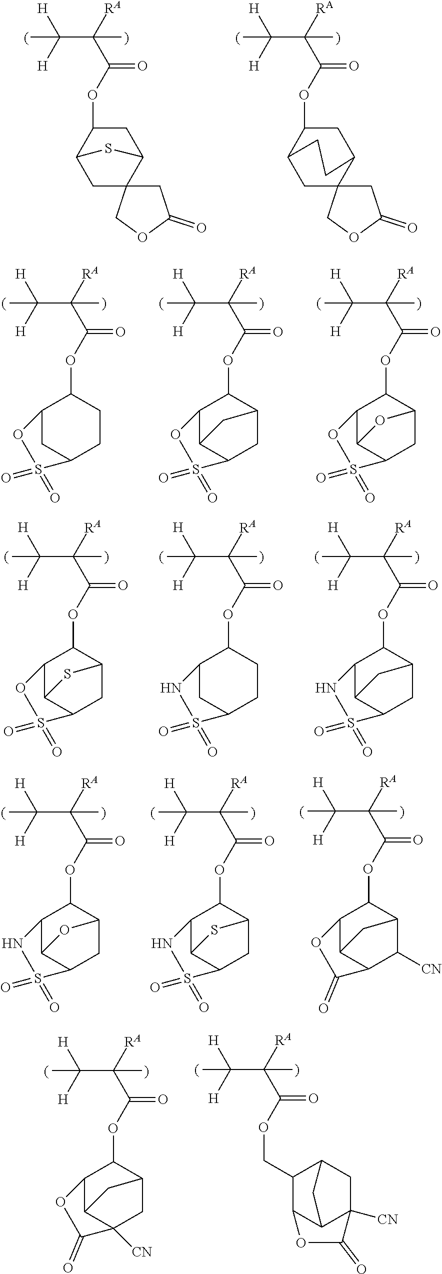

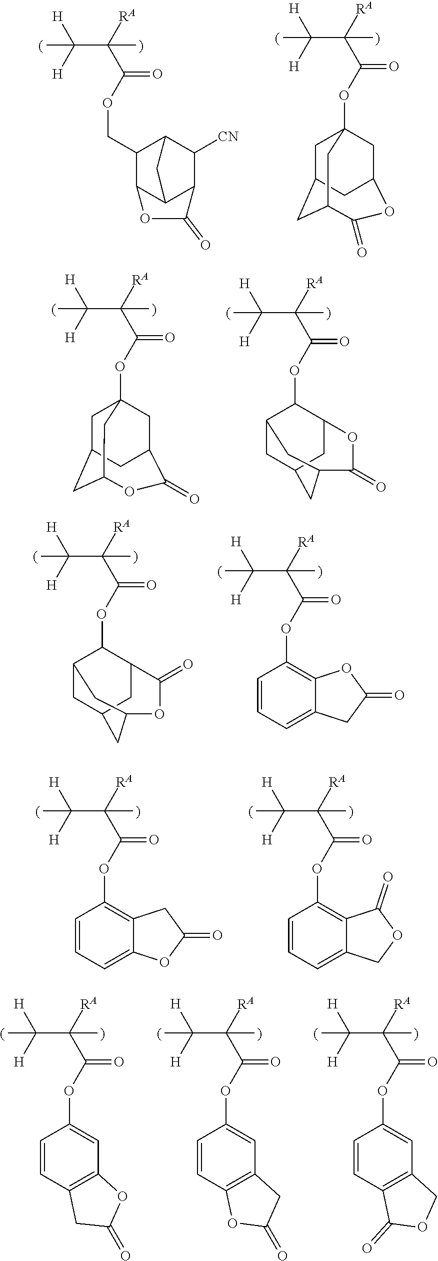

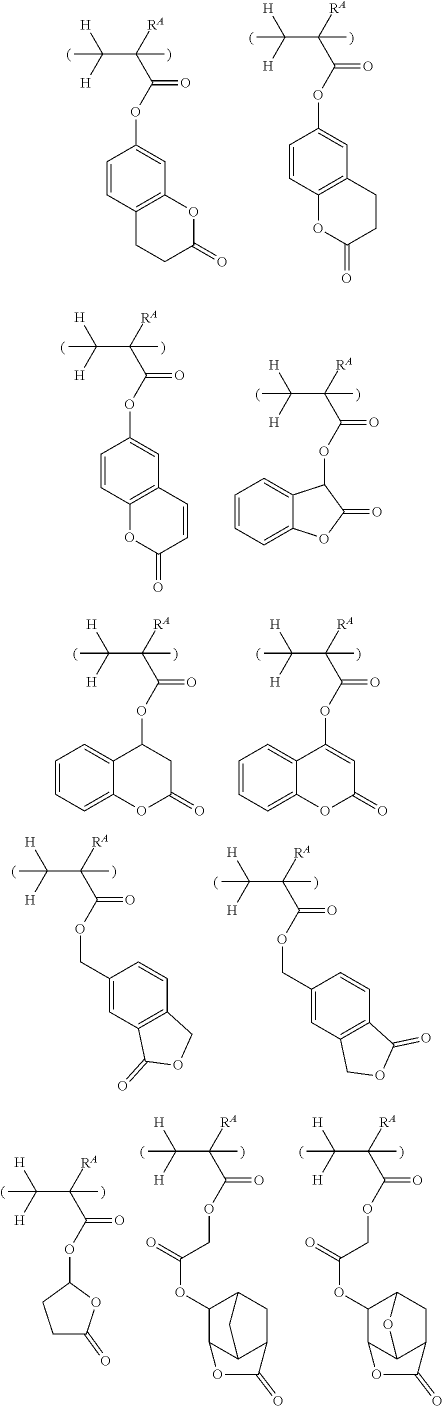

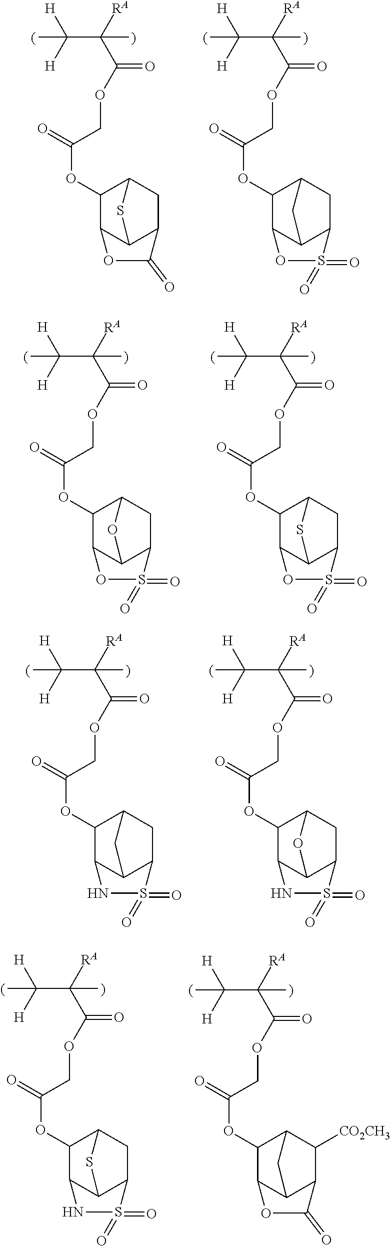

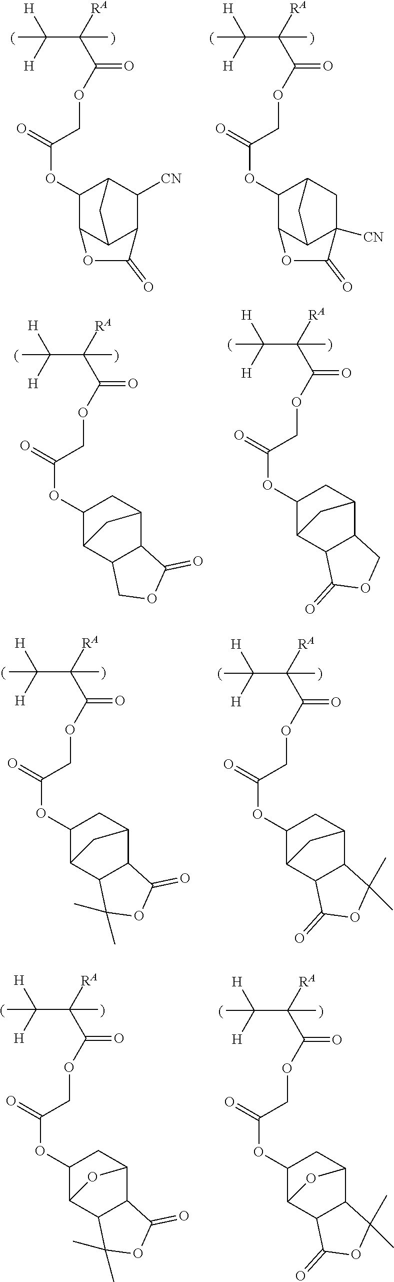

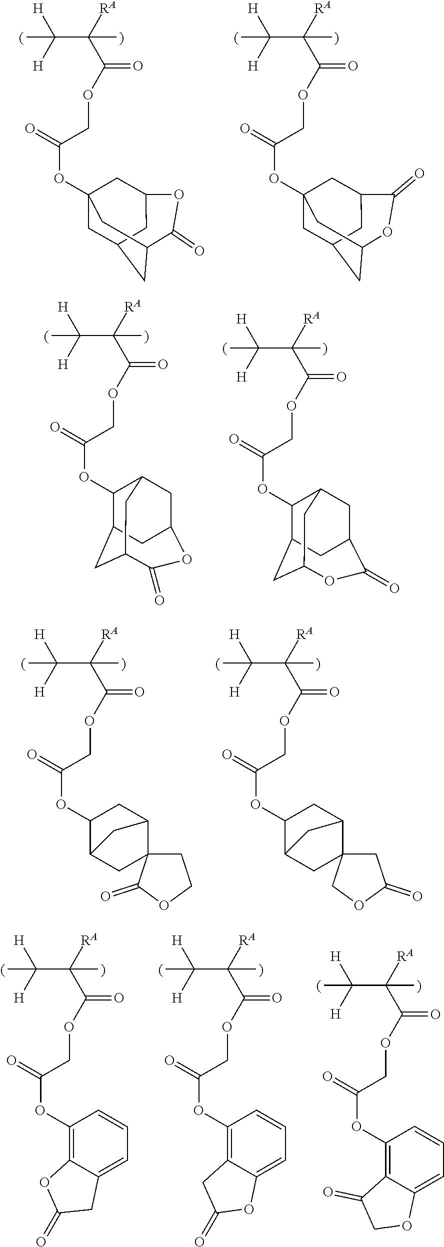

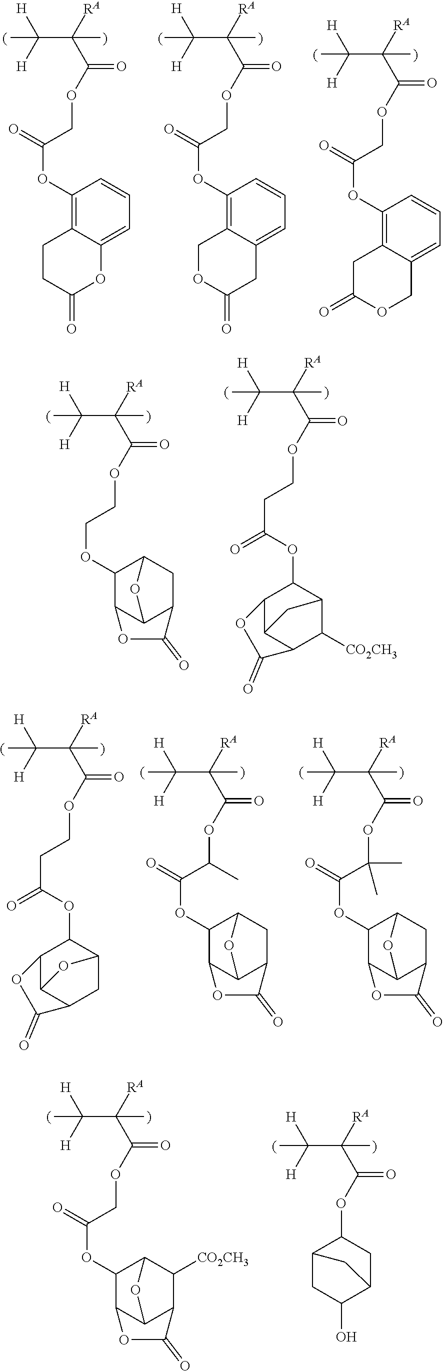

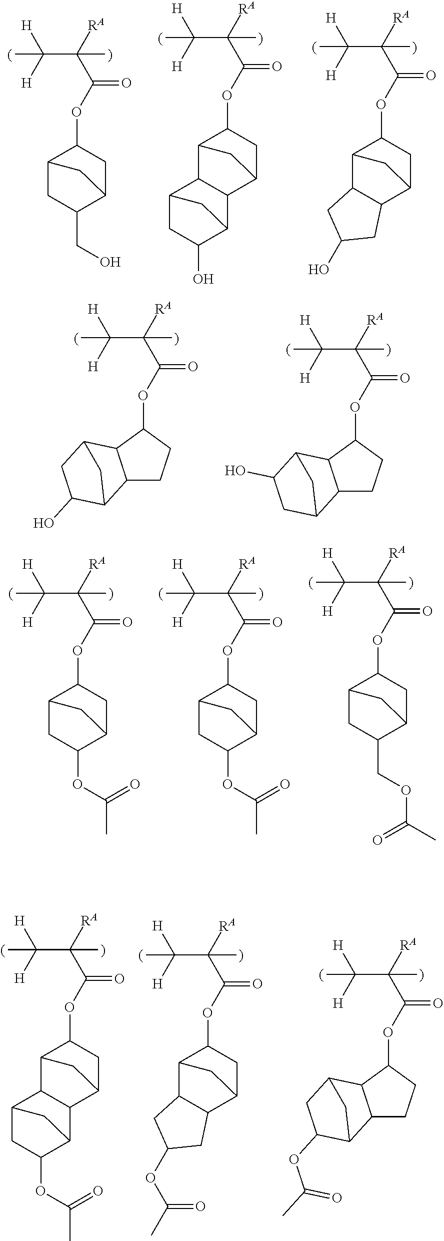

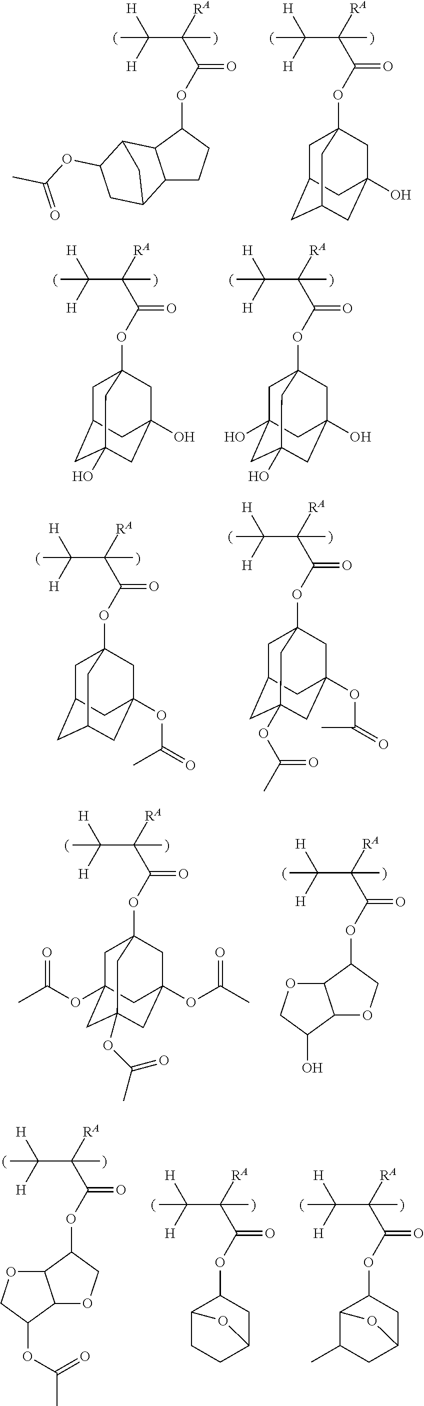

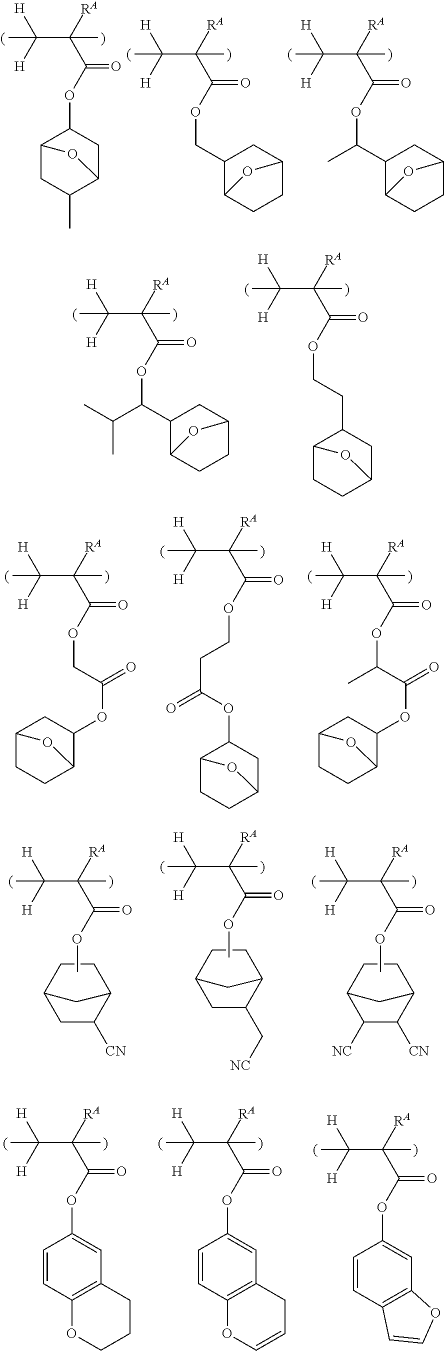

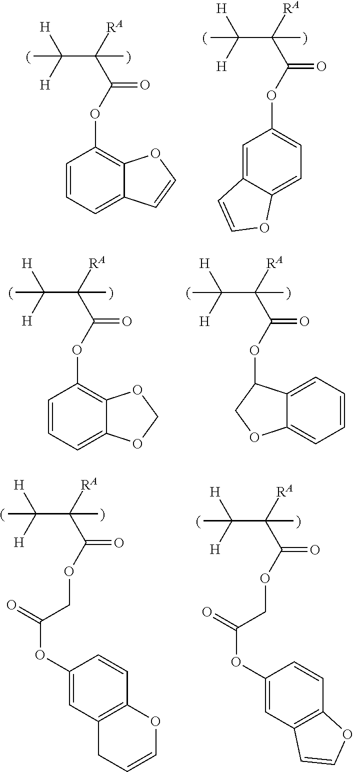

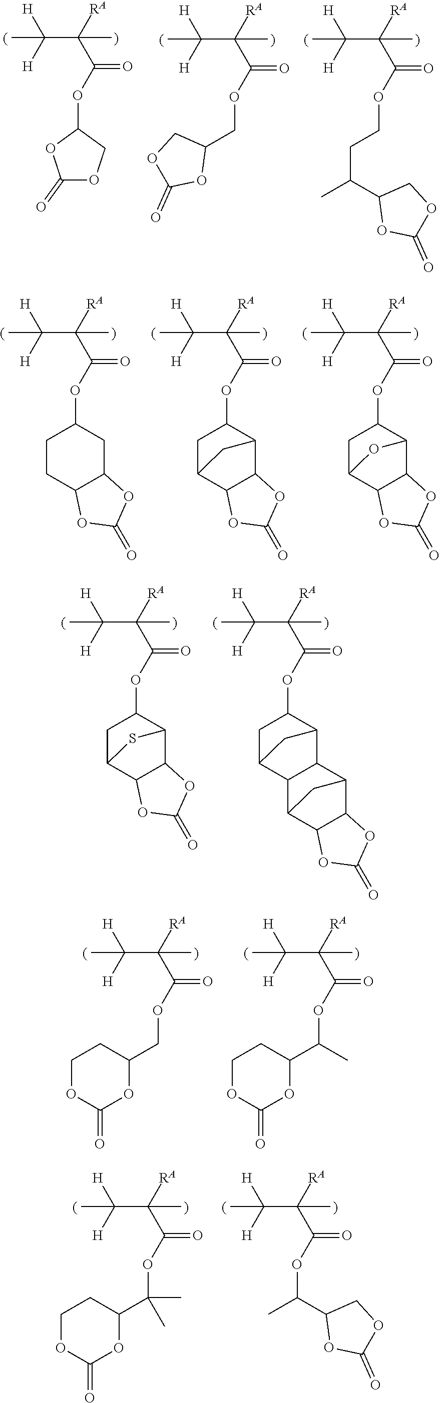

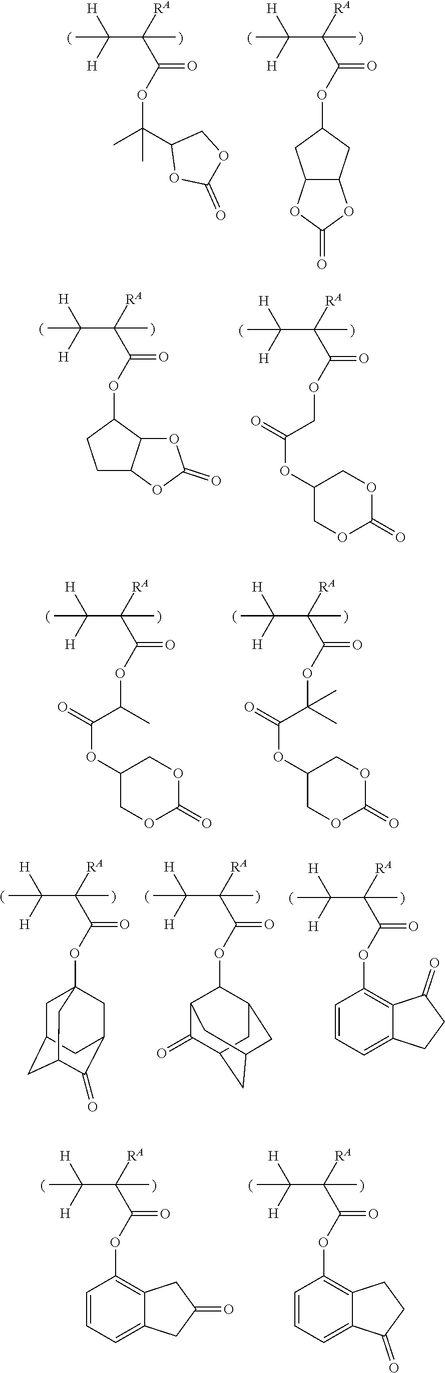

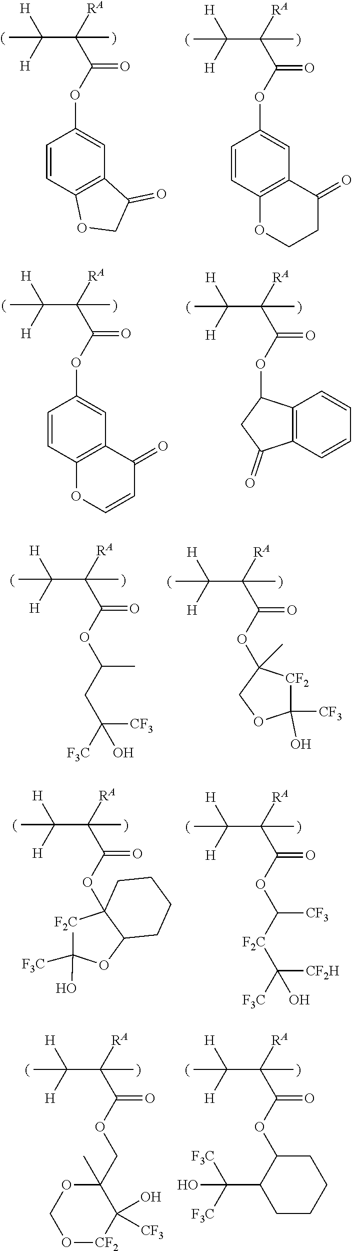

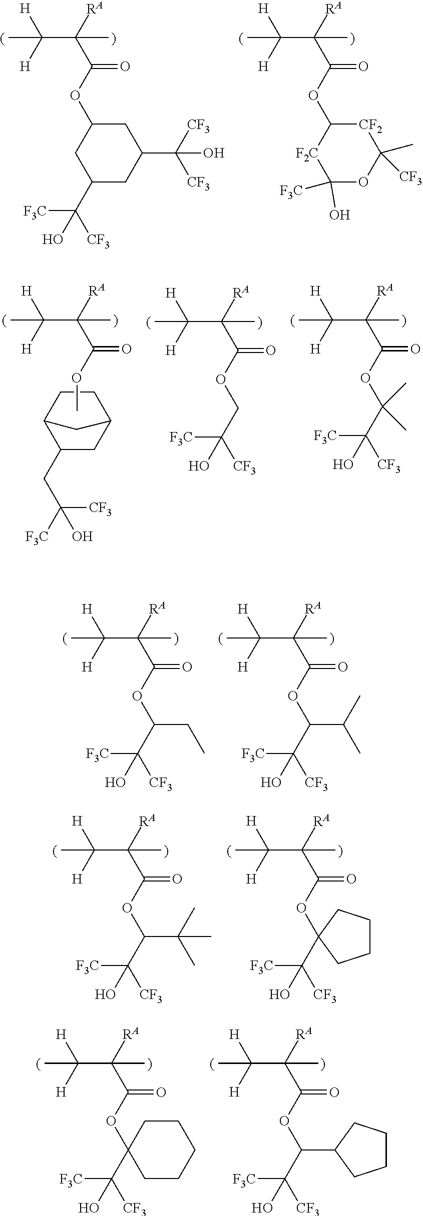

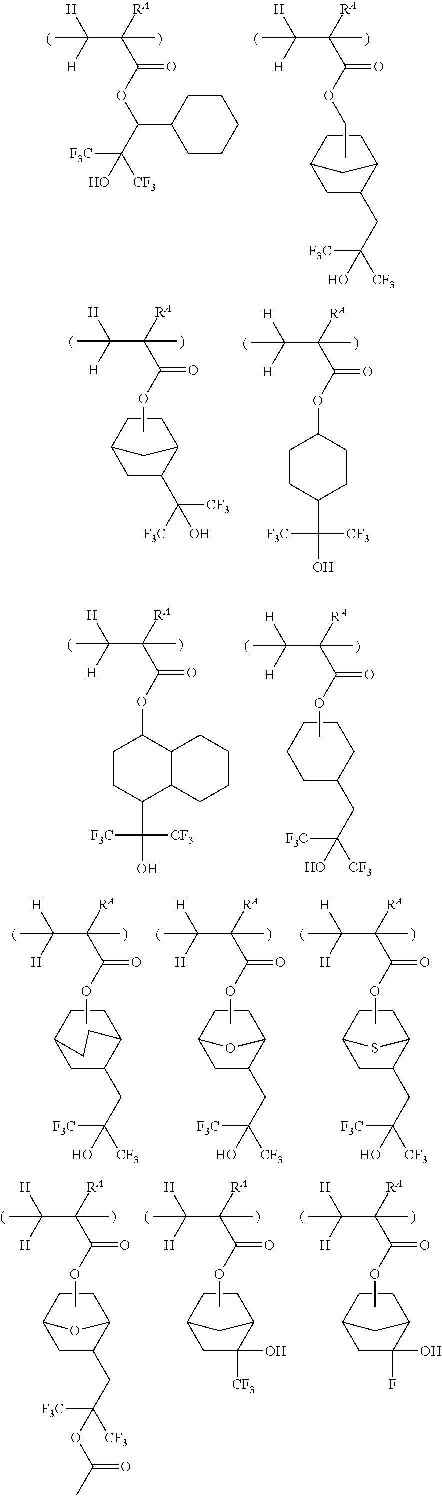

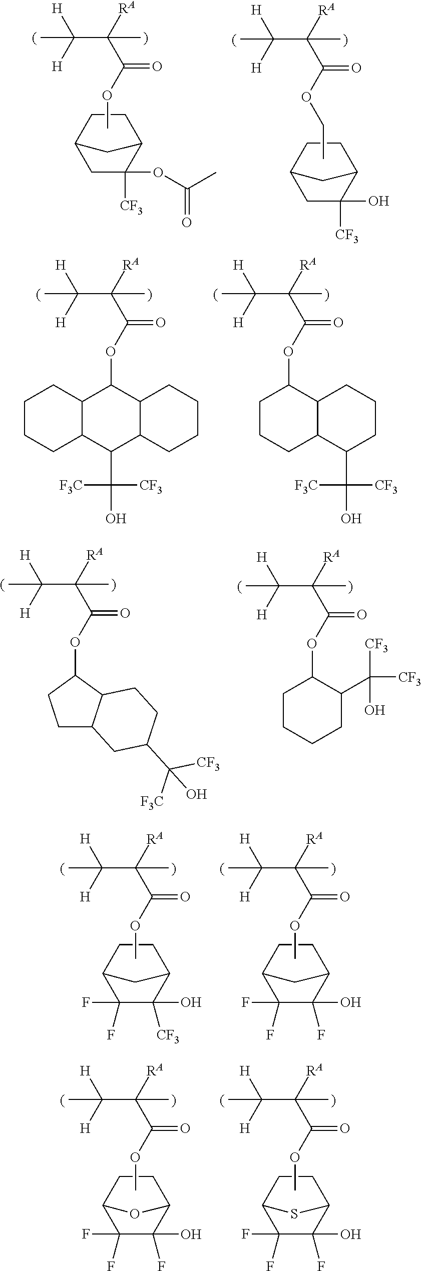

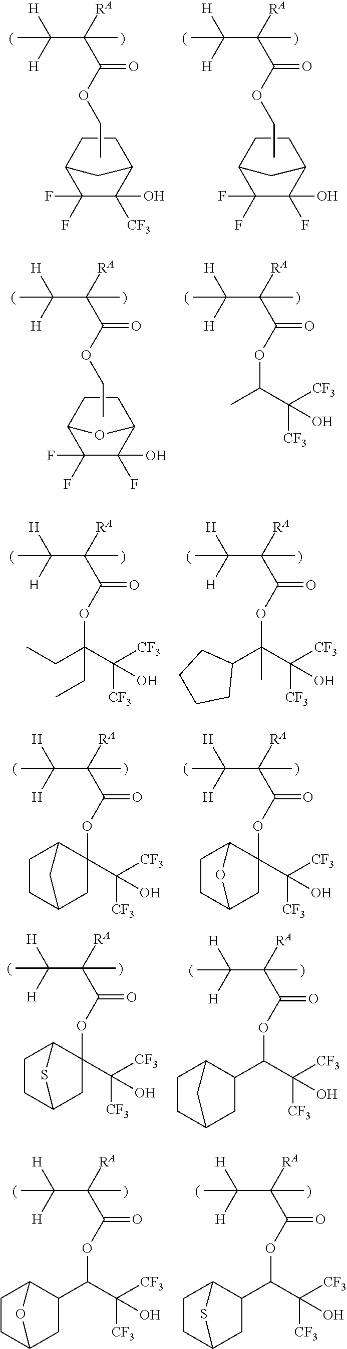

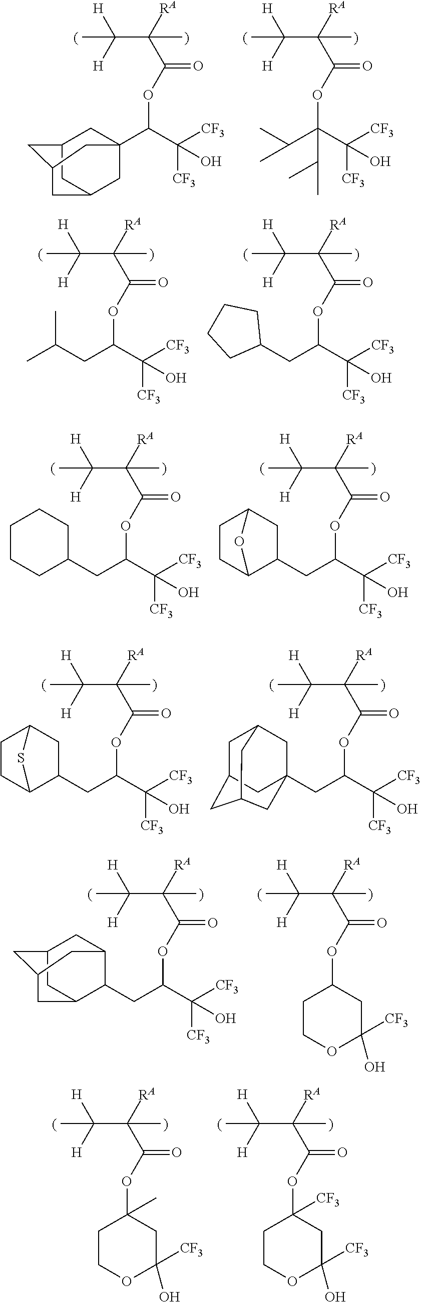

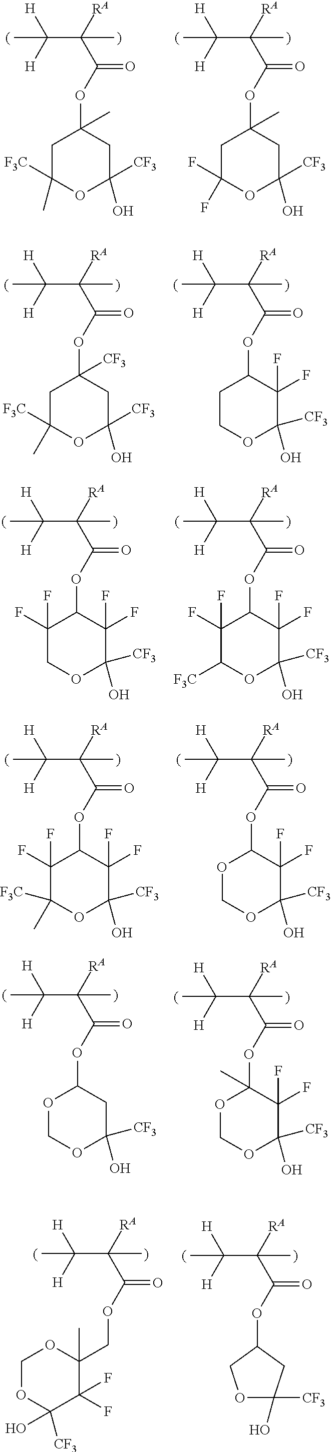

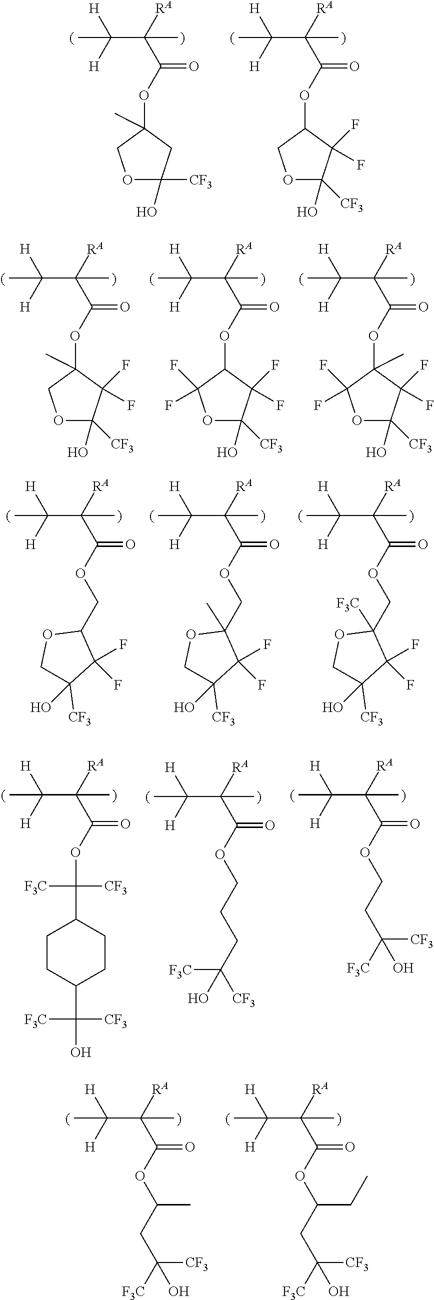

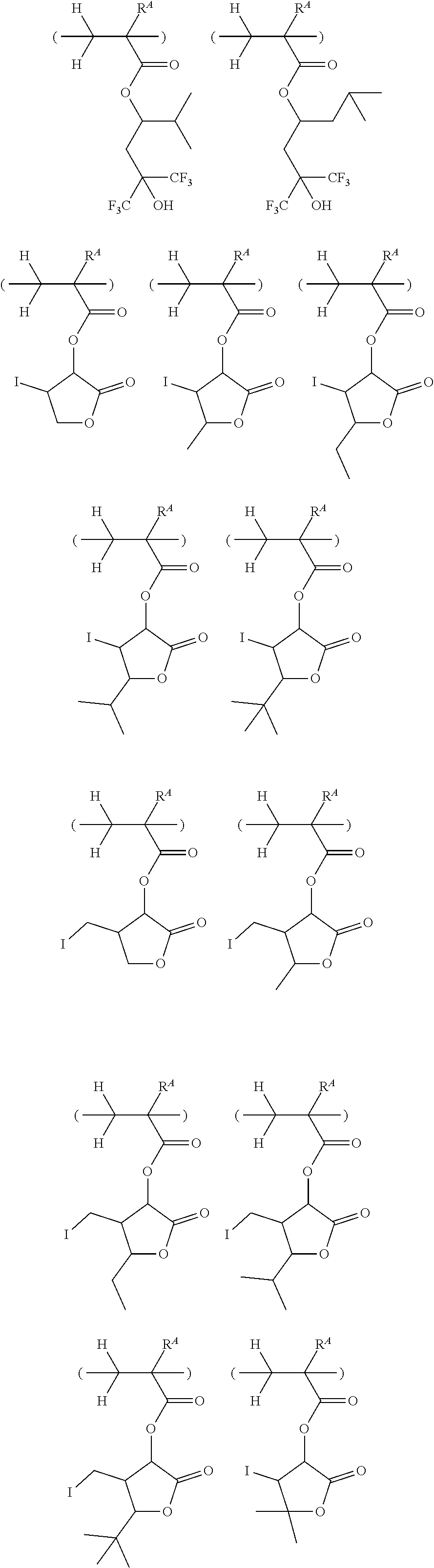

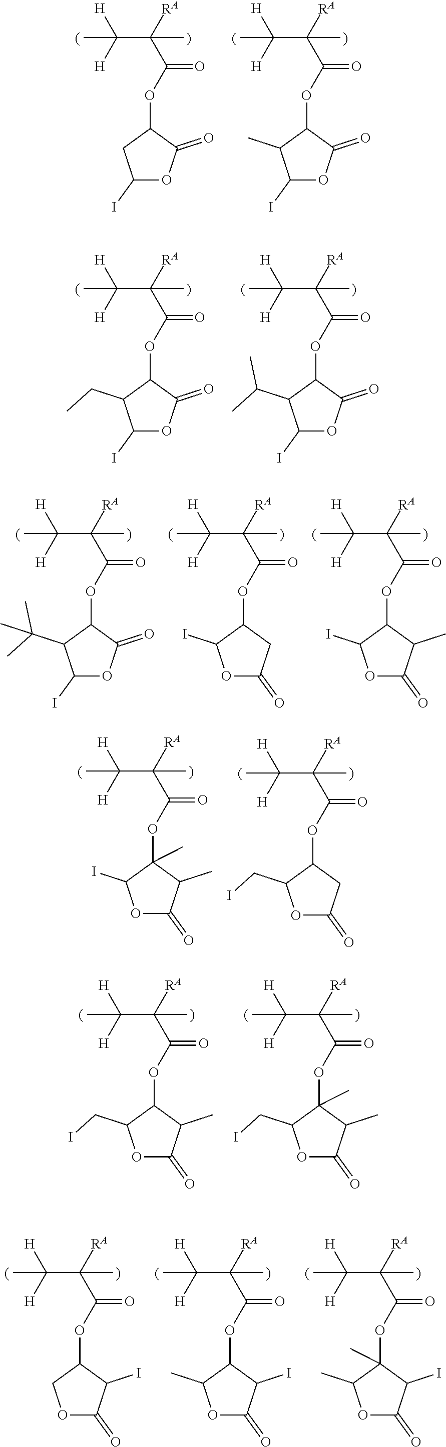

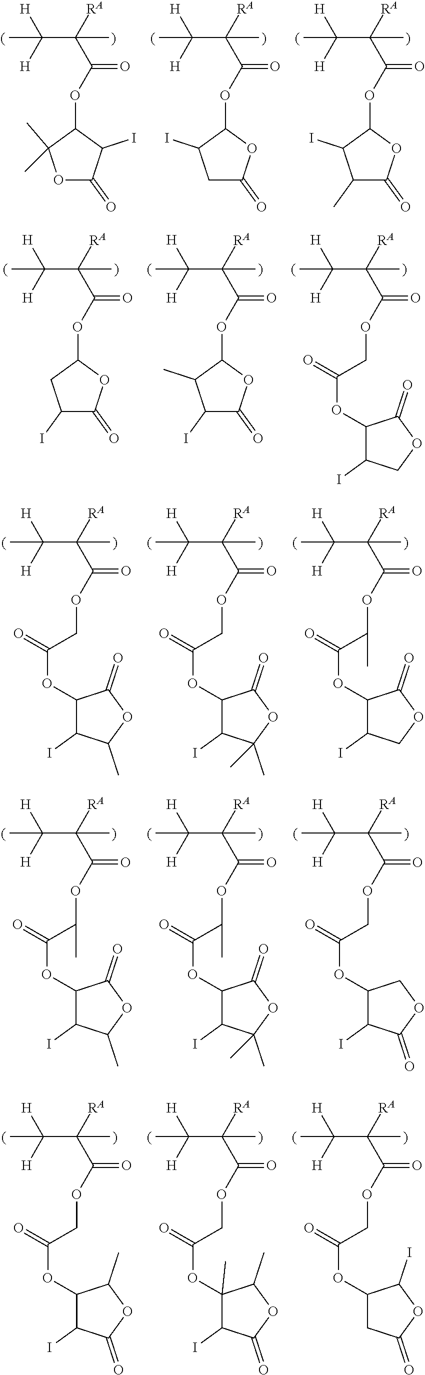

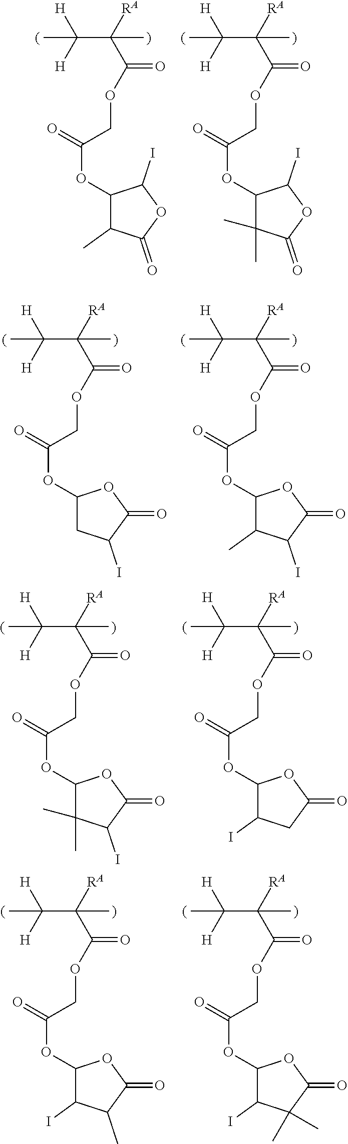

Repeating unit c1 or d1 preferably has a lactone ring, in particular, for ArF lithography, and preferably has a phenolic site for KrF lithography, EB lithography, and EUV lithography.

The polymer may further contain a repeating unit having a structure in which hydroxy groups are each protected with an acid-unstable group (hereinafter, also referred to as repeating unit e). Repeating unit e is not limited as long as it has one or two or more structures each including a protected hydroxy group and the protective groups are decomposed by the action of an acid to produce hydroxy groups, but is preferably one represented by the following formula (e1):

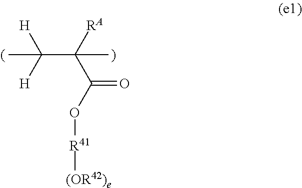

In the formula (e1), R^{A} is a hydrogen atom, a fluorine atom, a methyl group, or a trifluoromethyl group. R⁴¹ is an (e+1)-valent hydrocarbon group having 1 to 30 carbon atoms and optionally containing a heteroatom. R⁴² is an acid-unstable group. e is an integer of 1 to 4.

In the formula (e1), the acid-unstable group represented by R⁴² can be any one that deprotects by the action of an acid to generate a hydroxy group. The structure of R⁴² is not limited, but is preferably, for example, an acetal structure, a ketal structure, an alkoxycarbonyl group, or an alkoxymethyl group represented by the following formula (e2), and particularly preferably an alkoxymethyl group represented by the following formula (e2):

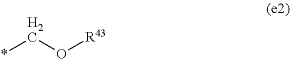

wherein * indicates bonding, and R⁴³ is a hydrocarbyl group having 1 to 15 carbon atoms.

Specific examples of the acid-unstable group represented by R⁴², and the alkoxymethyl group represented by the formula (e2), and repeating unit e include those described in JP 2020-111564 A and shown as examples in the description of repeating unit d.

The base polymer may further contain repeating unit f derived from indene, benzofuran, benzothiophene, acenaphthylene, chromone, coumarin, norbornadiene, or a derivative of any of these. Specific examples of monomers that give repeating unit f include, but are not limited to, those shown in the following:

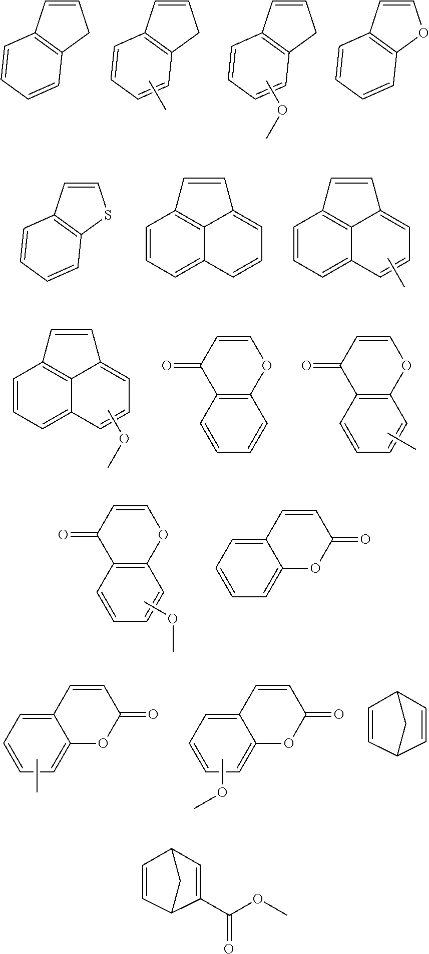

The base polymer may further contain repeating unit g derived from styrene, indan, vinylpyridine, or vinylcarbazole.

The content ratios of repeating units a, b1, b2, c1, d1, e, f, and g in the polymer of the present invention are preferably 0<a≤0.4, 0<b1≤0.8, 0≤b2≤0.8, 0<c1≤0.6, 0≤d1≤0.6, 0≤e≤0.3, 0≤f≤0.3, and 0≤g≤0.3, and more preferably 0<a≤0.3, 0<b1≤0.7, 0≤b2≤0.7, 0<c1≤0.5, 0≤d1≤0.5, 0≤e≤0.2, 0≤f≤0.2, and 0≤g≤0.2. It should be noted that a+b1+b2+c1+d1+e+f+g 1.0 is satisfied.

The weight-average molecular weight (Mw) of the polymer is preferably 1000 to 500000, more preferably 3000 to 100000, and further preferably 5000 to 20000. Mw in this range gives sufficient etching resistance, and does not cause the possibility of reduction in resolution due to failure in ensuring difference between the dissolution rates before and after exposure. In the present invention, Mw is a measurement in terms of polystyrene as determined by gel permeation chromatography (GPC) with use of tetrahydrofuran (THF) or N,N-dimethylformamide (DMF) as a solvent. GPC measurement is normally performed at room temperature around 23° C., but may be performed at a temperature higher or lower than that.

Moreover, the molecular weight distribution (Mw/Mn) of the polymer is preferably as narrow as 1.0 to 2.0 in order to obtain a resist composition that is suitably used for fine pattern dimensions because the influence of Mw/Mn tends to be more significant as pattern rules are finer. With Mw/Mn being in that range, less polymer having molecular weight lower or higher is present, and the possibility that an undesired matter is found on a pattern or a deteriorated pattern shape is formed is not caused after exposure.

Methods for synthesizing the polymer include a method of polymerizing a monomer that gives any of the repeating units described above by heating in an organic solvent with addition of a radical polymerization initiator.

Specific examples of the organic solvent to be used in the polymerization include toluene, benzene, THF, diethyl ether, dioxane, cyclohexane, cyclopentane, methyl ethyl ketone (MEK), propylene glycol monomethyl ether acetate (PGMEA), and γ-butyrolactone (GBL). Specific examples of the polymerization initiator include 2,2′-azobisisobutyronitrile (AIBN), 2,2′-azobis(2,4-dimethylvaleronitrile), dimethyl-2,2-azobis(2-methylpropionate), 1,1′-azobis(1-acetoxy-1-phenylethane), benzoyl peroxide, and lauroyl peroxide. The amount of the initiator to be added is preferably 0.01 to 25 mol % based on the total of the monomers to be polymerized. The reaction temperature is preferably 50 to 150° C., and more preferably 60 to 100° C. The reaction time is preferably 2 to 24 hours, and, for production efficiency, the reaction time is more preferably 2 to 12 hours.

The polymerization initiator may be added to the monomer solution to feed to a reactor; alternatively, an initiator solution may be prepared separately from the monomer solution to feed them independently to a reactor. Radicals generated from the initiator during stand-by time may progress polymerization reaction to produce an ultra-high-molecular-weight product, and hence the monomer solution and the initiator solution are preferably each independently prepared and added dropwise for quality control. The acid-unstable group introduced to a monomer may be used as it is; otherwise, the acid-unstable group may be protected or partially protected after polymerization. A known chain transfer agent such as dodecylmercaptan and 2-mercaptoethanol may be used in combination for molecular weight adjustment. In this case, the amount of the chain transfer agent to be added is preferably 0.01 to 20 mol % based on the total of the monomers to be polymerized.

To obtain a polymer having a much narrower polydispersity, living radical polymerization can be used. In a polymerization method for living radials that is called Reversible Addition Fragmentation chain Transfer (RAFT) polymerization, a radical at a polymer end is always living, and hence a block copolymer consisting of first repeating units and second repeating units can be formed through the process that polymerization is initiated with a first monomer and a second monomer is added after the first monomer has been consumed; even in using the RAFT polymerization for copolymerization, the radical concentration in the system is kept equilibrated, and hence the RAFT polymerization is also characterized in that, even when multiple monomers are copolymerized, a narrowly dispersed polymer with a narrow polydispersity is formed.

Examples of the organic solvent to be used in the RAFT polymerization include toluene, benzene, tetrahydrofuran (THF), diethyl ether, and dioxane. Examples of the polymerization initiator include 2,2′-azobisisobutyronitrile (AIBN), 2,2′-azobis(2,4-dimethylvaleronitrile), dimethyl 2,2-azobis(2-methylpropionate), benzoyl peroxide, and lauroyl peroxide. The temperature in the polymerization is preferably 50 to 80° C. The reaction time is preferably 2 to 100 hours, and more preferably 5 to 20 hours.

A chain transfer agent is needed for the RAFT polymerization, and specific examples thereof include 2-cyano-2-propyl benzothioate, 4-cyano-4-phenylcarbonothioylthiopentanoic acid, 2-cyano-2-propyl dodecyl trithiocarbonate, 4-cyano-4-[(dodecylsulfanylthiocarbonyl)sulfanyl]pentanoic acid, 2-(dodecylthiocarbonothioylthio)-2-methylpropanoic acid, cyanomethyl dodecyl thiocarbonate, cyanomethyl N-methyl-N-phenyl carbamothioate, bis(thiobenzoyl) disulfide, and bis(dodecylsulfanylthiocarbonyl) disulfide. Among these, 2-cyano-2-propyl benzothioate is most preferred.

For monomers containing a hydroxy group, the hydroxy group may be replaced in advance with an acetal group in polymerization, which is readily deprotected with an acid such as an ethoxyethoxy group, and deprotected with a weak acid and water after polymerization, or replaced in advance with an acetyl group, a formyl group, a pivaloyl group, or the like and subjected to alkaline hydrolysis after polymerization.

For copolymerization of hydroxystyrene or hydroxyvinylnaphthalene, hydroxystyrene or hydroxyvinylnaphthalene and another monomer may be heat-polymerized in an organic solvent with addition of a radical polymerization initiator; alternatively, acetoxystyrene or acetoxyvinylnaphthalene may be used for deprotection of the acetoxy group through alkaline hydrolysis after polymerization to form polyhydroxystyrene or hydroxypolyvinylnaphthalene.

Specific examples of the base applicable in the alkaline hydrolysis include aqueous ammonia and triethylamine. The reaction temperature is preferably −20 to 100° C., and more preferably 0 to 60° C. The reaction time is preferably 0.2 to 100 hours, and more preferably 0.5 to 20 hours.

The amount of each monomer in the monomer solution can be appropriately set to give the aforementioned preferred content ratios of the repeating units, for example.

After obtaining the polymer by the production method, the reaction solution given through the polymerization reaction may be used as a final product, and alternatively a powder obtained by adding the polymerization solution to a poor solvent and performing a purification process to give a powder such as reprecipitation may be used as a final product; however, using a polymer solution obtained by dissolving a powder given through such a purification process in a solvent as a final product is preferred for work efficiency and quality stabilization.

Specific examples of the solvent to be used in this case include those shown in paragraphs [0144] to [0145] of JP 2008-111103 A, more specifically, ketones such as cyclohexanone and methyl-2-n-pentyl ketone; alcohols such as 3-methoxybutanol, 3-methyl-3-methoxybutanol, 1-methoxy-2-propanol, and 1-ethoxy-2-propanol; ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono tert-butyl ether acetate; lactones such as GBL; alcohols such as diacetone alcohol (DAA); high-boiling-point alcoholic solvents such as diethylene glycol, propylene glycol, glycerin, 1,4-butanediol, and 1,3-butanediol; and mixed solvents of any of these.

The polymer concentration of the polymer solution is preferably 0.01 to 30% by mass, and more preferably 0.1 to 20% by mass.

The reaction solution and polymer solution are preferably subjected to filter filtration. Undesired matters and gel that may cause defects can be removed through filter filtration, and this is effective with respect to quality stabilization.

Examples of the material of the filter to be used for the filter filtration include fluorocarbon, cellulose, nylon, polyester, and hydrocarbon materials; and in the filtration process for the chemically amplified resist composition, a filter formed of fluorocarbon material, what is called Teflon (registered trademark), hydrocarbon material such as polyethylene and polypropylene, or nylon is preferred. A pore size that fits with desired cleanliness can be appropriately selected for the filter, and the pore size is preferably 100 nm or less, and more preferably 20 nm or less. One of such filters may be used alone; alternatively, a plurality of such filters may be used in combination. For the filtration, the solution may be passed only once, but the filtration is preferably performed multiple times by circulating the solution. The filtration process can be performed any times in any order in the production process for the polymer, and it is preferred to filter the reaction solution after the polymerization reaction, the polymer solution, or both of them.

### [Chemically Amplified Resist Composition]

[Base polymer (A)]

The chemically amplified resist composition of the present invention contains a base polymer containing the polymer described above as component (A).

For the polymer, one polymer may be used alone; alternatively, two or more polymers having different composition ratios, Mw, and/or Mw/Mn may be used in combination. Base polymer (A) may contain a hydrogen adduct of a ring-opening metathesis polymerization product in addition to the polymer, and those described in JP 2003-66612 A can be used therefor.

The amount of base polymer (A) contained in the chemically amplified resist composition of the present invention is not limited, and can be, for example, 0.1 to 10 parts by mass, and preferably 0.5 to 5 parts by mass based on 100 parts by mass of the composition.

### [Organic Solvent (B)]

The chemically amplified resist composition of the present invention may contain an organic solvent as component (B). Organic solvent (B) may be any organic solvent capable of dissolving the components described above and components described later without limitation. Specific examples of such organic solvents include ketones such as cyclopentanone, cyclohexanone, and methyl-2-n-pentyl ketone; alcohols such as 3-methoxybutanol, 3-methyl-3-methoxybutanol, 1-methoxy-2-propanol, and 1-ethoxy-2-propanol; keto-alcohols such as DAA; ethers such as PGME, ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono tert-butyl ether acetate; lactones such as GBL; and mixed solvents of any of these.

Preferred among those organic solvents are 1-ethoxy-2-propanol, PGMEA, cyclohexanone, GBL, DAA, and mixed solvents of any of these, which are particularly superior in ability to dissolve the base polymer as component (A).

The amount of organic solvent (B) contained in the chemically amplified resist composition of the present invention is preferably 200 to 5000 parts by mass, and more preferably 400 to 3500 parts by mass per 80 parts by mass of base polymer (A). For organic solvent (B), one organic solvent may be used alone; alternatively, two or more organic solvents may be used as a mixture.

### [Quencher (C)]

The chemically amplified resist composition of the present invention may contain a quencher as component (C). In the present invention, the term quencher refers to a material that prevents an acid generated from the photo-acid generator in the chemically amplified resist composition from diffusing to unexposed parts by trapping the acid for forming desired patterns.

Specific examples of quencher (C) include an onium salt represented by the following formula (1) or (2):

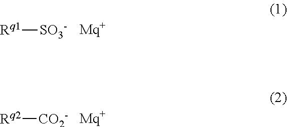

In the formula (1), R^{q1} is a hydrogen atom or a hydrocarbyl group having 1 to 40 carbon atoms and optionally containing a heteroatom, provided that each hydrogen atom bound to the carbon atom at the a position of the sulfo group is not replaced with a fluorine atom or a fluoroalkyl group. In the formula (2), R^{q2} is a hydrogen atom or a hydrocarbyl group having 1 to 40 carbon atoms and optionally containing a heteroatom.

Specific examples of the hydrocarbyl group represented by R^{q1}, having 1 to 40 carbon atoms, include an alkyl group having 1 to 40 carbon atoms such as a methyl group, an ethyl group, a n-propyl group, an isopropyl group, a n-butyl group, an isobutyl group, a sec-butyl group, a tert-butyl group, a n-pentyl group, a tert-pentyl group, a n-hexyl group, a n-octyl group, a 2-ethylhexyl group, a n-nonyl group, and a n-decyl group; a cyclic saturated hydrocarbyl group having 3 to 40 carbon atoms such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}]decyl group, and an adamantyl group; and an aryl group having 6 to 40 carbon atoms such as a phenyl group, a naphthyl group, and an anthracenyl group. Some or all of the hydrogen atoms of the hydrocarbyl group may be each replaced with a group containing a heteroatom such as an oxygen atom, a sulfur atom, a nitrogen atom, and a halogen atom, some of the —CH₂— moieties of the hydrocarbyl group may be each replaced with a group containing a heteroatom such as an oxygen atom, a sulfur atom, and a nitrogen atom, and as a result, for example, a hydroxy group, a fluorine atom, a chlorine atom, a bromine atom, an iodine atom, a cyano group, a carbonyl group, an ether bond, an ester bond, a sulfonic ester bond, a carbonate bond, a lactone ring, a sultone ring, a carboxylic anhydride (—C(═O)—O—C(═O)—), or a haloalkyl group may be contained.

Specific examples of the hydrocarbyl group represented by R^{q2} include not only the substituents shown as specific examples of R^{q1}, but also a fluorinated saturated hydrocarbyl group such as a trifluoromethyl group and a trifluoroethyl group, and a fluorinated aryl group such as a pentafluorophenyl group and a 4-trifluoromethylphenyl group.

Specific examples of the anion of the onium salt represented by the formula (1) include, but are not limited to, those shown in the following:

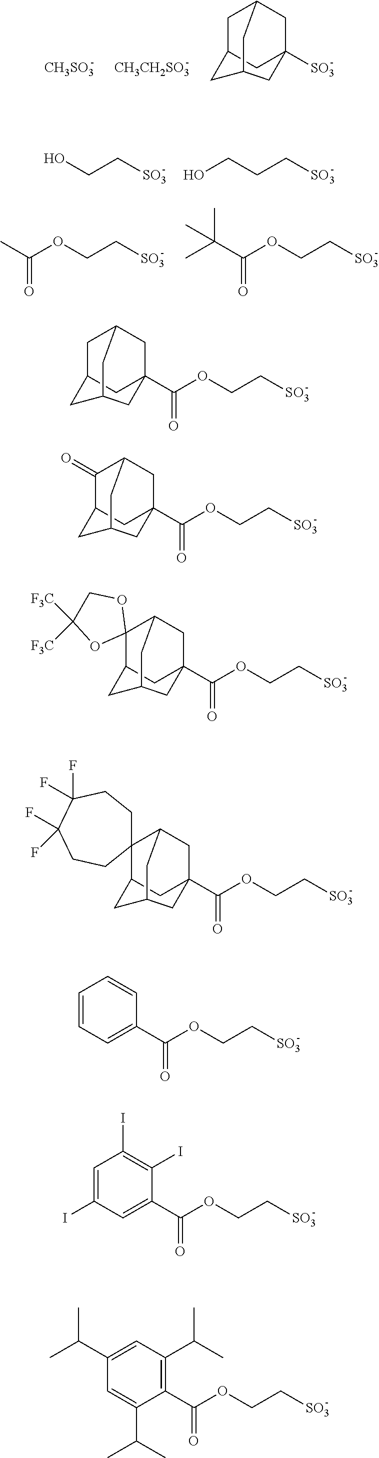

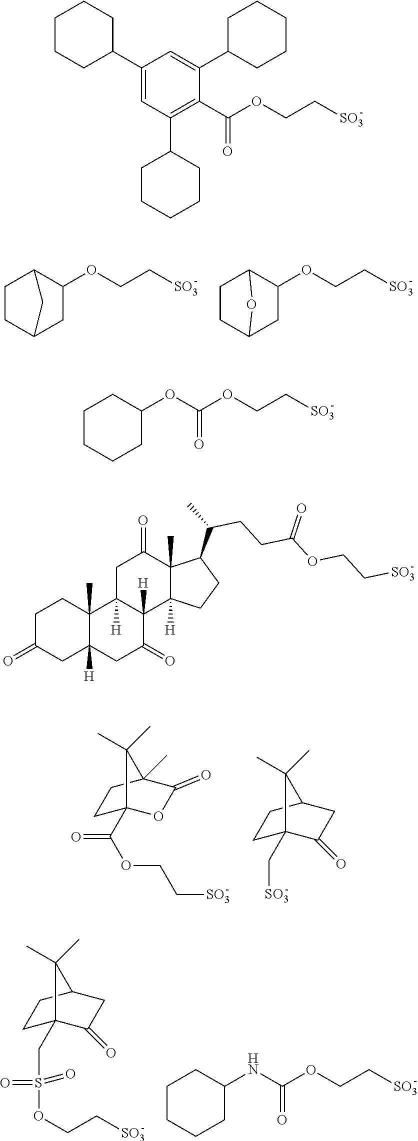

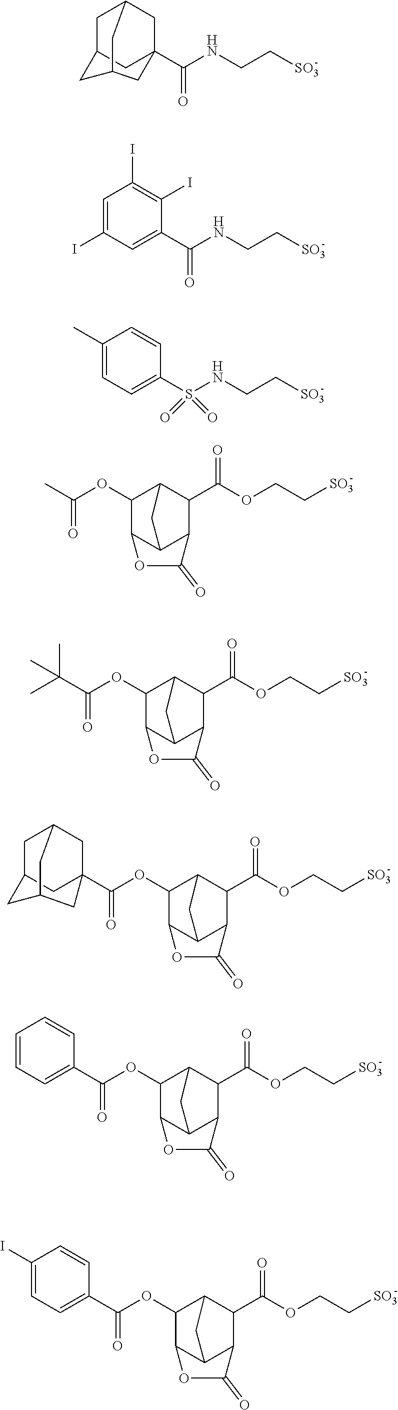

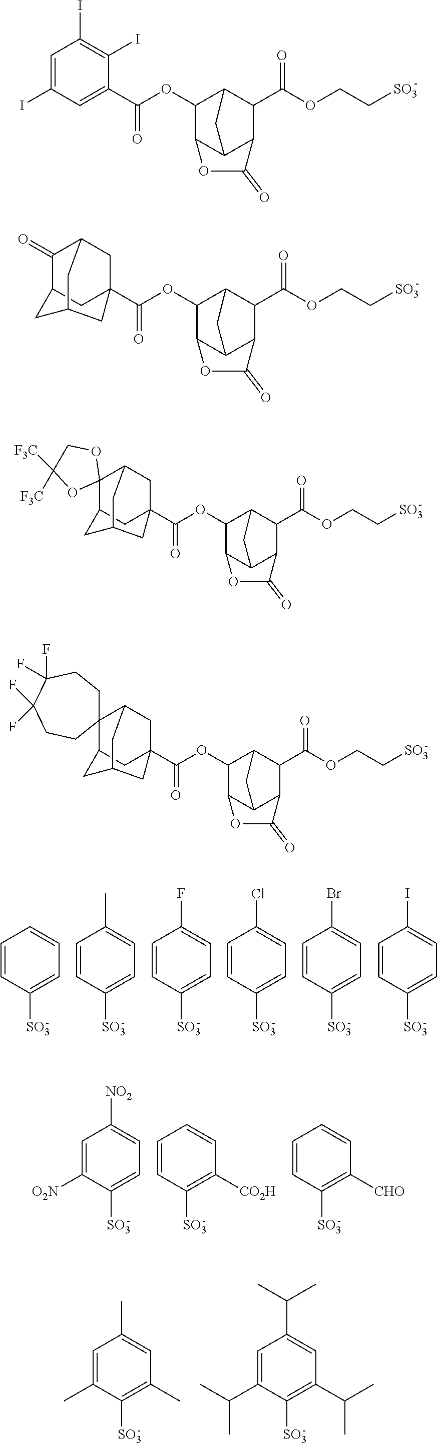

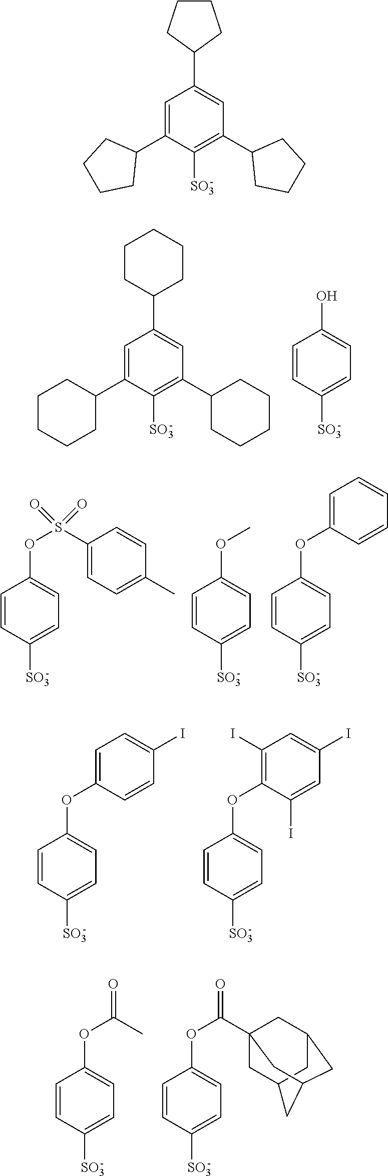

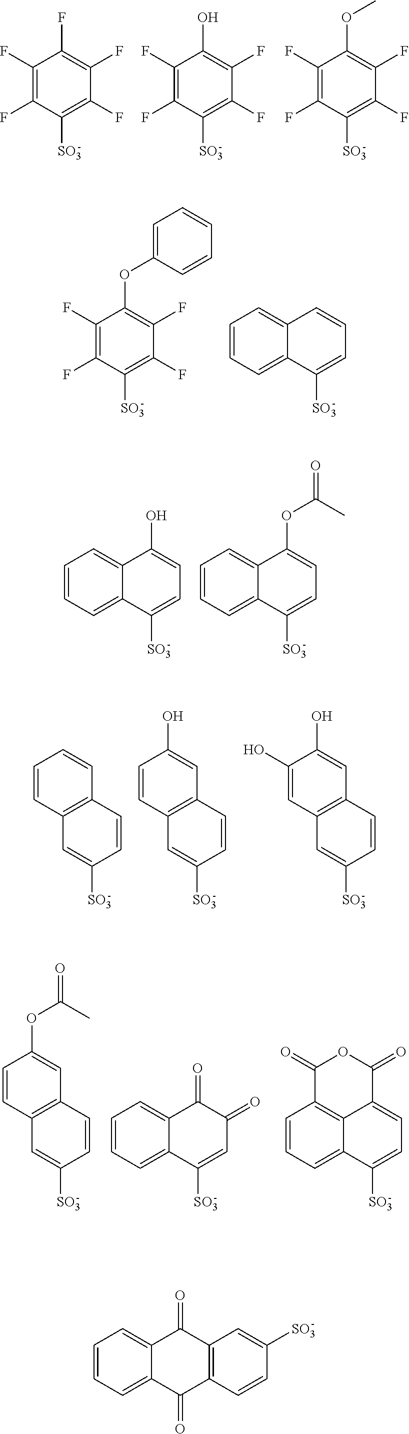

Specific examples of the anion of the onium salt represented by the formula (2) include, but are not limited to, those shown in the following:

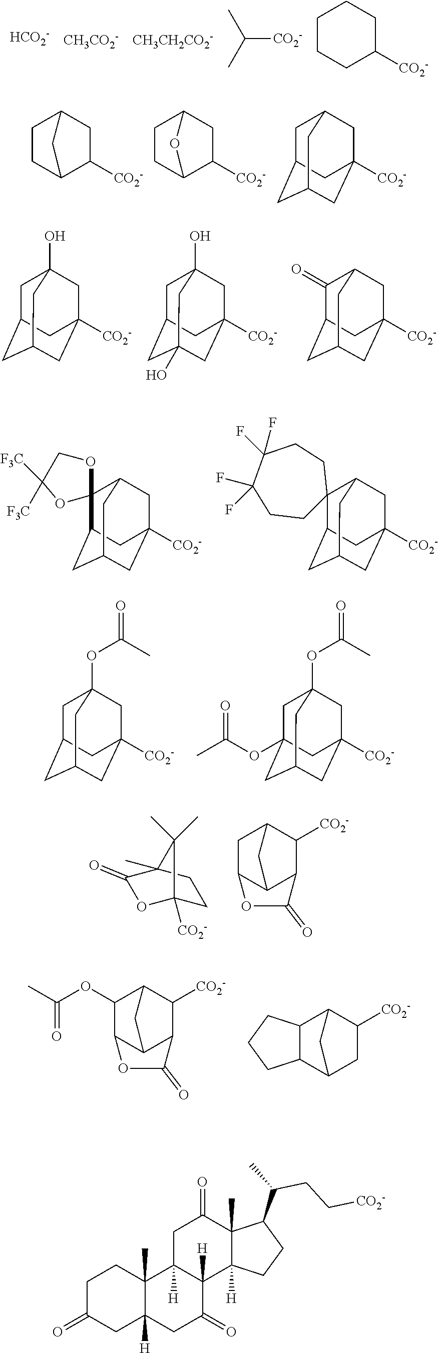

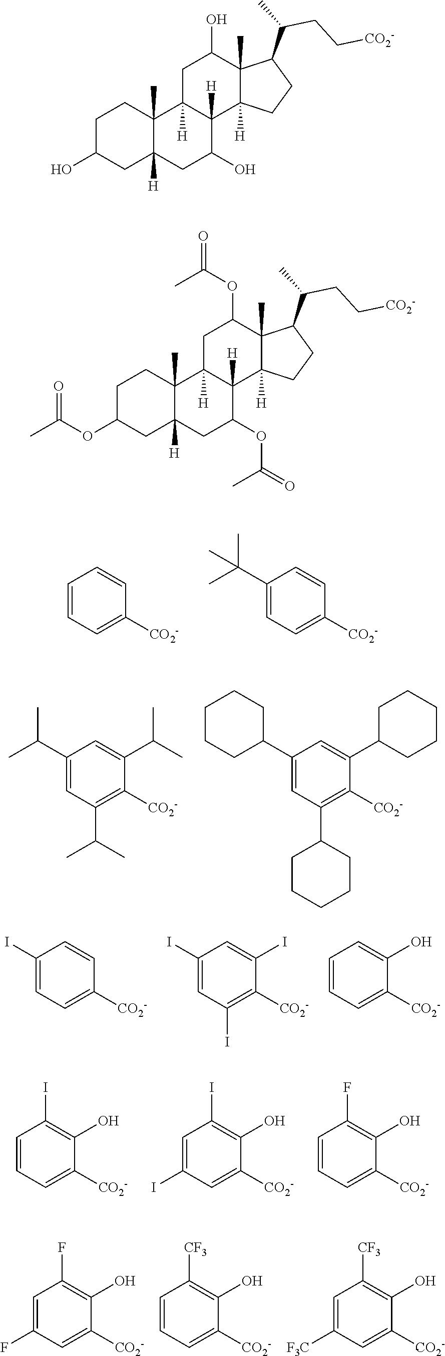

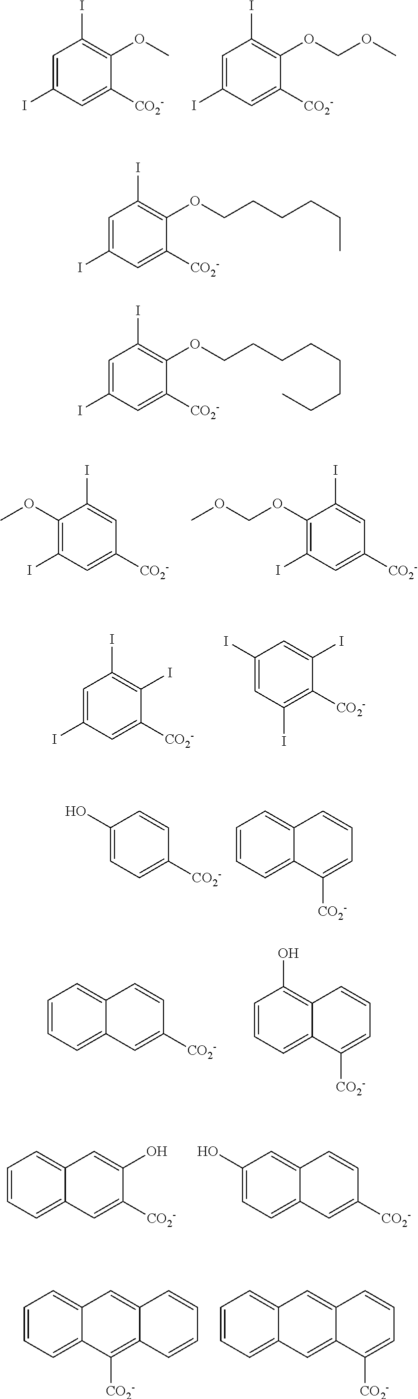

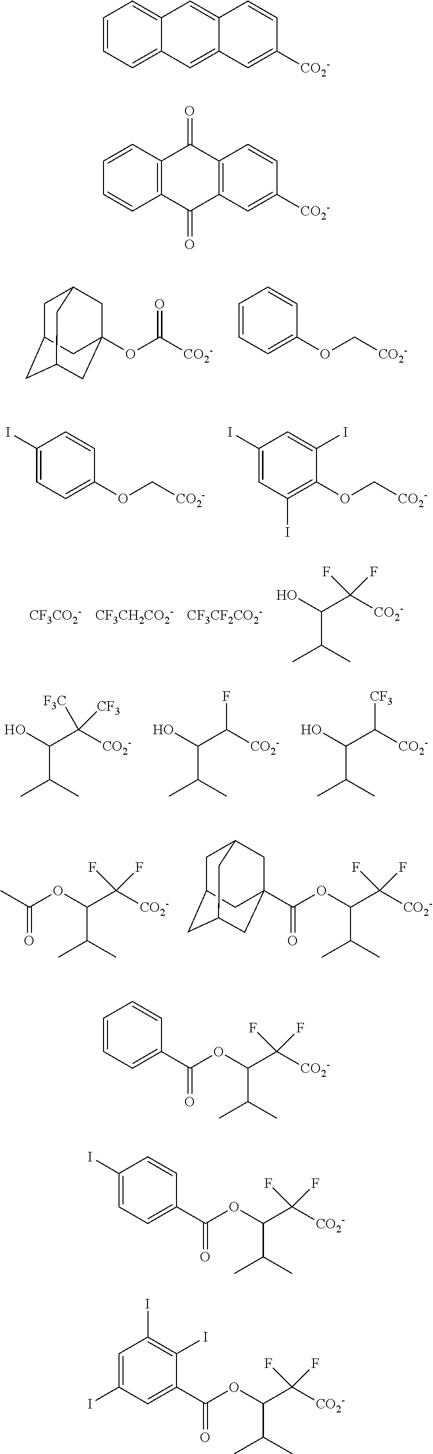

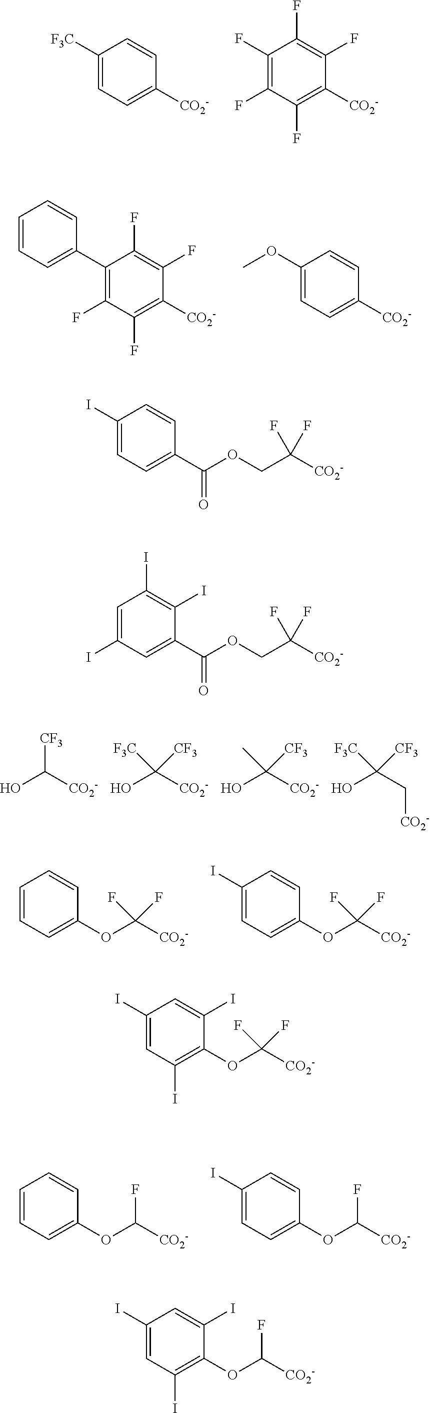

In the formulae (1) and (2), Mq⁺ is an onium cation. The onium cation is preferably the above-described sulfonium cation represented by the formula (cation-1), the above-described iodonium cation represented by the formula (cation-2), or an ammonium cation represented by the following formula (cation-3):

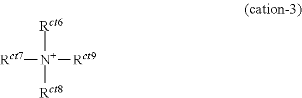

In the formula (cation-3), R^{ct6} to R^{ct9} are each independently a hydrocarbyl group having 1 to 40 carbon atoms and optionally containing a heteroatom. R^{ct6} and R^{ct7} are optionally bound together to form a ring together with the nitrogen atom to which they are bound. Specific examples of the hydrocarbyl group include those shown as examples of the hydrocarbyl group represented by R^{ct1} to R^{ct5} in the description of the formulae (cation-1) and (cation-2).

Specific examples of the ammonium cation represented by the formula (cation-3) include, but are not limited to, those shown in the following:

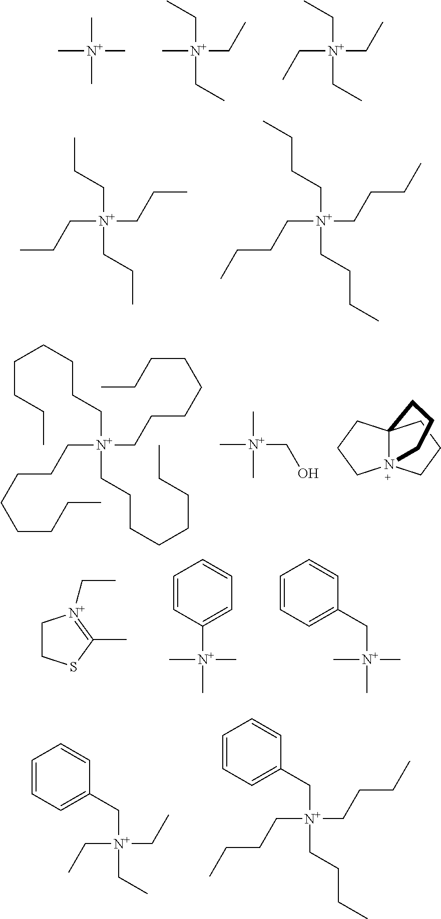

Specific examples of the onium salt represented by the formula (1) or (2) include any combinations of the aforementioned anions and cations. These onium salts are easily prepared through ion exchange reaction by using a known organic chemistry method. For the ion exchange reaction, for example, reference can be made to JP 2007-145797 A.

The onium salt represented by the formula (1) or (2) acts as a quencher in the chemically amplified resist composition of the present invention. This is due to the fact that each counteranion of the onium salt is a conjugate base of a weak acid. The term weak acid mentioned here refers to an acid that exhibits acidity such that the acid-unstable group of each acid-unstable group-containing unit used for the base polymer cannot be deprotected. The onium salt represented by the formula (1) or (2) acts as a quencher when used in combination with an onium salt photo-acid generator having, as a counteranion, a conjugate base of a strong acid such as sulfonic acid fluorinated at the a position. Specifically, if an onium salt that generates a strong acid such as sulfonic acid fluorinated at the a position and an onium salt that generates a weak acid such as sulfonic acid not fluorinated and carboxylic acid are used as a mixture, a strong acid generated from a photo-acid generator through high-energy ray irradiation collides with the onium salt having an unreacted weak acid anion, and then the weak acid is released as a result of salt exchange and an onium salt having a strong acid anion is formed. Through this process, the strong acid is exchanged with the weak acid, which has lower catalytic ability, and hence the acid is apparently deactivated; thus, acid diffusion can be controlled.

Applicable as quencher (C) are an onium salt described in JP 6848776 B, which has sulfonium cation and phenoxide anion moieties in the same molecule, an onium salt described in JP 6583136 B and JP 2020-200311 A, which has sulfonium cation and carboxylate anion moieties in the same molecule, and an onium salt described in JP 6274755 B, which has iodonium cation and carboxylate anion moieties in the same molecule.

Here, if the phot-acid generator that generates a strong acid is an onium salt, the strong acid generated through high-energy ray irradiation can be exchanged with a weak acid as described above, but the weak acid generated through high-energy ray irradiation is expected to be less likely to undergo salt exchange through collision with the unreacted onium salt that generates a strong acid. This is due to the phenomenon that the onium cation readily forms an ion pair with a stronger acid anion.

If the chemically amplified resist composition of the present invention contains the onium salt represented by the formula (1) or (2) as quencher (C), the amount of quencher (C) contained therein is preferably 0.1 to 20 parts by mass, and more preferably 0.1 to 10 parts by mass per 80 parts by mass of base polymer (A). The case that the onium salt quencher contained as component (C) falls within that range is preferred because good resolution results and remarkable reduction in sensitivity is not caused. For the onium salt represented by the formula (1) or (2), one onium salt may be used alone; alternatively, two or more onium salts may be used in combination.

The chemically amplified resist composition of the present invention may contain a nitrogen-containing compound as quencher (C). Specific examples of the nitrogen-containing compound as component (C) include any of primary, secondary, and tertiary amine compounds shown in paragraphs [0146] to [0164] of JP 2008-111103 A, especially, an amine compound having a hydroxy group, an ether bond, an ester bond, a lactone ring, a cyano group, or a sulfonic ester bond. In addition, a compound obtained by protecting a primary or secondary amine with a carbamate group, like a compound described in JP 3790649 B, can be shown as an example.

A sulfonic acid sulfonium salt having a nitrogen-containing substituent may be used as the nitrogen-containing compound. Such a compound functions as a quencher in unexposed parts, and in exposed parts loses the quencher ability through neutralization with an acid generated from the compound itself, in other words, functions as a photodegradable base. Use of a photodegradable base advantageously gives stronger contrast between exposed parts and unexposed parts. For the photodegradable base, for example, reference can be made to JP 2009-109595 A and JP 2012-46501 A.

If the chemically amplified resist composition of the present invention contains the nitrogen-containing compound as quencher (C), the amount of the nitrogen-containing compound contained therein is preferably 0.001 to 12 parts by mass, and more preferably 0.01 to 8 parts by mass per 80 parts by mass of base polymer (A). For the nitrogen-containing compound, one nitrogen-containing compound may be used alone; alternatively, two or more nitrogen-containing compounds may be used in combination.

### [Acid Generator (D)]

The chemically amplified resist composition of the present invention may further contain an acid generator, in addition to base polymer (A) as a polymer-bound photo-acid generator. Examples of the acid generator include a compound that generates an acid in response to an active ray or radiation (photo-acid generator). The photo-acid generator may be any compound that generates an acid through high-energy ray irradiation without limitation, and is preferably a compound that generates sulfonic acid, imidic acid, or methidic acid. Preferred photo-acid generators include sulfonium salt, iodonium salt, sulfonyldiazomethane, N-sulfonyloxyimide, and oxime-O-sulfonate acid generators. Specific examples of the acid generator include those shown in paragraphs [0122] to [0142] of JP 2008-111103 A.

For the photo-acid generator, a sulfonium salt represented by the following formula (3-1) and an iodonium salt represented by the following formula (3-2) can also be suitably used:

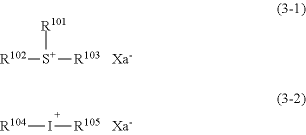

In the formulae (3-1) and (3-2), R¹⁰¹ to R¹⁰⁵ are each independently a halogen atom or a hydrocarbyl group having 1 to 20 carbon atoms and optionally containing a heteroatom. Specific examples of the halogen atom and hydrocarbyl group include those shown as examples of the halogen atom and hydrocarbyl group represented by any of R^{ct1} to R^{ct5} in the description of the formulae (cation-1) and (cation-2). Some or all of the hydrogen atoms of the hydrocarbyl group may be each replaced with a group containing a heteroatom such as an oxygen atom, a sulfur atom, a nitrogen atom, and a halogen atom, some of the —CH₂— moieties of the hydrocarbyl group may be each replaced with a group containing a heteroatom such as an oxygen atom, a sulfur atom, and a nitrogen atom, and as a result, for example, a hydroxy group, a fluorine atom, a chlorine atom, a bromine atom, an iodine atom, a cyano group, a nitro group, a carbonyl group, an ether bond, an ester bond, a sulfonic ester bond, a carbonate bond, a lactone ring, a sultone ring, a carboxylic anhydride (—C(═O)—O—C(═O)—), or a haloalkyl group may be contained. R¹⁰¹ and R¹⁰² are optionally bound together to form a ring together with the sulfur atom to which they are bound. Specific examples of the ring formed in this case include those shown as examples of the ring that can be formed through bonding of R^{ct1} and R^{ct2} together with the sulfur atom to which they are bound in the description of the formula (cation-1).

Specific examples of the cation of the sulfonium salt represented by the formula (3-1) include those shown as examples of the sulfonium cation represented by the formula (cation-1). Specific examples of the cation of the iodonium salt represented by the formula (3-2) include those shown as examples of the iodonium cation represented by the formula (cation-2).

In the formulae (3-1) and (3-2), Xa⁻ is an anion selected from the following formulae (3A) to (3D):

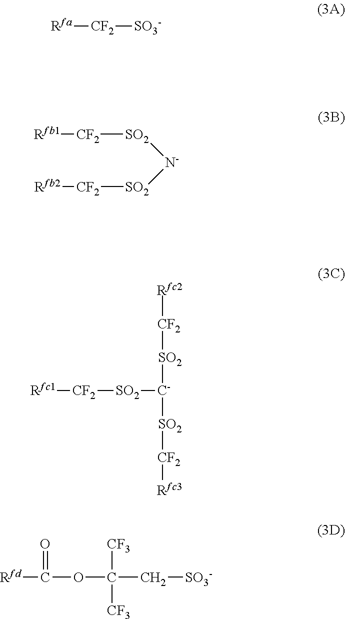

In the formula (3A), R^{fa} is a fluorine atom or a hydrocarbyl group having 1 to 40 carbon atoms and optionally containing a heteroatom. The hydrocarbyl group may be saturated or unsaturated, and may be linear, branched, or cyclic. Specific examples thereof include those to be shown as examples later in description of Ral in formula (3A′).

The anion represented by the formula (3A) is preferably one represented the following formula (3A′):

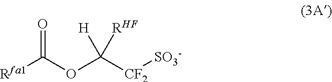

In the formula (3A′), RH is a hydrogen atom or a trifluoromethyl group, and preferably a trifluoromethyl group.

In the formula (3A′), Ral is a hydrocarbyl group having 1 to 38 carbon atoms and optionally containing a heteroatom. For example, the heteroatom is preferably an oxygen atom, a nitrogen atom, a sulfur atom, or a halogen atom, and more preferably an oxygen atom. The hydrocarbyl group is particularly preferably one having 6 to 30 carbon atoms for achievement of high resolution in fine patterning.

The hydrocarbyl group represented by R^{fa1}, having 1 to 38 carbon atoms, may be saturated or unsaturated, and may be linear, branched, or cyclic. Specific examples thereof include an alkyl group having 1 to 38 carbon atoms such as a methyl group, an ethyl group, a n-propyl group, an isopropyl group, a n-butyl group, an isobutyl group, a sec-butyl group, a tert-butyl group, a pentyl group, a neopentyl group, a hexyl group, a heptyl group, a 2-ethylhexyl group, a nonyl group, an undecyl group, a tridecyl group, a pentadecyl group, a heptadecyl group, and an icosyl group; a cyclic saturated hydrocarbyl group having 3 to 38 carbon atoms such as a cyclopentyl group, a cyclohexyl group, a 1-adamantyl group, a 2-adamantyl group, a 1-adamantylmethyl group, a norbornyl group, a norbornylmethyl group, a tricyclodecyl group, a tetracyclododecyl group, a tetracyclododecylmethyl group, and a dicyclohexylmethyl group; an unsaturated aliphatic hydrocarbyl group having 2 to 38 carbon atoms such as an allyl group and a 3-cyclohexenyl group; an aryl group having 6 to 38 carbon atoms such as a phenyl group, a 1-naphthyl group, and a 2-naphthyl group; an aralkyl group having 7 to 38 carbon atoms such as a benzyl group and a diphenylmethyl group; and a group given by combining any of these.

Some or all of the hydrogen atoms of the hydrocarbyl group may be each replaced with a group containing a heteroatom such as an oxygen atom, a sulfur atom, a nitrogen atom, and a halogen atom, some of the —CH₂— moieties of the hydrocarbyl group may be each replaced with a group containing a heteroatom such as an oxygen atom, a sulfur atom, and a nitrogen atom, and as a result, for example, a hydroxy group, a fluorine atom, a chlorine atom, a bromine atom, an iodine atom, a cyano group, a carbonyl group, an ether bond, an ester bond, a sulfonic ester bond, a carbonate bond, a lactone ring, a sultone ring, a carboxylic anhydride (—C(═O)—O—C(═O)—), or a haloalkyl group may be contained. The heteroatom is preferably an oxygen atom. Specific examples of the hydrocarbyl group containing a heteroatom include a tetrahydrofuryl group, a methoxymethyl group, an ethoxymethyl group, a methylthiomethyl group, an acetamidomethyl group, a trifluoroethyl group, a (2-methoxyethoxy)methyl group, an acetoxymethyl group, a 2-carboxy-1-cyclohexyl group, a 2-oxopropyl group, a 4-oxo-1-adamantyl group, and 3-oxocyclohexyl group.

Synthesis of sulfonium salts containing the anion represented by formula (3A′) is described in detail, for example, in JP 2007-145797 A, JP 2008-106045 A, JP 2009-7327 A, and JP 2009-258695 A. For example, sulfonium salts described in JP 2010-215608 A, JP 2012-41320 A, JP 2012-106986 A, and JP 2012-153644 A are also suitably used.

Specific examples of the anion represented by the formula (3A) include, but are not limited to, those shown below. In the following formulae, Ac is an acetyl group:

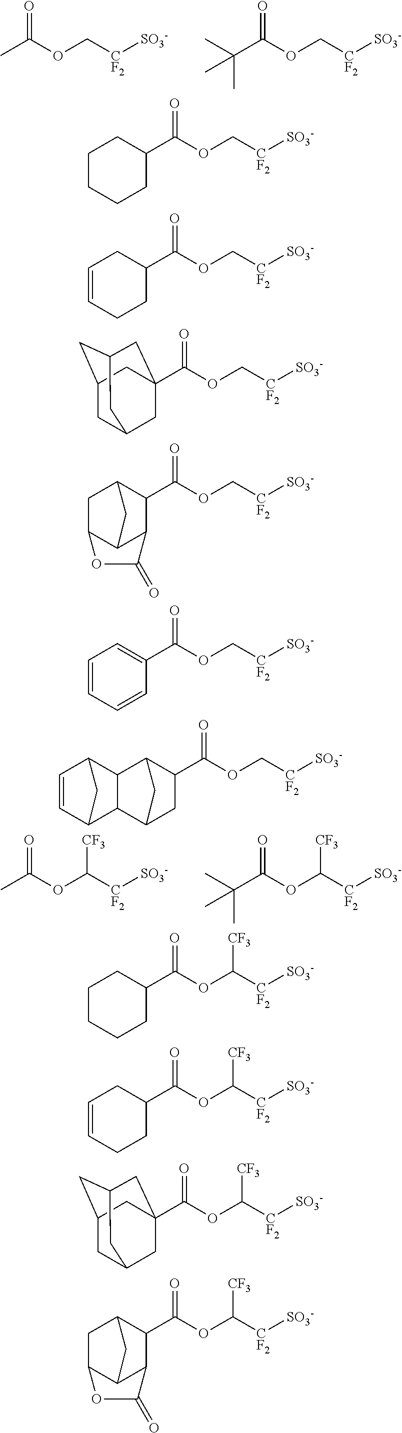

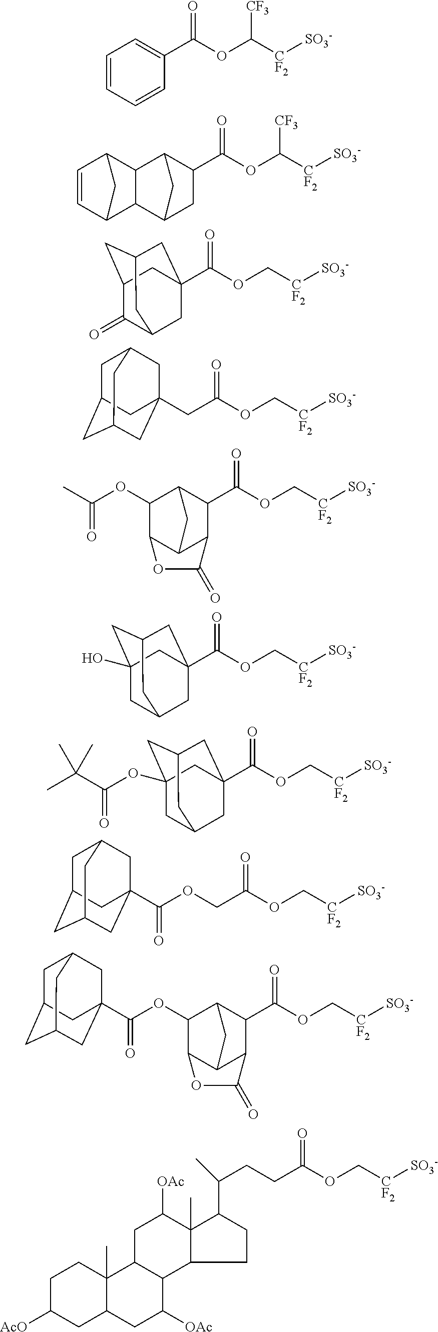

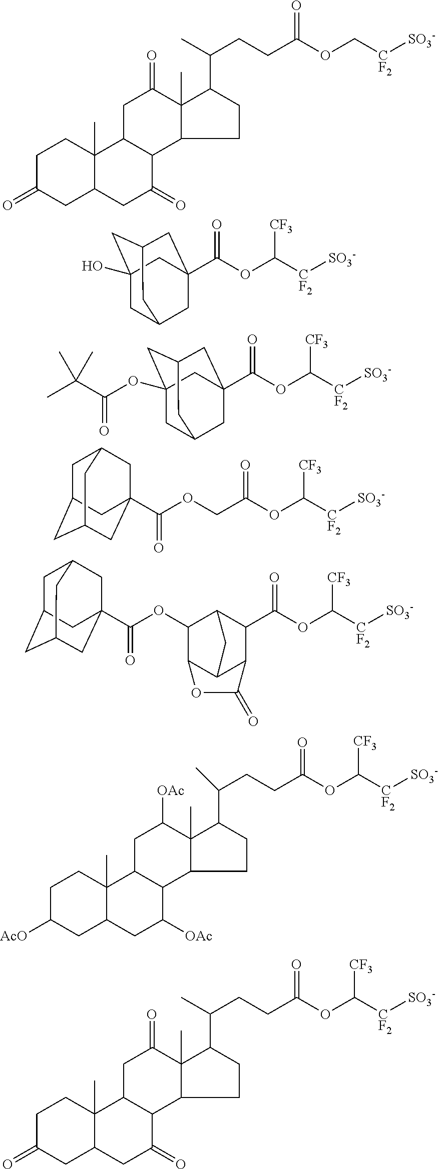

In the formula (3B), R^{fb1} and R^{fb2} are each independently a fluorine atom or a hydrocarbyl group having 1 to 40 carbon atoms and optionally containing a heteroatom. The hydrocarbyl group may be saturated or unsaturated, and may be linear, branched, or cyclic. Specific examples thereof include those shown as examples of the hydrocarbyl group represented by R^{fa1} in the formula (3A′). R^{fb1} and R^{fb2} are preferably each a fluorine atom or a linear fluorinated alkyl group having 1 to 4 carbon atoms. R^{fb1} and R^{fb2} are optionally bound together to form a ring together with the group to which they are bound (—CF₂—SO₂—N—SO₂—CF₂—), and in this case the group resulting from bonding R^{fb1} and R^{fb2} together is preferably a fluorinated ethylene group or a fluorinated propylene group.

In the formula (3C), R^{fc1}, R^{fc2}, and R^{fc3} are each independently a fluorine atom or a hydrocarbyl group having 1 to 40 carbon atoms and optionally containing a heteroatom. The hydrocarbyl group may be saturated or unsaturated, and may be linear, branched, or cyclic. Specific examples thereof include those shown as examples of the hydrocarbyl group represented by R^{fa1} in the formula (3A′). R^{fc1}, R^{fc2}, and R^{fc3} are preferably each a fluorine atom or a linear fluorinated alkyl group having 1 to 4 carbon atoms. R^{fc1} and R^{fc2} are optionally bound together to form a ring together with the group to which they are bound (—CF₂—SO₂—C—SO₂—CF₂—), and in this case the group resulting from bonding R^{fc1} and R^{fc2} together is preferably a fluorinated ethylene group or a fluorinated propylene group.

In the formula (3D), R^{fd} is a hydrocarbyl group having 1 to 40 carbon atoms and optionally containing a heteroatom. The hydrocarbyl group may be saturated or unsaturated, and may be linear, branched, or cyclic. Specific examples thereof include those shown as examples of the hydrocarbyl group represented by R^{fa1} in the formula (3A′).

Synthesis of sulfonium salts containing the anion represented by the formula (3D) is described in detail in JP 2010-215608 A and JP 2014-133723 A.

Specific examples of the anion represented by the formula (3D) include, but are not limited to, those shown in the following:

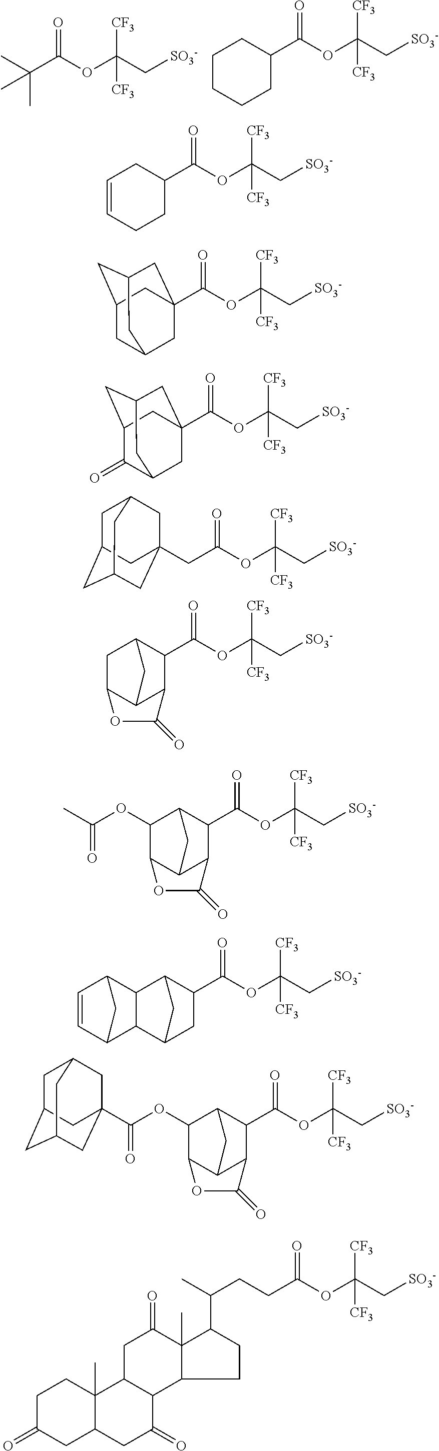

The photo-acid generator containing the anion represented by the formula (3D) has no fluorine atom at the a position of the sulfo group, but has acidity enough to cleave acid-unstable groups in the base polymer because it has the two trifluoromethyl groups at the R position. Accordingly, use as a photo-acid generator is acceptable.

For the photo-acid generator, one represented by the following formula (4) can also be suitably used.

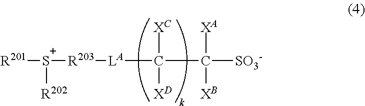

In the formula (4), R^{20′} and R²⁰² are each independently a hydrocarbyl group having 1 to 30 carbon atoms and optionally containing a heteroatom. R²⁰³ is a hydrocarbylene group having 1 to 30 carbon atoms and optionally containing a heteroatom. Any two of R²⁰¹, R²⁰², and R²⁰³ are optionally bound together to form a ring together with the sulfur atom to which the two are bound. Specific examples of the ring in this case include those shown as examples of the ring that can be formed through bonding of R^{ct1} and R^{ct2} together with the sulfur atom to which they are bound in the description of the formula (cation-1).

The hydrocarbyl group represented by R²⁰¹ and R²⁰², having 1 to 30 carbon atoms, may be saturated or unsaturated, and may be linear, branched, or cyclic. Specific examples thereof include an alkyl group having 1 to 30 carbon atoms such as a methyl group, an ethyl group, a n-propyl group, an isopropyl group, a n-butyl group, an isobutyl group, a sec-butyl group, a tert-butyl group, a n-pentyl group, a tert-pentyl group, a n-hexyl group, a n-octyl group, a 2-ethylhexyl group, a n-nonyl group, and a n-decyl group; a cyclic saturated hydrocarbyl group having 3 to 30 carbon atoms such as a cyclopentyl group, a cyclohexyl group, a cyclopentylmethyl group, a cyclopentylethyl group, cyclopentylbutyl group, a cyclohexylmethyl group, a cyclohexylethyl group, a cyclohexylbutyl group, a norbornyl group, an oxanorbornyl group, a tricyclo[5.2.1.0^{2,6}]decyl group, and an adamantyl group; an aryl group having 6 to 30 carbon atoms such as a phenyl group, a methylphenyl group, an ethylphenyl group, a n-propylphenyl group, an isopropylphenyl group, a n-butylphenyl group, an isobutylphenyl group, a sec-butylphenyl group, a tert-butylphenyl group, a naphthyl group, a methylnaphthyl group, an ethylnaphthyl group, a n-propylnaphthyl group, an isopropylnaphthyl group, a n-butylnaphthyl group, an isobutylnaphthyl group, a sec-butylnaphthyl group, a tert-butylnaphthyl group, and an anthracenyl group; and a group given by combining any of these. Some or all of the hydrogen atoms of the hydrocarbyl group may be each replaced with a group containing a heteroatom such as an oxygen atom, a sulfur atom, a nitrogen atom, and a halogen atom, some of the —CH₂— moieties of the hydrocarbyl group may be each replaced with a group containing a heteroatom such as an oxygen atom, a sulfur atom, and a nitrogen atom, and as a result, for example, a hydroxy group, a cyano group, a fluorine atom, a chlorine atom, a bromine atom, an iodine atom, a carbonyl group, an ether bond, an ester bond, a sulfonic ester bond, a carbonate bond, a lactone ring, a sultone ring, a carboxylic anhydride (—C(═O)—O—C(═O)—), or a haloalkyl group can be contained.

The hydrocarbylene group represented by R²⁰³, having 1 to 30 carbon atoms, may be saturated or unsaturated, and may be linear, branched, or cyclic. Specific examples thereof include an alkanediyl group having 1 to 30 carbon atoms such as a methanediyl group, an ethane-1,1-diyl group, an ethane-1,2-diyl group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group, and a heptadecane-1,17-diyl group; a cyclic saturated hydrocarbylene group having 3 to 30 carbon atoms such as a cyclopentanediyl group, a cyclohexanediyl group, a norbornanediyl group, and an adamantanediyl group; an arylene group having 6 to 30 carbon atoms such as a phenylene group, a methylphenylene group, an ethylphenylene group, a n-propylphenylene group, an isopropylphenylene group, a n-butylphenylene group, an isobutylphenylene group, a sec-butylphenylene group, a tert-butylphenylene group, a naphthylene group, a methylnaphthylene group, an ethylnaphthylene group, a n-propylnaphthylene group, an isopropylnaphthylene group, a n-butylnaphthylene group, an isobutylnaphthylene group, a sec-butylnaphthylene group, and a tert-butylnaphthylene group; and a group given by combining any of these. Some or all of the hydrogen atoms of the hydrocarbylene group may be each replaced with a group containing a heteroatom such as an oxygen atom, a sulfur atom, a nitrogen atom, and a halogen atom, some of the —CH₂— moieties of the hydrocarbylene group may be each replaced with a group containing a heteroatom such as an oxygen atom, a sulfur atom, and a nitrogen atom, and as a result, for example, a hydroxy group, a cyano group, a fluorine atom, a chlorine atom, a bromine atom, an iodine atom, a carbonyl group, an ether bond, an ester bond, a sulfonic ester bond, a carbonate bond, a lactone ring, a sultone ring, a carboxylic anhydride (—C(═O)—O—C(═O)—), or a haloalkyl group may be contained. The heteroatom is preferably an oxygen atom.

In the formula (4), L^{A} is a single bond, an ether bond, or a hydrocarbylene group having 1 to 20 carbon atoms and optionally containing a heteroatom. The hydrocarbylene group may be saturated or unsaturated, and may be linear, branched, or cyclic. Specific examples thereof include those shown as examples of the hydrocarbylene group represented by R²⁰³.

In the formula (4), X^{A}, X^{B}, X^{C}, and X^{D} are each independently a hydrogen atom, a fluorine atom, or a trifluoromethyl group. It should be noted that at least one of X^{A}, X^{B}, X^{C}, and X^{D} is a fluorine atom or a trifluoromethyl group.

In the formula (4), k is an integer of 0 to 3.

The photo-acid generator represented by the formula (4) is preferably one represented by the following formula (4′).

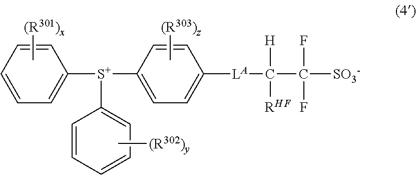

In the formula (4′), L^{A} is as specified above. R^{HF} is a hydrogen atom or a trifluoromethyl group, and preferably a trifluoromethyl group. R³⁰¹, R³⁰², and R³⁰³ are each independently a hydrocarbyl group having 1 to 20 carbon atoms and optionally containing a heteroatom. The hydrocarbyl group may be saturated or unsaturated, and may be linear, branched, or cyclic. Specific examples thereof include those shown as examples of the hydrocarbyl group represented by R^{fa1} in the formula (3A′). x and y are each independently an integer of 0 to 5. z is an integer of 0 to 4.

Specific examples of the photo-acid generator represented by the formula (4) include those shown as examples of a photo-acid generator represented by formula (2) in JP 2017-26980 A.

Among the photo-acid generators, a photo-acid generator containing the anion represented by the formula (3A′) or (3D) causes small acid diffusion and exhibits superior solubility in solvents as well, thus being particularly preferred. The photo-acid generator represented by the formula (4′) causes very small acid diffusion, thus being particularly preferred.

Applicable as other acid generators are a sulfonium salt and an iodonium salt represented by the following formulae (5-1) and (5-2), respectively, each containing an anion having an aromatic ring or aromatic rings substituted with an iodine atom or iodine atoms:

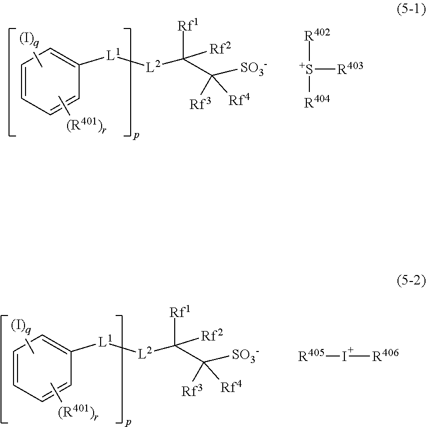

In the formulae (5-1) and (5-2), p is 1, 2, or 3. q and r are integers satisfying 1 q 5, 0 r 3, and 1 q+r 5. q is preferably 1, 2, or 3, and more preferably 2 or 3. r is preferably 0, 1, or 2.

In the formulae (5-1) and (5-2), L¹ is a single bond, an ether bond or an ester bond, or a saturated hydrocarbylene group having 1 to 6 carbon atoms and optionally containing an ether bond or an ester bond. The saturated hydrocarbylene group may be linear, branched, or cyclic.

In the formulae (5-1) and (5-2), L² is a single bond or a divalent linking group having 1 to 20 carbon atoms if p is 1, and L² is a (p+1)-valent linking group having 1 to 20 carbon atoms if p is 2 or 3, and the linking group may contain an oxygen atom, a sulfur atom, or a nitrogen atom.

In the formulae (5-1) and (5-2), R⁴⁰1 is: a hydroxy group, a carboxy group, a fluorine atom, a chlorine atom, a bromine atom, or an amino group; a hydrocarbyl group having 1 to 20 carbon atoms, hydrocarbyloxy group having 1 to 20 carbon atoms, hydrocarbylcarbonyl group having 2 to 20 carbon atoms, hydrocarbyloxycarbonyl group having 2 to 20 carbon atoms, hydrocarbylcarbonyloxy group having 2 to 20 carbon atoms, or hydrocarbylsulfonyloxy group having 1 to 20 carbon atoms, each optionally containing a fluorine atom, a chlorine atom, a bromine atom, a hydroxy group, an amino group, or an ether bond; or —N(R^{401A}) (R^{401B}), —N(R^{401C})—C(═O)—R^{401D}, or —N(R^{401C})—C(═O)—O—R^{401D}. R^{401A} and R^{401B} are each independently a hydrogen atom or a saturated hydrocarbyl group having 1 to 6 carbon atoms. R^{401C} is a hydrogen atom or a saturated hydrocarbyl group having 1 to 6 carbon atoms, and may contain a halogen atom, a hydroxy group, a saturated hydrocarbyloxy group having 1 to 6 carbon atoms, a saturated hydrocarbylcarbonyl group having 2 to 6 carbon atoms, or a saturated hydrocarbylcarbonyloxy group having 2 to 6 carbon atoms. R^{401D} is an aliphatic hydrocarbyl group having 1 to 16 carbon atoms, an aryl group having 6 to 14 carbon atoms, or an aralkyl group having 7 to 15 carbon atoms, and may contain a halogen atom, a hydroxy group, a saturated hydrocarbyloxy group having 1 to 6 carbon atoms, a saturated hydrocarbylcarbonyl group having 2 to 6 carbon atoms, or a saturated hydrocarbylcarbonyloxy group having 2 to 6 carbon atoms. The aliphatic hydrocarbyl group may be saturated or unsaturated, and may be linear, branched, or cyclic. The hydrocarbyl group, hydrocarbyloxy group, hydrocarbylcarbonyl group, hydrocarbyloxycarbonyl group, hydrocarbylcarbonyloxy group, and hydrocarbylsulfonyloxy group may be each linear, branched, or cyclic. If p and/or r are/is 2 or more, R⁴⁰¹ groups are identical to or different from each other.

Among them, for example, R⁴⁰¹ is preferably a hydroxy group, —N(R^{401C})—C(═O)—R^{401D}, —N(R^{401C})—C(═O)—O—R^{401D}, a fluorine atom, a chlorine atom, a bromine atom, a methyl group, or a methoxy group.

In the formulae (5-1) and (5-2), Rf¹ to Rf⁴ are each independently a hydrogen atom, a fluorine atom, or a trifluoromethyl group, and at least one of them is a fluorine atom or a trifluoromethyl group. Rf¹ and Rf² may be combined to form a carbonyl group. In particular, both of Rf³ and Rf⁴ are preferably fluorine atoms.

In the general formulae (5-1) and (5-2), R⁴⁰² to R⁴⁰⁶ are each independently a halogen atom or a hydrocarbyl group having 1 to 20 carbon atoms and optionally containing a heteroatom. The hydrocarbyl group may be saturated or unsaturated, and may be linear, branched, or cyclic. Specific examples thereof include those shown as examples of the hydrocarbyl group represented by R^{ct1} to R^{ct5} in the description of the formulae (cation-1) and (cation-2). Some or all of the hydrogen atoms of the hydrocarbyl group may be each replaced with a hydroxy group, a carboxy group, a halogen atom, a cyano group, a nitro group, a mercapto group, a sultone ring, a sulfo group, or a sulfonium salt-containing group, and some of the —CH₂— moieties of the hydrocarbyl group may be each replaced with an ether bond, an ester bond, a carbonyl group, an amide bond, a carbonate bond, or a sulfonic ester bond. R⁴⁰² and R⁴⁰³ are optionally bound together to form a ring together with the sulfur atom to which they are bound. Specific examples of the ring in this case include those shown as examples of the ring that can be formed through bonding of R^{ct1} and R^{ct2} together with the sulfur atom to which they are bound in the description of the formula (cation-1).

Specific examples of the cation of the sulfonium salt represented by the formula (5-1) include those shown as examples of the sulfonium cation represented by the formula (cation-1). Likewise, examples of the cation of the iodonium salt represented by the formula (5-2) include those shown as examples of the iodonium cation represented by the formula (cation-2).

Specific examples of the anion of the onium salt represented by the formula (5-1) or (5-2) include, but are not limited to, those shown in the following:

[chemical structure]

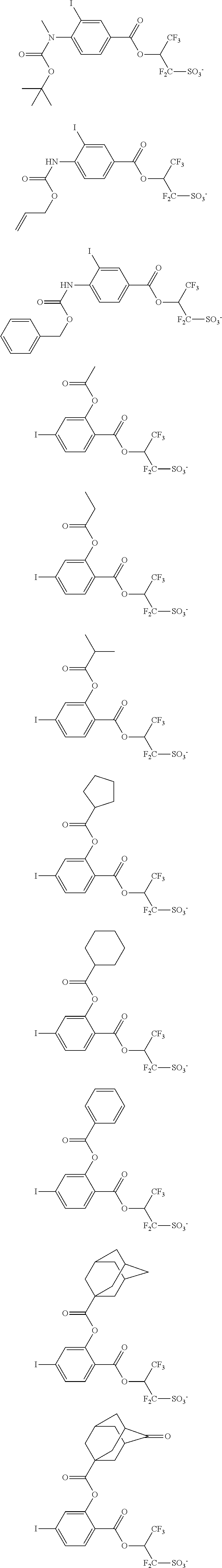

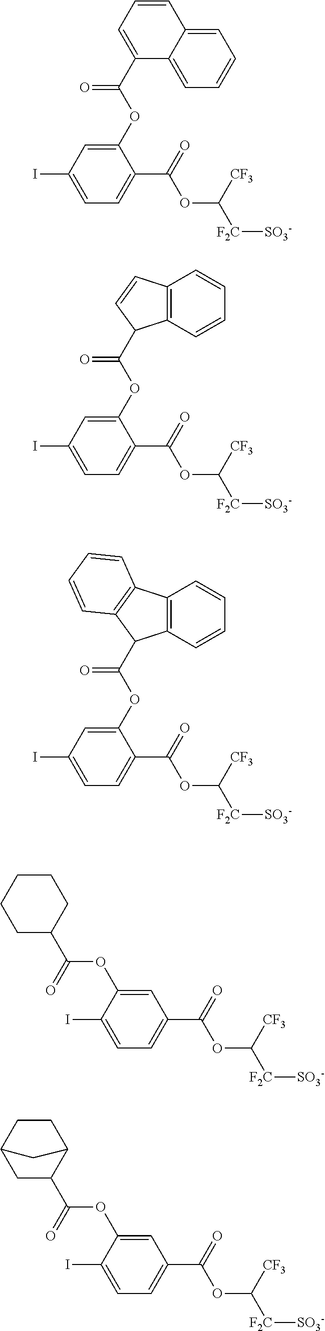

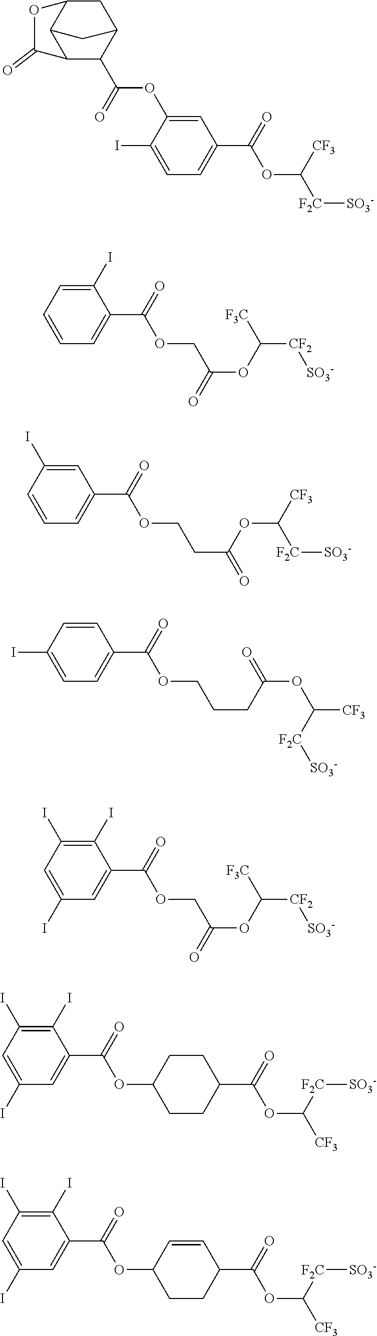

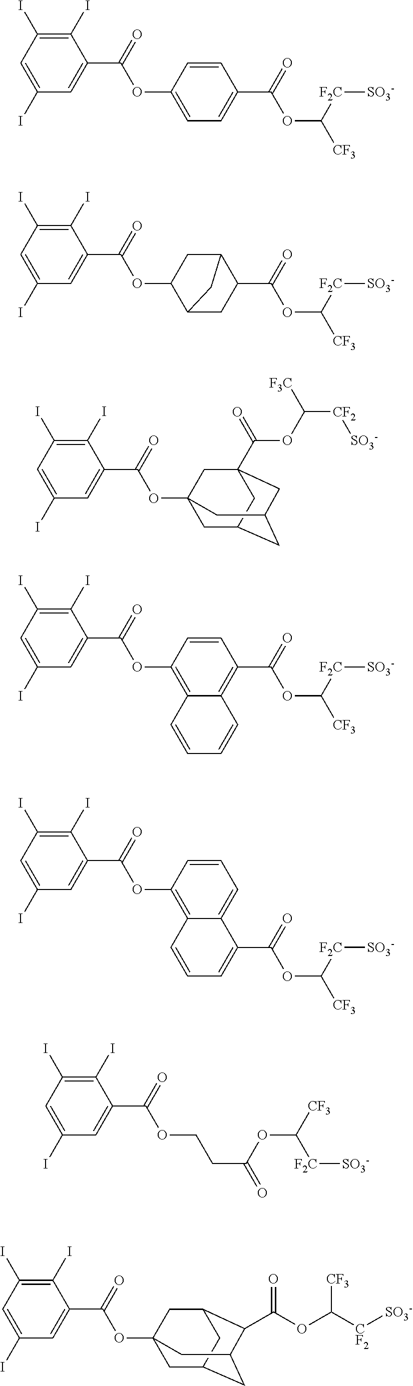

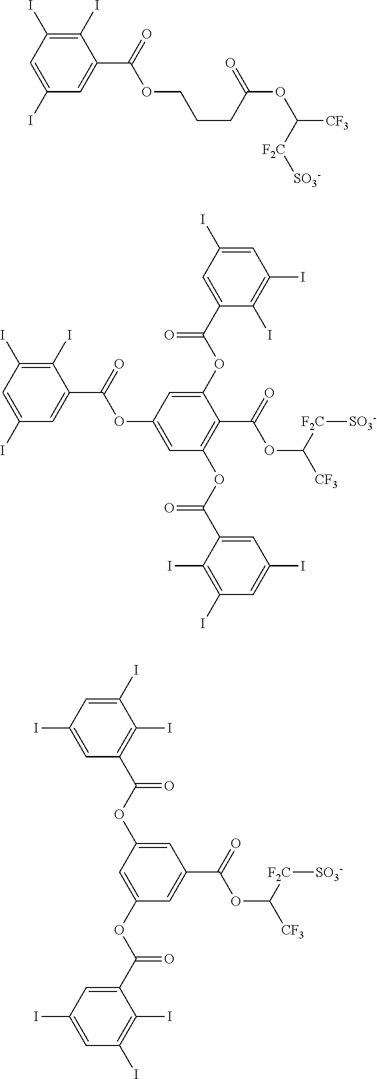

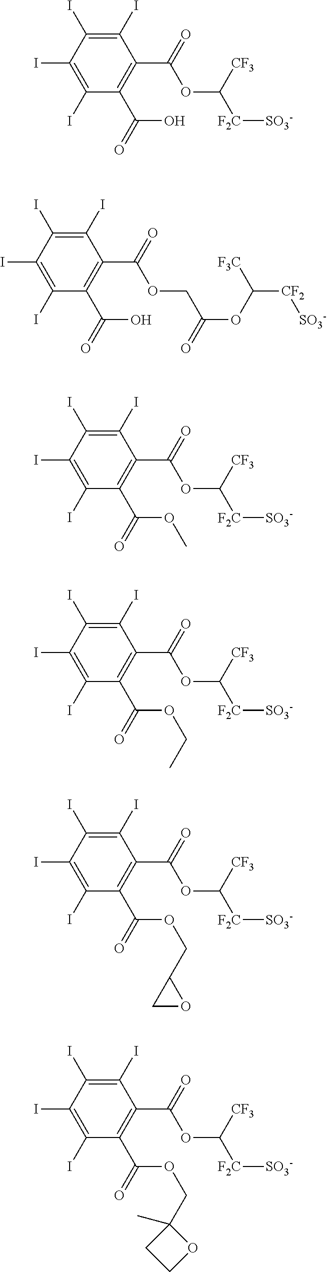

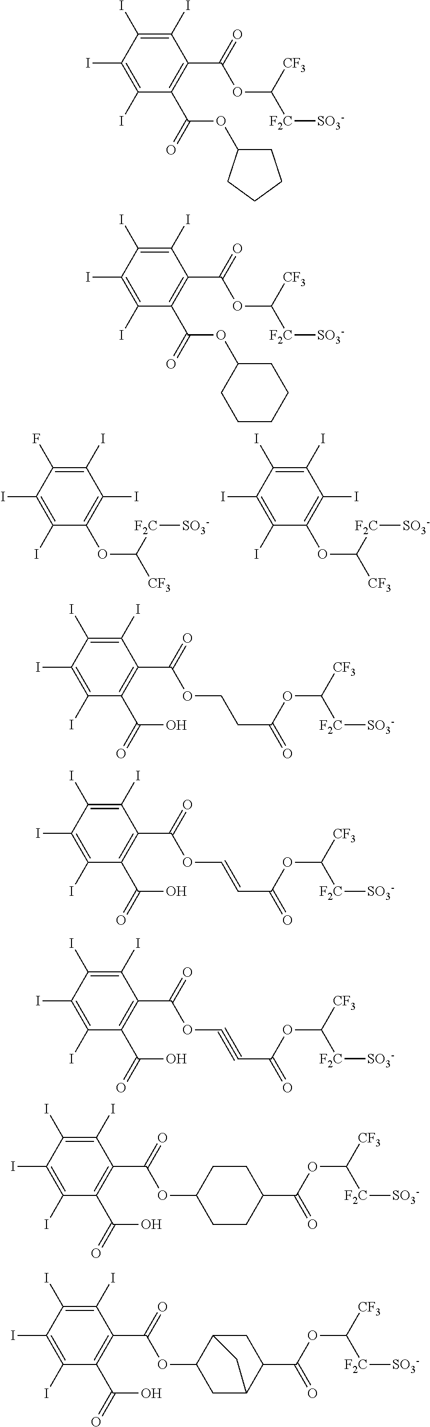

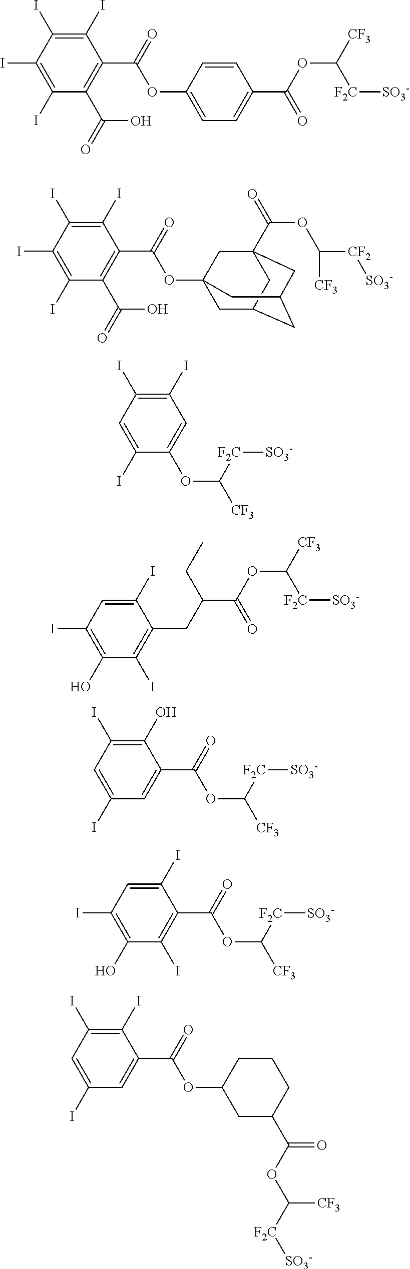

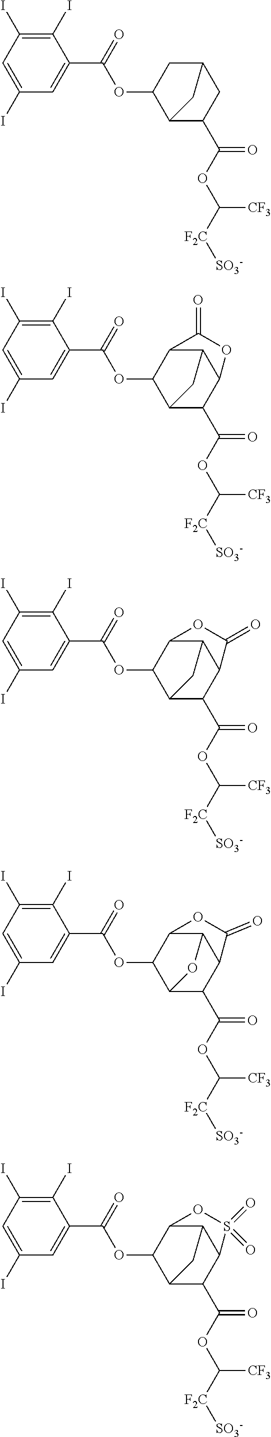

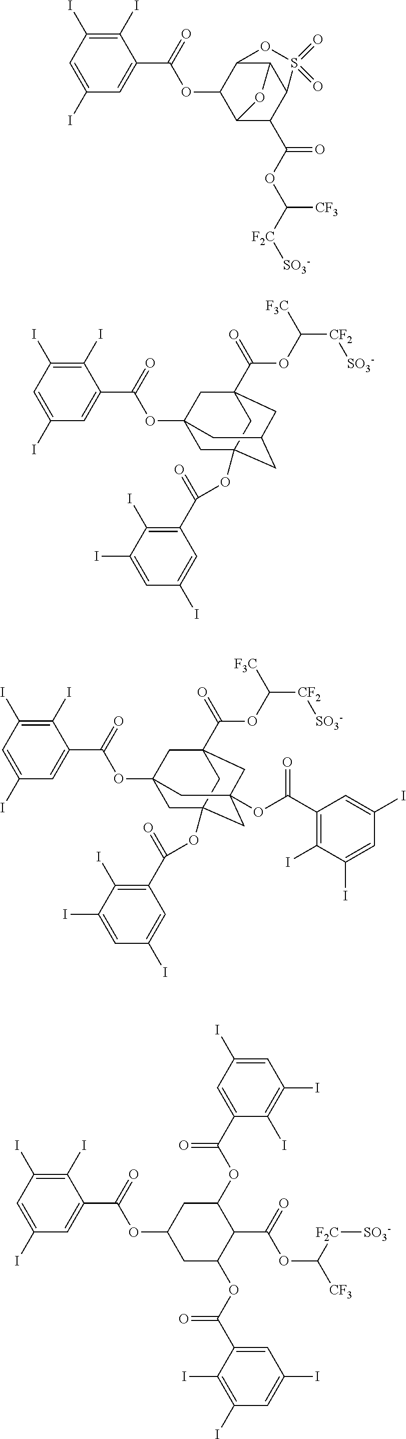

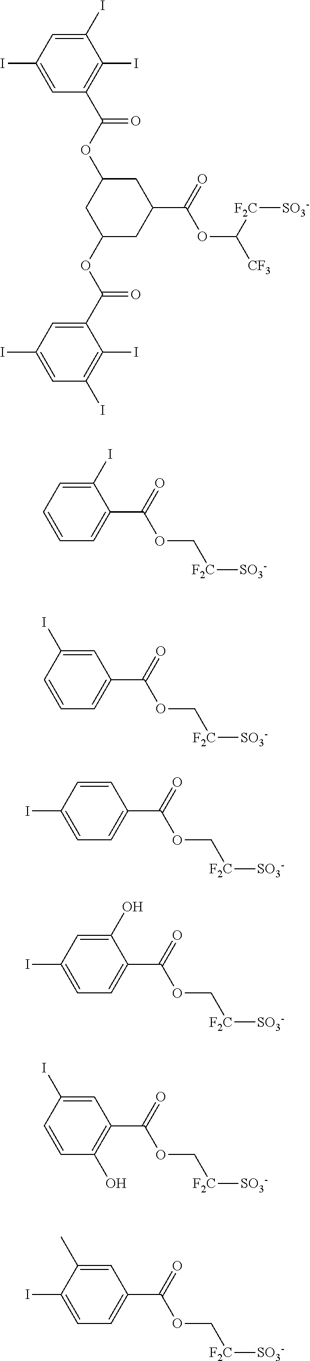

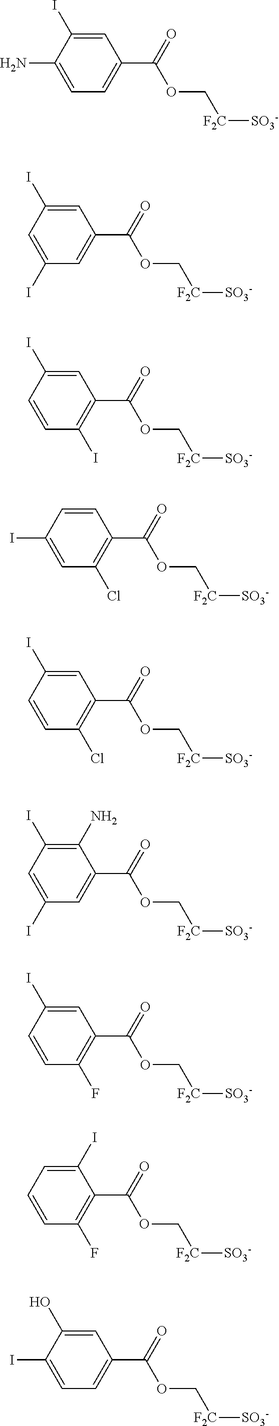

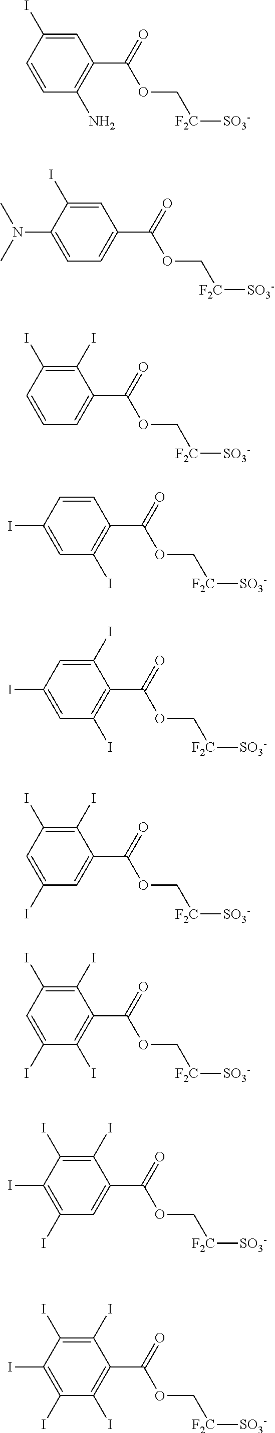

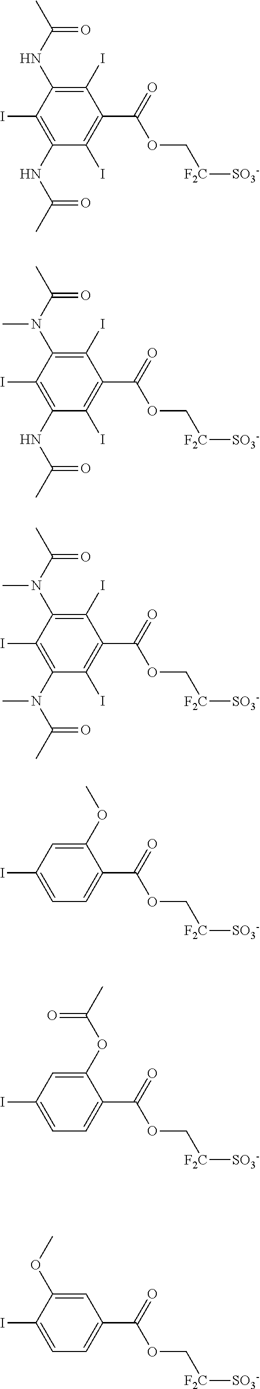

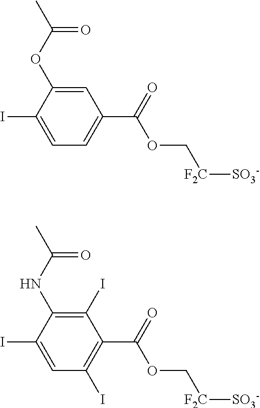

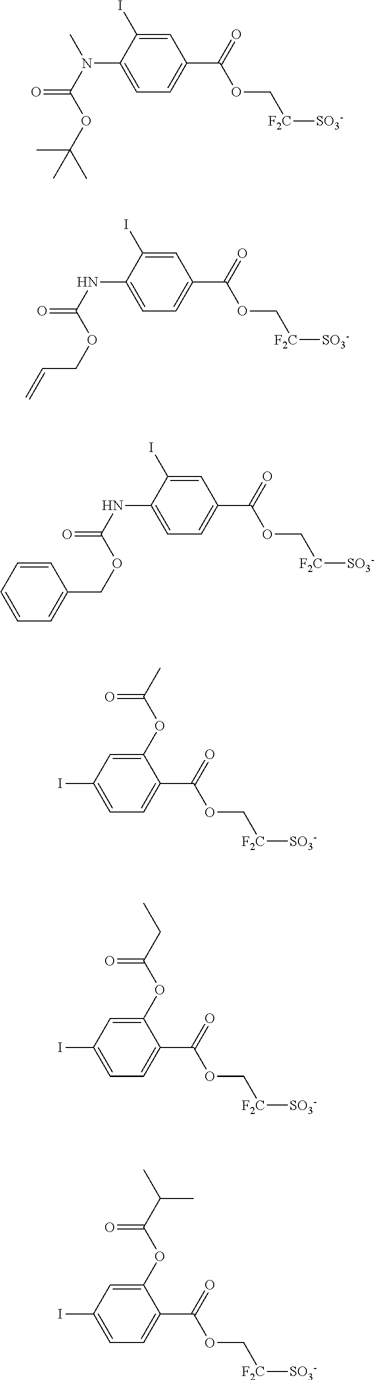

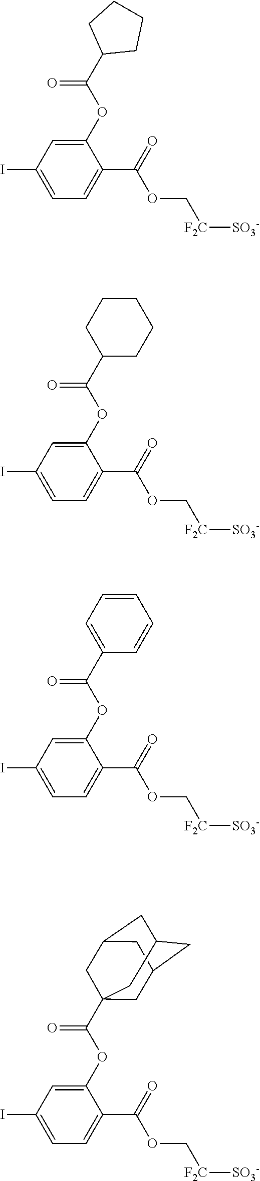

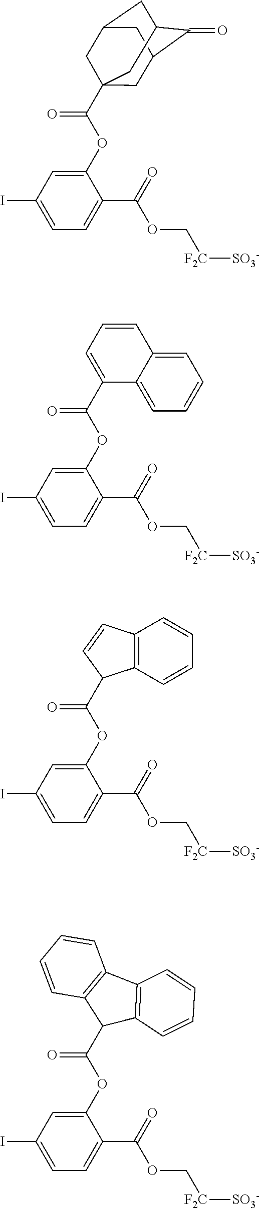

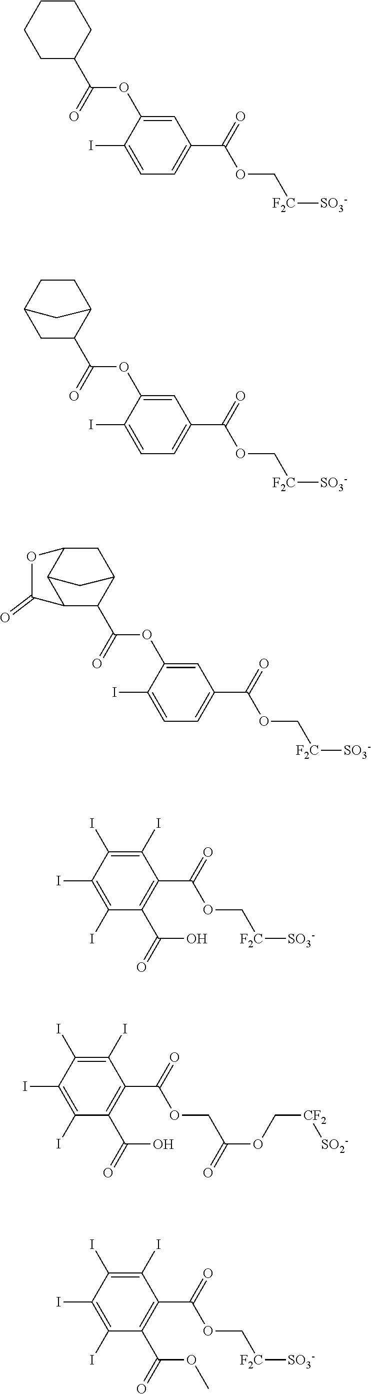

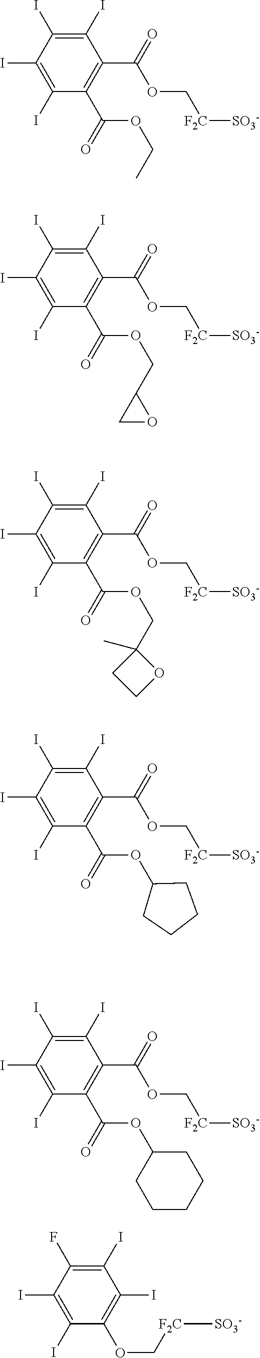

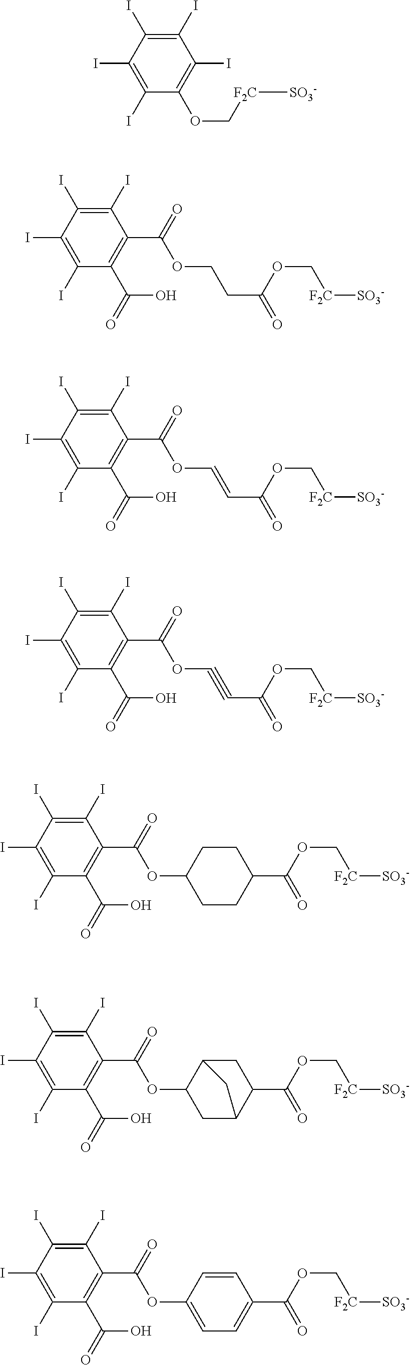

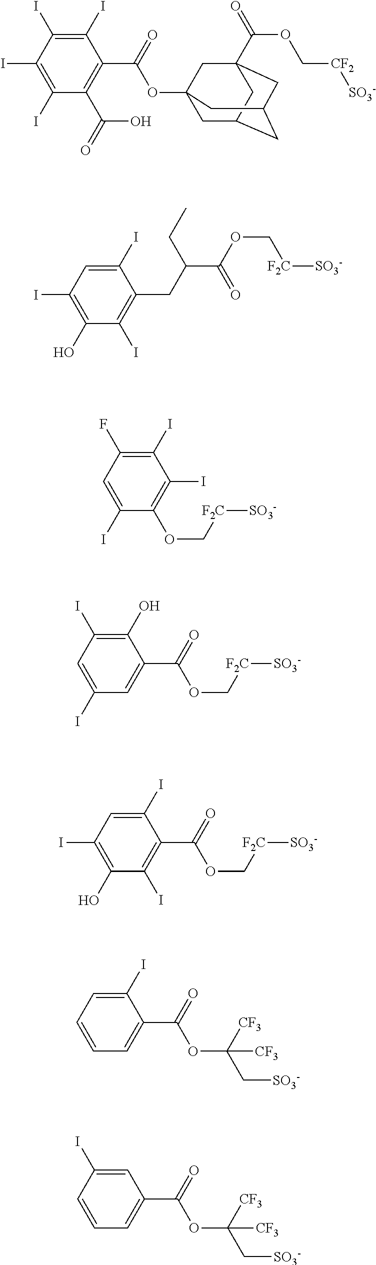

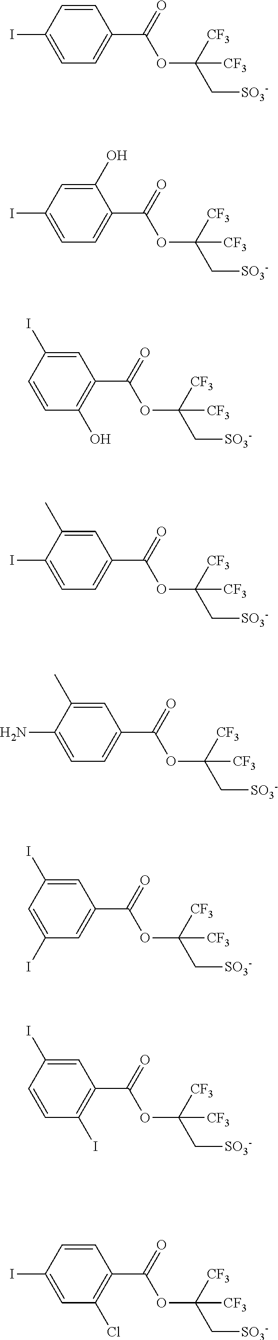

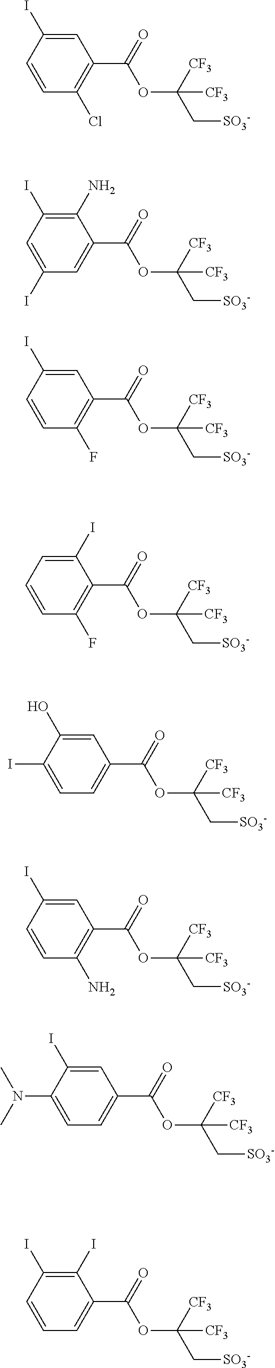

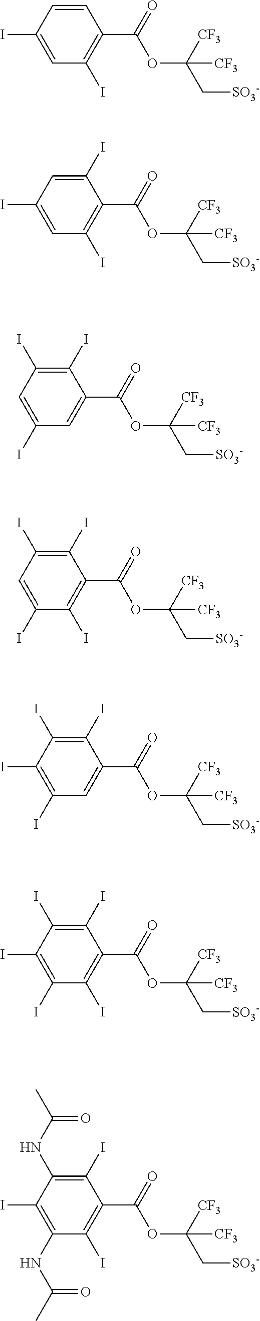

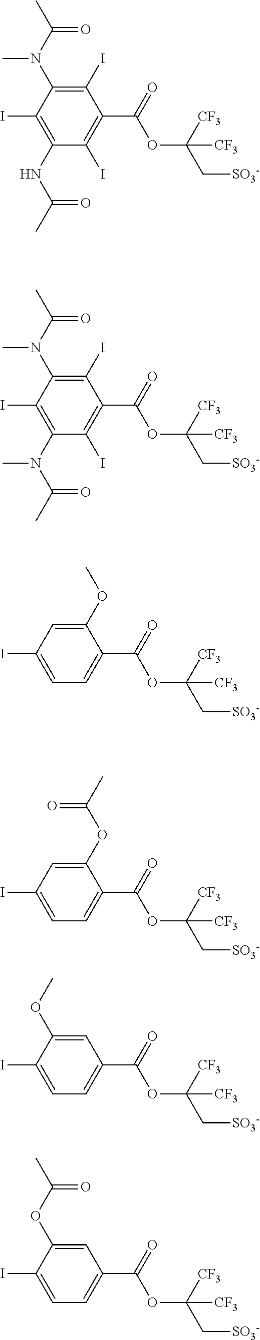

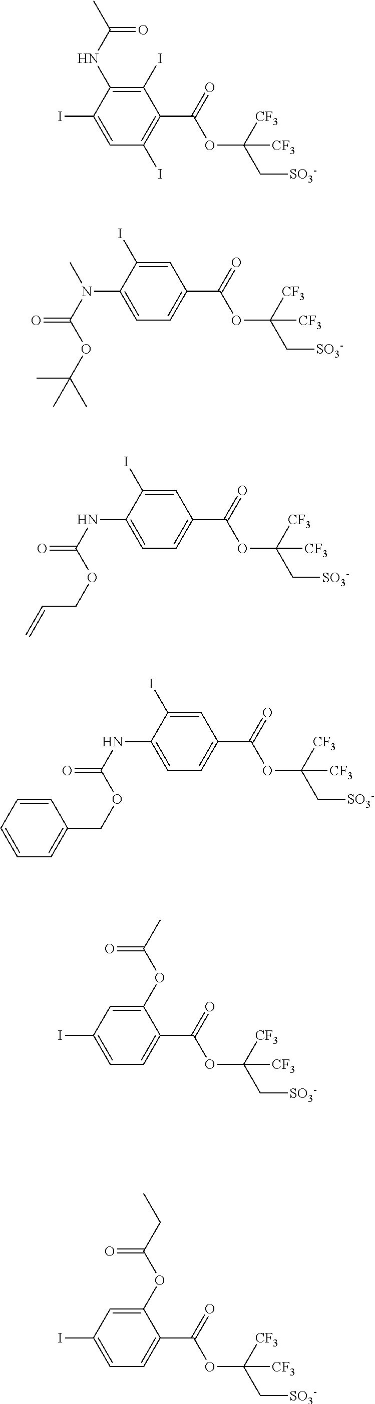

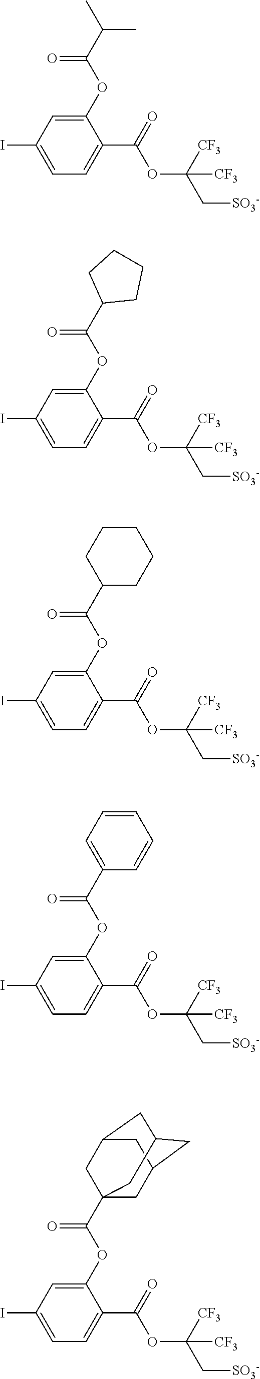

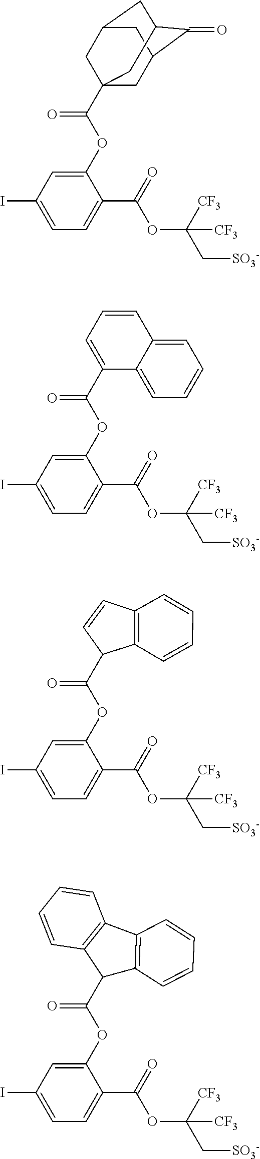

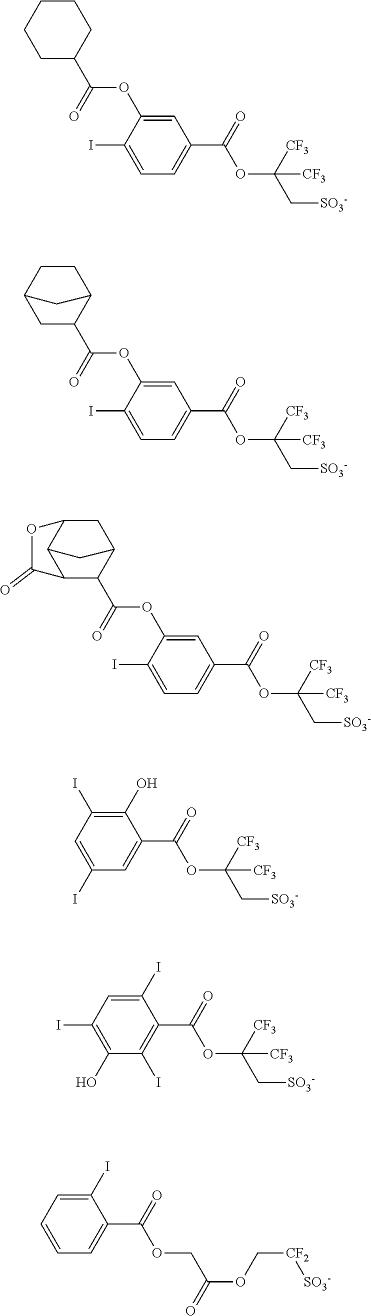

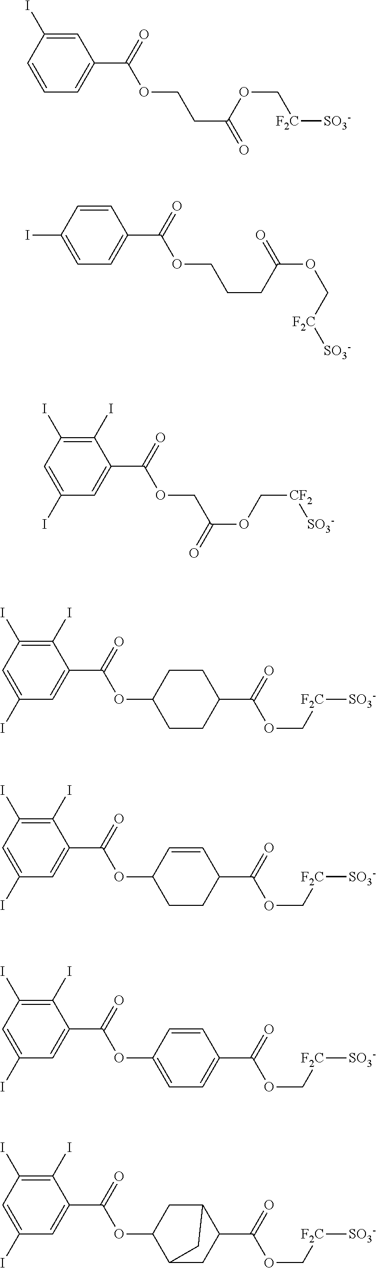

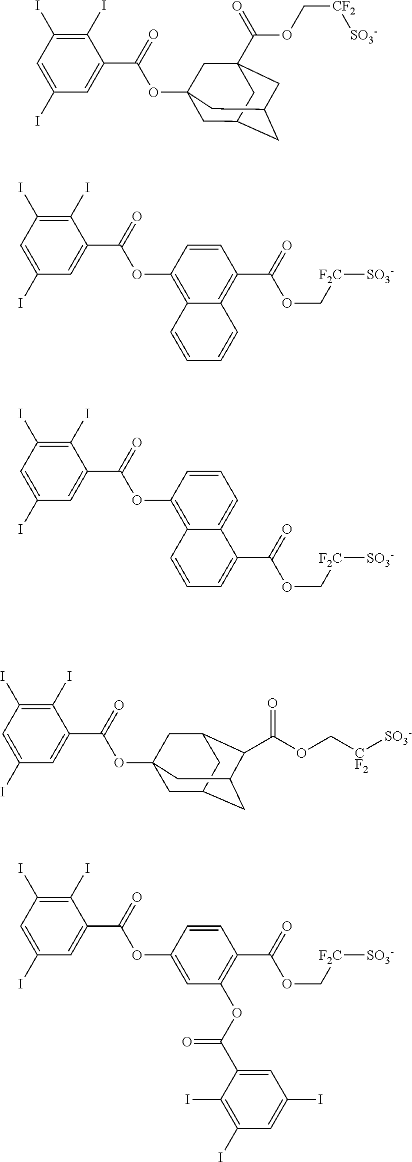

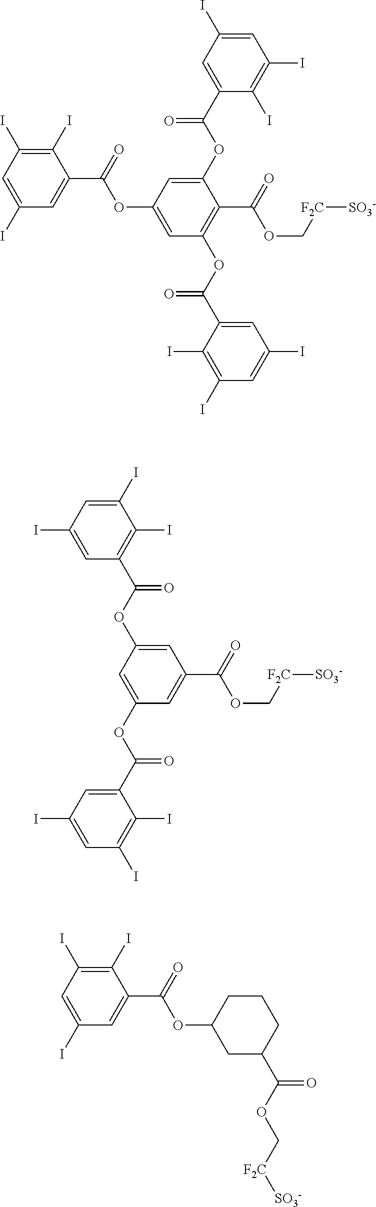

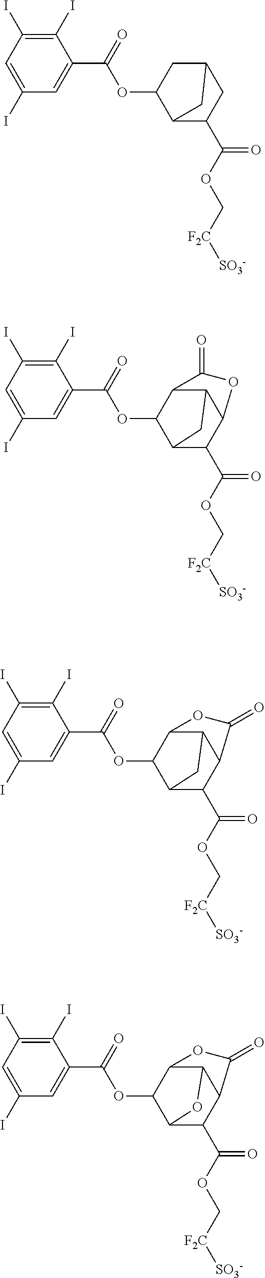

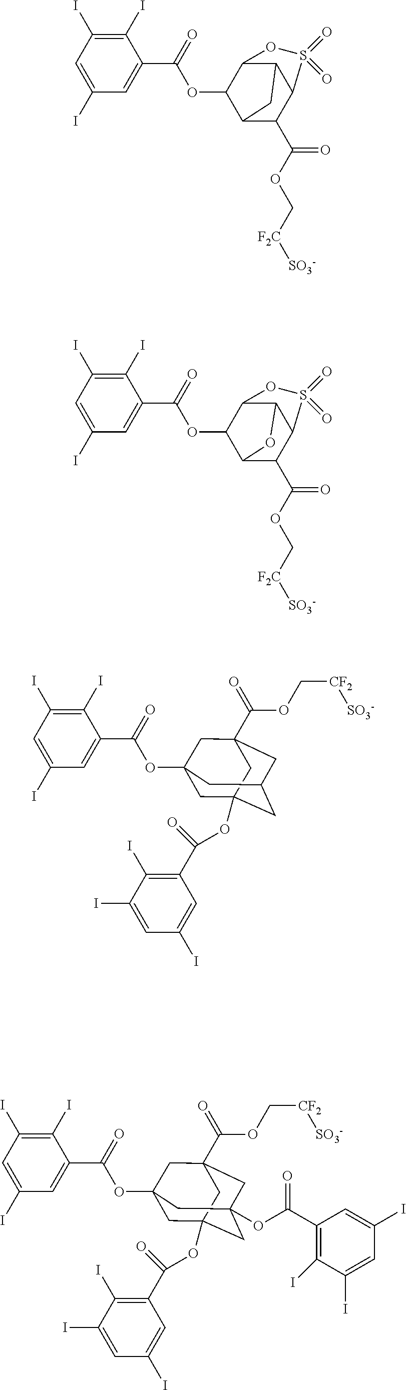

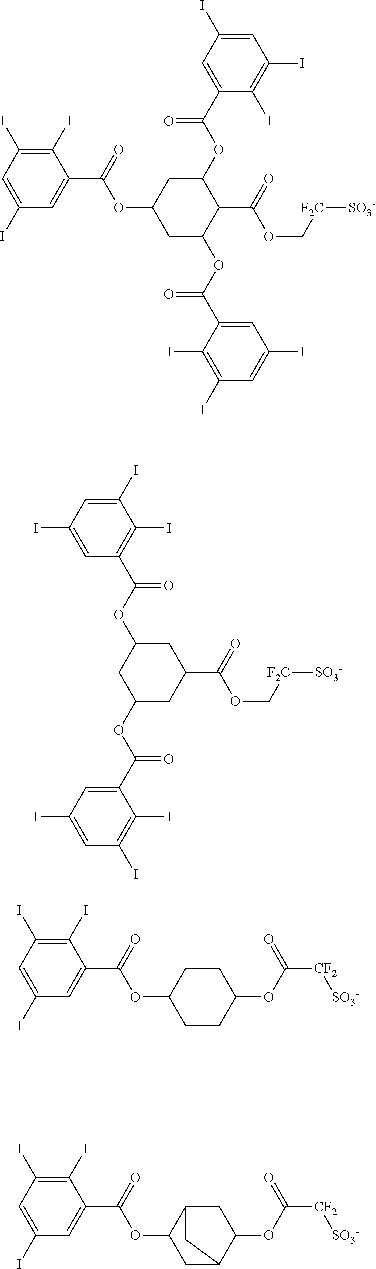

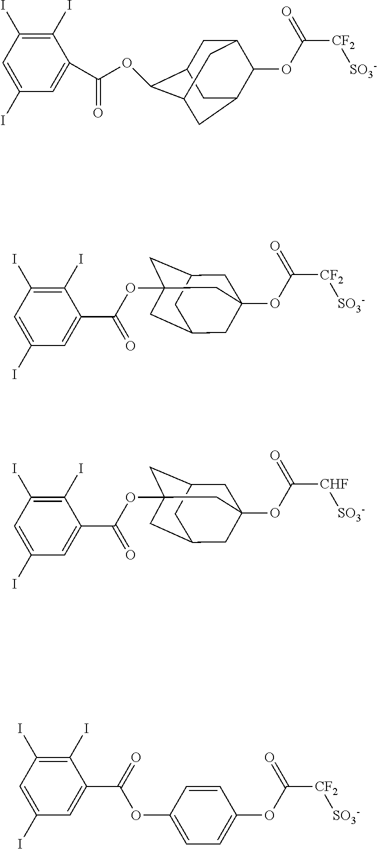

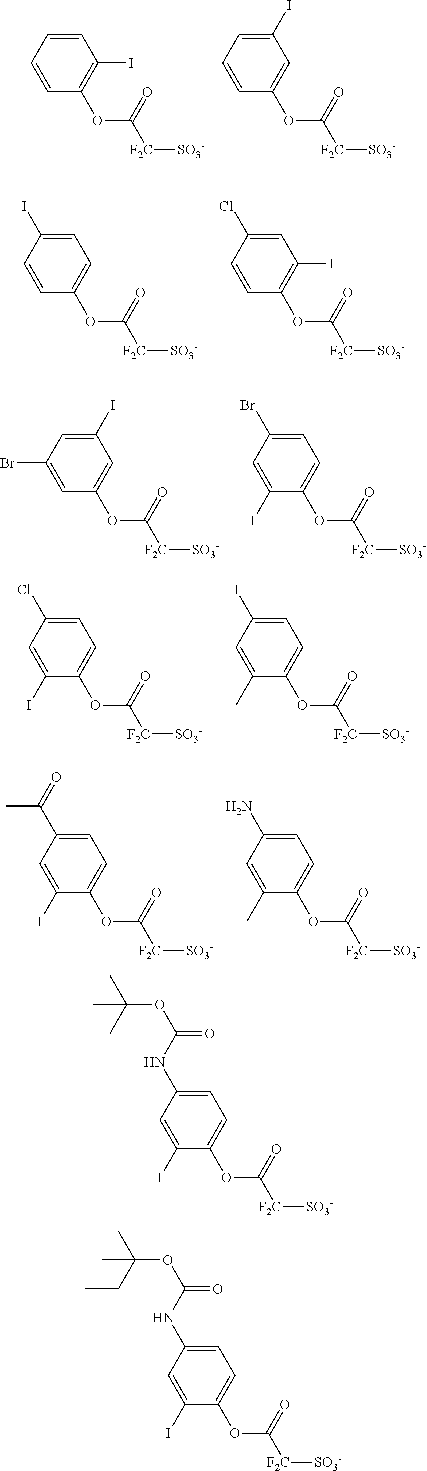

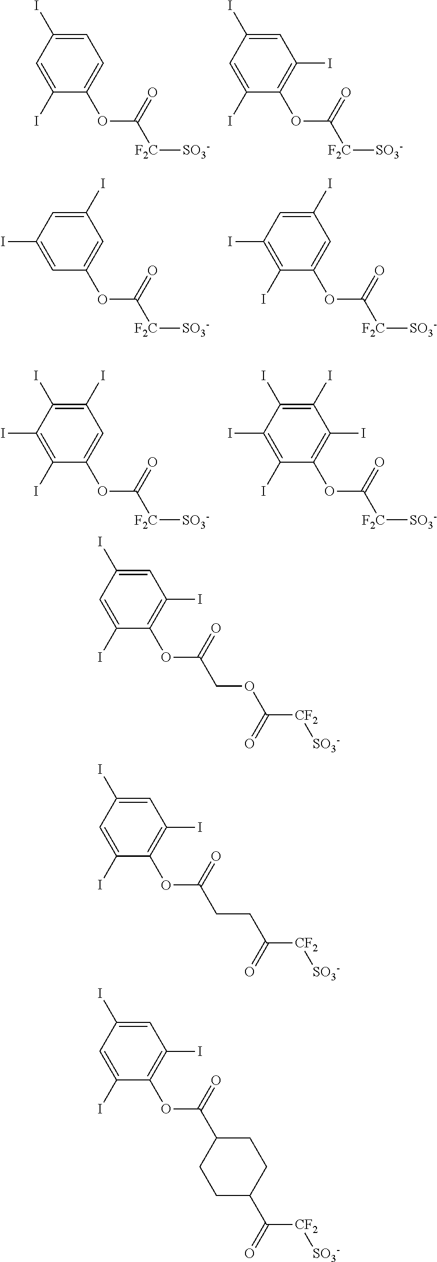

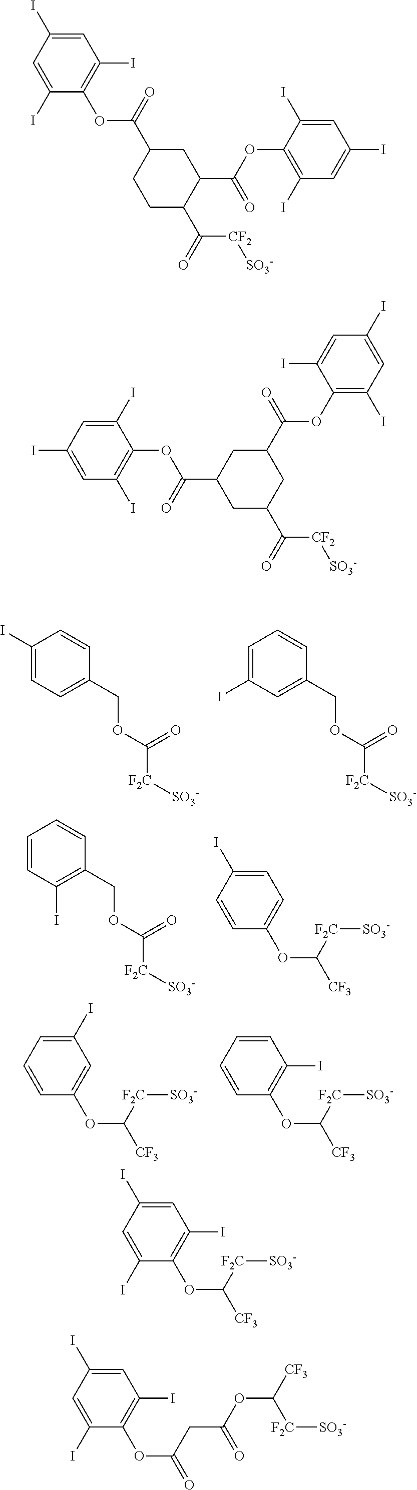

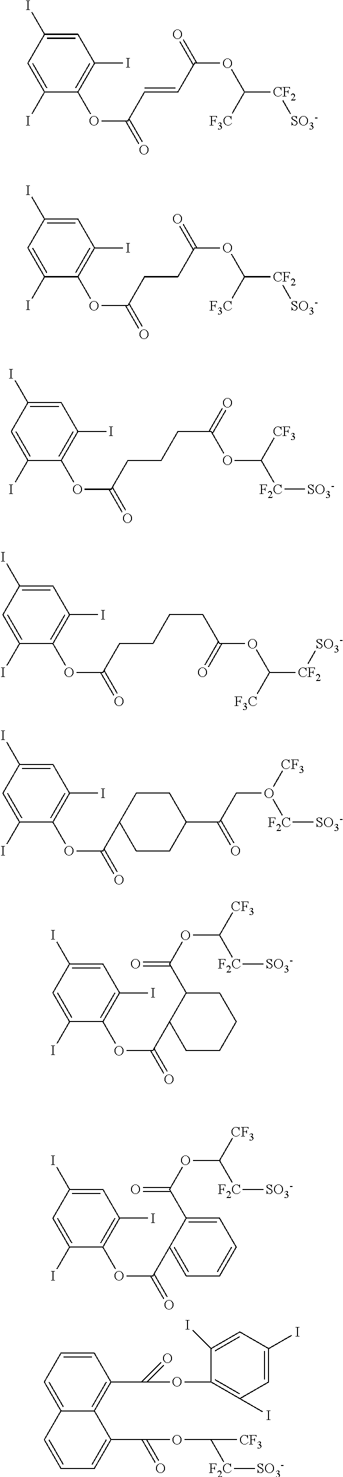

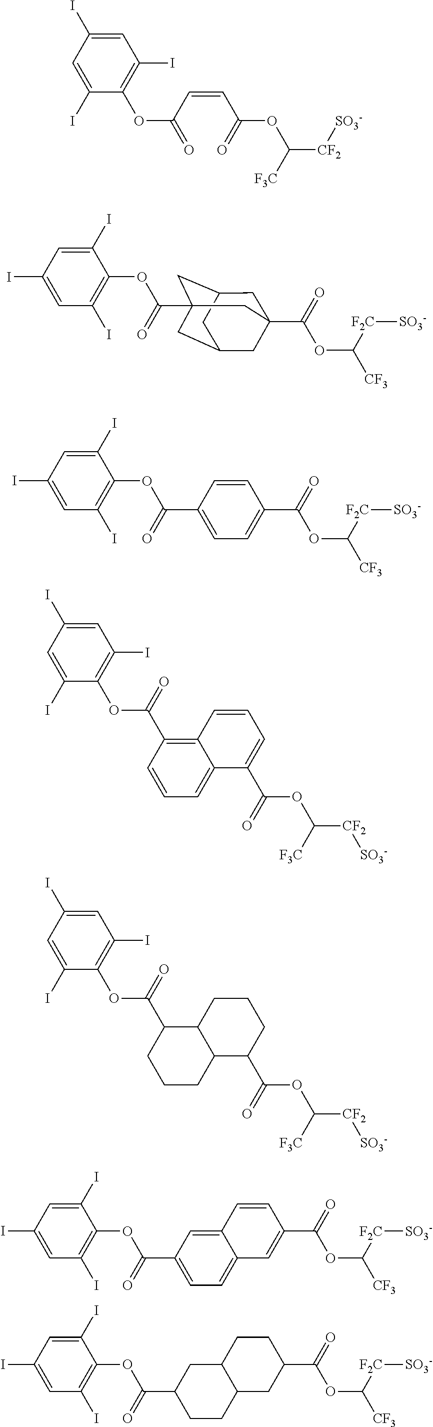

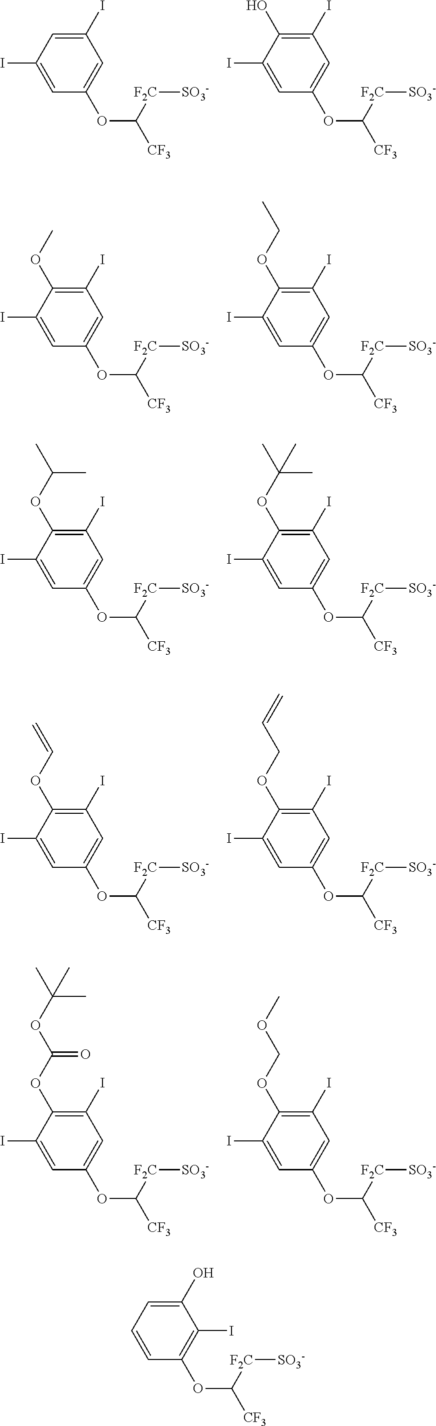

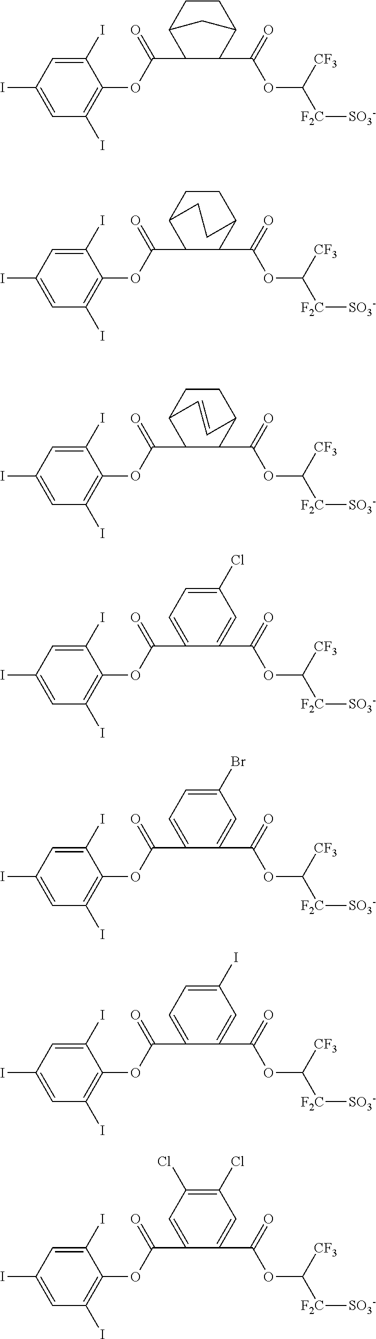

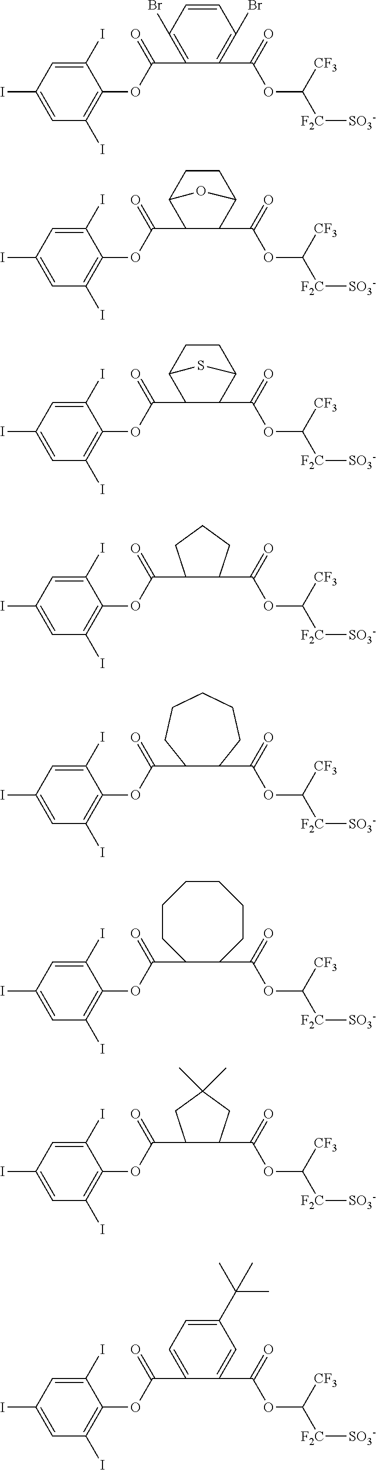

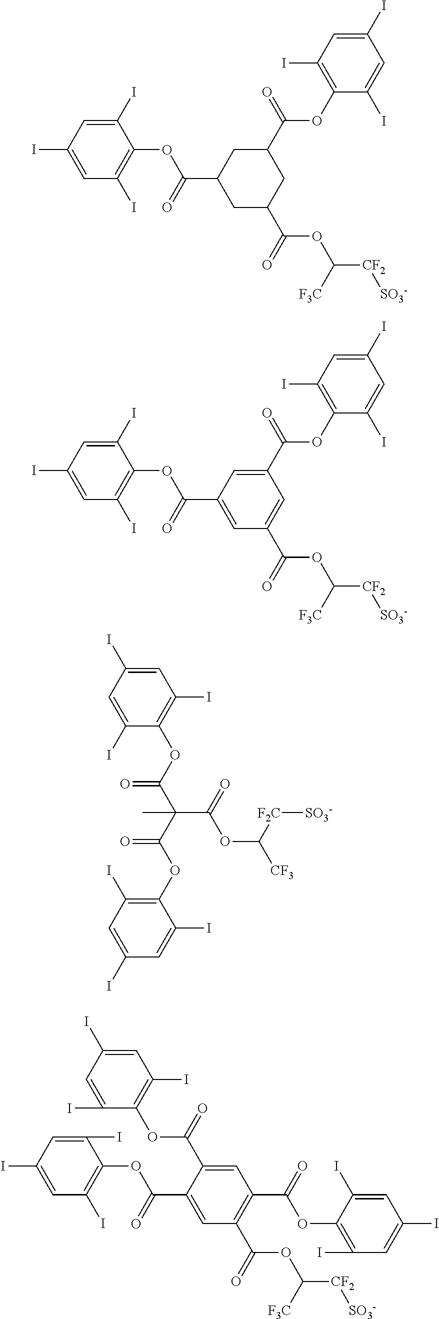

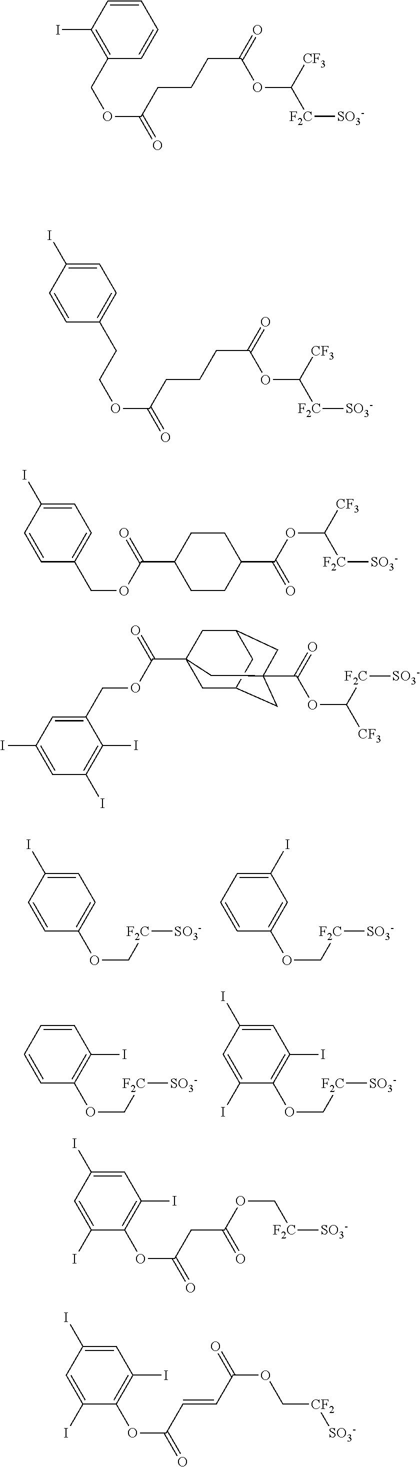

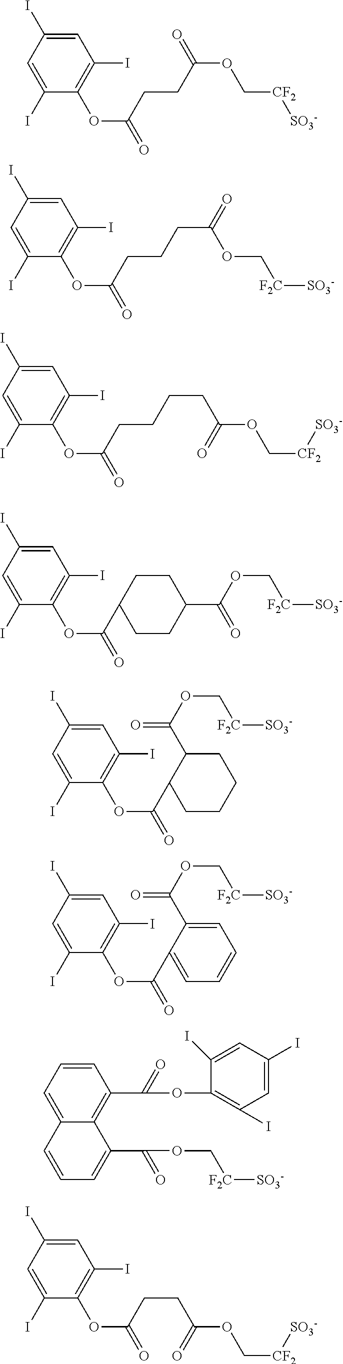

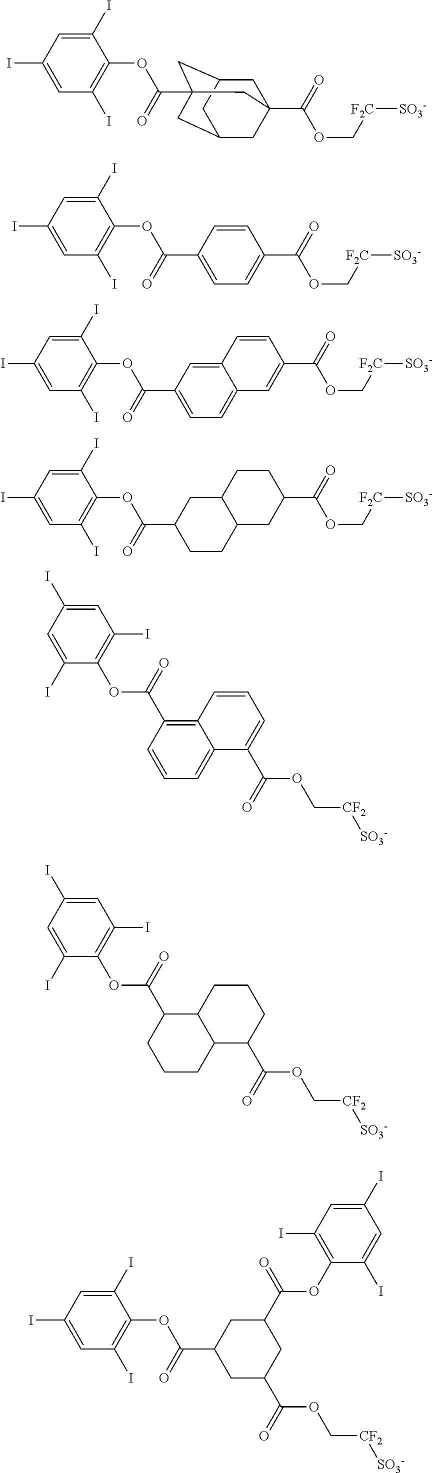

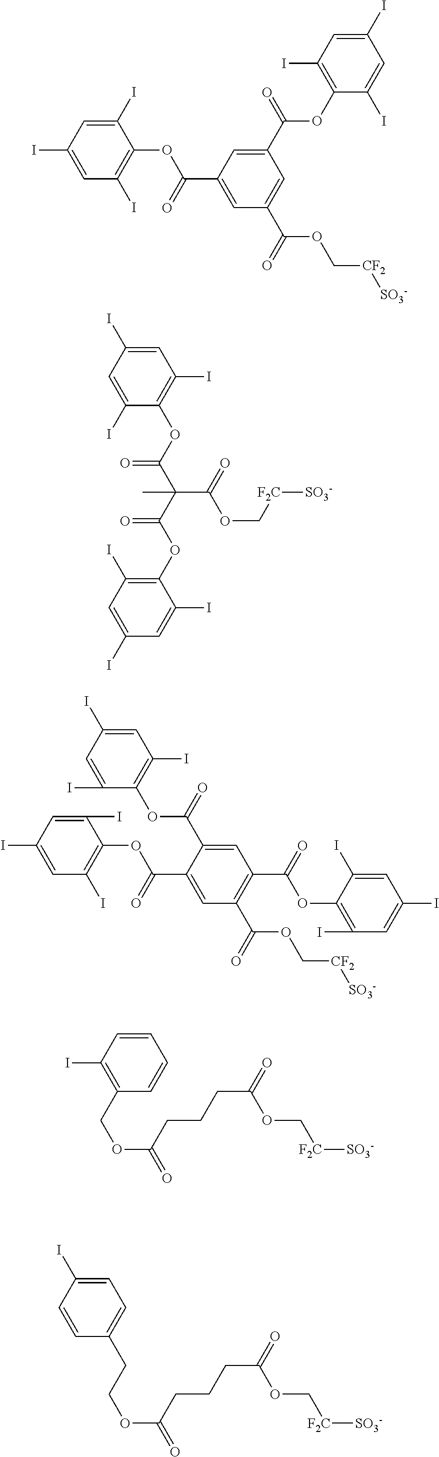

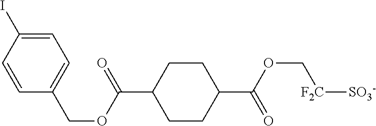

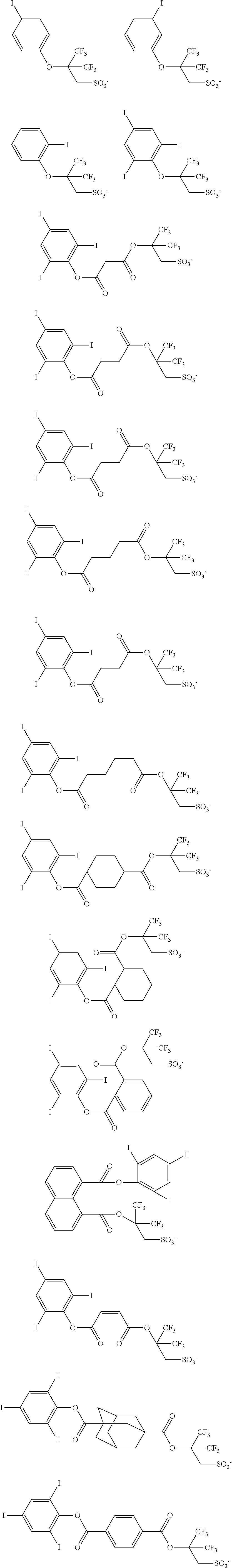

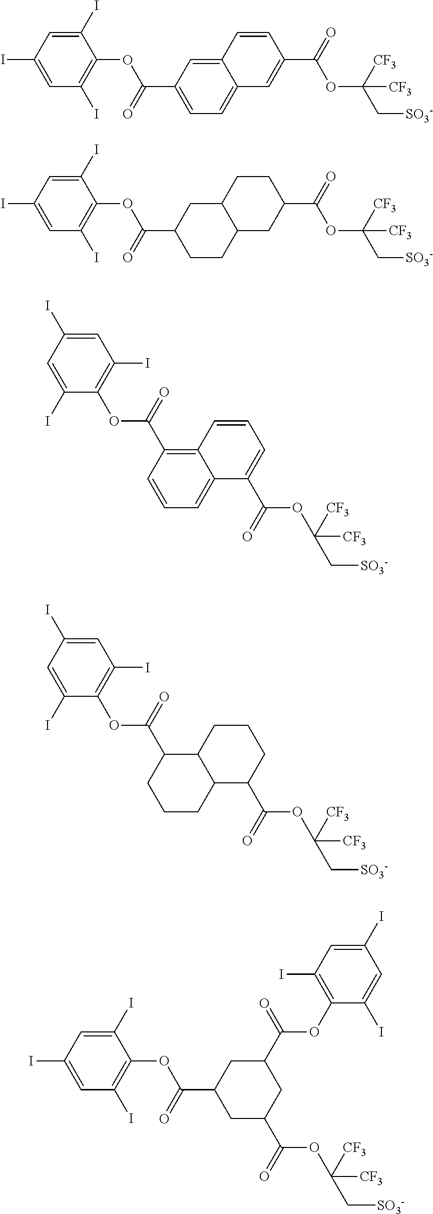

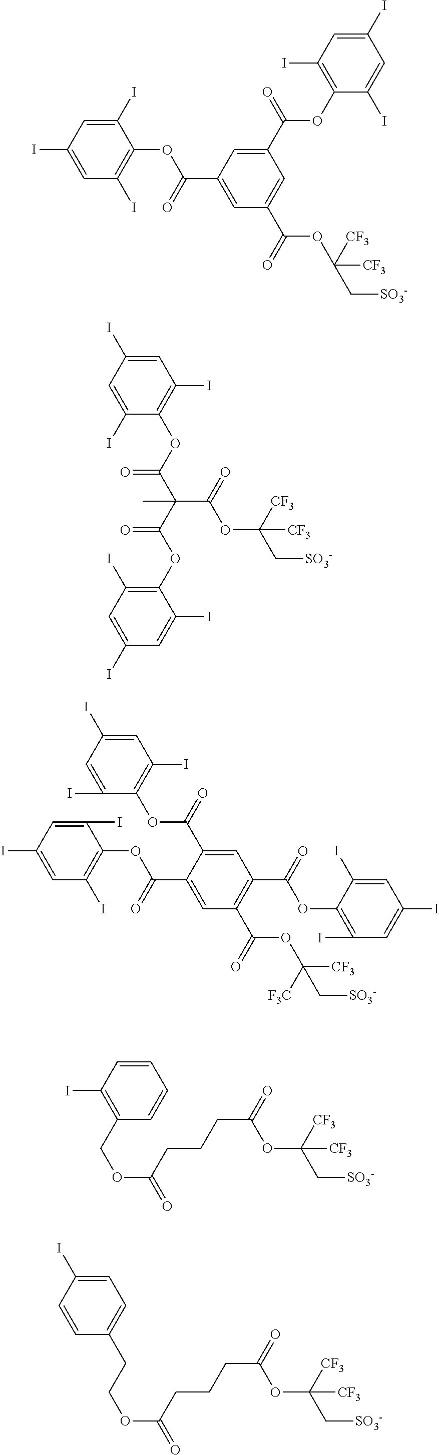

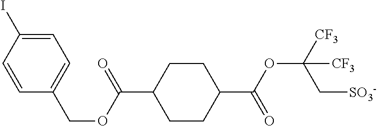

If the chemically amplified resist composition of the present invention contains acid generator (D), the amount of acid generator (D) contained therein is preferably 0.1 to 40 parts by mass, and more preferably 0.5 to 20 parts by mass per 80 parts by mass of base polymer (A). The case that the amount of the acid generator to be added as component (D) falls within that range is preferred because good resolution results and problems of undesired matters are reliably avoided after development of a resist film or during peeling-off of a resist film. For acid generator (D), one acid generator may be used alone; alternatively, two or more acid generators may be used in combination.

### [Surfactant (E)]

The chemically amplified resist composition of the present invention may further contain a surfactant as component (E). Surfactant (E) is preferably a surfactant that is insoluble or poorly soluble in water and soluble in alkaline developers, or a surfactant that is insoluble or poorly soluble in water and alkaline developers. For such surfactants, reference can be made to those described in JP 2010-215608 A and JP 2011-16746 A.

For example, among the surfactants described in the publications, FC-4430 (manufactured by 3M Company), Surflon (registered trademark) 5-381 (manufactured by AGC Seimi Chemical Co., Ltd.), OLFINE (registered trademark) E1004 (manufactured by Nissin Chemical Industry Co., Ltd.), KH-20 and KH-30 (manufactured by AGC Seimi Chemical Co., Ltd.), and an oxetane ring-opening polymerization product represented by the following formula (surf-1) are preferred as the surfactant that is insoluble or poorly soluble in water and alkaline developers:

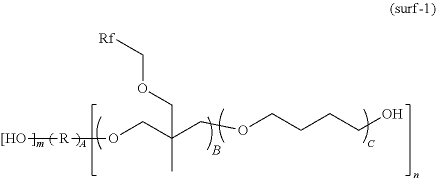

Here, R, Rf, A, B, C, m, and n are applied only to the formula (surf-1), independently of the description shown above. R is a di- to tetravalent aliphatic group having 2 to 5 carbon atoms. Examples of the aliphatic group include an ethylene group, a 1,4-butylene group, a 1,2-propylene group, a 2,2-dimethyl-1,3-propylene group, and a 1,5-pentylene group as divalent aliphatic groups, and the following groups as tri- or tetravalent aliphatic groups:

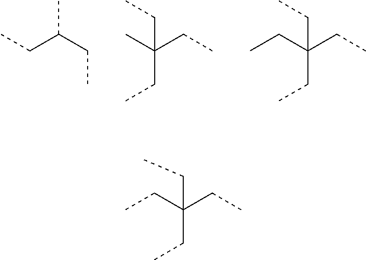

wherein each dashed line indicates bonding, and these are partial structures derived from glycerol, trimethylolethane, trimethylolpropane, and pentaerythritol, respectively.

Among them, for example, a 1,4-butylene group and a 2,2-dimethyl-1,3-propylene group are preferred.

Rf is a trifluoromethyl group or a pentafluoroethyl group, and preferably a trifluoromethyl group. m is an integer of 0 to 3, n is an integer of 1 to 4, and the sum of n and m is the valence of R, being an integer of 2 to 4. A is 1. B is an integer of 2 to 25, and preferably an integer of 4 to 20. C is an integer of 0 to 10, and preferably 0 or 1. The constituent units shown in the formula (surf-1) are not intended to specify the order of them, and may be bound in a block-by-block manner or randomly. Production of surfactants based on partially fluorinated oxetane ring-opening polymerization products is described in detail in, for example, U.S. Pat. No. 5,650,483 A.

In the case that no resist protection film is used in ArF immersion lithography, the surfactant that is insoluble or poorly soluble in water and soluble in alkaline developers functions to reduce the permeation and leaching of water by orienting on the surface of a resist film. Accordingly, this surfactant is useful for inhibiting the elution of water-soluble components from resist films to reduce damage to the exposure device, and also useful in development with alkaline aqueous solution after exposure or after post-exposure bake (PEB) because the surfactant solubilizes at that time and is less likely to form undesired matters causative of defects. Such surfactants, which have a characteristic of being insoluble or poorly soluble in water and soluble in alkaline developers and are polymeric surfactants, are also called hydrophobic resins, and in particular, surfactants that have high water repellency and give enhanced water slipperiness are preferred.

Specific examples of such polymeric surfactants include those containing at least one selected from repeating units represented by the following formulae (6A) to (6E):

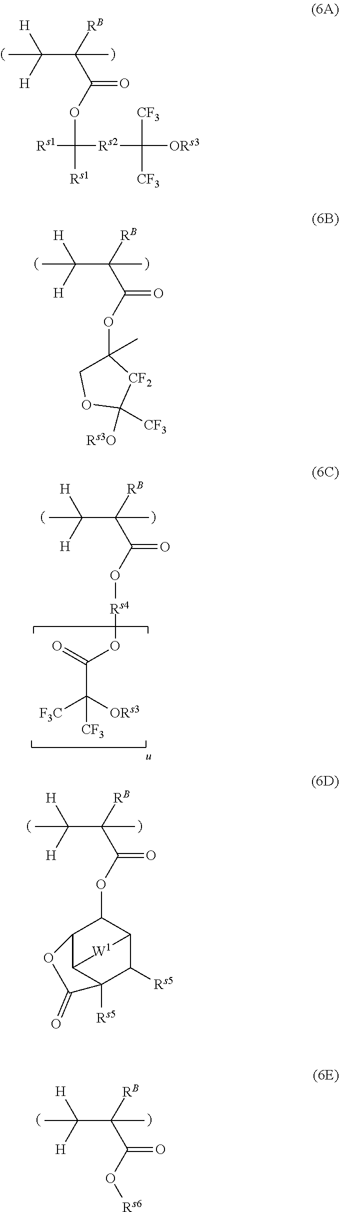

In the formulae (6A) to (6E), R^{B} is a hydrogen atom, a fluorine atom, a methyl group, or a trifluoromethyl group. W¹ is —CH₂—, —CH₂CH₂—, —O—, or two —H atoms that separate from each other. R^{s1} groups are each independently a hydrogen atom or a hydrocarbyl group having 1 to 10 carbon atoms. R^{s2} is a single bond or a linear or branched hydrocarbylene group having 1 to 5 carbon atoms. R^{s3} groups are each independently a hydrogen atom, a hydrocarbyl group or fluorinated hydrocarbyl group having 1 to 15 carbon atoms, or an acid-unstable group. If R^{s3} is a hydrocarbyl group or fluorinated hydrocarbyl group, an ether bond or a carbonyl group is optionally present between a carbon-carbon bond. R^{s4} is a (u+1)-valent hydrocarbon group or fluorinated hydrocarbon group having 1 to 20 carbon atoms. u is an integer of 1 to 3. R^{s3} groups are each independently a hydrogen atom or a group represented by —C(═O)—O—R^{sa}. R^{sa} is a fluorinated hydrocarbyl group having 1 to 20 carbon atoms. R^{s6} is a hydrocarbyl group or fluorinated hydrocarbyl group having 1 to 15 carbon atoms, wherein an ether bond or a carbonyl group is optionally present between a carbon-carbon bond therein.

The hydrocarbyl group represented by R^{s1}, having 1 to 10 carbon atoms, is preferably a saturated hydrocarbyl group, and may be linear, branched, or cyclic. Specific examples thereof include an alkyl group having 1 to 10 carbon atoms such as a methyl group, an ethyl group, a n-propyl group, an isopropyl group, a n-butyl group, an isobutyl group, a sec-butyl group, a tert-butyl group, a n-pentyl group, a n-hexyl group, a n-heptyl group, a n-octyl group, a n-nonyl group, and a n-decyl group; and a cyclic saturated hydrocarbyl group having 3 to 10 carbon atoms such as a cyclopropyl group, a cyclobutyl group, a cyclopentyl group, a cyclohexyl group, an adamantyl group, and a norbornyl group. Among them, hydrocarbyl groups having 1 to 6 carbon atoms are preferred.

The hydrocarbylene group represented by R^{s2} is preferably a saturated hydrocarbylene group, and may be linear, branched, or cyclic. Specific examples thereof include a methylene group, an ethylene group, a propylene group, a butylene group, and a pentylene group.

The hydrocarbyl group represented by R^{s3} or R^{s6} may be saturated or unsaturated, and may be linear, branched, or cyclic. Specific examples thereof include a saturated hydrocarbyl group and an aliphatic unsaturated hydrocarbyl group such as an alkenyl group and an alkynyl group, and a saturated hydrocarbyl group is preferred. Specific examples of the saturated hydrocarbyl group include not only those shown as examples of the hydrocarbyl group represented by R^{s1}, but also an undecyl group, a dodecyl group, a tridecyl group, a tetradecyl group, and a pentadecyl group. Specific examples of the fluorinated hydrocarbyl group represented by R^{s3} or R^{s6} include a group given by replacing each of some or all of the hydrogen atoms bound to the carbon atoms of the aforementioned hydrocarbyl group with a fluorine atom. As described above, an ether bond or a carbonyl group is optionally present between a carbon-carbon bond in the hydrocarbyl group.

Specific examples of the acid-unstable group represented by R^{s3} include the aforementioned groups represented by the formulae (AL-1) to (AL-3), a trialkylsilyl group each alkyl group of which is an alkyl group having 1 to 6 carbon atoms, and an oxo group-containing alkyl group having 4 to 20 carbon atoms.

The (u+1)-valent hydrocarbon group or fluorinated hydrocarbon group represented by R^{s4} may be linear, branched, or cyclic, and specific examples thereof include a group given by further eliminating u hydrogen atoms from, for example, the aforementioned hydrocarbyl group or fluorinated hydrocarbyl group.

The fluorinated hydrocarbyl group represented by R^{sa} is preferably saturated one, and may be linear, branched, or cyclic. Specific examples thereof include a group given by replacing each of some or all of the hydrogen atoms of the hydrocarbyl group with a fluorine atom, and specific examples thereof include a trifluoromethyl group, a 2,2,2-trifluoroethyl group, a 3,3,3-trifluoro-1-propyl group, a 3,3,3-trifluoro-2-propyl group, a 2,2,3,3-tetrafluoropropyl group, a 1,1,1,3,3,3-hexafluoroisopropyl group, a 2,2,3,3,4,4,4-heptafluorobutyl group, a 2,2,3,3,4,4,5,5-octafluoropentyl group, a 2,2,3,3,4,4,5,5,6,6,7,7-dodecafluoroheptyl group, a 2-(perfluorobutyl)ethyl group, a 2-(perfluorohexyl)ethyl group, a 2-(perfluorooctyl)ethyl group, and a 2-(perfluorodecyl)ethyl group.

Specific examples of repeating units represented by any of the formulae (6A) to (6E) include, but are not limited, those shown below. In the following formulae, R^{B} is as specified above:

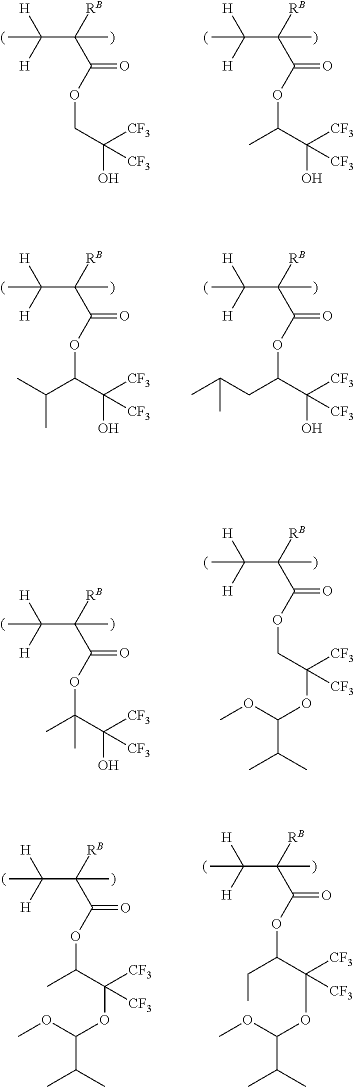

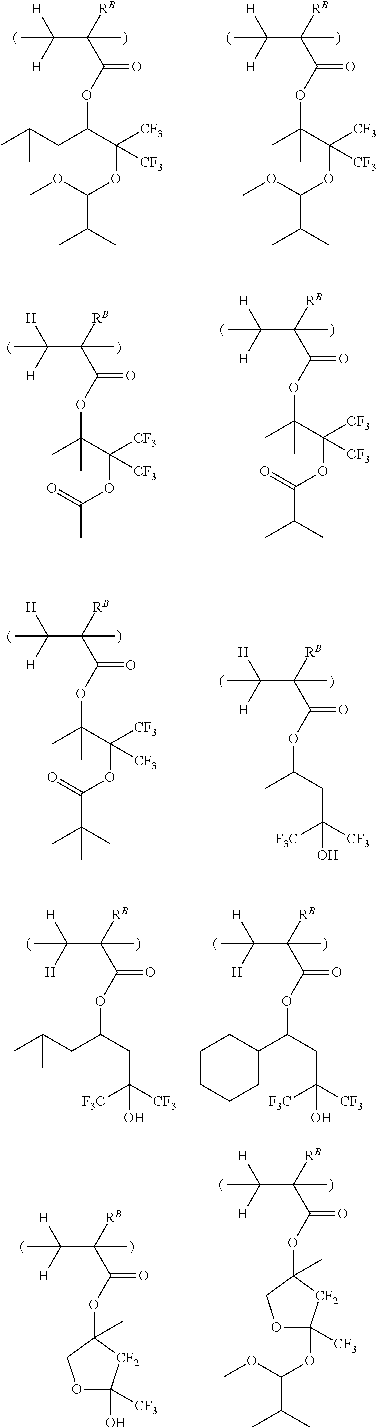

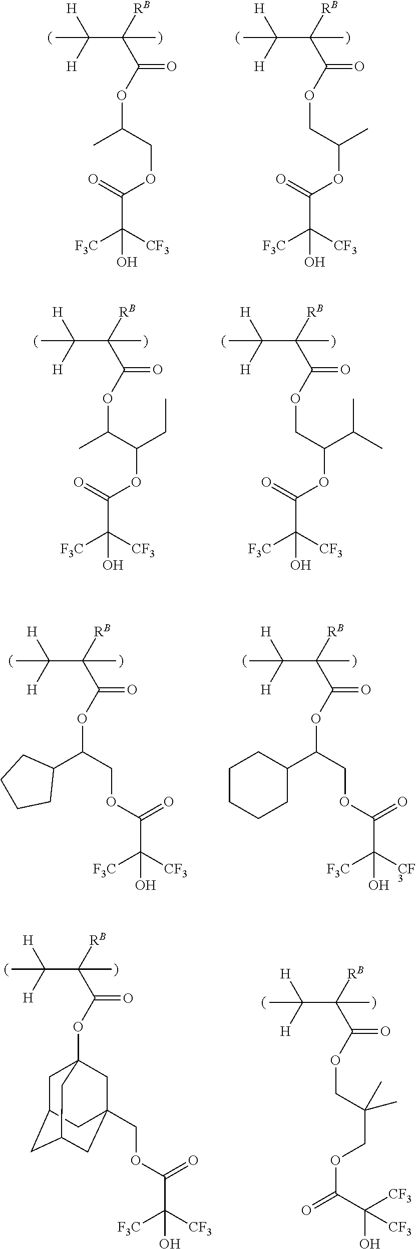

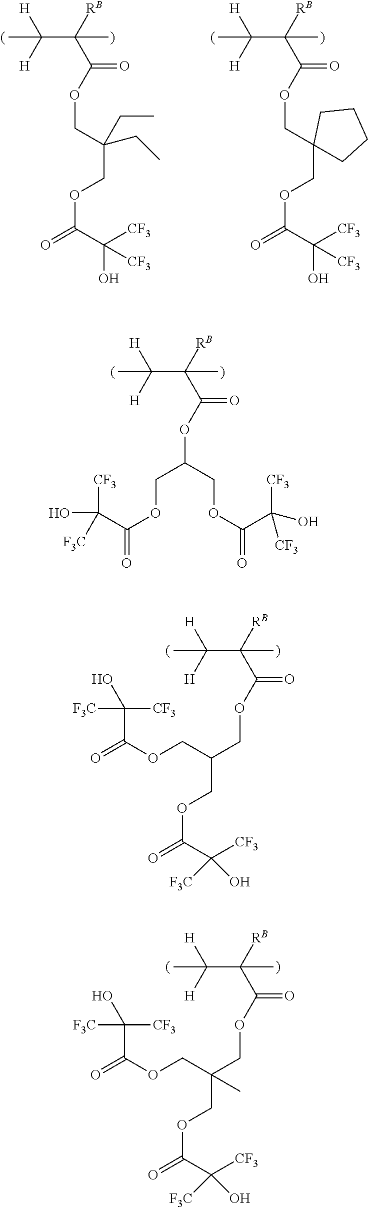

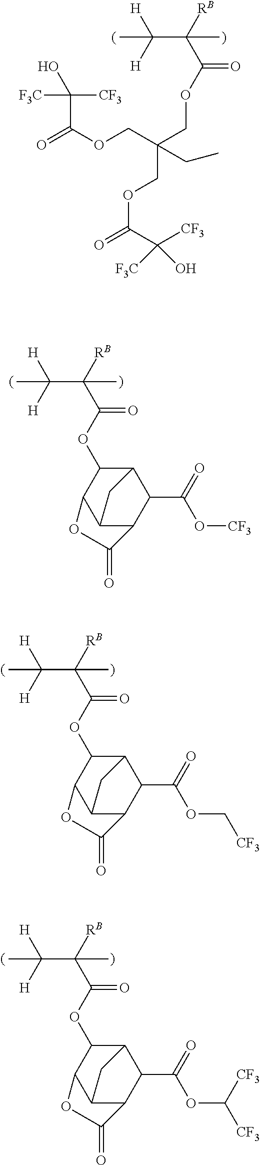

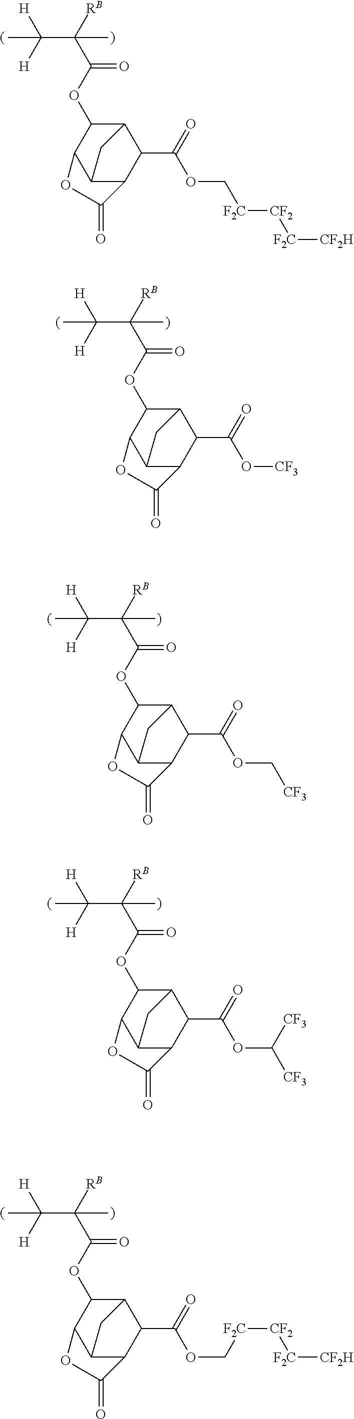

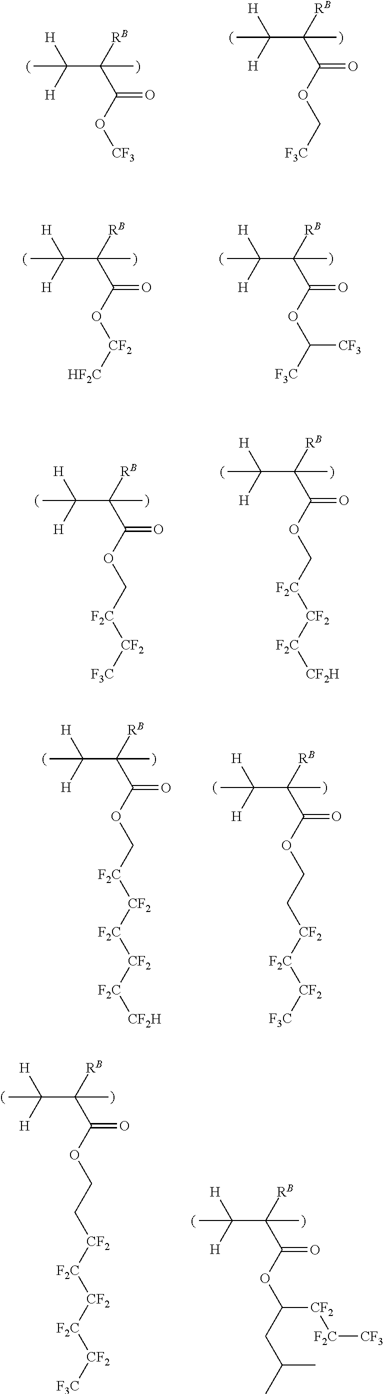

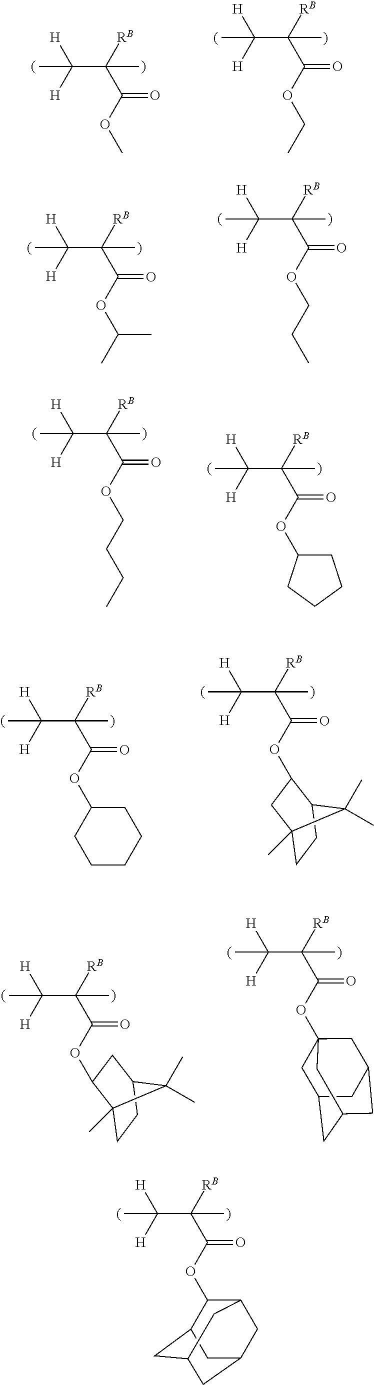

The polymeric surfactant may further contain an additional repeating unit other than the repeating units represented by the formulae (6A) to (6E). Specific examples of the additional repeating unit include a repeating unit derived from methacrylic acid or an α-trifluoromethylacrylic acid derivative. The amount of the repeating units, contained in the polymeric surfactant, represented by the formulae (6A) to (6E) is preferably 20 mol % or more, more preferably 60 mol % or more, and further preferably 100 mol % to the total of the repeating units.

The Mw of the polymeric surfactant is preferably 1000 to 500000, and more preferably 3000 to 100000. The Mw/Mn is preferably 1.0 to 2.0, and more preferably 1.0 to 1.6.

Methods for synthesizing the polymeric surfactant include a method of polymerizing the repeating units represented by the formulae (6A) to (6E) and, as necessary, monomers containing unsaturated bonds that give additional repeating units by heating in an organic solvent with addition of a radical initiator. Specific examples of the organic solvent to be used in the polymerization include toluene, benzene, THF, diethyl ether, and dioxane. Specific examples of the polymerization initiator include AIBN, 2,2′-azobis(2,4-dimethylvaleronitrile), dimethyl 2,2-azobis(2-methylpropionate), benzoyl peroxide, and lauroyl peroxide. The reaction temperature is preferably 50 to 100° C. The reaction time is preferably 4 to 24 hours. The acid-unstable group introduced to a monomer may be used as it is; otherwise, the acid-unstable group may be protected or partially protected after polymerization.

In the case that the polymeric surfactant is synthesized, a known chain transfer agent such as dodecylmercaptan and 2-mercaptoethanol may be used for molecular weight adjustment. In this case, the amount of the chain transfer agent to be added is preferably 0.01 to 10 mol % based on the total number of moles of the monomers to be polymerized.

If the chemically amplified resist composition of the present invention contains surfactant (E), the amount of surfactant (E) contained therein is preferably 0.1 to 50 parts by mass, and more preferably 0.5 to 10 parts by mass per 80 parts by mass of base polymer (A). With the amount of surfactant (E) contained being 0.1 parts by mass or more, sufficiently improved receding contact angle is achieved between the surface of the resist film and water; with the amount of surfactant (E) contained being 50 parts by mass or less, the surface of the resist film has low dissolution rates to developer, and the height of formed fine patterns is sufficiently maintained. For surfactant (E), one surfactant may be used alone; alternatively, two or more surfactants may be used in combination.

### [Dissolution Inhibitor (F)]

The chemically amplified resist composition of the present invention may further contain a dissolution inhibitor as component (F). In the case that the chemically amplified resist composition of the present invention is positive-type, the difference between dissolution rates in exposed parts and in unexposed parts can be more increased by blending a dissolution inhibitor, and more enhanced resolution is successfully achieved.

Specific examples of the dissolution inhibitor include a compound given by replacing each of the hydrogen atoms of phenolic hydroxy groups of a compound having a molecular weight preferably of 100 to 1000, more preferably of 150 to 800, and containing two or more phenolic hydroxy groups in the molecule with an acid-unstable group at a proportion of 0 to 100 mol % to the total, or a compound given by replacing each of the hydrogen atoms of carboxy groups of a compound containing carboxy groups in the molecule with an acid-unstable group at a proportion of 50 to 100 mol % on average to the total. Specific examples thereof include a compound given by replacing each of the hydrogen atoms of hydroxy groups or carboxy groups of bisphenol A, trisphenol, phenolphthalein, cresol-novolac, naphthalenecarboxylic acid, adamantanecarboxylic acid, or cholic acid with an acid-unstable group, for example, those shown in paragraphs [0155] to [0178] of JP 2008-122932 A.

If the chemically amplified resist composition of the present invention contains dissolution inhibitor (F), the amount of dissolution inhibitor (F) contained therein is preferably 0 to 50 parts by mass, and more preferably 5 to 40 parts by mass per 80 parts by mass of base polymer (A).

### [Additional Component (G)]

The chemically amplified resist composition of the present invention may contain, for example, a compound that is decomposed by an acid and generates an acid (acid amplifier compound), an organic acid derivative, a fluorine-substituted alcohol, or a water repellency enhancer as additional component (G). For the acid amplifier compound, reference can be made to compounds described in JP 2009-269953 A or JP 2010-215608 A. If the acid amplifier compound is contained, the amount of the acid amplifier compound contained is preferably 0 to 5 parts by mass, and more preferably 0 to 3 parts by mass per 80 parts by mass of base polymer (A). If the amount of the acid amplifier compound contained is excessively large, control of acid diffusion is difficult, and the degradation of resolution and the degradation of pattern shapes may occur. For the organic acid derivative and fluorine-substituted alcohol, reference can be made to compounds described in JP 2009-269953 A or JP 2010-215608 A.

The water repellency enhancer can be used for immersion lithography without use of any top coat. For example, the water repellency enhancer is preferably a polymer containing a fluorinated alkyl group, in particular, a polymer of specific structure containing a 1,1,1,3,3,3-hexafluoro-2-propanol residue, and more preferably those shown as examples, for example, in JP 2007-297590 A and JP 2008-111103 A. The water repellency enhancer needs to be dissolved in an alkaline developer or an organic solvent developer. The aforementioned specific water repellency enhancer having a 1,1,1,3,3,3-hexafluoro-2-propanol residue exhibits good solubility in developers. Polymers containing a repeating unit containing an amino group or an amine salt have high effects as the water repellency enhancer to inhibit the evaporation of an acid during PEB to prevent the opening failure of hole patterns after development. If the chemically amplified resist composition of the present invention contains the water repellency enhancer, the amount of the water repellency enhancer contained therein is preferably 0 to 20 parts by mass, and more preferably 0.5 to 10 parts by mass per 80 parts by mass of base polymer (A).

### [Patterning Method]

In using the chemically amplified resist composition of the present invention for production of integrated circuits of any type, known lithographic techniques can be applied. Patterning methods therewith include a method including: a step of forming a resist film on a substrate by using the aforementioned chemically amplified resist composition; a step of exposing the resist film to a high-energy ray; and a step of developing the exposed resist film by using a developer.

First, the chemically amplified resist composition of the present invention is applied onto a substrate for integrated circuit production (e.g., Si, SiO₂, SiN, SiON, TiN, WSi, BPSG, SOG, an organic anti-reflection film) or a substrate for mask circuit production (e.g., Cr, CrO, CrON, MoSi₂, SiO₂) to give a coating film thickness of 0.01 to 2.0 μm by a proper application method such as spin coating, roll coting, flow coating, dip coating, spray coating, and doctor coating. The resultant is prebaked on a hot plate preferably at 60 to 150° C. for 10 seconds to 30 minutes, more preferably at 80 to 120° C. for 30 seconds to 20 minutes, to form a resist film.

Then, the resist film is exposed by using a high-energy ray. Examples of the high-energy ray include an ultraviolet ray, a far-ultraviolet ray, an EB, an EUV having a wavelength of 3 to 15 nm, an X-ray, a soft X-ray, excimer laser light, a γ-ray, and synchrotron radiation. If an ultraviolet ray, a far-ultraviolet ray, an EUV, an X-ray, a soft X-ray, excimer laser light, a γ-ray, synchrotron radiation, or the like is used as the high-energy ray, irradiation is performed directly or with use of a mask to form an intended pattern to give a dose preferably of about 1 to 200 mJ/cm², more preferably of about 10 to 100 mJ/cm². If an EB is used as the high-energy ray, drawing is performed directly or with use of a mask to form an intended pattern to give a dose preferably of about 0.1 to 100 μC/cm², more preferably of about 0.5 to 50 μC/cm². The chemically amplified resist composition of the present invention is suitable particularly for fine patterning with ArF excimer laser light having a wavelength of 193 nm, KrF excimer laser light having a wavelength of 248 nm, an EB or an EUV (extreme ultraviolet ray) having a wavelength of 3 to 15 nm, an X-ray, a soft X-ray, a γ-ray, or synchrotron radiation among high-energy rays.

After the exposure, PEB may be performed on a hot plate preferably at 60 to 150° C. for 10 seconds to 30 minutes, more preferably at 80 to 120° C. for 30 seconds to 20 minutes.

After the exposure or PEB, development is performed by a conventional method such as a dip method, a puddle method, and a spray method with a developer of 0.1 to 10% by mass, preferably 2 to 5% by mass alkaline aqueous solution of tetramethylammonium hydroxide (TMAH), tetraethylammonium hydroxide, tetrapropylammonium hydroxide, tetrabutylammonium hydroxide, or the like for 3 seconds to 3 minutes, preferably for 5 seconds to 2 minutes, and as a result parts irradiated with light dissolve in the developer and unexposed parts remain undissolved, and an intended positive-type pattern is formed on the substrate.

Alternatively, a negative-type pattern can be obtained by using an organic solvent developer in place of the alkaline aqueous solution. Specific examples of the developer to be used in this case include 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, benzyl formate, phenylethyl formate, methyl 3-phenylpropionate, benzyl propionate, ethyl phenylacetate, and 2-phenylethyl acetate. One of these organic solvents may be used alone; alternatively, two or more thereof may be used as a mixture.

At the completion of development, rinsing may be performed. Preferred as the rinsing solution is a solvent that is miscible with the developer and does not dissolve the resist film. For the solvent, an alcohol having 3 to 10 carbon atoms, an ether compound having 8 to 12 carbon atoms, an alkane, alkene, or alkyne having 6 to 12 carbon atoms, or an aromatic solvent is preferably used.

Specific examples of the alcohol having 3 to 10 carbon atoms include n-propyl alcohol, isopropyl alcohol, 1-butyl alcohol, 2-butyl alcohol, isobutyl alcohol, tert-butyl alcohol, 1-pentanol, 2-pentanol, 3-pentanol, tert-pentyl alcohol, neopentyl alcohol, 2-methyl-1-butanol, 3-methyl-1-butanol, 3-methyl-3-pentanol, cyclopentanol, 1-hexanol, 2-hexanol, 3-hexanol, 2,3-dimethyl-2-butanol, 3,3-dimethyl-1-butanol, 3,3-dimethyl-2-butanol, 2-ethyl-1-butanol, 2-methyl-1-pentanol, 2-methyl-2-pentanol, 2-methyl-3-pentanol, 3-methyl-1-pentanol, 3-methyl-2-pentanol, 3-methyl-3-pentanol, 4-methyl-1-pentanol, 4-methyl-2-pentanol, 4-methyl-3-pentanol, cyclohexanol, and 1-octanol.

Specific examples of the ether compound having 8 to 12 carbon atoms include di-n-butyl ether, diisobutyl ether, di-sec-butyl ether, di-n-pentyl ether, diisopentyl ether, di-sec-pentyl ether, di-tert-pentyl ether, and di-n-hexyl ether.

Specific examples of the alkane having 6 to 12 carbon atoms include hexane, heptane, octane, nonane, decane, undecane, dodecane, methylcyclopentane, dimethylcyclopentane, cyclohexane, methylcyclohexane, dimethylcyclohexane, cycloheptane, cyclooctane, and cyclononane. Specific examples of the alkene having 6 to 12 carbon atoms include hexene, heptene, octene, cyclohexene, methylcyclohexene, dimethylcyclohexene, cycloheptene, and cyclooctene. Specific examples of the alkyne having 6 to 12 carbon atoms include hexyne, heptyne, and octyne.

Specific examples of the aromatic solvent include toluene, xylene, ethylbenzene, isopropylbenzene, tert-butylbenzene, and mesitylene.

The generation of collapse and defects of resist patterns can be reduced by rinsing. The rinsing is not necessarily essential, and the amount of usage of the solvent can be reduced by omitting rinsing.

Hole patterns and trench patterns after development can be shrunk by thermal flow, a RELACS technique, or a DSA technique. If a shrinking agent is applied onto a hole pattern, the diffusion of an acid catalyst from the resist film during bake causes the shrinking agent to crosslink on the surface of the resist film, and the shrinking agent attaches to the side walls of the hole pattern. The bake temperature is preferably 70 to 180° C., and more preferably 80 to 170° C., and the bake time is preferably 10 to 300 seconds; thereby, excessive portions of the shrinking agent are removed, and the hole pattern is down-sized.

### EXAMPLES

The following specifically describes the present invention by showing examples and comparative examples, but the present invention is not limited by the following examples. An apparatus used is as follows:
MALDI TOF-MS: S3000 manufactured by JEOL Ltd.
UPLC: ACQUITY UPLC H-Class Plus manufactured by Waters Corporation

Used as raw materials for synthesis of onium salt monomers were the following carboxylic acids (SM-a1 to a6), fluorostyrenes (SM-b1 to b2), fluoro alcohol derivatives (SM-c1 to c4), and sulfonium salts and iodonium salt (SM-d1 to d5).

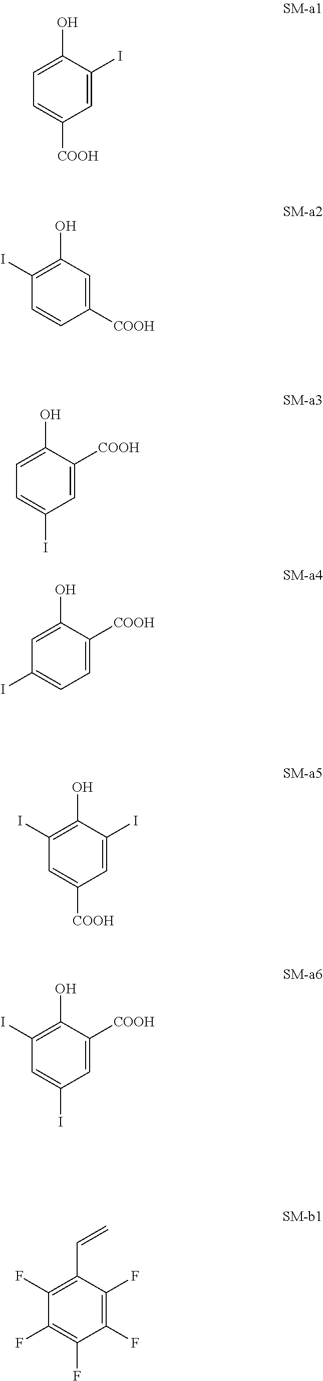

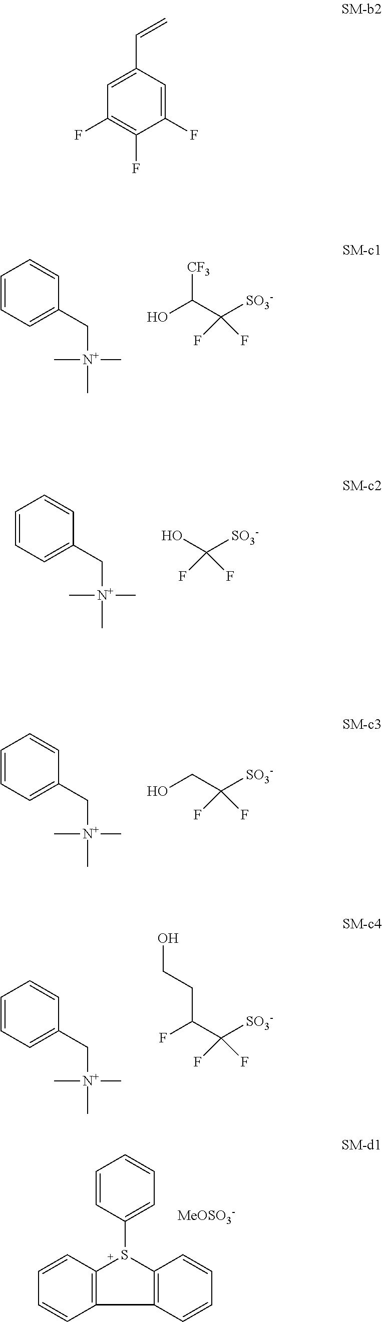

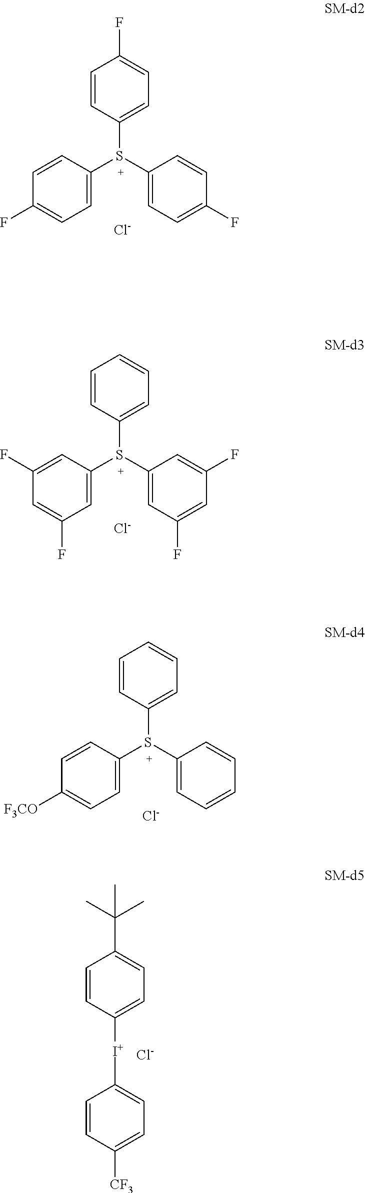

### [1] Synthesis of Onium Salt Monomer

### [Example 1-1] Synthesis of Onium Salt Monomer (a-1)

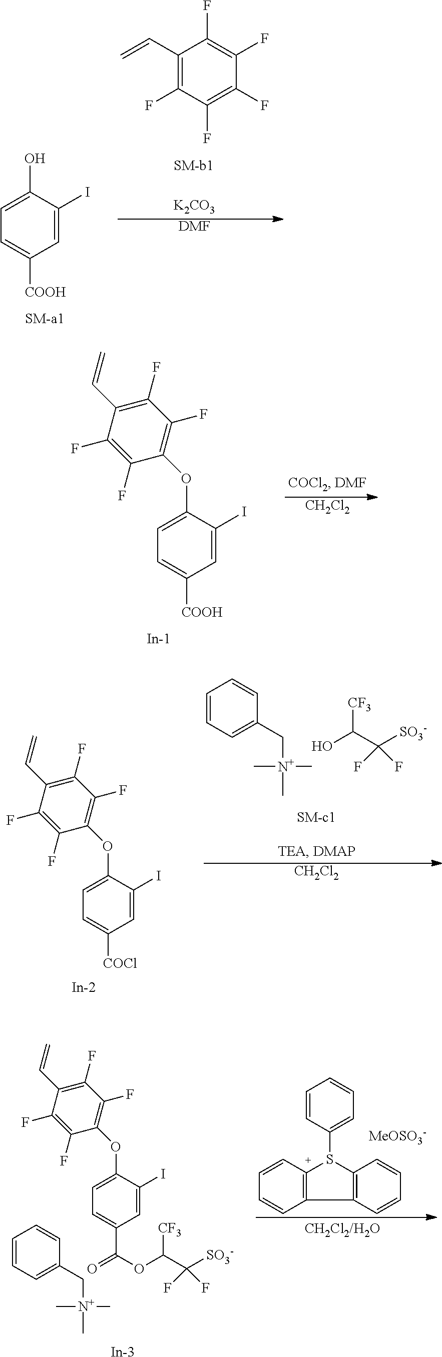

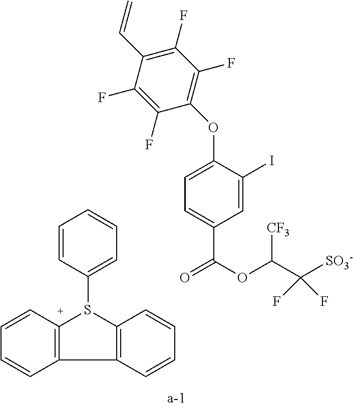

(1) Synthesis of Intermediate in-1

Under a nitrogen atmosphere, 32.3 g of raw material (SM-a1), 19.6 g of potassium carbonate, and 200 g of DMF (N,N-dimethylformamide) were added in a reaction vessel, and homogenously dispersed at an internal temperature of 40° C. After that, 25.0 g of raw material (SM-b1) that had been dissolved in advance in 50 g of DMF was added dropwise over 30 minutes. After the dropwise addition, the temperature in the reaction vessel was increased to 60° C., and reaction was performed for 6 hours. Thereafter, the reaction solution was cooled, and then added dropwise to a mixed solution of 300 ml of MIBK and 300 g of 5% HCl aqueous solution for neutralization. After the neutralization, the resultant was transferred into a separatory funnel to separate and remove the aqueous layer, washing was performed five times with 100 ml of ultrapure water, and the organic phase was recovered. After the organic phase was subjected to distillation, recrystallization was performed with IPE (diisopropyl ether) to afford 35.8 g of intermediate (In-1) as white crystals (% yield: 67%).

(2) Synthesis of Intermediate in-2

Under a nitrogen atmosphere, 35.0 g of intermediate (In-1), 200 g of methylene chloride, and 0.05 g of DMF were added to form a homogenous dispersion. With cooling in a water bath with cooling in an ice bath, 11.2 g of oxazoyl chloride was added dropwise. After the dropwise addition, reaction was performed at room temperature for 12 hours. After completion of the reaction, low-boiling-point components remaining in the solution were distilled off, the resultant was diluted again with methylene chloride, and 160.5 g of intermediate (In-2) was collected as 25% methylene chloride solution (100% in terms of % yield). This was used for the subsequent step without purification or the like.

(3) Synthesis of Intermediate in-3

Under a nitrogen atmosphere, 30.0 g of raw material (SM-c1), 10.6 g of TEA (triethylamine), 1.1 g of DMAP (4-dimethylaminopyridine), and methylene chloride (150 g) were added to a reaction vessel to form a homogenous solution. With cooling in an ice bath, 160.0 g (corresponding to 40.0 g of solid contents) of 25 wt % methylene chloride solution of intermediate In-2 was added dropwise over 30 minutes. After completion of the dropwise addition, reaction was performed at room temperature for 12 hours, and 100 g of 3% HCl aqueous solution was added to terminate the reaction. After the termination of the reaction, the resultant was transferred into a separatory funnel to separate and remove the aqueous layer, washing was further performed five times with 100 ml of ultrapure water, and the organic phase was recovered. After the organic phase was subjected to distillation, recrystallization was performed with IPE to afford 64.1 g of intermediate (In-3) as white crystals (% yield: 82%).

### (4) Synthesis of Onium Salt Monomer a-1

Under a nitrogen atmosphere, 60.0 g of intermediate (In-3), 34.3 g of (SM-d1), 300 g of methylene chloride, and 150 g of ultrapure water were added to a reaction vessel, the resultant was stirred for 30 minutes, and the organic layer was then transferred into a separatory funnel to remove the aqueous layer. Further, washing was repeated five times with 150 g of ultrapure water, and the organic phase was then recovered, and concentrated under reduced pressure. The concentrated solution was purified by silica gel chromatography to afford 60.5 g of the target monomer, onium salt monomer (a-1), as white crystals (% yield: 82%).

### Maldi Tof-Ms:

POSITIVE M⁺261 (corresponding to C₁₈H₁₃S⁺)
NEGATIVE M⁻648 (corresponding to C₁₇H₈F₆IO₆S₁⁻)

### [Examples 1-2 to 1-6] Synthesis of Onium Salt Monomers (a-2) to (a-6)

Raw materials for monomers to be synthesized were appropriately selected from the carboxylic acids (SM-a1 to a6), fluorostyrenes (SM-b1 to b2), fluoro alcohol derivatives (SM-c1 to c4), sulfonium salts and iodonium salt (SM-d1 to d5), and onium salt monomers (a-2) to (a-6) were synthesized therewith through the same synthesis route as in the synthesis of onium salt monomer (a-1) in [Example 1-1]. The following shows the structural formulae of onium salt monomers (a-1) to (a-6) synthesized.

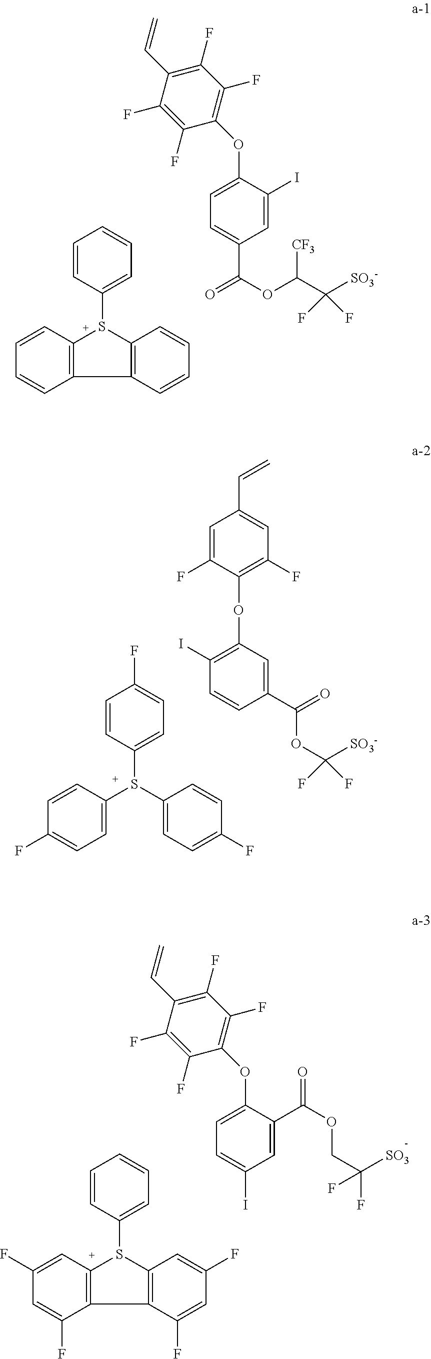

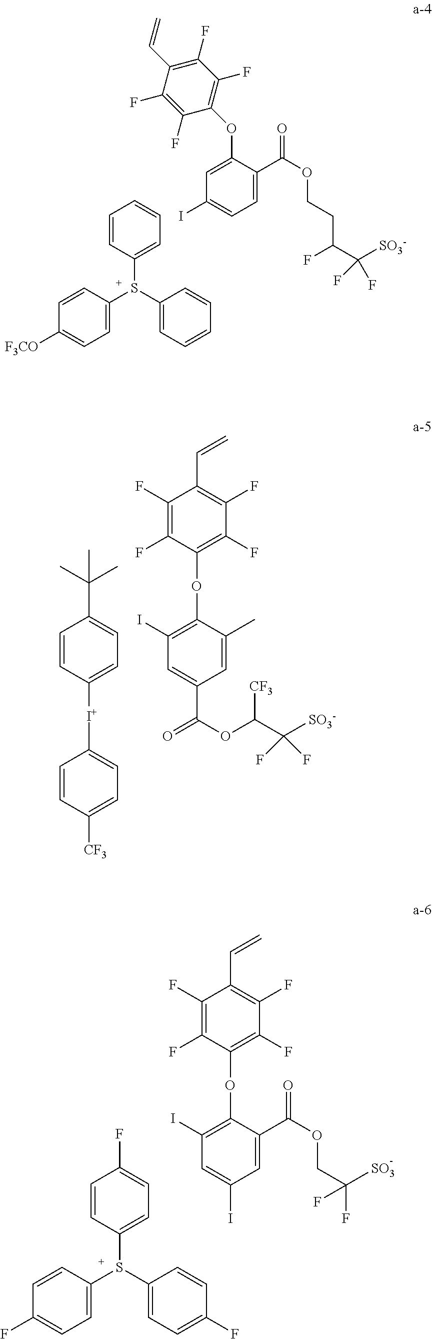

### [Comparative Examples 1-1 to 1-4] Synthesis of Comparative Onium Salt Monomers (Ca-1) to (Ca-4)

Comparative onium salt monomers (ca-1) to (ca-4) represented by the following formulae were synthesized by using the corresponding raw materials and known organic synthesis reactions:

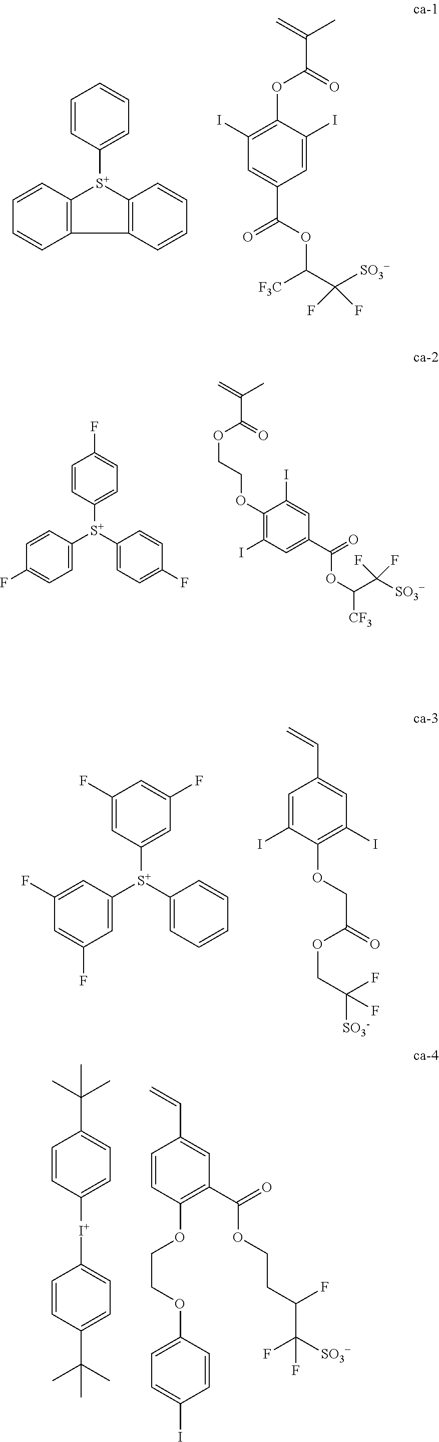

### [2] Synthesis of Base Polymer

Among monomers used for synthesis of base polymers, those other than onium salt monomers (a-1) to (a-6) and comparative monomers (ca-1) to (ca-4) are as follows:

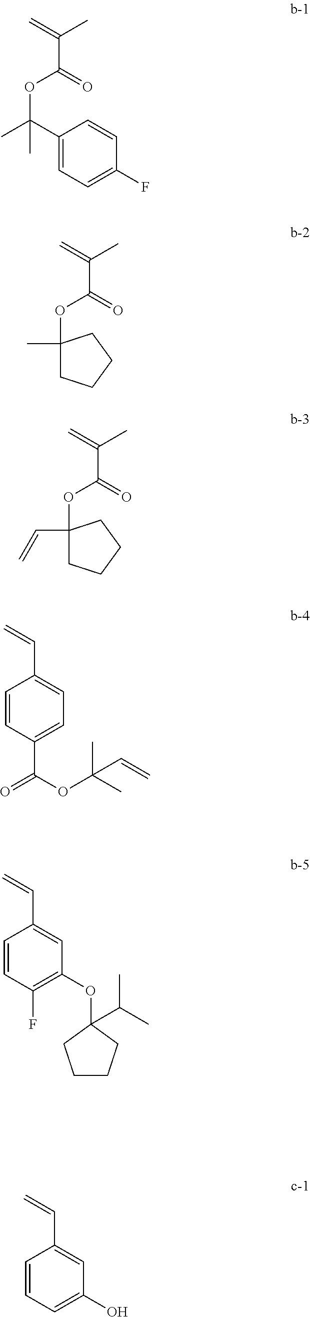

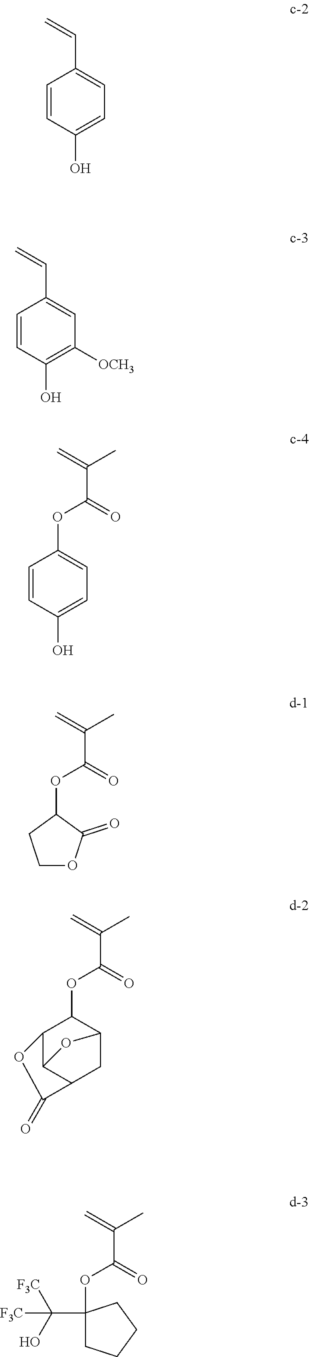

### [Example 2-1] Synthesis of Polymer (P-1)

Under a nitrogen atmosphere, 22.2 g of monomer (a-1), 21.5 g of monomer (b-1), 6.3 g of monomer (c-1), 2.02 g of V-601, and 70 g of MEK (methyl ethyl ketone) were added to a flask to prepare a monomer-polymerization initiator solution. To another reaction vessel under a nitrogen atmosphere, 23 g of MEK was added and heated to 80° C. with stirring, and then the monomer-polymerization initiator solution was added thereto dropwise over 4 hours. After completion of the dropwise addition, stirring was continued for 2 hours while the temperature of the polymerization solution was kept at 80° C., and the resultant was then cooled to room temperature. The resulting polymerization solution was added dropwise to 1500 g of hexane vigorously stirred, and a polymer precipitated was separated by filtration. The resulting polymer was washed twice with 300 g of hexane, and then vacuum-dried at 50° C. for 20 hours to afford polymer (P-1) in the form of white powder (yield: 48.5 g, % yield: 97%). The Mw and Mw/Mn of polymer (P-1) were 9100 and 1.71, respectively. The Mw is a measurement in terms of polystyrene as determined by GPC with use of DMF as a solvent.

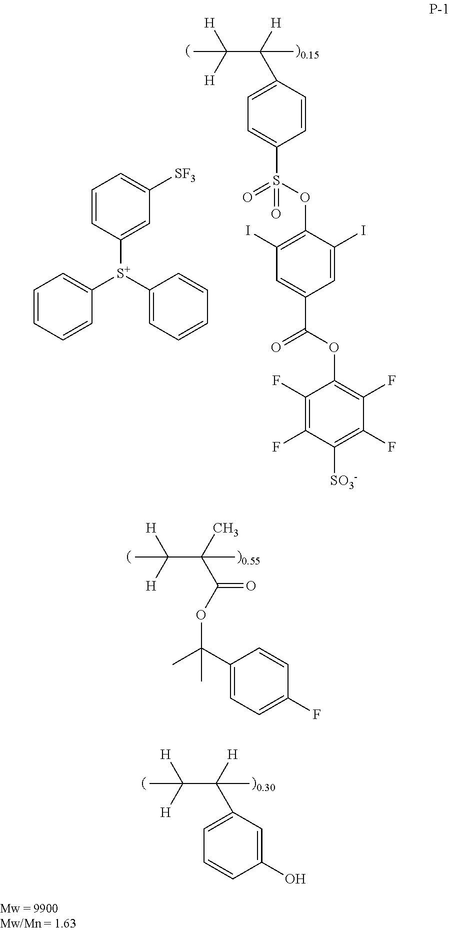

### [Examples 2-2 to 2-23, Comparative Examples 2-1 to 2-8] Synthesis of Polymers (P-2) to (P-23) and Comparative Polymers (CP-1) to (CP-8)

Polymers shown in Tables 1 and 2 were synthesized in the same manner as in Example 2-1 except that the types and blend ratios of monomers were changed. Note that polymer (P-24) was synthesized by the above-described polymerization method with a RAFT agent, and the synthesis result is shown together in Tables 1 and 2. In addition, the remaining amounts of onium salt monomers as quantified by UPLC are shown together.

TABLE 1

| | | | | | | | | | | | Remaining |
|---|---|---|---|---|---|---|---|---|---|---|---|
| | | | | | | | | | | | amount |
| | | | | | | | | | | | of onium |
| | | Introduction | | Introduction | | Introduction | | Introduction | | | salt |
| | Unit | ratio | Unit | ratio | Unit | ratio | Unit | ratio | | Mw/ | monomer |
| Polymer | a | (mol %) | b | (mol %) | c | (mol %) | d | (mol %) | Mw | Mn | (wt %) |
| p-1 | a-1 | 15 | b-1 | [symbol] | c-1 | 30 | | | 9100 | 1.71 | 0.1 |
| p-2 | a-2 | 15 | b-1 | [symbol] | c-1 | 30 | | | 9000 | [symbol] | 0.2 |
| p-3 | a-3 | 15 | b-1 | [symbol] | c-1 | 30 | | | [symbol] | 1.69 | less than |
| | | | | | | | | | | | 0.1 |
| p-4 | a-4 | 15 | b-1 | [symbol] | c-2 | 30 | | | 9400 | [symbol] | less than |
| | | | | | | | | | | | 0.1 |
| p-5 | a-5 | 15 | b-1 | [symbol] | c-2 | 30 | | | 9200 | 1.72 | less than |
| | | | | | | | | | | | 0.1 |
| p-6 | a-6 | 15 | b-1 | [symbol] | c-2 | 30 | | | 9200 | 1.69 | less than |
| | | | | | | | | | | | 0.1 |
| p-7 | a-1 | 15 | b-2 | [symbol] | c-3 | 30 | | | [symbol] | 1.72 | 0.1 |
| p-8 | a-2 | 15 | b-2 | [symbol] | c-3 | 30 | | | 9200 | [symbol] | 0.1 |
| p-9 | a-3 | 15 | b-2 | [symbol] | c-3 | 30 | | | 9200 | [symbol] | less than |
| | | | | | | | | | | | 0.1 |
| p-10 | a-4 | 15 | b-3 | [symbol] | c-4 | 30 | | | [symbol] | 1.69 | less than |
| | | | | | | | | | | | 0.1 |
| p-11 | a-5 | 15 | b-3 | [symbol] | c-4 | 30 | | | [symbol] | 1.72 | 0.1 |
| p-12 | a-6 | 15 | b-3 | [symbol] | c-4 | 30 | | | 9000 | 1.72 | less than |
| | | | | | | | | | | | 0.1 |
| p-13 | a-1 | 10 | b-4 | [symbol] | c-1 | 30 | d-1 | 10 | 9000 | 1.71 | 0.1 |
| p-14 | a-2 | 10 | b-4 | [symbol] | c-1 | 30 | d-2 | 10 | 9100 | 1.73 | 0.1 |
| p-15 | a-3 | 20 | b-4 | [symbol] | c-1 | 30 | d-3 | 10 | 9100 | 1.74 | less than |
| | | | | | | | | | | | 0.1 |
| p-16 | a-4 | 20 | b-5 | [symbol] | c-1 | [symbol] | d-1 | 10 | 9400 | 1.7 | lees than |
| | | | | | | | | | | | 0.1 |
| p-17 | a-5 | 15 | b-5 | [symbol] | c-1 | [symbol] | d-2 | [symbol] | 9100 | 1.72 | 0.2 |
| p-18 | a-6 | 15 | b-5 | [symbol] | c-1 | [symbol] | d-3 | [symbol] | [symbol] | 1.74 | less than |
| | | | | | | | | | | | 0.1 |
| p-19 | a-1 | 10 | b-1 | [symbol] | c-1 | 30 | | | 9200 | 1.68 | 0.1 |
| | a-2 | 5 | | | | | | | | | |
| p-20 | a-3 | 15 | b-1 | [symbol] | c-1 | 30 | | | 8900 | 1.72 | less than |
| | | | b-2 | [symbol] | | | | | | | 0.1 |
| p-21 | a-4 | 20 | b-3 | [symbol] | c-2 | 10 | | | 9000 | 1.73 | less than |
| | | | | | c-3 | 10 | | | | | 0.1 |
| p-22 | a-5 | 10 | b-4 | [symbol] | c-4 | [symbol] | d-1 | [symbol] | [symbol] | 1.7 | less than |
| | | | | | | | d-2 | [symbol] | | | 0.1 |
| p-23 | a-1 | 10 | b-5 | [symbol] | c-1 | 25 | d-3 | 5 | 9000 | 1.72 | less than |
| | a-6 | 5 | b-1 | [symbol] | | | | | | | 0.1 |
| p-24 | a-1 | 15 | b-1 | [symbol] | c-1 | 30 | | | 9200 | 1.37 | 0.1 |

[symbol]

indicates data missing or illegible when filed

TABLE 2

| | | | | | | | | | | | Remaining |
|---|---|---|---|---|---|---|---|---|---|---|---|
| | | | | | | | | | | | amount |
| | | | | | | | | | | | of onium |
| | | Introduction | | Introduction | | Introduction | | Introduction | | | salt |
| | Unit | ratio | Unit | ratio | Unit | ratio | Unit | ratio | | Mw/ | monomer |
| Polymer | a | (mol %) | b | (mol %) | c | (mol %) | d | (mol %) | Mw | Mn | (wt %) |
| CP-1 | Ca-1 | 15 | b-1 | 55 | c-1 | 30 | | | 9200 | 1.71 | 0.3 |
| CP-2 | Ca-2 | 15 | b-1 | 55 | c-2 | 30 | | | 9000 | 1.72 | 0.3 |
| CP-3 | Ca-3 | 15 | b-1 | 55 | c-2 | 30 | | | 9100 | 1.7 | 0.3 |
| CP-4 | Ca-4 | 15 | b-1 | 55 | c-2 | 30 | | | 8900 | 1.73 | 0.3 |
| CP-5 | Ca-1 | 10 | b-4 | 50 | c-1 | 30 | d-1 | 10 | 9300 | 1.7 | 0.2 |
| CP-6 | Ca-2 | 15 | b-5 | 55 | c-1 | 25 | d-2 | 5 | 9100 | 1.69 | 0.3 |
| CP-7 | Ca-3 | 15 | b-5 | 55 | c-1 | 25 | d-3 | 5 | 9400 | 1.73 | 0.3 |
| CP-8 | Ca-4 | 20 | b-5 | 45 | c-1 | 25 | d-1 | 10 | 9200 | 1.72 | 0.4 |

### [Example 2-24] Synthesis of Polymer (P-24)

Under a nitrogen atmosphere, 22.2 g of monomer (a-1), 21.5 g of monomer (b-1), 6.3 g of monomer (c-1), 2.02 g of V-601, and 70 g of MEK (methyl ethyl ketone) were added to a flask to prepare a monomer-polymerization initiator solution. To another reaction vessel under a nitrogen atmosphere, 23 g of MEK and 3.0 g of a RAFT agent (2-cyano-2-propanyl dodecyl carbonotrithioate) were added and heated to 80° C. with stirring, and then the monomer-polymerization initiator solution was added thereto dropwise over 4 hours. After the dropwise addition, the polymerization solution was stirred for 4 hours with the temperature thereof kept at 80° C., and then further stirred for 3 hours with addition of 7.10 g of 1-dodecanethiol as an agent for terminal processing, and cooled to room temperature. The resulting polymerization solution was added dropwise to 1500 g of hexane vigorously stirred, and a polymer precipitated was separated by filtration. The resulting polymer was washed twice with 300 g of hexane, and vacuum-dried at 50° C. for 20 hours to afford polymer (P-24) in the form of white powder (yield: 50.5 g). The Mw and Mw/Mn of polymer (P-24) were 9200 and 1.37, respectively. Note that the Mw is a measurement in terms of polystyrene as determined by GPC with use of DMF as a solvent.

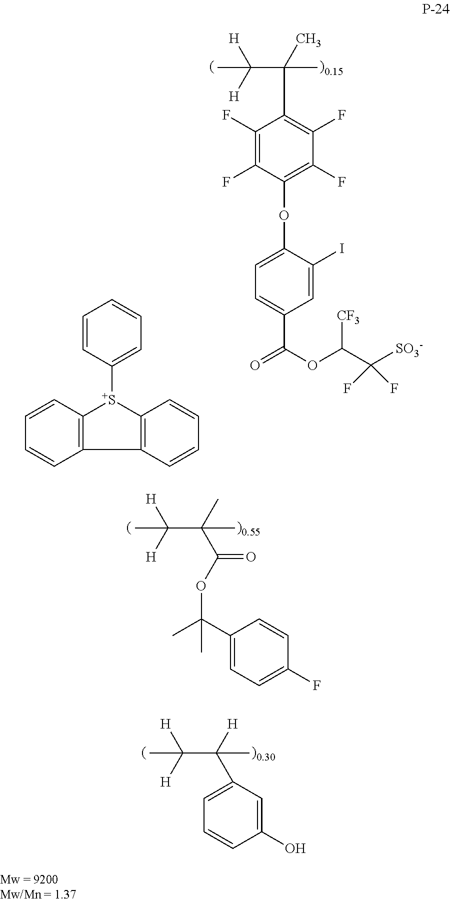

### [3] Test on Solubility of Monomers in Solvents

### Examples 3-1 to 3-6, Comparative Examples 3-1 to 3-4

Test on solubility in solvents was conducted for onium salt monomers (a-1) to (a-6) and monomers (Ca-1) to (Ca-4) as comparative examples. Used for the test on solubility in solvents were PGME (propylene glycol monomethyl ether), DAA (diacetone alcohol), propylene glycol monomethyl ether acetate (PGMEA), ethyl lactate (EL), cyclohexanone (CyHO), and GBL (γ-butyrolactone). The test was conducted in a manner that monomers (a-1) to (a-6) and (Ca-1) to (Ca-4) were each mixed with different solvents to give 10 wt % or 20 wt %, and the states of dissolution were visually checked at room temperature. For test results, a case with complete dissolution at up to 20 wt % was rated as “Good”, a case with complete dissolution at up to 10 wt % as “Fair”, and a case with undissolved matter remained even at 10 wt % as “Poor”. Table 3 summarizes the results.

TABLE 3

| | Monomer | PGME | DAA | PGMEA | EL | CyHO | GBL |
|---|---|---|---|---|---|---|---|
| Example 3-1 | a-1 | Good | Good | Fair | Good | Good | Good |
| Example 3-2 | a-2 | Good | Good | Fair | Fair | Good | Good |
| Example 3-3 | a-3 | Good | Good | Good | Good | Good | Good |
| Example 3-4 | a-4 | Good | Good | Good | Good | Good | Good |
| Example 3-5 | a-5 | Good | Good | Fair | Good | Good | Good |
| Example 3-6 | a-6 | Good | Good | Good | Good | Good | Good |
| Comparative | Ca-1 | Fair | Good | Poor | Fair | Fair | Good |
| Example 3-1 | | | | | | | |
| Comparative | Ca-2 | Fair | Good | Poor | Fair | Fair | Good |
| Example 3-2 | | | | | | | |
| Comparative | Ca-3 | Fair | Good | Poor | Fair | Fair | Good |
| Example 3-3 | | | | | | | |
| Comparative | Ca-4 | Fair | Good | Poor | Fair | Fair | Good |
| Example 3-4 | | | | | | | |

As shown in Table 3, it is understood that the monomer of the present invention (Examples 3-1 to 3-6) exhibited solubilities of 10 wt % or more in all of the solvents, and thus improved solubilities in solvents compared with the comparative examples (Comparative Examples 3-1 to 3-4), each having no fluorine substituent in the aromatic ring of the styrene structure. Moreover, it is understood from comparison between Example 3-2 and Examples 3-1 and 3-3 to 3-6, which differ in number of fluorine substituents in the styrene structure, that the configuration in which all of the substituents in the aromatic ring were fluorine gave improved solubility in solvents. Furthermore, comparison between Examples 3-1 and 3-5 and Examples 3-3, 3-4, and 3-6 shows that further improved solubility in solvents is successfully given by disposing a substituent at a specific substitution position in the styrene structure, as described above.

### [4] Preparation of Chemically Amplified Resist Compositions

### Examples 4-1 to 4-24, Comparative Examples 4-1 to 4-8

Specific components selected from the base polymers of the present invention (P-1 to P-24), comparative base polymers (CP-1 to CP-8), acid generators (PAG-1, PAG-2), and quenchers (SQ-1 to SQ-4) at a composition listed in Tables 4 and 5 shown below were dissolved in a solvent containing 0.01% by mass of FC-4430 manufactured by 3M Company as a surfactant to prepare a solution, and the solution was filtered through a 0.2-μm Teflon (registered trademark) filter to prepare chemically amplified resist compositions (R-1 to R-24, CR-1 to CR-8).

TABLE 4

| | | Base | | Acid | | | |
|---|---|---|---|---|---|---|---|
| | | polymer | Quencher | generator | Solvent 1 | Solvent 2 | Solvent 3 |
| | Resist | (part by | (part by | (part by | (part by | (part by | (part by |
| | composition | mass) | mass) | mass) | mass) | mass) | mass) |
| Example 4-1 | R-1 | P-1(80) | SQ-1(7.6) | — | PGMEA(2000) | DAA(500) | EL(2500) |
| Example 4-2 | R-2 | P-2(80) | SQ-1(7.6) | — | PGMEA(2000) | DAA(500) | EL(2500) |
| Example 4-3 | R-3 | P-3(80) | SQ-2(8.0) | — | PGMEA(2000) | DAA(500) | EL(2500) |
| Example 4-4 | R-4 | P-4(80) | SQ-3(8.4) | — | PGMEA(2000) | DAA(500) | EL(2500) |
| Example 4-5 | R-5 | P-5(80) | SQ-4(8.2) | — | PGMEA(2000) | DAA(500) | EL(2500) |
| Example 4-6 | R-6 | P-6(80) | SQ-1(7.6) | — | PGMEA(2000) | DAA(500) | EL(2500) |
| Example 4-7 | R-7 | P-7(80) | SQ-1(7.5) | — | PGMEA(2000) | DAA(500) | EL(2500) |
| Example 4-8 | R-8 | P-8(80) | SQ-1(7.5) | — | PGMEA(2000) | DAA(500) | EL(2500) |
| Example 4-9 | R-9 | P-9(80) | SQ-3(8.8) | — | PGMEA(2000) | DAA(500) | EL(2500) |
| Example 4-10 | R-10 | P-10(80) | SQ-3(8.8) | — | PGMEA(2000) | DAA(500) | EL(2500) |
| Example 4-11 | R-11 | P-11(80) | SQ-3(7.8) | — | PGMEA(2000) | DAA(500) | EL(2500) |
| Example 4-12 | R-12 | P-12(80) | SQ-3(7.6) | — | PGMEA(2000) | DAA(500) | EL(2500) |
| Example 4-13 | R-13 | P-13(80) | SQ-2(8.0) | PAG-1(10) | PGMEA(2000) | DAA(500) | EL(2500) |
| Example 4-14 | R-14 | P-14(80) | SQ-2(8.0) | PAG-2(15) | PGMEA(2000) | DAA(500) | EL(2500) |
| Example 4-15 | R-15 | P-15(80) | SQ-2(8.2) | — | PGMEA(2000) | DAA(500) | EL(2500) |
| Example 4-16 | R-16 | P-16(80) | SQ-2(8.2) | — | PGMEA(2000) | DAA(500) | EL(2500) |
| Example 4-17 | R-17 | P-17(80) | SQ-4(7.8) | — | PGMEA(2000) | DAA(500) | EL(2500) |
| Example 4-18 | R-18 | P-18(80) | SQ-4(7.8) | — | PGMEA(2000) | DAA(500) | EL(2500) |
| Example 4-19 | R-19 | P-19(80) | SQ-4(7.8) | — | PGMEA(2000) | DAA(500) | EL(2500) |
| Example 4-20 | R-20 | P-20(80) | SQ-4(9.0) | — | PGMEA(2000) | DAA(500) | EL(2500) |
| Example 4-21 | R-21 | P-21(80) | SQ-1(7.8) | | PGMEA(2000) | DAA(500) | EL(2500) |
| Example 4-22 | R-22 | P-22(80) | SQ-1(7.6) | PAG-1(10) | PGMEA(2000) | DAA(500) | EL(2500) |
| Example 4-23 | R-23 | P-23(80) | SQ-1(7.6) | | PGMEA(2000) | DAA(500) | EL(2500) |
| Example 4-24 | R-24 | P-24(80) | SQ-3(9.0) | | PGMEA(2000) | DAA(500) | EL(2500) |

TABLE 5

| | | Base | | Acid | | | |
|---|---|---|---|---|---|---|---|
| | | polymer | Quencher | generator | Solvent 1 | Solvent 2 | Solvent 3 |
| | Resist | (part by | (part by | (part by | (part by | (part by | (part by |
| | composition | mass) | mass) | mass) | mass) | mass) | mass)) |
| Comparative | CR-1 | CP- | SQ- | — | PGMEA(2000) | DAA(500) | EL(2500) |
| Example 4-1 | | 1(80) | 1(7.6) | | | | |
| Comparative | CR-2 | CP- | SQ- | — | PGMEA(2000) | DAA(500) | EL(2500) |
| Example 4-2 | | 2(80) | 1(7.8) | | | | |
| Comparative | CR-3 | CP- | SQ- | — | PGMEA(2000) | DAA(500) | EL(2500) |
| Example 4-3 | | 3(80) | 1(7.6) | | | | |
| Comparative | CR-4 | CP- | SQ- | — | PGMEA(2000) | DAA(500) | EL(2500) |
| Example 4-4 | | 4(80) | 1(7.6) | | | | |
| Comparative | CR-5 | CP- | SQ- | PAG-1(10) | PGMEA(2000) | DAA(500) | EL(2500) |
| Example 4-5 | | 5(80) | 2(7.6) | | | | |
| Comparative | CR-6 | CP- | SQ- | — | PGMEA(2000) | DAA(500) | EL(2500) |
| Example 4-6 | | 6(80) | 3(8.8) | | | | |
| Comparative | CR-7 | CP- | SQ- | — | PGMEA(2000) | DAA(500) | EL(2500) |
| Example 4-7 | | 7(80) | 4(8.5) | | | | |
| Comparative | CR-8 | CP- | SQ- | — | PGMEA(2000) | DAA(500) | EL(2500) |
| Example 4-8 | | 8(80) | 4(8.5) | | | | |

The solvents, quenchers (SQ-1 to SQ-4), and acid generators (PAG-1, PAG-2) in Tables 4 and 5 are as follows.
Solvents:
   PGMEA (propylene glycol monomethyl ether acetate)
   DAA (diacetone alcohol)
   EL (ethyl lactate)
Quenchers: SQ-1 to SQ-4

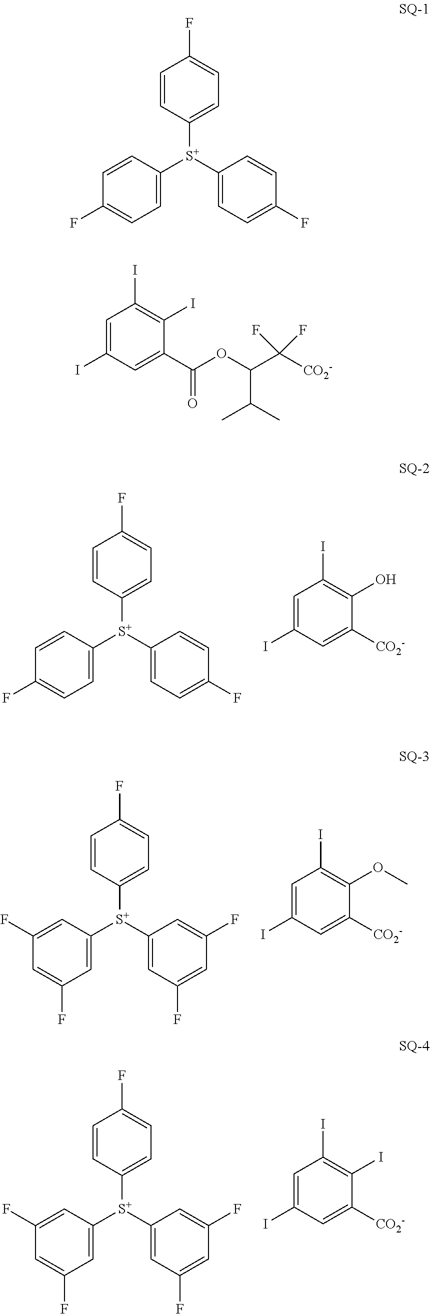

Acid generators: PAG-1, PAG-2

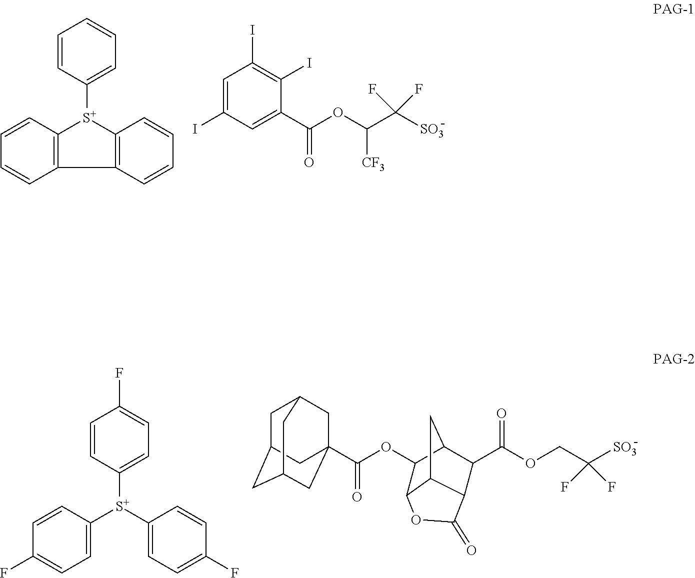

### [5] Evaluation for EUV Lithography (1)

### Examples 5-1 to 5-24, Comparative Examples 5-1 to 5-8

Onto a Si substrate on which the silicon-containing spin-on hard mask SHB-A940 (silicon content: 43% by mass) manufactured by Shin-Etsu Chemical Co., Ltd. with a film thickness of 20 nm had been formed, a chemically amplified resist composition (R-1 to R-24, CR-1 to CR-8) shown in Tables 4 and 5 was applied by spin coating, and prebaked by using a hot plate at 100° C. for 60 seconds to produce a resist film having a thickness of 50 nm. The resist film was exposed by using the EUV scanner NXE3400 (NA: 0.33, σ: 0.9/0.6, dipole illumination) manufactured by ASML with an LS pattern of 18 nm in on-wafer dimension and 36 nm in pitch while the dose and focus were varied (dose pitch: 1 mJ/cm², focus pitch: 0.020 μm), and after the exposure subjected to PEB at a temperature shown in Tables 6 and 7 for 60 seconds. Thereafter, puddle developing was performed with 2.38% by mass TMAH aqueous solution for 30 seconds, and the resultant was rinsed with a surfactant-containing rinsing material and spin-dried, giving a positive-type pattern.

The resulting LS pattern was observed with a critical dimension SEM (CG6300) manufactured by Hitachi High-Tech Corporation, and the sensitivity, EL, LWR, and collapse limit were evaluated with methods shown below. The results are shown in Tables 6 and 7.

### [Evaluation of Sensitivity]

An optimum dose, Eop (mJ/cm²), that gave an LS pattern of 18 nm in line width and 36 nm in pitch was determined as sensitivity. The smaller this value, the higher the sensitivity.

### [Evaluation of EL]

From a dose that allowed formation of the LS pattern within ±10% of the space width of 18 nm (16.2 to 19.8 nm), the EL (unit: %) was determined by using an expression shown below. The larger this value, the better the performance.

$$EL ( \% ) = ( \vert {E}_{1} - {E}_{2} \vert / Eop ) \times 100$$

E₁: optimum dose to give LS pattern of 16.2 nm in line width and 36 nm in pitch
E₂: optimum dose to give LS pattern of 19.8 nm in line width and 36 nm in pitch
Eop: optimum dose to give LS pattern of 18 nm in line width and 36 nm in pitch

### [Evaluation of LWR]

For the LS pattern obtained by irradiating at Eop, dimensions were measured at 10 points in the longitudinal direction of the line, the standard deviation (o) was calculated from the results and multiplied by 3, and the resulting value (3σ) was determined as the LWR. As this value is smaller, a pattern with smaller roughness and more homogenous line width is given.

### [Evaluation of Line Pattern Collapse Limit]

For the LS pattern, line dimensions were measured at 10 points in the longitudinal direction at different doses at the optimum focus. The smallest line dimension given without break-down was determined as the collapse limit dimension. The smaller this value, the more superior the collapse limit.

TABLE 6

| | | PEB | | | | |
|---|---|---|---|---|---|---|
| | Resist | temper- | Optimum | | | Collapse |
| | compo- | ature | dose | EL | LWR | limit |
| | sition | (° C.) | (mJ/cm²) | (%) | (nm) | (nm) |
| Example 5-1 | R-1 | 95 | 36 | 19 | 2.8 | 12.4 |
| Example 5-2 | R-2 | 95 | 38 | 17 | 3.0 | 12.5 |
| Example 5-3 | R-3 | 95 | 36 | 19 | 2.8 | 12.3 |
| Example 5-4 | R-4 | 95 | 35 | 19 | 2.7 | 12.2 |
| Example 5-5 | R-5 | 95 | 34 | 18 | 2.6 | 12.4 |
| Example 5-6 | R-6 | 95 | 36 | 19 | 2.8 | 12.2 |
| Example 5-7 | R-7 | 100 | 36 | 19 | 2.8 | 12.4 |
| Example 5-8 | R-8 | 100 | 37 | 17 | 3.0 | 12.6 |
| Example 5-9 | R-9 | 100 | 35 | 18 | 2.6 | 12.1 |
| Example 5-10 | R-10 | 95 | 35 | 19 | 2.7 | 12.2 |
| Example 5-11 | R-11 | 95 | 35 | 18 | 2.6 | 12.3 |
| Example 5-12 | R-12 | 95 | 35 | 19 | 2.6 | 12.2 |
| Example 5-13 | R-13 | 95 | 34 | 19 | 2.7 | 12.3 |
| Example 5-14 | R-14 | 95 | 37 | 16 | 3.1 | 12.7 |
| Example 5-15 | R-15 | 95 | 36 | 19 | 2.8 | 12.1 |
| Example 5-16 | R-16 | 100 | 36 | 17 | 2.9 | 12.1 |
| Example 5-17 | R-17 | 100 | 35 | 18 | 2.7 | 12.2 |
| Example 5-18 | R-18 | 100 | 35 | 19 | 2.6 | 12.2 |
| Example 5-19 | R-19 | 95 | 35 | 17 | 2.9 | 12.6 |
| Example 5-20 | R-20 | 100 | 35 | 18 | 2.7 | 12.3 |
| Example 5-21 | R-21 | 95 | 36 | 19 | 2.8 | 12.8 |
| Example 5-22 | R-22 | 95 | 35 | 16 | 3.0 | 12.9 |
| Example 5-23 | R-23 | 95 | 36 | 18 | 2.8 | 12.7 |
| Example 5-24 | R-24 | 95 | 36 | 19 | 2.6 | 12.0 |

TABLE 7

| | | PEB | | | | |
|---|---|---|---|---|---|---|
| | Resist | temper- | Optimum | | | Collapse |
| | compo- | ature | dose | EL | LWR | limit |
| | sition | (° C.) | (mJ/cm²) | (%) | (nm) | (nm) |
| Comparative | CR-1 | 95 | 40 | 13 | 3.3 | 13.2 |
| Example 5-1 | | | | | | |
| Comparative | CR-2 | 95 | 39 | 14 | 3.4 | 13.4 |
| Example 5-2 | | | | | | |
| Comparative | CR-3 | 95 | 40 | 13 | 3.3 | 13.3 |
| Example 5-3 | | | | | | |
| Comparative | CR-4 | 95 | 41 | 15 | 3.5 | 13.6 |
| Example 5-4 | | | | | | |
| Comparative | CR-5 | 95 | 40 | 13 | 3.4 | 13.5 |
| Example 5-5 | | | | | | |
| Comparative | CR-6 | 100 | 39 | 15 | 3.3 | 13.2 |
| Example 5-6 | | | | | | |
| Comparative | CR-7 | 100 | 41 | 14 | 3.5 | 13.5 |
| Example 5-7 | | | | | | |
| Comparative | CR-8 | 100 | 42 | 13 | 3.3 | 13.2 |
| Example 5-8 | | | | | | |

It was found from the results shown in Tables 6 and 7 that the chemically amplified resist compositions each obtained with a base polymer containing a repeating unit derived from the onium salt monomer of the present invention were superior in EL and LWR, with good sensitivity. In addition, they exhibited small collapse limit values, and thus were confirmed to be resistant to pattern collapse even in fine patterning. Accordingly, the chemically amplified resist composition of the present invention was demonstrated to be suitable as a material for EUV lithography. Further, comparison between Examples 5-1 and 5-24 showed a result that the chemically amplified resist composition in Example 5-24, which was given by using a narrow-polydispersity polymer synthesized by RAFT polymerization, exhibited superior LWR to that in Example 5-1, which was given by using a polymer synthesized by normal radical polymerization, even though the compositions of the polymers were the same.

### [6] Evaluation for EUV Lithography (2)

### Examples 6-1 to 6-24, Comparative Examples 6-1 to 6-8

Onto a Si substrate on which the silicon-containing spin-on hard mask SHB-A940 (silicon content: 43% by mass) manufactured by Shin-Etsu Chemical Co., Ltd. with a film thickness of 20 nm had been formed, a chemically amplified resist composition (R-1 to R-24, CR-1 to CR-8) shown in Tables 4 and 5 was applied by spin coating, and prebaked by using a hot plate at 105° C. for 60 seconds to produce a resist film having a thickness of 50 nm. This was exposed by using the EUV scanner NXE3400 (NA: 0.33, σ: 0.9/0.6, quadrupole illumination, mask with hole pattern having on-wafer dimension of 46 nm in pitch, +20% bias) manufactured by ASML, PEB was performed by using a hot plate at a temperature shown in Tables 8 and 9 for 60 seconds, and development was performed with 2.38% by mass TMAH aqueous solution for 30 seconds to form a hole pattern having a dimension of 23 nm.

A dose that allowed formation with a hole dimension of 23 nm was measured as the sensitivity by using a critical dimension SEM (CG6300) manufactured by Hitachi High-Tech Corporation, and the dimensions of 50 holes at that time were measured, the standard deviation (o) was calculated from the results and multiplied by 3, and the resulting value (3σ) was determined as the critical dimension uniformity (CDU). The results are shown in Tables 8 and 9.

TABLE 8

| | | PEB | | |
|---|---|---|---|---|
| | Resist | temperature | Optimum dose | CDU |
| | composition | (° C.) | (mJ/cm²) | (nm) |
| Example 6-1 | R-1 | 95 | 26 | 2.6 |
| Example 6-2 | R-2 | 95 | 26 | 2.7 |
| Example 6-3 | R-3 | 95 | 26 | 2.6 |
| Example 6-4 | R-4 | 95 | 25 | 2.4 |
| Example 6-5 | R-5 | 95 | 24 | 2.4 |
| Example 6-6 | R-6 | 95 | 26 | 2.5 |
| Example 6-7 | R-7 | 100 | 26 | 2.6 |
| Example 6-8 | R-8 | 100 | 27 | 2.7 |
| Example 6-9 | R-9 | 100 | 25 | 2.4 |
| Example 6-10 | R-10 | 95 | 25 | 2.4 |
| Example 6-11 | R-11 | 95 | 25 | 2.4 |
| Example 6-12 | R-12 | 95 | 25 | 2.3 |
| Example 6-13 | R-13 | 95 | 24 | 2.5 |
| Example 6-14 | R-14 | 95 | 27 | 2.8 |
| Example 6-15 | R-15 | 95 | 26 | 2.6 |
| Example 6-16 | R-16 | 100 | 25 | 2.6 |
| Example 6-17 | R-17 | 100 | 25 | 2.5 |
| Example 6-18 | R-18 | 100 | 25 | 2.3 |
| Example 6-19 | R-19 | 95 | 25 | 2.7 |
| Example 6-20 | R-20 | 100 | 25 | 2.4 |
| Example 6-21 | R-21 | 95 | 26 | 2.6 |
| Example 6-22 | R-22 | 95 | 25 | 2.7 |
| Example 6-23 | R-23 | 95 | 26 | 2.6 |
| Example 6-24 | R-24 | 95 | 26 | 2.3 |

TABLE 9

| | | PEB | | |
|---|---|---|---|---|
| | Resist | temperature | Optimum dose | CDU |
| | composition | (° C.) | (mJ/cm²) | (nm) |
| Comparative | CR-1 | 95 | 31 | 3.0 |
| Example 6-1 | | | | |
| Comparative | CR-2 | 95 | 29 | 3.1 |
| Example 6-2 | | | | |
| Comparative | CR-3 | 95 | 30 | 3.0 |
| Example 6-3 | | | | |
| Comparative | CR-4 | 95 | 32 | 3.2 |
| Example 6-4 | | | | |
| Comparative | CR-5 | 95 | 30 | 3.1 |
| Example 6-5 | | | | |
| Comparative | CR-6 | 100 | 29 | 3.0 |
| Example 6-6 | | | | |
| Comparative | CR-7 | 100 | 31 | 3.3 |
| Example 6-7 | | | | |
| Comparative | CR-8 | 100 | 32 | 3.2 |
| Example 6-8 | | | | |

It was found from the results shown in Tables 8 and 9 that the chemically amplified resist composition of the present invention had good sensitivity and was superior in CDU. Further, comparison between Examples 6-1 and 6-24 showed a result that the chemically amplified resist composition in Example 6-24, which was given by using a narrow-polydispersity polymer synthesized by RAFT polymerization, exhibited superior CDU to that in Example 6-1, which was given by using a polymer synthesized by normal radical polymerization, even though the compositions of the polymers were the same.

### [7] Evaluation of Defects

### Examples 7-1 to 7-24, Comparative Examples 7-1 to 7-8

The LS patterns of 36 nm in pitch that were given in the evaluation for lithography were observed by using the bright-field defect inspector KLA2905 (name of apparatus) manufactured by KLA-Tencor Corporation. Comparison was made on numbers of pattern defects per unit area (defects/cm²). A value of 5.0 or more was rated as “Poor”, a value of 1.0 or more and less than 5.0 as “Fair”, and a value of less than 1.0 as “Good”. Smaller values indicate better performance. The results are shown in Tables 10 and 11.

TABLE 10

| | Resist | |
|---|---|---|
| | composition | Pattern defect |
| Example 7-1 | R-1 | Good |
| Example 7-2 | R-2 | Fair |
| Example 7-3 | R-3 | Good |
| Example 7-4 | R-4 | Good |
| Example 7-5 | R-5 | Good |
| Example 7-6 | R-6 | Good |
| Example 7-7 | R-7 | Good |
| Example 7-8 | R-8 | Fair |
| Example 7-9 | R-9 | Good |
| Example 7-10 | R-10 | Good |
| Example 7-11 | R-11 | Good |
| Example 7-12 | R-12 | Good |
| Example 7-13 | R-13 | Good |
| Example 7-14 | R-14 | Fair |
| Example 7-15 | R-15 | Good |
| Example 7-16 | R-16 | Good |
| Example 7-17 | R-17 | Good |
| Example 7-18 | R-18 | Good |
| Example 7-19 | R-19 | Fair |
| Example 7-20 | R-20 | Good |
| Example 7-21 | R-21 | Good |
| Example 7-22 | R-22 | Good |
| Example 7-23 | R-23 | Good |
| Example 7-24 | R-24 | Good |

TABLE 11

| | Resist | Pattern |
|---|---|---|
| | composition | defect |
| Comparative | CP-1 | Poor |
| Example 7-1 | | |
| Comparative | CP-2 | Poor |
| Example 7-2 | | |
| Comparative | CP-3 | Poor |
| Example 7-3 | | |
| Comparative | CP-4 | Poor |
| Example 7-4 | | |
| Comparative | CP-5 | Fair |
| Example 7-5 | | |
| Comparative | CP-6 | Poor |
| Example 7-6 | | |
| Comparative | CP-7 | Poor |
| Example 7-7 | | |
| Comparative | CP-8 | Fair |
| Example 7-8 | | |

It was found from the results shown in Tables 10 and 11 that the chemically amplified resist compositions in Examples 7-1 to 7-24, each being the chemically amplified resist composition of the present invention, exhibited smaller numbers of pattern defects than those in Comparative Examples 7-1 to 7-8 exhibited.

The present description includes the following inventions.
[1]: An onium salt monomer represented by the following formula (a):

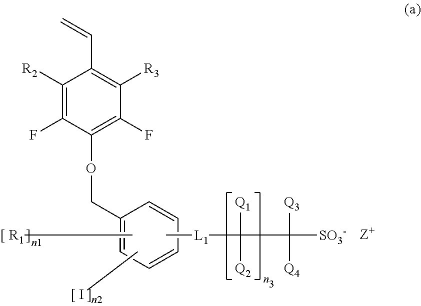

wherein R₁ is a halogen atom being not iodine, a cyano group, a nitro group, or a hydrocarbyl group having 1 to 20 carbon atoms and optionally containing a heteroatom; R₂ and R₃ are each a hydrogen atom or a fluorine atom; n1 is an integer of 0 to 3, n2 is an integer of 1 to 4, and n3 is an integer of 0 to 4; L₁ is a single bond, an ether bond, an ester bond, a sulfonic ester bond, a sulfonamide bond, a carbonate bond, or a carbamate bond; Q₁ and Q₂ are each independently a hydrogen atom, a saturated hydrocarbyl group having 1 to 6 carbon atoms, a fluorine atom, or a fluorinated saturated hydrocarbyl group having 1 to 6 carbon atoms; Q₃ and Q₄ are each independently a hydrogen atom, a saturated hydrocarbyl group having 1 to 6 carbon atoms, a fluorine atom, or a fluorinated saturated hydrocarbyl group having 1 to 6 carbon atoms; and Z⁺ is an onium cation.
[2]: The onium salt monomer according to [1], wherein the onium salt monomer is represented by the following formula (a1):

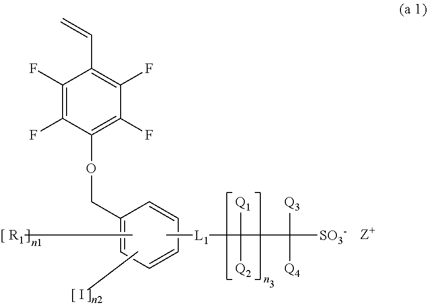

wherein n1 to n3, R₁, L₁, Q₁ to Q₄, and Z⁺ are as specified above.
[3]: The onium salt monomer according to [1] or [2], wherein the onium salt monomer is represented by the following formula (a2):

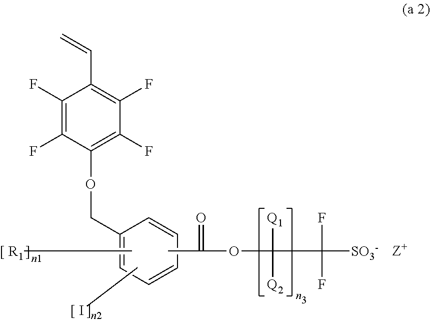

wherein n1 to n3, R₁, Q₁, Q₂, and Z⁺ are as specified above.
[4]: The onium salt monomer according to any one of [1] to [3], wherein Z⁺ is a sulfonium cation represented by the following formula (cation-1) or an iodonium cation represented by the following formula (cation-2):

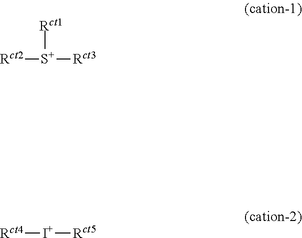

wherein R^{ct1} to R^{ct5} are each independently a halogen atom or a hydrocarbyl group having 1 to 30 carbon atoms and optionally containing a heteroatom; and R^{ct1} and R^{ct2} are optionally bound together to form a ring together with the sulfur atom to which the two groups are bound.
[5]: A polymer comprising a repeating unit derived from the onium salt monomer according to any one of [1] to [4].
[6]: The polymer according to [5], wherein the polymer further comprises a repeating unit represented by the following formula (b1) or (b2):

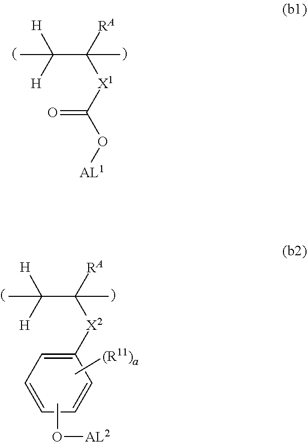

wherein R^{A} groups are each independently a hydrogen atom, a fluorine atom, a methyl group, or a trifluoromethyl group;
   X¹ is a single bond, a phenylene group, a naphthylene group, *—C(═O)—O—X¹¹—, or *—C(═O)—NH—X^{1l}—, wherein the phenylene group or naphthylene group is optionally substituted with an alkoxy group having 1 to 10 carbon atoms and optionally containing a fluorine atom or with a halogen atom, X¹¹ is a saturated hydrocarbylene group having 1 to 10 carbon atoms, a phenylene group, or a naphthylene group, and the saturated hydrocarbylene group optionally contains a hydroxy group, an ether bond, an ester bond, or a lactone ring;
   X² is a single bond, *—C(═O)—O—, or *—C(═O)—NH—;
   * indicates bonding to a carbon atom of a main chain;
   AL¹ and AL² are each independently an acid-unstable group;
   R ¹¹ is a halogen atom, a cyano group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; and
   a is an integer of 0 to 4.
[7]: The polymer according to [5] or [6], wherein the polymer further comprises a repeating unit represented by the following formula (c1):

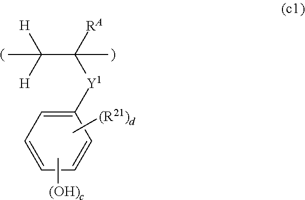

wherein R^{A} is a hydrogen atom, a fluorine atom, a methyl group, or a trifluoromethyl group;
   Y¹ is a single bond, *—C(═O)—O—, or *—C(═O)—NH—, wherein * indicates bonding to a carbon atom of a main chain;
   R²¹ is a halogen atom, a nitro group, a cyano group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; and
   c is an integer of 1 to 4, and d is an integer of 0 to 3, provided that 1≤c+d≤5 is satisfied.
[8]: The polymer according to any one of [5] to [7], wherein the polymer further comprises a repeating unit represented by the following formula (d1):

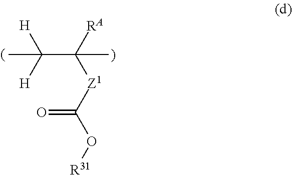

wherein R^{A} is a hydrogen atom, a fluorine atom, a methyl group, or a trifluoromethyl group;
   Z¹ is a single bond, a phenylene group, a naphthylene group, *—C(═O)—O—Z¹¹—, or *—C(═O)—NH—Z¹¹—, wherein the phenylene group or naphthylene group is optionally substituted with an alkoxy group having 1 to 10 carbon atoms and optionally containing a fluorine atom or with a halogen atom; * indicates bonding to a carbon atom of a main chain, Z¹¹ is a saturated hydrocarbylene group having 1 to 10 carbon atoms, a phenylene group, or a naphthylene group, and the saturated hydrocarbylene group optionally contains a hydroxy group, an ether bond, an ester bond, or a lactone ring; and
   R³¹ is a hydrogen atom or a group having 1 to 20 carbon atoms and containing at least one or more structures selected from a hydroxy group being not a phenolic hydroxy group, a cyano group, a carbonyl group, a carboxy group, an ether bond, an ester bond, a sulfonic ester bond, a carbonate bond, a lactone ring, a sultone ring, and a carboxylic anhydride (—C(═O)—O—C(═O)—).
[9]: A chemically amplified resist composition comprising (A) a base polymer containing the polymer according to any one of [5] to [8].
[10]: The chemically amplified resist composition according to [9], wherein the chemically amplified resist composition further comprises any one or more of (B) an organic solvent, (C) a quencher, (D) an acid generator, (E) a surfactant, and (F) a dissolution inhibitor.
[11]: A patterning method comprising: a step of forming a resist film on a substrate by using the chemically amplified resist composition according to [9] or [10]; a step of exposing the resist film to a high-energy ray; and a step of developing the exposed resist film by using a developer.
[12]: The patterning method according to [11], wherein ArF excimer laser light having a wavelength of 193 nm, KrF excimer laser light having a wavelength of 248 nm, an electron beam, or an extreme ultraviolet ray having a wavelength of 3 to 15 nm is used as the high-energy ray.

The present invention is not limited to the above embodiments. The embodiments are examples, and any embodiment that has substantially the same configuration as the technical idea described in Claims of the present invention and exerts the same action and effect is included in the technical scope of the present invention.

1. An onium salt monomer represented by the following formula (a): 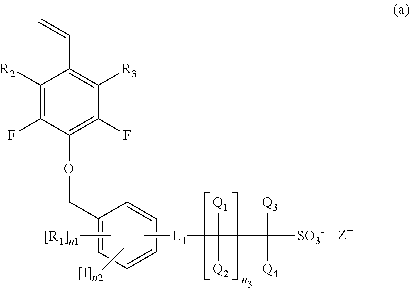
wherein R₁ is a halogen atom being not iodine, a cyano group, a nitro group, or a hydrocarbyl group having 1 to 20 carbon atoms and optionally containing a heteroatom;
R₂ and R₃ are each a hydrogen atom or a fluorine atom; n1 is an integer of 0 to 3, n2 is an integer of 1 to 4, and n3 is an integer of 0 to 4; L₁ is a single bond, an ether bond, an ester bond, a sulfonic ester bond, a sulfonamide bond, a carbonate bond, or a carbamate bond;
Q₁ and Q₂ are each independently a hydrogen atom, a saturated hydrocarbyl group having 1 to 6 carbon atoms, a fluorine atom, or a fluorinated saturated hydrocarbyl group having 1 to 6 carbon atoms; Q₃ and Q₄ are each independently a hydrogen atom, a saturated hydrocarbyl group having 1 to 6 carbon atoms, a fluorine atom, or a fluorinated saturated hydrocarbyl group having 1 to 6 carbon atoms; and Z⁺ is an onium cation.

2. The onium salt monomer according to claim 1, wherein the onium salt monomer is represented by the following formula (a1): 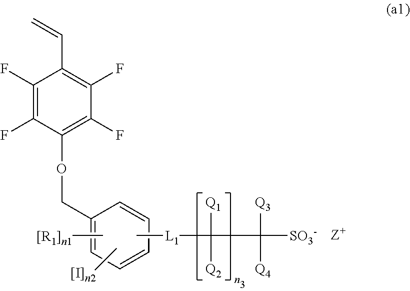
wherein n1 to n3, R₁, L₁, Q₁ to Q₄, and Z⁺ are as specified above.

3. The onium salt monomer according to claim 2, wherein the onium salt monomer is represented by the following formula (a2): 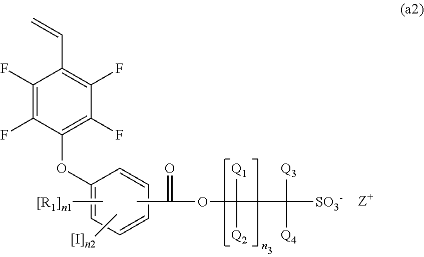
wherein n1 to n3, R₁, Q₁, Q₂, and Z⁺ are as specified above.

4. The onium salt monomer according to claim 1, wherein Z⁺ is a sulfonium cation represented by the following formula (cation-1) or an iodonium cation represented by the following formula (cation-2): 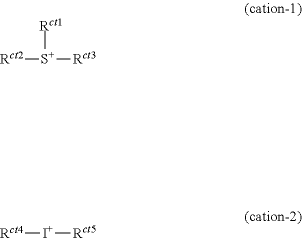
wherein R^{ct1} to R^{ct5} are each independently a halogen atom or a hydrocarbyl group having 1 to 30 carbon atoms and optionally containing a heteroatom; and R^{ct1} and R^{ct2} are optionally bound together to form a ring together with the sulfur atom to which the two groups are bound.

5. A polymer comprising a repeating unit derived from the onium salt monomer according to claim 1.

6. A polymer comprising a repeating unit derived from the onium salt monomer according to claim 2.

7. A polymer comprising a repeating unit derived from the onium salt monomer according to claim 3.

8. A polymer comprising a repeating unit derived from the onium salt monomer according to claim 4.

9. The polymer according to claim 5, wherein the polymer further comprises a repeating unit represented by the following formula (b1) or (b2): 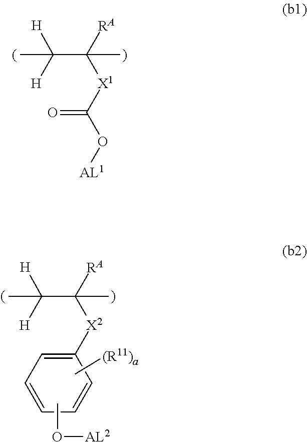
wherein R^{A} groups are each independently a hydrogen atom, a fluorine atom, a methyl group, or a trifluoromethyl group;
X¹ is a single bond, a phenylene group, a naphthylene group, *—C(═O)—O—X¹¹—, or *—C(═O)—NH—X¹¹—, wherein the phenylene group or naphthylene group is optionally substituted with an alkoxy group having 1 to 10 carbon atoms and optionally containing a fluorine atom or with a halogen atom, X¹¹ is a saturated hydrocarbylene group having 1 to 10 carbon atoms, a phenylene group, or a naphthylene group, and the saturated hydrocarbylene group optionally contains a hydroxy group, an ether bond, an ester bond, or a lactone ring;
X² is a single bond, *—C(═O)—O—, or *—C(═O)—NH—;
* indicates bonding to a carbon atom of a main chain;
AL¹ and AL² are each independently an acid-unstable group;
R¹¹ is a halogen atom, a cyano group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; and
a is an integer of 0 to 4.

10. The polymer according to claim 5, wherein the polymer further comprises a repeating unit represented by the following formula (c1): 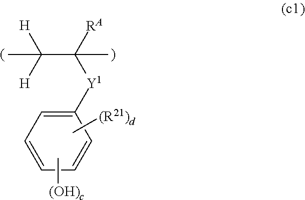
wherein R^{A} is a hydrogen atom, a fluorine atom, a methyl group, or a trifluoromethyl group;
Y¹ is a single bond, *—C(═O)—O—, or *—C(═O)—NH—, wherein * indicates bonding to a carbon atom of a main chain;
R²¹ is a halogen atom, a nitro group, a cyano group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; and
c is an integer of 1 to 4, and d is an integer of 0 to 3, provided that 1≤c+d≤5 is satisfied.

11. The polymer according to claim 5, wherein the polymer further comprises a repeating unit represented by the following formula (d1): 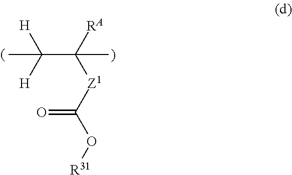
wherein R^{A} is a hydrogen atom, a fluorine atom, a methyl group, or a trifluoromethyl group;
Z¹ is a single bond, a phenylene group, a naphthylene group, *—C(═O)—O—Z¹¹—, or *—C(═O)—NH—Z¹¹—, wherein the phenylene group or naphthylene group is optionally substituted with an alkoxy group having 1 to 10 carbon atoms and optionally containing a fluorine atom or with a halogen atom; * indicates bonding to a carbon atom of a main chain, Z¹¹ is a saturated hydrocarbylene group having 1 to 10 carbon atoms, a phenylene group, or a naphthylene group, and the saturated hydrocarbylene group optionally contains a hydroxy group, an ether bond, an ester bond, or a lactone ring; and
R³¹ is a hydrogen atom or a group having 1 to 20 carbon atoms and containing at least one or more structures selected from a hydroxy group being not a phenolic hydroxy group, a cyano group, a carbonyl group, a carboxy group, an ether bond, an ester bond, a sulfonic ester bond, a carbonate bond, a lactone ring, a sultone ring, and a carboxylic anhydride (—C(═O)—O—C(═O)—).

12. A chemically amplified resist composition comprising (A) a base polymer containing the polymer according to claim 5.

13. A chemically amplified resist composition comprising (A) a base polymer containing the polymer according to claim 6.

14. A chemically amplified resist composition comprising (A) a base polymer containing the polymer according to claim 7.

15. A chemically amplified resist composition comprising (A) a base polymer containing the polymer according to claim 8.

16. The chemically amplified resist composition according to claim 12, wherein the chemically amplified resist composition further comprises any one or more of (B) an organic solvent, (C) a quencher, (D) an acid generator, (E) a surfactant, and (F) a dissolution inhibitor.

17. A patterning method comprising: a step of forming a resist film on a substrate by using the chemically amplified resist composition according to claim 12; a step of exposing the resist film to a high-energy ray; and a step of developing the exposed resist film by using a developer.

18. A patterning method comprising: a step of forming a resist film on a substrate by using the chemically amplified resist composition according to claim 13; a step of exposing the resist film to a high-energy ray; and a step of developing the exposed resist film by using a developer.

19. A patterning method comprising: a step of forming a resist film on a substrate by using the chemically amplified resist composition according to claim 14; a step of exposing the resist film to a high-energy ray;
and a step of developing the exposed resist film by using a developer.

20. The patterning method according to claim 17, wherein ArF excimer laser light having a wavelength of 193 nm, KrF excimer laser light having a wavelength of 248 nm, an electron beam, or an extreme ultraviolet ray having a wavelength of 3 to 15 nm is used as the high-energy ray.
